(19) United States
(12) Patent Application Publication
(10) Pub. No.: US 2024/0141351 A1
(43) Pub. Date: May 2, 2024

(54) ALPHA-1 ANTITRYPSIN (AAT) RNAI AGENTS, COMPOSITIONS INCLUDING AAT RNAI AGENTS, AND METHODS OF USE

(71) Applicant: Arrowhead Pharmaceuticals, Inc., Pasadena, CA (US)

(72) Inventors: Zhen Li, San Diego, CA (US); Rui Zhu, San Diego, CA (US); Christine I. Wooddell, Madison, WI (US); Tao Pei, Middleton, WI (US)

(73) Assignee: Arrowhead Pharmaceuticals, Inc., Pasadena, CA (US)

(21) Appl. No.: 18/530,795

(22) Filed: Dec. 6, 2023

Related U.S. Application Data
(63) Continuation of application No. 17/523,173, filed on Nov. 10, 2021, now Pat. No. 11,884,920.
Application No. 17/523,173 is a division of application No. 16/568,387, filed on Sep. 12, 2019, now Pat. No. 11,203,756.
Application No. 16/568,387 is a division of application No. 15/867,107, filed on Jan. 10, 2018, now Pat. No. 10,450,565.
(60) Provisional application No. 62/596,232, filed on Dec. 8, 2017.
(60) Provisional application No. 62/486,720, filed on Apr. 18, 2017.
(60) Provisional application No. 62/444,452, filed on Jan. 10, 2017.

(51) Int. Cl. C12N 15/113; A61K 9/00; A61K 31/713; A61P 1/16; C12Q 1/6883; G01N 33/68

(52) U.S. Cl. CPC C12N 15/113; A61K 9/0019; A61K 31/713; A61P 1/16; C12Q 1/6883; G01N 33/6893; C12N 2310/14; C12N 2310/315; C12N 2310/317; C12N 2310/32; C12N 2310/321; C12N 2310/322; C12N 2310/333; C12N 2310/334; C12N 2310/335; C12N 2310/336; C12N 2310/343; C12N 2310/346; C12N 2310/351; C12N 2320/31; C12Q 2600/156; C12Q 2600/158; G01N 2333/8125; G01N 2800/085

(57) ABSTRACT

RNAi agents for inhibiting the expression of the alpha-1 antitrypsin (AAT) gene, compositions including AAT RNAi agents, and methods of use are described. Also disclosed are pharmaceutical compositions including one or more AAT RNAi agents together with one or more excipients capable of delivering the RNAi agent(s) to a liver cell in vivo. Delivery of the AAT RNAi agent(s) to liver cells in vivo inhibits AAT gene expression and treats diseases associated with AAT deficiency such as chronic hepatitis, cirrhosis, hepatocellular carcinoma, transaminitis, cholestasis, fibrosis, and fulminant hepatic failure.

### CROSS REFERENCE TO RELATED APPLICATIONS

This application is a divisional of U.S. patent application Ser. No. 15/867,107, filed on Jan. 10, 2018, and claims priority to U.S. Provisional Patent Application Ser. No. 62/596,232, filed on Dec. 8, 2017, U.S. Provisional Patent Application Ser. No. 62/486,720, filed on Apr. 18, 2017, and U.S. Provisional Patent Application Ser. No. 62/444,452, filed on Jan. 10, 2017, the contents of each of which are incorporated herein by reference in their entirety.

### FIELD OF THE INVENTION

Disclosed herein are RNA interference (RNAi) agents for inhibition of alpha-1 antitrypsin gene expression, compositions that include alpha-1 antitrypsin RNAi agents, and methods of use thereof.

### BACKGROUND

Alpha-1 antitrypsin (AAT, al-antitrypsin, or A1AT) deficiency is an inherited, autosomal codominant genetic disorder that causes misfolding of the AAT protein and poor secretion of the misfolded protein leading to lung and liver diseases. AAT deficiency (AATD) occurs with a frequency of about 1 in every 1,500 to 3,500 individuals and most often affects persons with European ancestry.

Alpha-1 Antitrypsin is a protease inhibitor belonging to the serpin superfamily. Normal AAT protein is a circulating glycoprotein protease inhibitor primarily synthesized in the liver by hepatocytes and secreted into the blood. The known physiologic function of AAT is to inhibit neutrophil proteases, which serves to protect host tissues from non-specific injury during periods of inflammation.

The most clinically significant form of AATD, a genetic disorder associated with liver disease in children and adults, and pulmonary disease in adults, is caused by the Z mutation. The Z mutant allele (PiZ), through a single point mutation, renders the mutant Z form AAT protein (the “Z-AAT protein”) prone to abnormal folding causing intracellular retention. The mutant Z-AAT protein monomers are able to form chains of polymers that amass into aggregates, which are sometimes referred to as “globules.” The misfolded Z-AAT protein is ineffective in traversing the secretory pathway, and instead polymerizes and accumulates in the endoplasmic reticulum (ER) of hepatocytes. The polymeric globule masses stress the ER and trigger continuous hepatocyte injury, leading to fibrosis, cirrhosis, and increased risk of hepatocellular carcinoma. Further, the absence of circulating anti-protease activity leaves the lung vulnerable to injury by neutrophil elastase, resulting in the development of respiratory complications such as emphysema.

Individuals with the homozygous PiZZ genotype have severe deficiency of functional AAT, which leads to pulmonary disease. Weekly use of AAT augmentation therapy, using purified human AAT, results in near normal plasma levels of AAT in subjects with AATD, and helps prevent lung damage in affected individuals. However, while the administration of purified AAT can ameliorate or help prevent lung damage caused by the absence of endogenously secreted AAT, AATD patients remain vulnerable to endoplasmic reticulum liver storage disease caused by the deposition and accumulation of excessive abnormally folded AAT protein. Accumulated Z-AAT protein in the globule conformation in hepatocytes is a well-known characteristic of AATD liver disease and is believed to lead to proteotoxic effects that are responsible for inducing liver injury, including liver cell damage and death and chronic liver injury, in individuals with AATD. (see, e.g., D. Lindblad et al., Hepatology 2007, 46: 1228-1235). Patients with AATD often develop liver disease, which can be severe or fatal, even in infancy. Clinical presentations of injury in the liver include chronic hepatitis, cirrhosis, hepatocellular carcinoma, transaminitis, cholestasis, fibrosis, and even fulminant hepatic failure.

There is currently no clinically approved treatment to prevent the onset or slow the progression of liver disease due to AATD. Further, while U.S. Patent Application Publication No. 2015/0361427 discloses certain RNAi agents capable of inhibiting the expression of an AAT gene, there remains a need for novel and effective AAT RNAi agents having improved potency that can selectively, efficiently, and safely inhibit the expression of an AAT gene, thereby preventing and potentially reversing Z-AAT accumulation-related liver injury and fibrosis. Similarly, while U.S. Patent Application Publication No. 2015/0011607 to Brown et al. (“Brown '607”) discloses various sequences for inhibiting expression of an AAT gene, Brown teaches the use of longer double-stranded constructs (referred to in Brown as DsiRNAs), which according to Brown have been found to give “unexpected effective results in terms of potency and duration of action” as compared to 19-23mer siRNA agents. (See, e.g., Brown '607 at paragraph [0376]). Moreover, many of the sequences disclosed in Brown '607 are designed to be used in DsiRNA constructs that are designed to target different locations of an AAT mRNA as compared to the sequences disclosed in the present invention. Such differences lead to different binding affinity to the AAT mRNA and produces a different cleavage site, which can impact the inhibitory effect of the compound, while also potentially leading to additional off-target issues (see. e.g., Piotr J. Kamola et al., PLoS Comput Biol, 2015, 11(12):e1004656 at FIG. 1 (illustrating the mechanism of siRNA-Mediated Gene Silencing)). For example, nothing in Brown '607 teaches or suggests the design of an RNAi agent (of any length) wherein the 5′ terminal nucleobase or nucleotide of the antisense strand would be aligned with the position that is 19 nucleotides downstream (towards the 3′ end) from position 1000 on an AAT gene (SEQ ID NO: 1). Put different, and again solely as an example involving one such potential AAT RNAi agent sequence, nothing in Brown '607 teaches or suggests the design of an RNAi agent wherein the 5′ terminal nucleobase of the antisense strand of an RNAi agent corresponds to position 1018 on an AAT gene (SEQ ID NO: 1). Further, nothing in Brown '607 teaches or suggests the modified AAT RNAi agent constructs disclosed herein.

### SUMMARY

There exists a need for novel AAT-specific RNA interference (RNAi) agents (also herein termed RNAi agent, RNAi trigger, or trigger) that are able to selectively and efficiently inhibit the expression of an AAT gene. Further, there exists a need for compositions of novel AAT-specific RNAi agents for the treatment of diseases associated with AAT deficiency.

Because liver damage resulting from AATD occurs through a gain-of-function mechanism, inhibition of AAT gene expression is useful in preventing accumulation of the Z-AAT protein in the liver. Further, the reduction or removal of the Z-AAT polymer aggregates reduces the ER stress in hepatocytes, and offers additional advantages in reducing the likelihood of occurrence of liver cell damage and assisting in the treatment of liver cell damage and chronic liver injury such as fibrosis, cirrhosis, hepatocellular carcinoma, and other conditions and diseases caused by AATD. Reduction of inflammatory Z-AAT protein, which has been clearly defined as the cause of progressive liver disease in AATD patients, is important as it can slow or halt the progression of liver disease and allow fibrotic tissue repair.

In general, the disclosure features novel AAT RNAi agents, compositions comprising the AAT RNAi agents, and methods for inhibiting the expression of an AAT gene in vivo and/or in vitro using AAT RNAi agents and compositions that include AAT RNAi agents. Further described herein are methods of treatment of AATD-related diseases using the AAT RNAi agents described herein and compositions that include AAT RNAi agents.

The AAT RNAi agents and methods disclosed herein can provide for the treatment of AATD, including the treatment of conditions and diseases caused by AATD, such as chronic hepatitis, cirrhosis, hepatocellular carcinoma, and fulminant hepatic failure. The AAT RNAi agents disclosed herein, when administered to a subject, can prevent and/or reverse Z-AAT accumulation-related liver injury and fibrosis. The AAT RNAi agents described herein may be administered to a subject, e.g., a human or animal subject, by any suitable methods known in the art, such as subcutaneous injection or intravenous administration.

In one aspect, the disclosure features RNAi agents for inhibiting the expression of an alpha-1 antitrypsin (AAT) gene, wherein the RNAi agent comprises a sense strand and an antisense strand. Also described herein are compositions comprising an RNAi agent capable of inhibiting the expression of an alpha-1 antitrypsin gene, wherein the RNAi agent comprises a sense strand and an antisense strand, and at least one pharmaceutically acceptable excipient.

Each AAT RNAi agent described herein includes a sense strand and an antisense strand. The sense strand and the antisense strand can be partially, substantially, or fully complementary to each other. The length of the RNAi agent sense and antisense strands described herein each can be 16 to 30 nucleotides in length. In some embodiments, the sense and antisense strands are independently 17 to 26 nucleotides in length. In some embodiments, the sense and antisense strands are independently 21 to 26 nucleotides in length. In some embodiments, the sense and antisense strands are independently 21 to 24 nucleotides in length. In some embodiments, the sense and/or antisense strands are independently 16, 17, 18, 19, 20, 21, 22, 23, 24, 25, 26, 27, 28, 29, or 30 nucleotides in length. The sense and antisense strands can be either the same length or different lengths. The RNAi agents described herein, upon delivery to a cell expressing AAT, inhibit the expression of one or more AAT genes in vivo or in vitro.

An AAT RNAi agent includes a sense strand (also referred to as a passenger strand), and an antisense strand (also referred to as a guide strand). A sense strand of the AAT RNAi agents described herein includes a nucleotide sequence having at least 85% identity to a core stretch of at least 16 consecutive nucleotides to a sequence in an AAT mRNA. In some embodiments, the sense strand core stretch having at least 85% identity to a sequence in an AAT mRNA is 16, 17, 18, 19, 20, 21, 22, or 23 nucleotides in length. An antisense strand of an AAT RNAi agent includes a nucleotide sequence having at least 85% complementarity over a core stretch of at least 16 consecutive nucleotides to a sequence in an AAT mRNA and the corresponding sense strand. In some embodiments, the antisense strand core stretch having at least 85% complementarity to a sequence in an AAT mRNA or the corresponding sense strand is 16, 17, 18, 19, 20, 21, 22, or 23 nucleotides in length.

In some embodiments, the AAT RNAi agents disclosed herein target a portion of an AAT gene having the sequence of any of the sequences disclosed in Table 1.

Examples of AAT RNAi agent sense strands and antisense strands that can be used in AAT RNAi agents are provided in Tables 2, 3, 4 and 5. Examples of duplexes that include AAT RNAi agent are provided in Table 6. Examples of 19-nucleotide core stretch sequences that may consist of or may be included in the sense strands and antisense strands of certain AAT RNAi agents disclosed herein, are provided in Table 2.

In another aspect, the disclosure features methods for delivering AAT RNAi agents to liver cells in a subject, such as a mammal, in vivo. In some embodiments, one or more AAT RNAi agents are delivered to target cells or tissues using any oligonucleotide delivery technology known in the art. Nucleic acid delivery methods include, but are not limited to, encapsulation in liposomes, iontophoresis, or incorporation into other vehicles, such as hydrogels, cyclodextrins, biodegradable nanocapsules, and bioadhesive microspheres, proteinaceous vectors, or Dynamic Polyconjugates™ (DPCs) (see, for example WO 2000/053722, WO 2008/0022309, WO 2011/104169, and WO 2012/083185, each of which is incorporated herein by reference). In some embodiments, a delivery vehicle, such as a polymer, an amphipathic polymer, a membrane active polymer, a peptide, such as a melittin or melittin-like peptide, a reversibly modified polymer or peptide, or a lipid, can be used with the AAT RNAi agents disclosed herein.

In some embodiments, an AAT RNAi agent is delivered to target cells or tissues by covalently linking or conjugating the RNAi agent to a targeting group such as an asialoglycoprotein receptor ligand. In some embodiments, an asialoglycoprotein receptor ligand includes, consists of, or consists essentially of, a galactose or galactose-derivative cluster. In some embodiments, an AAT RNAi agent is linked to a targeting ligand comprising the galactose-derivative N-acetyl-galactosamine. In some embodiments, a galactose-derivative cluster includes an N-acetyl-galactosamine trimer or an N-acetyl-galactosamine tetramer. In some embodiments, a galactose derivative cluster is an N-acetyl-galactosamine trimer or an N-acetyl-galactosamine tetramer. Example targeting groups useful for delivering RNAi agents are disclosed, for example, in U.S. patent Application Ser. No. 15/452.324 and WO 2017/156012, which are incorporated by reference herein in their entirety.

A targeting group can be linked to the 3′ or 5′ end of a sense strand or an antisense strand of an AAT RNAi agent. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, a targeting group is linked to the RNAi agent via a linker.

A targeting group, with or without a linker, can be linked to the 5′ or 3′ end of any of the sense and/or antisense strands disclosed in Tables 2, 3, 4, and 5. A linker, with or without a targeting group, can be attached to the 5′ or 3′ end of any of the sense and/or antisense strands disclosed in Tables 2, 3, 4, and 5.

In another aspect, the disclosure features compositions that include one or more AAT RNAi agents that have the duplex structures disclosed in Table 6.

In some embodiments, described herein are compositions that include a combination or cocktail of at least two AAT RNAi agents having different nucleotide sequences. In some embodiments, the two or more different AAT RNAi agents are each separately and independently linked to targeting groups. In some embodiments, the two or more different AAT RNAi agents are each linked to targeting groups that include or consist of targeting ligands that include one or more moieties that target an asialoglycoprotein receptor. In some embodiments, the two or more different AAT RNAi agents are each linked to targeting groups that include or consist of targeting ligands that include one or more galactose-derivatives. In some embodiments, the two or more different AAT RNAi agents are each linked to targeting groups that include or consist of targeting ligands that include one or more N-acetyl-galactosamines. In some embodiments, when two or more RNAi agents are included in a composition, each RNAi agent is independently linked to the same targeting group. In some embodiments, when two or more RNAi agents are included in a composition, each RNAi agent is independently linked to a different targeting group, such as targeting groups having different chemical structures.

In some embodiments, targeting groups are linked to the AAT RNAi agents without the use of an additional linker. In some embodiments, the targeting group is designed having a linker readily present to facilitate the linkage to an AAT RNAi agent. In some embodiments, when two or more RNAi agents are included in a composition, the two or more RNAi agents may be linked to their respective targeting groups using the same linkers. In some embodiments, when two or more RNAi agents are included in a composition, the two or more RNAi agents are linked to their respective targeting groups using different linkers.

In another aspect, the disclosure features methods for inhibiting alpha-1 antitrypsin gene expression in a subject, the methods comprising administering to the subject an amount of an AAT RNAi agent capable of inhibiting the expression of an AAT gene, wherein the AAT RNAi agent comprises a sense strand and an antisense strand.

Also described herein are methods for the treatment of a condition or disease caused by AATD, comprising administering to a subject a therapeutically effective amount of an RNAi agent described herein. Further described are methods for inhibiting expression of an AAT gene, wherein the methods include administering to a cell an AAT RNAi agent described herein.

In some embodiments, disclosed herein are methods for the treatment of AATD (including the treatment of a condition or disease caused by AATD), the methods comprising administering to a subject a therapeutically effective amount of an RNAi agent having an antisense strand comprising the sequence of any of the sequences in Tables 2, 3, or 4.

In some embodiments, disclosed herein are methods for inhibiting expression of an AAT gene, the methods comprising administering to a cell an AAT RNAi agent that includes an antisense strand comprising the sequence of any of the sequences in Tables 2, 3, or 4.

In some embodiments, disclosed herein methods for the treatment of AATD (including the treatment of a condition or disease caused by AATD), the methods comprising administering to a subject a therapeutically effective amount of an RNAi agent that includes a sense strand comprising the sequence of any of the sequences in Tables 2, 3, or 5.

In some embodiments, disclosed herein are methods for inhibiting expression of an AAT gene, wherein the methods include administering to a cell an AAT RNAi agent that includes a sense strand comprising the sequence of any of the sequences in Tables 2, 3, or 5.

In some embodiments, disclosed herein are methods for the treatment of AATD (including the treatment of a condition or disease caused by AATD), wherein the methods include administering to a subject a therapeutically effective amount of an RNAi agent that includes a sense strand comprising the sequence of any of the sequences in Table 5, and an antisense strand comprising any of the sequences in Table 4.

In some embodiments, disclosed herein are methods for inhibiting expression of an AAT gene, wherein the methods include administering to a subject a therapeutically effective amount of an RNAi agent that includes a sense strand comprising the sequence of any of the sequences in Table 5, and an antisense strand comprising any of the sequences in Table 4.

In some embodiments, disclosed herein are methods of inhibiting expression of an AAT gene, wherein the methods include administering to a subject an AAT RNAi agent that includes a sense strand consisting of the nucleobase sequence of any of the sequences in Table 5, and the antisense strand consisting of the nucleobase sequence of any of the sequences in Table 4. In other embodiments, disclosed herein are methods of inhibiting expression of an AAT gene, wherein the methods include administering to a subject an AAT RNAi agent that includes a sense strand consisting of the modified sequence of any of the modified sequences in Table 5, and the antisense strand consisting of the modified sequence of any of the modified sequences in Table 4.

In some embodiments, disclosed herein are methods for inhibiting expression of an AAT gene in a cell, wherein the methods include administering one or more AAT RNAi agents having the duplex structure set forth in Table 6.

In some embodiments, the AAT RNAi agents disclosed herein have structures that include, consist of, or consist essentially of, the structure shown in any one of FIG. 1, FIG. 2, FIG. 3, FIG. 4, FIG. 5, FIG. 6, FIG. 7, or FIG. 8.

The AAT RNAi agents disclosed herein are designed to target specific positions on an AAT gene (SEQ ID NO: 1). As defined herein, an antisense strand sequence is designed to target an AAT gene at a given position on the gene when the 5′ terminal nucleobase of the antisense strand would be aligned with the position that is 19 nucleotides downstream (towards the 3′ end) from the position on the gene when base pairing to the gene. For example, as illustrated in Tables 1, 2, and 3 herein, an antisense strand sequence designed to target an AAT gene at position 1000 requires that when base pairing to the gene, the 5′ terminal nucleobase of the antisense strand is aligned with position 1018 of the AAT gene. As provided herein, an AAT RNAi agent does not require that the nucleobase at position 1 (5′→3′) of the antisense strand be complementary to the gene, provided that there is at least 85% complementarity (e.g., at least 90, 91, 92, 93, 94, 95, 96, 97, 98, 99, or 1000% complementarity) of the antisense strand and the gene across a core stretch sequence of at least 16 consecutive nucleotides. For example, for an AAT RNAi agent disclosed herein that is designed to target position 1000 of an AAT gene, the 5′ terminal nucleobase of the antisense strand of the of the AAT RNAi agent must be aligned with position 1018 of the gene; however, the 5′ terminal nucleobase of the antisense strand may be, but is not required to be, complementary to position 1018 of an AAT gene, provided that there is at least 85% complementarity (e.g., at least 90, 91, 92, 93, 94, 95, 96, 97, 98, 99, or 100% complementarity) of the antisense strand and the gene across a core stretch sequence of at least 16 consecutive nucleotides. As shown by, among other things, the various examples disclosed herein, the specific site of binding of the gene by the antisense strand of the AAT RNAi agent (e.g., whether the AAT RNAi agent is designed to target an AAT gene at position 1000, at position 1142, or at some other position) is highly important to the level of inhibition achieved by the AAT RNAi agent.

In some embodiments, the antisense strand sequence is designed to have a sequence target position 1000 of an AAT gene (SEQ ID NO. 1).

In some embodiments, the antisense strand of an AAT RNAi agent comprises or consists of the nucleobase sequence of UGUUAAACAUGCCUAAACGCU (SEQ ID NO: 801), wherein at least one or more nucleotides is a modified nucleotide. In some embodiments, the antisense strand of an AAT RNAi agent comprises or consists of the nucleobase sequence of UGUUAAACAUGCCUAAACGCU (SEQ ID NO: 801), wherein all or substantially all of the nucleotides are modified nucleotides.

In some embodiments, the antisense strand of an AAT RNAi agent comprises or consists of the nucleobase sequence of UGUUAAACAUGCCUAAACGUU (SEQ ID NO: 794), wherein at least one or more nucleotides is a modified nucleotide. In some embodiments, the antisense strand of an AAT RNAi agent comprises or consists of the nucleobase sequence of UGUUAAACAUGCCUAAACGUU (SEQ ID NO: 794), wherein all or substantially all of the nucleotides are modified nucleotides.

In some embodiments, the antisense strand of an AAT RNAi agent comprises or consists of the nucleobase sequence of UGUUAAACAUGCCUAAACGCUU (SEQ ID NO: 839), wherein at least one or more nucleotides is a modified nucleotide. In some embodiments, the antisense strand of an AAT RNAi agent comprises or consists of the nucleobase sequence of UGUUAAACAUGCCUAAACGCUU (SEQ ID NO: 839), wherein all or substantially all of the nucleotides are modified nucleotides.

In some embodiments, the antisense strand of an AAT RNAi agent comprises or consists of the nucleobase sequence of UGUUAAACAUGCCUAAACGCG (SEQ ID NO: 800), wherein at least one or more nucleotides is a modified nucleotide. In some embodiments, the antisense strand of an AAT RNAi agent comprises or consists of the nucleobase sequence of UGUUAAACAUGCCUAAACGCG (SEQ ID NO: 800), wherein all or substantially all of the nucleotides are modified nucleotides.

In some embodiments, the antisense strand of an AAT RNAi agent comprises or consists of the nucleobase sequence of UGUUAAACAUGCCUAAACG (SEQ ID NO: 80), wherein one or more nucleotides is a modified nucleotide. In some embodiments, the antisense strand of an AAT RNAi agent comprises or consists of the nucleobase sequence of UGUUAAACAUGCCUAAACG (SEQ ID NO: 80), wherein all or substantially all of the nucleotides are modified nucleotides.

In some embodiments, the antisense strand of an AAT RNAi agent comprises or consists of the nucleobase sequence of AGUUAAACAUGCCUAAACG (SEQ ID NO: 81), wherein one or more nucleotides is a modified nucleotide. In some embodiments, the antisense strand of an AAT RNAi agent comprises or consists of the nucleobase sequence of AGUUAAACAUGCCUAAACG (SEQ ID NO: 81), wherein all or substantially all of the nucleotides are modified nucleotides.

In some embodiments, the sense strand of an AAT RNAi agent comprises or consists of the nucleobase sequence of CGUUUAGGCAUGUUUAACA (SEQ ID NO: 429), wherein one or more nucleotides is a modified nucleotide. In some embodiments, the sense strand of an AAT RNAi agent comprises or consists of the nucleobase sequence of CGUUUAGGCAUGUUUAACA (SEQ ID NO: 429), wherein all or substantially all of the nucleotides are modified nucleotides.

In some embodiments, the sense strand of an AAT RNAi agent comprises or consists of the nucleobase sequence of CGUUUAGGCAUGUUUAACU (SEQ ID NO: 430), wherein one or more nucleotides is a modified nucleotide. In some embodiments, the sense strand of an AAT RNAi agent comprises or consists of the nucleobase sequence of CGUUUAGGCAUGUUUAACU (SEQ ID NO: 430), wherein all or substantially all of the nucleotides are modified nucleotides.

In some embodiments, the sense strand of an AAT RNAi agent comprises or consists of the nucleobase sequence of CGUUUAGGCAUGUUUAACA (SEQ ID NO: 429), wherein one or more nucleotides is a modified nucleotide, and the antisense strand of an AAT RNAi agent comprises or consists of the nucleobase sequence of UGUUAAACAUGCCUAAACG (SEQ ID NO: 80), wherein one or more nucleotides is a modified nucleotide.

In some embodiments, the sense strand of an AAT RNAi agent comprises or consists of the nucleobase sequence of CGUUUAGGCAUGUUUAACU (SEQ ID NO: 430), wherein one or more nucleotides is a modified nucleotide, and the antisense strand of an AAT RNAi agent comprises or consists of the nucleobase sequence of AGUUAAACAUGCCUAAACG (SEQ ID NO: 81), wherein one or more nucleotides is a modified nucleotide.

In some embodiments, the antisense strand of an AAT RNAi agent comprises or consists of the nucleobase sequence of UGUUAAACAUGCCUAAACGUU (SEQ ID NO: 794), wherein at least one or more nucleotides is a modified nucleotide, and the sense strand of an AAT RNAi agent comprises or consists of the nucleobase sequence of CGUUUAGGCAUGUUUAACAUU (SEQ ID NO: 857).

In some embodiments, the antisense strand of an AAT RNAi agent comprises or consists of the nucleobase sequence of UGUUAAACAUGCCUAAACGCUU (SEQ ID NO: 839), wherein at least one or more nucleotides is a modified nucleotide, and the sense strand of an AAT RNAi agent comprises or consists of the nucleobase sequence of GCGUUUAGGCAUGUUUAACAUU (SEQ ID NO: 885).

In some embodiments, the antisense strand of an AAT RNAi agent comprises or consists of the nucleobase sequence of UGUUAAACAUGCCUAAACGCG (SEQ ID NO: 800), wherein at least one or more nucleotides is a modified nucleotide, and the sense strand of an AAT RNAi agent comprises or consists of the nucleobase sequence of CGCGUUUAGGCAUGUUUAACA (SEQ ID NO: 864).

In some embodiments, the antisense strand of an AAT RNAi agent comprises or consists of the nucleobase sequence of UGUUAAACAUGCCUAAACGCU (SEQ ID NO: 801), wherein at least one or more nucleotides is a modified nucleotide, and the sense strand of an AAT RNAi agent comprises or consists of the nucleobase sequence of AGCGUUUAGGCAUGUUUAACA (SEQ ID NO: 866).

In some embodiments, the antisense strand of an AAT RNAi agent comprises or consists of the nucleobase sequence of UGUUAAACAUGCCUAAACGUU (SEQ ID NO: 794) differing by 0, 1, 2, or 3 nucleotides, wherein at least one or more nucleotides is a modified nucleotide, and the sense strand of an AAT RNAi agent comprises or consists of the nucleobase sequence of CGUUUAGGCAUGUUUAACAUU (SEQ ID NO: 857) differing by 0, 1, 2, or 3 nucleotides.

In some embodiments, the antisense strand of an AAT RNAi agent comprises or consists of the nucleobase sequence of UGUUAAACAUGCCUAAACGCUU (SEQ ID NO: 839) differing by 0, 1, 2, or 3 nucleotides, wherein at least one or more nucleotides is a modified nucleotide, and the sense strand of an AAT RNAi agent comprises or consists of the nucleobase sequence of GCGUUUAGGCAUGUUUAACAUU (SEQ ID NO: 885) differing by 0, 1, 2, or 3 nucleotides.

In some embodiments, the antisense strand of an AAT RNAi agent comprises or consists of the nucleobase sequence of UGUUAAACAUGCCUAAACGCG (SEQ ID NO: 800) differing by 0, 1, 2, or 3 nucleotides, wherein at least one or more nucleotides is a modified nucleotide, and the sense strand of an AAT RNAi agent comprises or consists of the nucleobase sequence of CGCGUUUAGGCAUGUUUAACA (SEQ ID NO: 864) differing by 0, 1, 2, or 3 nucleotides.

In some embodiments, the antisense strand of an AAT RNAi agent comprises or consists of the nucleobase sequence of UGUUAAACAUGCCUAAACGCU (SEQ ID NO: 801) differing by 0, 1, 2, or 3 nucleotides, wherein at least one or more nucleotides is a modified nucleotide, and the sense strand of an AAT RNAi agent comprises or consists of the nucleobase sequence of AGCGUUUAGGCAUGUUUAACA (SEQ ID NO: 866) differing by 0, 1, 2, or 3 nucleotides.

In some embodiments, the AAT RNAi agent comprises, consists of, or consists essentially of the duplex structure of AD04824.

In some embodiments, the AAT RNAi agent comprises, consists of, or consists essentially of the duplex structure of AD(4825.

In some embodiments, the AAT RNAi agent comprises, consists of, or consists essentially of the duplex structure of AD04826. In some embodiments, the AAT RNAi agent comprises, consists of, or consists essentially of the duplex structure of AD(4827.

In some embodiments, the AAT RNAi agent comprises, consists of, or consists essentially of the duplex structure of AD04828.

In some embodiments, the AAT RNAi agent comprises, consists of, or consists essentially of the duplex structure of AD04829.

In some embodiments, the AAT RNAi agent comprises, consists of, or consists essentially of the duplex structure of AD04830.

In some embodiments, the AAT RNAi agent comprises, consists of, or consists essentially of the duplex structure of AD(4831.

In some embodiments, the AAT RNAi agent comprises, consists of, or consists essentially of the duplex AD04832.

In some embodiments, the AAT RNAi agent comprises, consists of, or consists essentially of the duplex structure of AD04833.

In some embodiments, the AAT RNAi agent comprises, consists of, or consists essentially of the duplex structure of AD04834.

In some embodiments, the AAT RNAi agent comprises, consists of, or consists essentially of the duplex structure of AD04835.

In some embodiments, the AAT RNAi agent comprises, consists of, or consists essentially of the duplex structure of AD(4836.

In some embodiments, the AAT RNAi agent comprises, consists of, or consists essentially of the duplex structure of AD04837.

In some embodiments, the antisense strand of an AAT RNAi agent comprises or consists of the nucleobase sequence of UGUUAAACAUGCCUAAACG (SEQ ID NO: 80), wherein one or more nucleotides is a modified nucleotide, and wherein SEQ ID NO: 80 is located at positions 1 to 19 (5′→3′) of the antisense strand. In some embodiments, the antisense strand of an AAT RNAi agent comprises or consists of the nucleobase sequence of UGUUAAACAUGCCUAAACG (SEQ ID NO: 80), wherein all or substantially all of the nucleotides are modified nucleotides, and wherein SEQ ID NO: 80 is located at positions 1 to 19 (5′→3′) of the antisense strand.

In some embodiments, the antisense strand of an AAT RNAi agent comprises or consists of the nucleobase sequence of AGUUAAACAUGCCUAAACG (SEQ ID NO: 81), wherein one or more nucleotides is a modified nucleotide, and wherein SEQ ID NO: 81 is located at positions 1 to 19 (5′→3′) of the antisense strand. In some embodiments, the antisense strand of an AAT RNAi agent comprises or consists of the nucleobase sequence of AGUUAAACAUGCCUAAACG (SEQ ID NO: 81), wherein all or substantially all of the nucleotides are modified nucleotides, and wherein SEQ ID NO: 81 is located at positions 1 to 19 (5′→3′) of the antisense strand.

In some embodiments, the sense strand of an AAT RNAi agent comprises the nucleobase sequence of CGUUUAGGCAUGUUUAACA (SEQ ID NO: 429), wherein one or more nucleotides is a modified nucleotide, and wherein position 19 of SEQ ID NO: 429 forms a base pair with the nucleotide located at the 5′ terminal end of the antisense strand. In some embodiments, the sense strand of an AAT RNAi agent comprises or consists of the nucleobase sequence of CGUUUAGGCAUGUUUAACA (SEQ ID NO: 429), wherein all or substantially all of the nucleotides are modified nucleotides, and wherein position 19 of SEQ ID NO: 429 forms a base pair with the nucleotide located at the 5′ terminal end of the antisense strand.

In some embodiments, the sense strand of an AAT RNAi agent comprises the nucleobase sequence of CGUUUAGGCAUGUUUAACU (SEQ ID NO: 430), wherein one or more nucleotides is a modified nucleotide, and wherein position 19 of SEQ ID NO: 430 forms a base pair with the nucleotide located at the 5′ terminal end of the antisense strand. In some embodiments, the sense strand of AN AAT RNAI agent comprises the nucleobase sequence of CGUUUAGGCAUGUUUAACU (SEQ ID NO: 430), wherein all or substantially all of the nucleotides are modified nucleotides, and wherein position 19 of SEQ ID NO: 430 forms a base pair with the nucleotide located at the 5′ terminal end of the antisense strand.

In some embodiments, the antisense strand of an AAT RNAi agent comprises or consists of the nucleobase sequence of UGUUAAACAUGCCUAAACGUU (SEQ ID NO: 794), wherein at least one or more nucleotides is a modified nucleotide, and wherein SEQ ID NO: 794 is located at positions 1 to 21 (5′→3′) of the antisense strand. In some embodiments, the antisense strand of an AAT RNAi agent comprises or consists of the nucleobase sequence of UGUUAAACAUGCCUAAACGUU (SEQ ID NO: 794), wherein all or substantially all of the nucleotides are modified nucleotides, and wherein SEQ ID NO: 794 is located at positions 1 to 21 (5′→3′) of the antisense strand.

In some embodiments, the antisense strand of an AAT RNAi agent comprises or consists of the nucleobase sequence of UGUUAAACAUGCCUAAACGCUU (SEQ ID NO: 839), wherein at least one or more nucleotides is a modified nucleotide, and wherein SEQ ID NO: 839 is located at positions 1 to 22 (5′→3′) of the antisense strand. In some embodiments, the antisense strand of an AAT RNAi agent comprises or consists of the nucleobase sequence of UGUUAAACAUGCCUAAACGCUU (SEQ ID NO: 839), wherein all or substantially all of the nucleotides are modified nucleotides, and wherein SEQ ID NO: 839 is located at positions 1 to 22 (5′→3′) of the antisense strand.

In some embodiments, the antisense strand of an AAT RNAi agent comprises or consists of the nucleobase sequence of UGUUAAACAUGCCUAAACGCG (SEQ ID NO: 800), wherein at least one or more nucleotides is a modified nucleotide, and wherein SEQ ID NO: 800 is located at positions 1 to 21 (5′→3′) of the antisense strand. In some embodiments, the antisense strand of an AAT RNAi agent comprises or consists of the nucleobase sequence of UGUUAAACAUGCCUAAACGCG (SEQ ID NO; 800), wherein all or substantially all of the nucleotides are modified nucleotides, and wherein SEQ ID NO: 800 is located at positions 1 to 21 (5′→3′) of the antisense strand.

In some embodiments, the antisense strand of an AAT RNAi agent comprises or consists of the nucleobase sequence of UGUUAAACAUGCCUAAACGCU (SEQ ID NO: 801), wherein at least one or more nucleotides is a modified nucleotide, and wherein SEQ ID NO: 801 is located at positions 1 to 21 (5′→3′) of the antisense strand. In some embodiments, the antisense strand of an AAT RNAi agent comprises or consists of the nucleobase sequence of UGUUAAACAUGCCUAAACGCU (SEQ ID NO: 801), wherein all or substantially all of the nucleotides are modified nucleotides, and wherein SEQ ID NO: 801 is located at positions 1 to 21 (5′→3′) of the antisense strand.

In some embodiments, the antisense strand of an AAT RNAi agent comprises or consists of the nucleobase sequence of UGUUAAACAUGCCUAAACGUU (SEQ ID NO: 794), wherein at least one or more nucleotides is a modified nucleotide, and wherein SEQ ID NO: 794 is located at the 5′ the terminal end of the antisense strand, and wherein sense strand of an AAT RNAi agent comprises or consists of the nucleobase sequence of CGUUUAGGCAUGUUUAACAUU (SEQ ID NO: 857).

In some embodiments, the antisense strand of an AAT RNAi agent comprises or consists of the nucleobase sequence of UGUUAAACAUGCCUAAACGCUU (SEQ ID NO: 839), wherein at least one or more nucleotides is a modified nucleotide, and wherein SEQ ID NO: 839 is located at the 5′ the terminal end of the antisense strand, and the sense strand of an AAT RNAi agent comprises or consists of the nucleobase sequence of GCGUUUAGGCAUGUUUAACAUU (SEQ ID NO: 885).

In some embodiments, the antisense strand of an AAT RNAi agent comprises or consists of the nucleobase sequence of UGUUAAACAUGCCUAAACGCG (SEQ ID NO: 800), wherein at least one or more nucleotides is a modified nucleotide, and wherein SEQ ID NO: 800 is located at the 5′ the terminal end of the antisense strand, and the sense strand of an AAT RNAi agent comprises or consists of the nucleobase sequence of CGCGUUUAGGCAUGUUUAACA (SEQ ID NO: 864).

In some embodiments, the antisense strand of an AAT RNAi agent comprises or consists of the nucleobase sequence of UGUUAAACAUGCCUAAACGCU (SEQ ID NO: 801), wherein at least one or more nucleotides is a modified nucleotide, and wherein SEQ ID NO: 801 is located at the 5′ the terminal end of the antisense strand, and the sense strand of an AAT RNAi agent comprises or consists of the nucleobase sequence of AGCGUUUAGGCAUGUUUAACA (SEQ ID NO: 866).

In some embodiments, the antisense strand of an AAT RNAi agent comprises or consists of the nucleobase sequence of UGUUAAACAUGCCUAAACGUU (SEQ ID NO: 794) differing by 0, 1, 2, or 3 nucleotides, wherein at least one or more nucleotides is a modified nucleotide, and wherein SEQ ID NO: 794 is located at the 5′ the terminal end of the antisense strand, and the sense strand of an AAT RNAi agent comprises or consists of the nucleobase sequence of CGUUUAGGCAUGUUUAACAUU (SEQ ID NO: 857) differing by 0, 1, 2, or 3 nucleotides.

In some embodiments, the antisense strand of an AAT RNAi agent comprises or consists of the nucleobase sequence of UGUUAAACAUGCCUAAACGCUU (SEQ ID NO: 839) differing by 0, 1, 2, or 3 nucleotides, wherein at least one or more nucleotides is a modified nucleotide, and wherein SEQ ID NO: 839 is located at the 5′ the terminal end of the antisense strand, and the sense strand of an AAT RNAi agent comprises or consists of the nucleobase sequence of GCGUUUAGGCAUGUUUAACAUU (SEQ ID NO: 885) differing by 0, 1, 2, or 3 nucleotides.

In some embodiments, the antisense strand of an AAT RNAi agent comprises or consists of the nucleobase sequence of UGUUAAACAUGCCUAAACGCG (SEQ ID NO: 800) differing by 0, 1, 2, or 3 nucleotides, wherein at least one or more nucleotides is a modified nucleotide, and wherein SEQ ID NO: 800 is located at the 5′ the terminal end of the antisense strand, and the sense strand of an AAT RNAi agent comprises or consists of the nucleobase sequence of CGCGUUUAGGCAUGUUUAACA (SEQ ID NO: 864) differing by 0, 1, 2, or 3 nucleotides.

In some embodiments, the antisense strand of an AAT RNAi agent comprises or consists of the nucleobase sequence of UGUUAAACAUGCCUAAACGCU (SEQ ID NO: 801) differing by 0, 1, 2, or 3 nucleotides, wherein at least one or more nucleotides is a modified nucleotide, and wherein SEQ ID NO: 801 is located at the 5′ the terminal end of the antisense strand, and the sense strand of an AAT RNAi agent comprises or consists of the nucleobase sequence of AGCGUUUAGGCAUGUUUAACA (SEQ ID NO: 866) differing by 0, 1, 2, or 3 nucleotides.

The AAT RNAi agents described herein can include one or more modified nucleotides. The AAT RNAi agents described herein can also include one or more phosphorothioate internucleoside linkages.

The AAT RNAi agents described herein can also include one or more targeting groups or linking groups. In some embodiments, the AAT RNAi agents disclosed herein include one or more targeting groups. In some embodiments, the targeting groups are comprised of an asialoglycoprotein receptor ligand. In some embodiments, the asialoglycoprotein receptor ligand comprises a galactose or galactose-derivative cluster. In some embodiments, the galactose-derivative cluster comprises N-acetyl-galactosamine. In some embodiments, the targeting ligand comprises an N-acetyl-galactosamine trimer. In some embodiments, a targeting group is conjugated to the sense strand of the AAT RNAi agents disclosed herein.

In some embodiments, the antisense strand of an AAT RNAi agent comprises or consists of the sequence (5′→3′) usGfsusUfaAfaCfaUfgCfcUfaAfaCfgusu (SEQ ID NO: 913), wherein a, c, g, and u are 2′-O-methyl adenosine, cytidine, guanosine, or uridine, respectively: Af, Cf, Gf, and Uf are 2′-fluoro adenosine, cytidine, guanosine, or uridine, respectively; and s is a phosphorothioate linkage, and the sense strand is at least substantially complementary to the antisense strand.

In some embodiments, the antisense strand of an AAT RNAi agent comprises or consists of the sequence (5′+3′) usGfsusUfaAfaCfaUfgCfcUfaAfaCfgcusu (SEQ ID NO: 958), wherein a, c, g, and u are 2′-O-methyl adenosine, cytidine, guanosine, or uridine, respectively: Af, Cf, Gf, and Uf are 2′-fluoro adenosine, cytidine, guanosine, or uridine, respectively; and s is a phosphorothioate linkage, and the sense strand is at least substantially complementary to the antisense strand.

In some embodiments, the antisense strand of an AAT RNAi agent comprises or consists of the sequence (5′→3′) usGfsuUfaAfaCfaUfgCfcUfaAfaCfgsCfsg (SEQ ID NO: 959), wherein a, c, g, and u are 2′-O-methyl adenosine, cytidine, guanosine, or uridine, respectively: Af, Cf, Gf, and Uf are 2′-fluoro adenosine, cytidine, guanosine, or uridine, respectively; and s is a phosphorothioate linkage, and the sense strand is at least substantially complementary to the antisense strand.

In some embodiments, the antisense strand of an AAT RNAi agent comprises or consists of the sequence (5′→3′) usGfsuUfaAfacaugCfcUfaAfaCfgCfsu (SEQ ID NO: 960), wherein a, c, g, and u are 2′-O-methyl adenosine, cytidine, guanosine, or uridine, respectively; Af, Cf. Gf. and Uf are 2′-fluoro adenosine, cytidine, guanosine, or uridine, respectively; and s is a phosphorothioate linkage, and the sense strand is at least substantially complementary to the antisense strand.

In some embodiments, the antisense strand of an AAT RNAi agent comprises or consists of the sequence (5′→3′) usGfsusUfaAfaCfaUfgCfcUfaAfaCfgusu (SEQ ID NO: 913) and the sense strand of an AAT RNAi agent comprises or consists of the sequence (5′-+3′) cguuuaGfGfCfauguuuaacausu (SEQ ID NO: 1276), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; s is a phosphorothioate linkage; wherein optionally present on the sense strand is one, two, or more inverted abasic deoxyribose (invAb); and wherein optionally linked to the 5′ terminal end of the sense strand is a targeting ligand that includes N-acetyl-galactosamine.

In some embodiments, the antisense strand of an AAT RNAi agent comprises or consists of the sequence (5′→3′) usGfsusUfaAfaCfaUfgCfcUfaAfaCfgcusu (SEQ ID NO: 958) and the sense strand of an AAT RNAi agent comprises or consists of the sequence (5′→3′) gcguuuaGfGfCfauguuuaacausu (SEQ ID NO: 1277), wherein a, c, g, and u are 2′-O-methyl adenosine, cytidine, guanosine, or uridine, respectively: Af, Cf, Gf, and Uf are 2′-fluoro adenosine, cytidine, guanosine, or uridine, respectively; s is a phosphorothioate linkage: wherein optionally present on the sense strand is one, two, or more inverted abasic deoxyribose (invAb); and wherein optionally linked to the 5′ terminal end of the sense strand is a targeting ligand that includes N-acetyl-galactosamine.

In some embodiments, the antisense strand of an AAT RNAi agent comprises or consists of the sequence (5′→3′) usGfsuUfaAfaCfaUfgCfcUfaAfaCfgsCfsg (SEQ ID NO: 959) and the sense strand of an AAT RNAi agent comprises or consists of the sequence (5′→3′) cgcguuuaGfGfCfauguuuaaca (SEQ ID NO: 1278), wherein a, c, g, and u are 2′-O-methyl adenosine, cytidine, guanosine, or uridine, respectively; Af. Cf. Gf, and Uf are 2′-fluoro adenosine, cytidine, guanosine, or uridine, respectively; s is a phosphorothioate linkage; wherein optionally present on the sense strand is one, two, or more inverted abasic deoxyribose (invAb): and wherein optionally linked to the 5′ terminal end of the sense strand is a targeting ligand that includes N-acetyl-galactosamine.

In some embodiments, the antisense strand of an AAT RNAi agent comprises or consists of the sequence (5′→3′) usGfsuUfaAfacaugCfcUfaAfaCfgCfsu (SEQ ID NO: 960) and the sense strand of an AAT RNAi agent comprises or consists of the sequence (5′→3′) agcguuuaGfGfCfauguuuaaca (SEQ ID NO: 1279), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; s is a phosphorothioate linkage: wherein optionally present on the sense strand is one, two, or more inverted abasic deoxyribose (invAb); and wherein optionally linked to the 5′ terminal end of the sense strand is a targeting ligand that includes N-acetyl-galactosamine.

In some embodiments, the antisense strand of an AAT RNAi agent comprises or consists of the sequence (5′→3′) usGfsusUfaAfaCfaUfgCfcUfaAfaCfgusu (SEQ ID NO: 913) and the sense strand of an AAT RNAi agent comprises or consists of (5′→3′) (NAG37)s(invAb)scguuuaGfGfCfauguuuaacausu(invAb) (SEQ ID NO: 1028), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; s is a phosphorothioate linkage: (invAb) is inverted abasic deoxyribose (invAb): and (NAG37) is the targeting ligand that includes N-acetyl-galactosamine having the structure shown in Table 7 herein.

In some embodiments, the antisense strand of an AAT RNAi agent comprises or consists of the sequence (5′→3′) usGfsusUfaAfaCfaUfgCfcUfaAfaCfgcusu (SEQ ID NO: 958) and the sense strand of an AAT RNAi agent comprises or consists of the sequence (5′→3′) (NAG37)s(invAb)sgcguuuaGfGfCfauguuuaacausu(invAb) (SEQ ID NO: 1030), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; s is a phosphorothioate linkage; (invAb) is inverted abasic deoxyribose (invAb); and (NAG37) is the targeting ligand that includes N-acetyl-galactosamine having the structure shown in Table 7 herein.

In some embodiments, the antisense strand of an AAT RNAi agent comprises or consists of the sequence (5′→3′) usGfsuUfaAfaCfaUfgCfcUfaAfaCfgsCfsg (SEQ ID NO: 959) and the sense strand of an AAT RNAi agent comprises or consists of the sequence (5′→3′) (NAG37)s(invAb)scgcguuuaGfGfCfauguuuaacas(invAb)(SEQ ID NO: 1024), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; s is a phosphorothioate linkage; (invAb) is inverted abasic deoxyribose (invAb); and (NAG37) is the targeting ligand that includes N-acetyl-galactosamine having the structure shown in Table 7 herein.

In some embodiments, the antisense strand of an AAT RNAi agent comprises or consists of the sequence (5′→3′) usGfsuUfaAfacaugCfcUfaAfaCfgCfsu (SEQ ID NO: 960) and the sense strand of an AAT RNAi agent comprises or consists of the sequence (5′→3′) (NAG37)s(invAb)sagcguuuaGfGfCfauguuuaacas(invAb)(SEQ ID NO: 1033), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; s is a phosphorothioate linkage; (invAb) is inverted abasic deoxyribose (invAb); and (NAG37) is the targeting ligand that includes N-acetyl-galactosamine having the structure shown in Table 7 herein.

In some embodiments, the AAT RNAi agents described herein can include one or more targeting groups having the structure of (PAZ), (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s. (NAG32), (NAG32)s. (NAG33), (NAG33)s. (NAG34), (NAG34)s. (NAG35), (NAG35)s, (NAG36), (NAG36)s, (NAG37), (NAG37)s. (NAG38), (NAG38)s, (NAG39), (NAG39)s, as defined herein in Table 7.

In some embodiments, the AAT RNAi agents described herein include one targeting group at the 5′ end of the sense strand having the structure of (PAZ), (NAG25), (NAG25)s, (NAG26), (NAG26)s, (NAG27), (NAG27)s, (NAG28), (NAG28)s, (NAG29), (NAG29)s, (NAG30), (NAG30)s, (NAG31). (NAG31)s, (NAG32), (NAG32)s, (NAG33), (NAG33)s, (NAG34). (NAG34)s, (NAG35), (NAG35)s, (NAG36), (NAG36)s, (NAG37), (NAG37)s, (NAG38), (NAG38)s, (NAG39), (NAG39)s, as defined herein in Table 7.

The AAT RNAi agents disclosed herein can be incorporated into a composition comprising one or more disclosed AAT RNAi agent and at least one pharmaceutically acceptable excipient. In some embodiments, the compositions disclosed herein comprising one or more of the disclosed AAT RNAi agents and at least one pharmaceutically acceptable excipient is a pharmaceutical composition.

The pharmaceutical compositions comprising one or more AAT RNAi agents can be administered in a number of ways depending upon whether local or systemic treatment is desired. Administration can be, but is not limited to, intravenous, intraarterial, subcutaneous, intraperitoneal, subdermal (e.g., via an implanted device), and intraparenchymal administration. In some embodiments, the pharmaceutical compositions described herein are administered by subcutaneous injection.

In some embodiments, the compositions comprising one or more disclosed AAT RNAi agents and at least one pharmaceutically acceptable excipient can further comprise one or more additional therapeutics or treatments.

In some embodiments, the compositions described herein comprising one or more AAT RNAi agents are packaged in a kit, container, pack, dispenser, pre-filled syringes, or vials. In some embodiments, the compositions described herein are administered parenterally.

The AAT RNAi agents and compositions comprising same that are disclosed herein can be administered to a subject to inhibit the expression of the alpha-1 antitrypsin gene in the subject. In some embodiments, the subject is a human. In some embodiments, the subject is a human that has been diagnosed with having AATD.

In some embodiments, disclosed herein are methods for inhibiting expression of an AAT gene in a cell, the methods comprising administering an AAT RNAi agent that has an antisense strand that is at least partially complementary to the portion of an AAT mRNA having any one of the sequences listed in Table 1.

The AAT RNAi agents and compositions comprising same disclosed herein may be administered to a subject for the treatment of AATD (including a condition or disease caused by alpha-1 antitrypsin deficiency). The condition or disease that may be treated, prevented, and/or managed by administration of the AAT RNAi agents and compositions comprising same disclosed herein include chronic hepatitis, cirrhosis, hepatocellular carcinoma, transaminitis, cholestasis, fibrosis, or fulminant hepatic failure.

As used herein, the terms “oligonucleotide” and “polynucleotide” mean a polymer of linked nucleosides each of which can be independently modified or unmodified.

As used herein, an “RNAi agent” or “RNAi trigger” means a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. As used herein. RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short interfering RNAs (siRNAs), double-strand RNAs (dsRNA), micro RNAs (miRNAs), short hairpin RNAs (shRNA), and dicer substrates. The antisense strand of the RNAi agents described herein is at least partially complementary to the mRNA being targeted (e.g. AAT mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base”, “nucleotide base,” or “nucleobase,” is a heterocyclic pyrimidine or purine compound, which is a standard constituent of all nucleic acids, and includes the bases that form the nucleotides adenine (A), guanine (G), cytosine (C), thymine (T), and uracil (U). A nucleobase may further be modified to include, without limitation, universal bases, hydrophobic bases, promiscuous bases, size-expanded bases, and fluorinated bases. As used herein, the term “nucleotide” can include a modified nucleotide (such as, for example, a nucleotide mimic, abasic residue (Ab), or a surrogate replacement moiety).

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that all (100%) of the nucleobases or nucleotides in a contiguous sequence of a first polynucleotide will hybridize with the same number of nucleobases or nucleotides in a contiguous sequence of a second polynucleotide. The contiguous sequence may comprise all or a part of a first or second nucleotide sequence.

As used herein, “partially complementary” means that in a hybridized pair of nucleobase sequences, at least 70%, but not all, of the bases in a contiguous sequence of a first polynucleotide will hybridize with the same number of bases in a contiguous sequence of a second polynucleotide.

As used herein, “substantially complementary” means that in a hybridized pair of nucleobase sequences, at least 85%, but not all, of the bases in a contiguous sequence of a first polynucleotide will hybridize with the same number of bases in a contiguous sequence of a second polynucleotide. The terms “complementary,” “fully complementary,” “partially complementary,” and “substantially complementary” herein are used with respect to the nucleobase or nucleotide matching between the sense strand and the antisense strand of an RNAi agent, or between the antisense strand of an RNAi agent and a sequence of an AAT mRNA.

As used herein, the term “substantially identical” or “substantially identity” as applied to nucleic acid sequence means that a nucleic acid sequence comprises a sequence that has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and treatment” may include the prevention, management, prophylactic treatment, and/or inhibition of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means that delivering the RNAi agent to the cell in a manner that enables the RNAi agent to have the expected biological activity. e.g., sequence-specific inhibition of gene expression.

Unless stated otherwise, use of the symbol [symbol] as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### BRIEF DESCRIPTION OF THE DRAWINGS

FIGS. 1A to 1E represent the chemical duplex structure of AD04828 shown as a sodium salt.

FIGS. 2A to 2E represent the chemical duplex structure of AD04828 shown as a free acid.

FIGS. 3A to 3E represent the chemical duplex structure of AD04831 shown as a sodium salt.

FIG. 4A to 4E represent the chemical duplex structure of AD04831 shown as a free acid.

FIG. 5A to 5E represent the chemical duplex structure of AD04836 shown as a sodium salt.

FIG. 6A to 6E represent the chemical duplex structure of AD04836 shown as a free acid.

FIG. 7A to 7E represent the chemical duplex structure of AD04837 shown as a sodium salt.

FIG. 8A to 8E represent the chemical duplex structure of AD04837 shown as a free acid.

FIG. 9 is a bar graph showing average normalized cynomolgus monkey (cyno) AAT (cAAT) serum levels in cynos (n=3) following a single subcutaneous administration of 3 mg/kg of either AD04824, AD04825, AD04826, or AD(4827, according to Example 4. AAT serum levels were normalized to average pre-treatment values. Experimental error is shown as standard deviation.

FIG. 10 is a bar graph showing average normalized cAAT serum levels in cynos (n=2 or n=3) following a single subcutaneous administration of 3 mg/kg of either AD04828, AD04836, AD04831, or AD04837, according to Example 5. AAT serum levels were normalized to average pre-treatment values. Experimental error is shown as standard deviation.

FIG. 11, is a bar graph showing the results of a western blot analysis of the soluble fractions (Z-AAT monomer) from livers of PiZ mice dosed with either saline or NAG-conjugated AAT RNAi agent having the duplex structure AD04837, dosed for 8 weeks q2w, normalized to baseline control, according to Example 7. Individual mouse measurements are shown grouped by treatment group, and experimental error is shown as standard deviation.

FIG. 12, is a bar graph showing the results of a western blot analysis of the insoluble fractions (Z-AAT polymer) from livers of PiZ mice dosed with either saline or NAG-conjugated AAT RNAi agent having the duplex structure AD04837, according to Example 7. Individual mouse measurements are shown grouped by treatment group, and experimental error is shown as standard deviation.

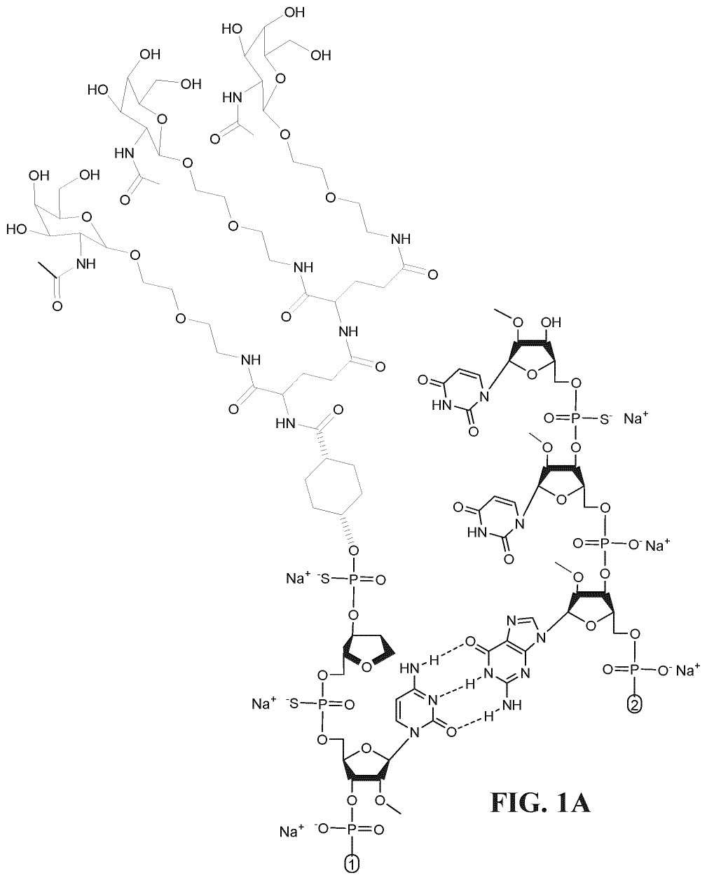

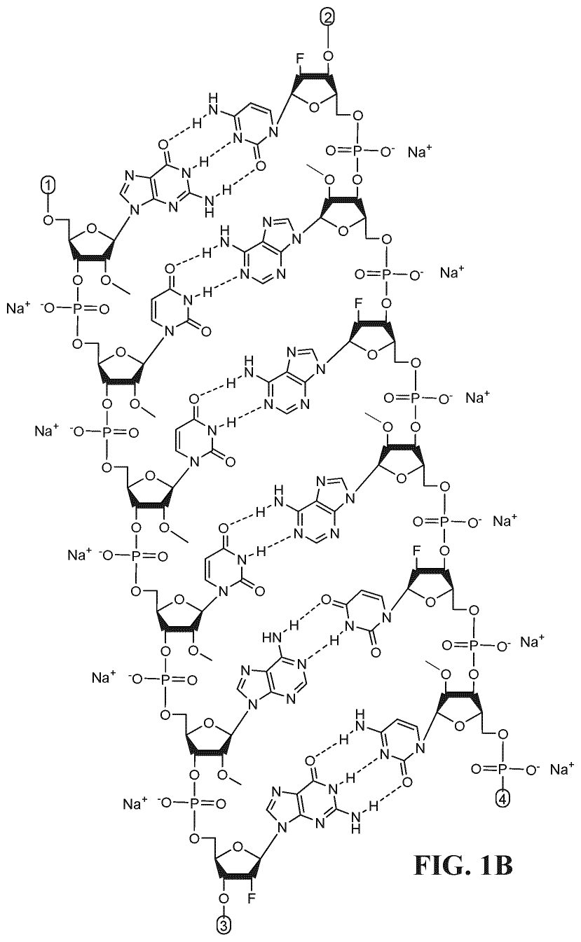

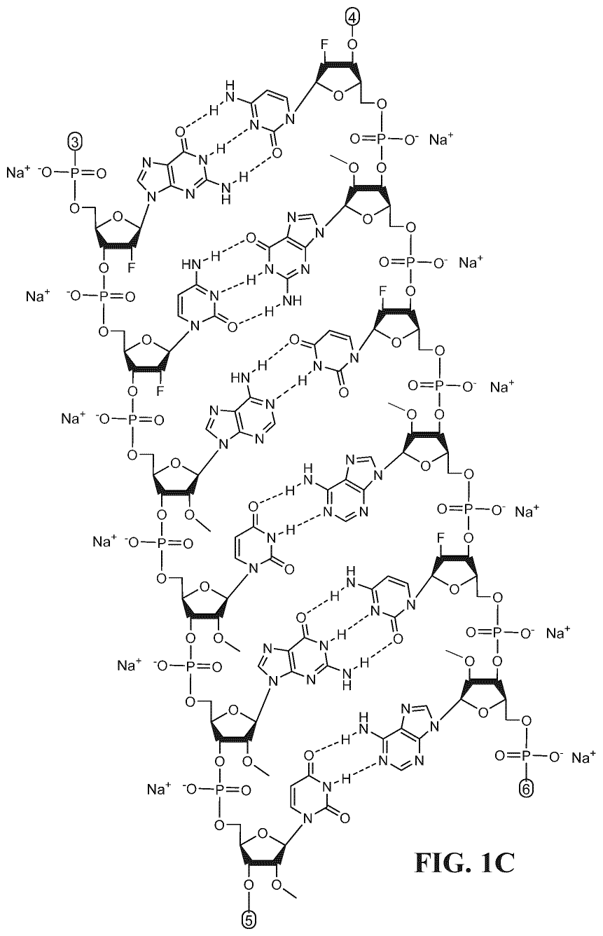

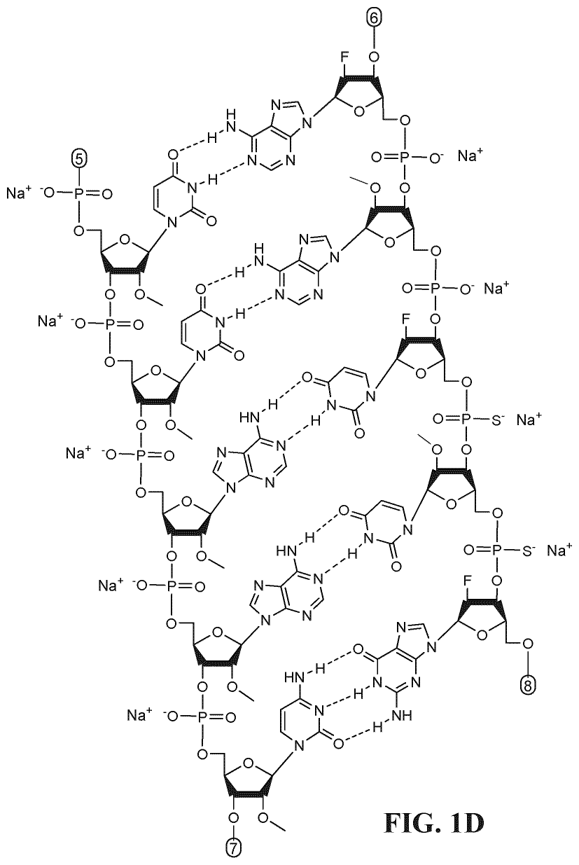

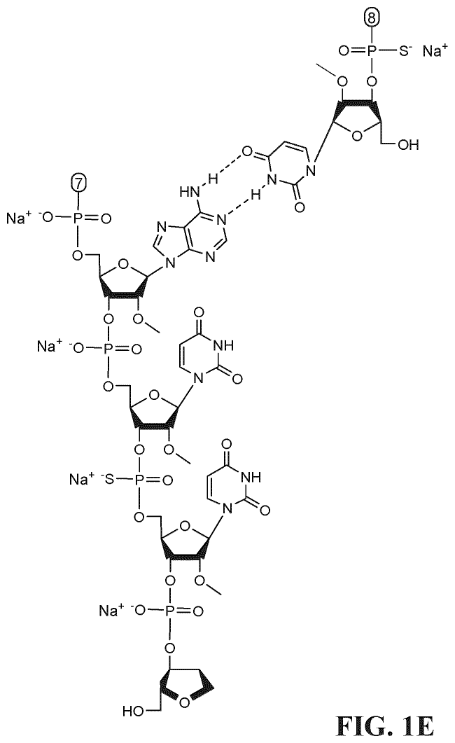

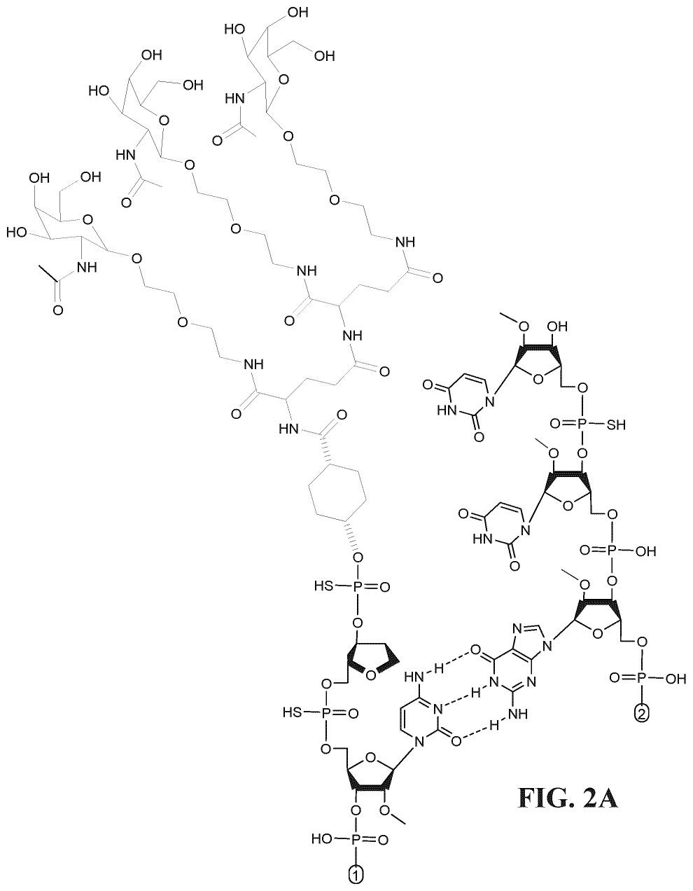

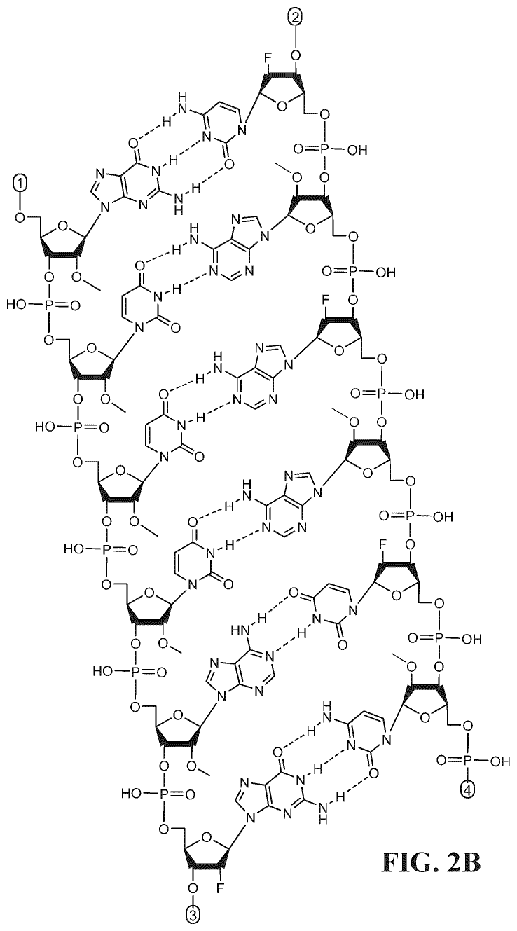

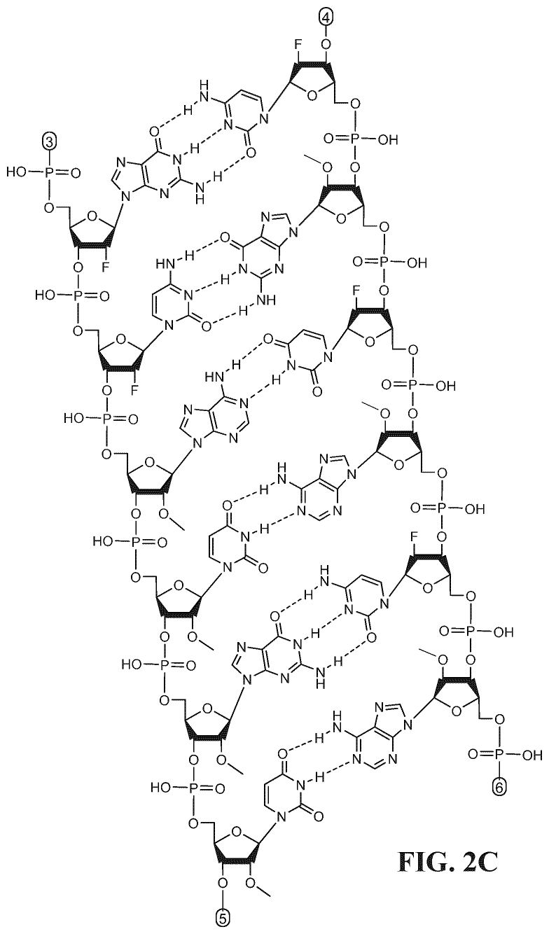

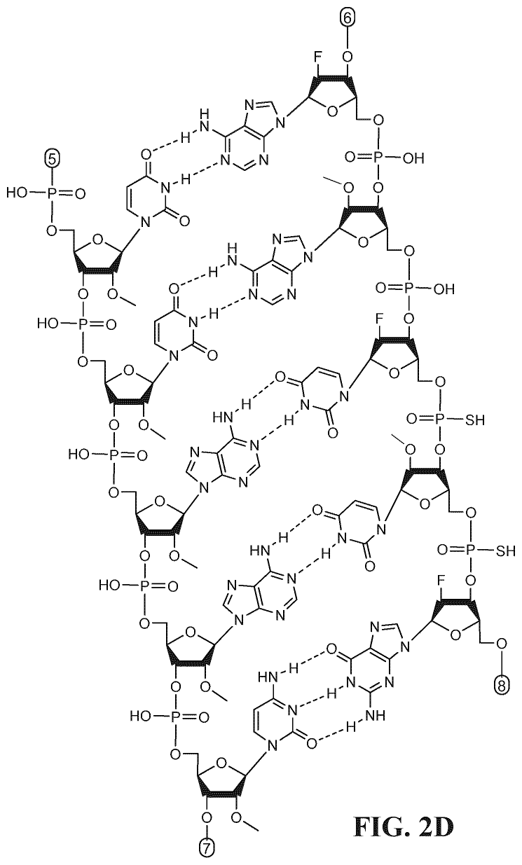

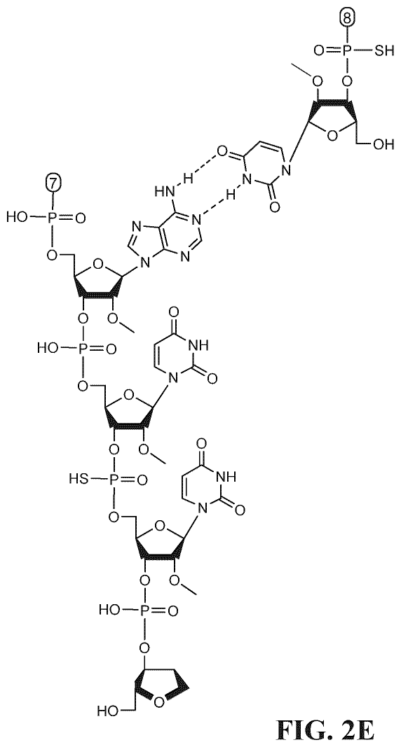

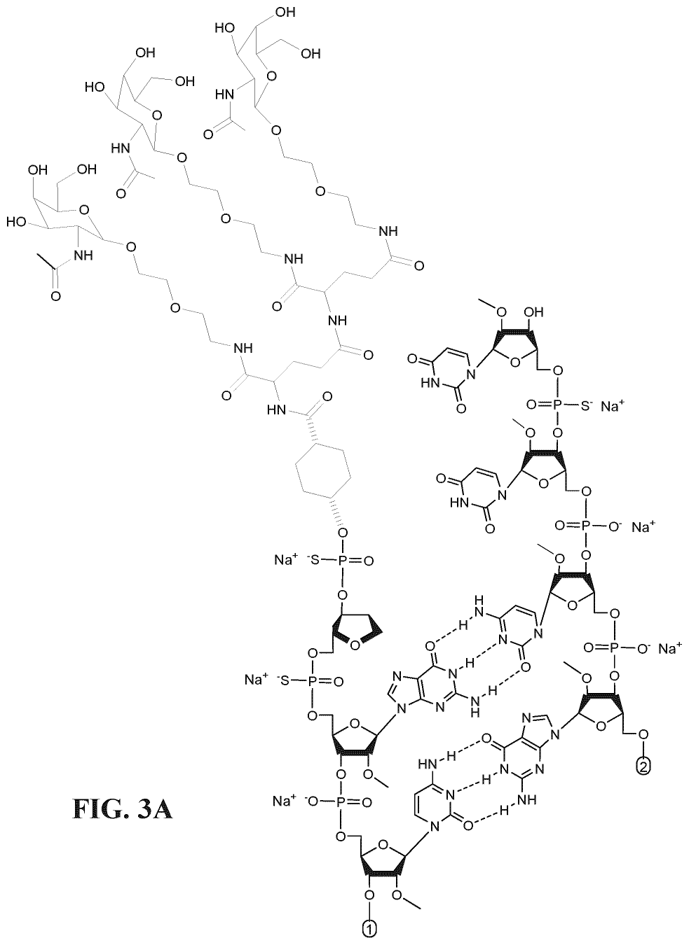

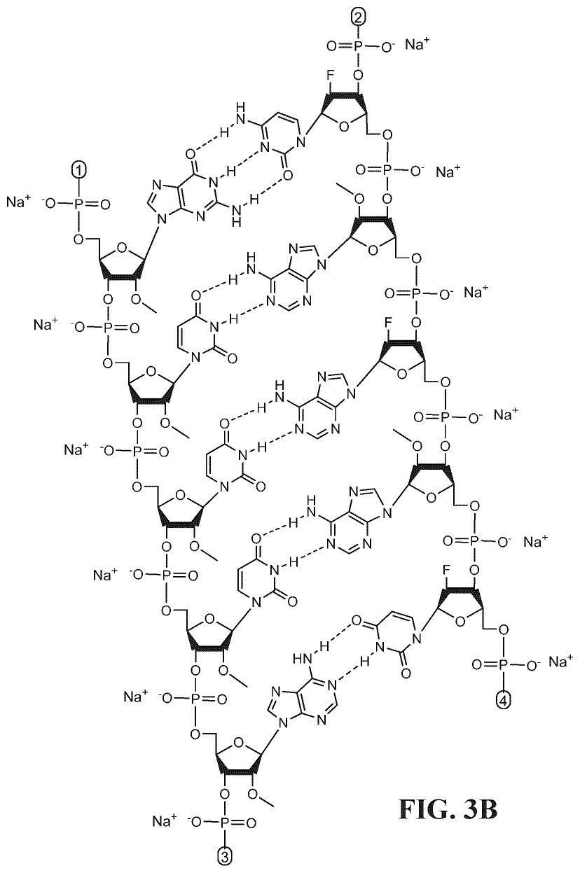

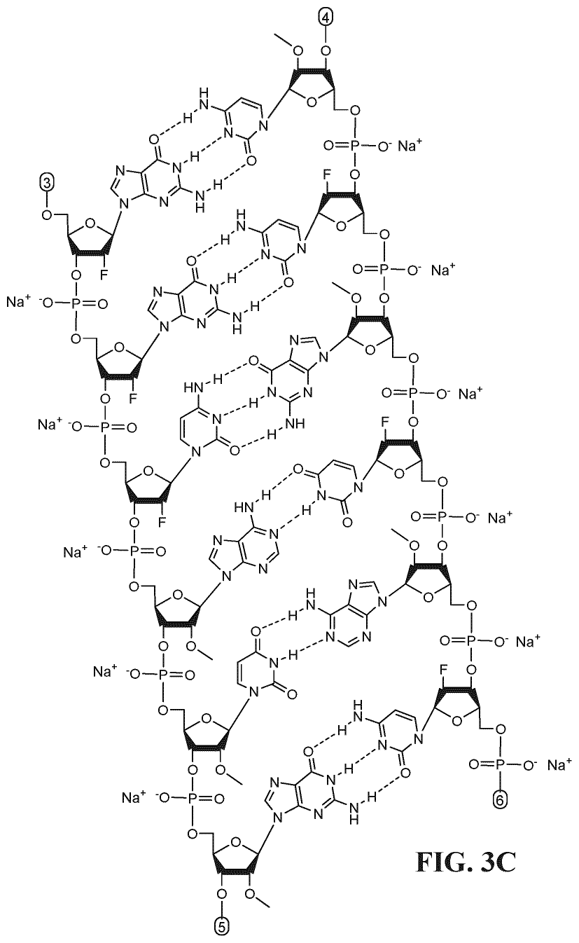

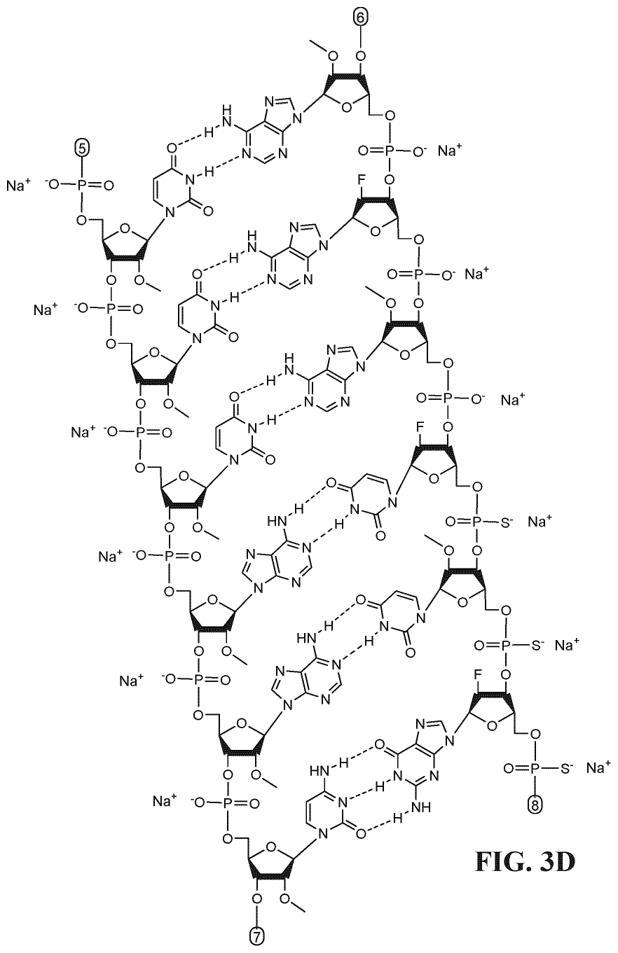

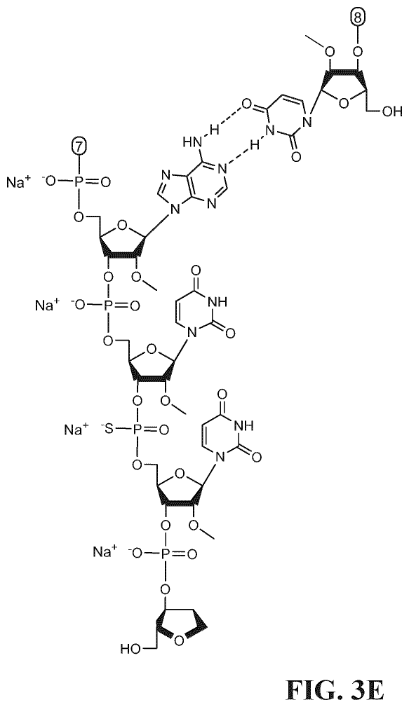

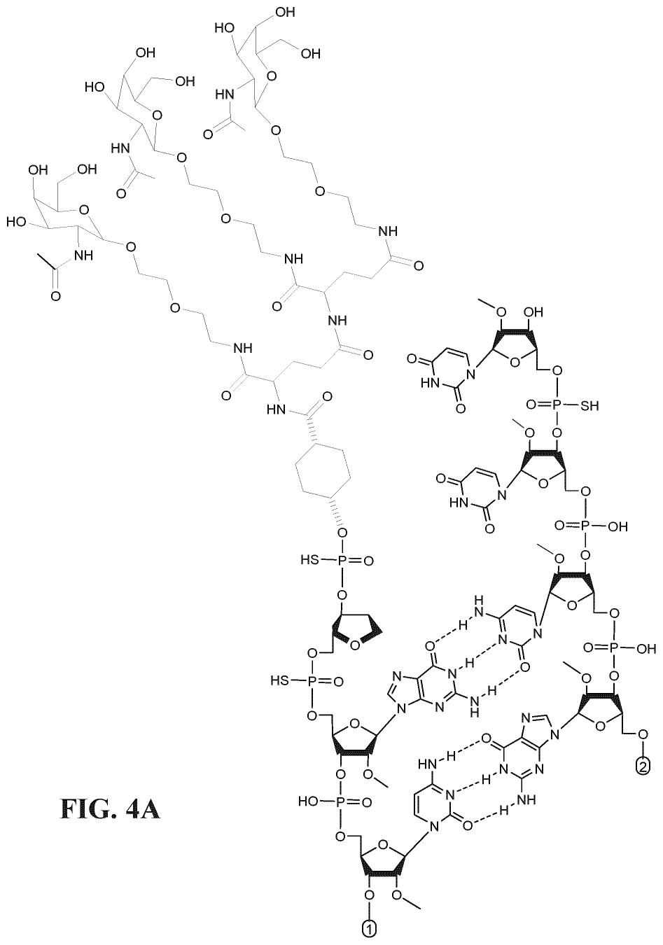

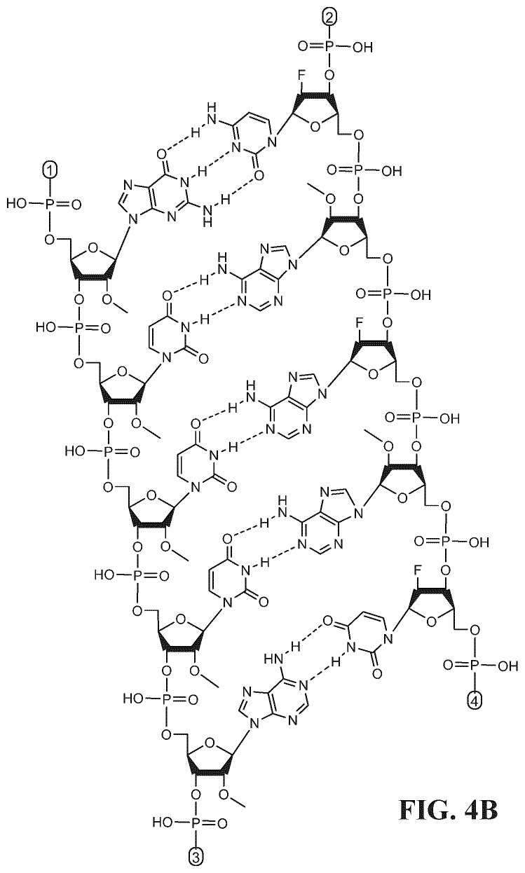

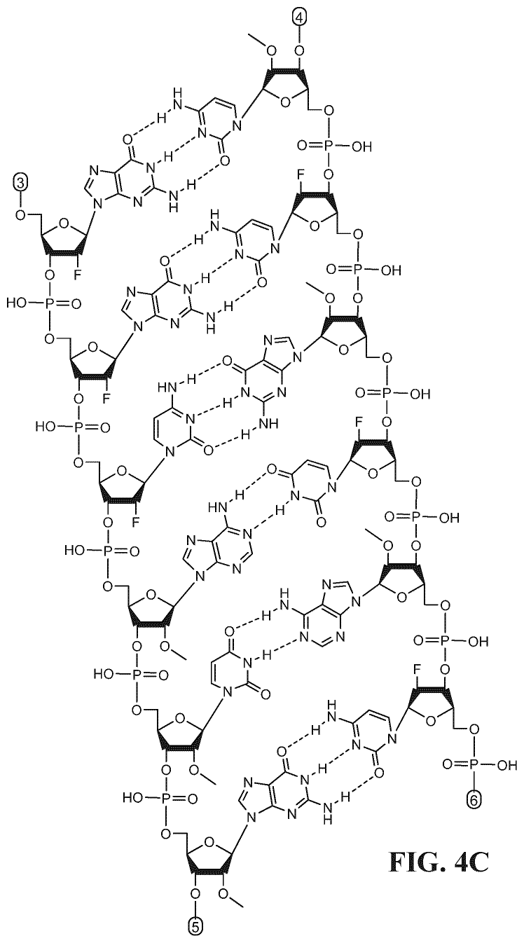

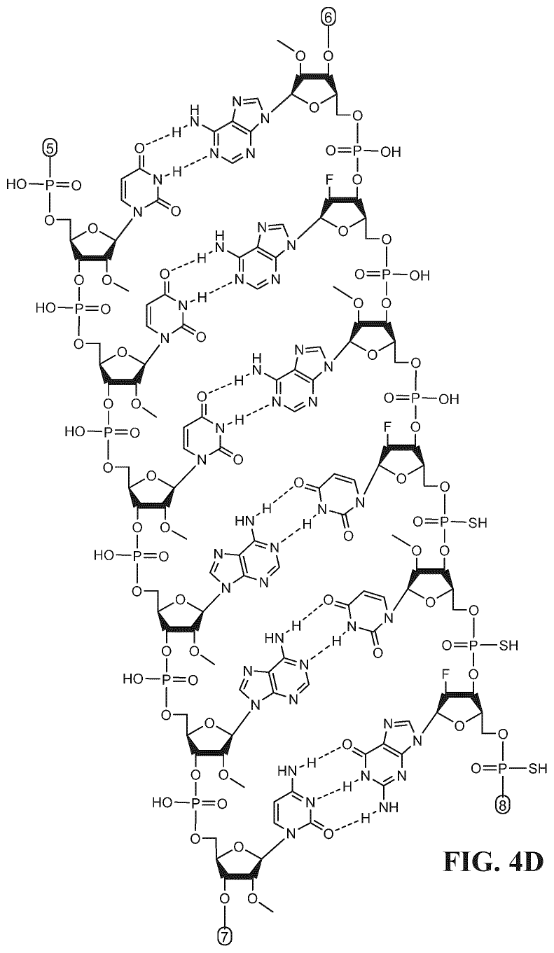

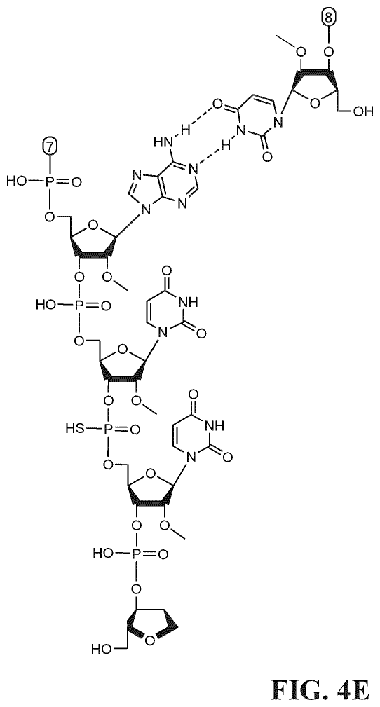

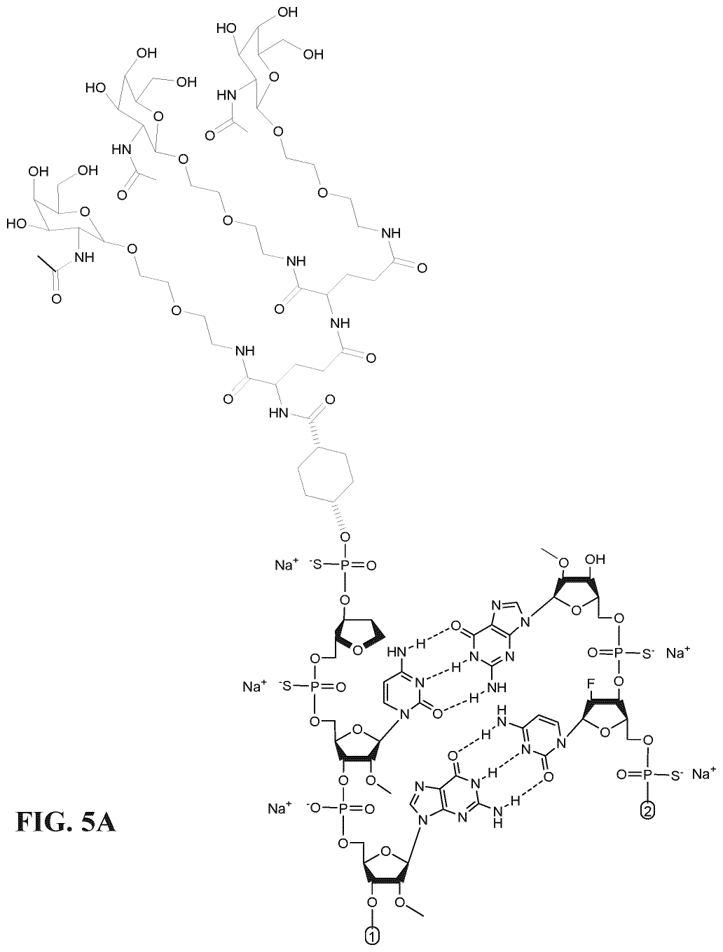

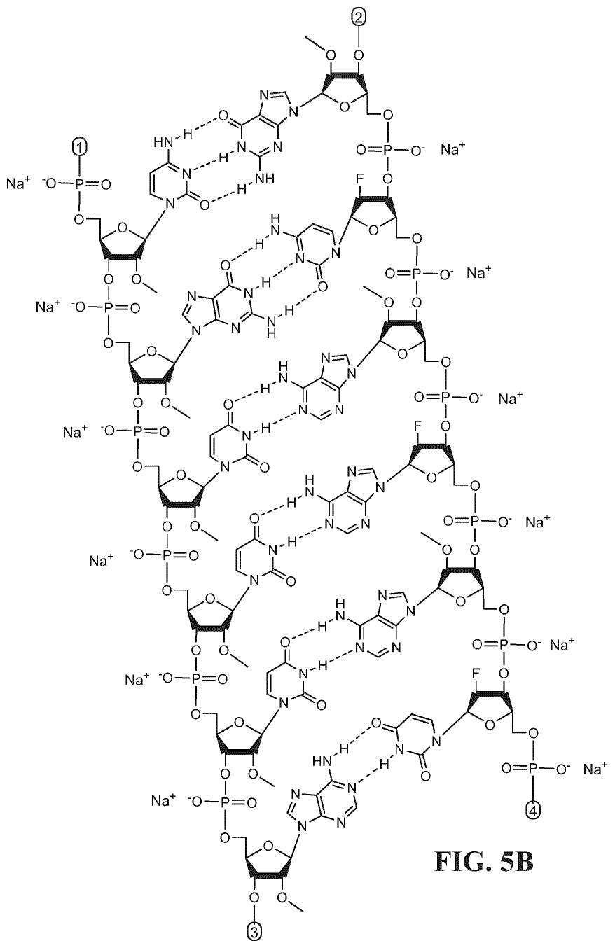

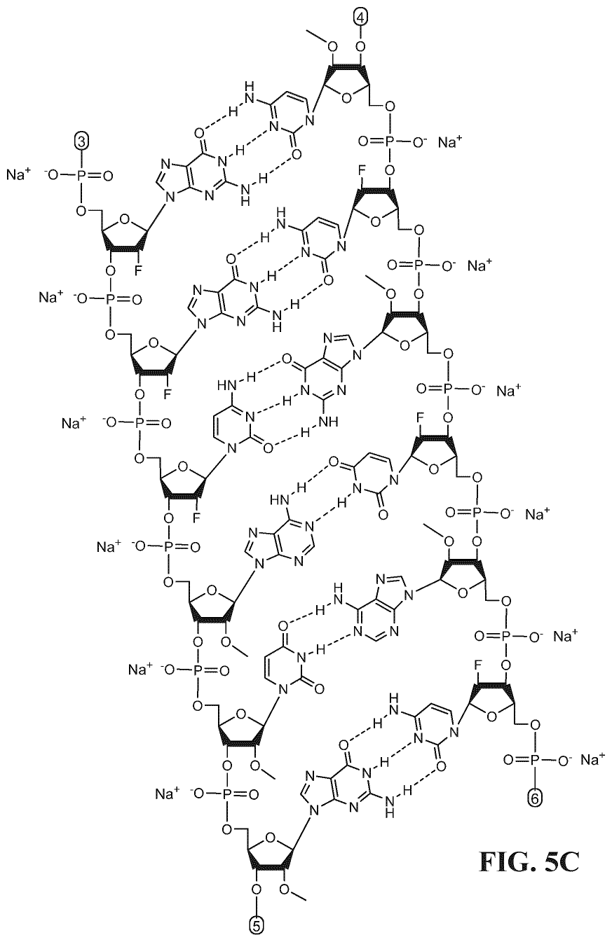

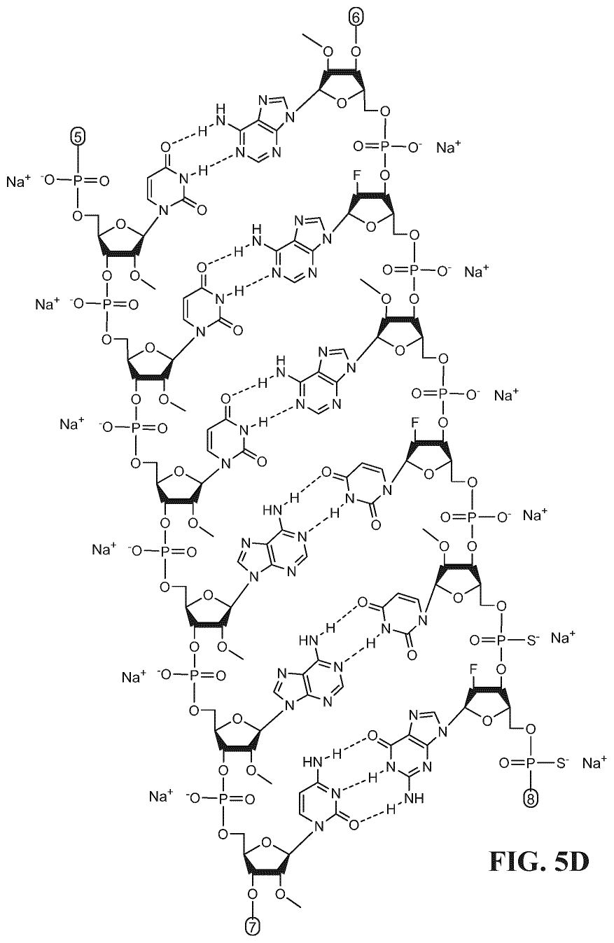

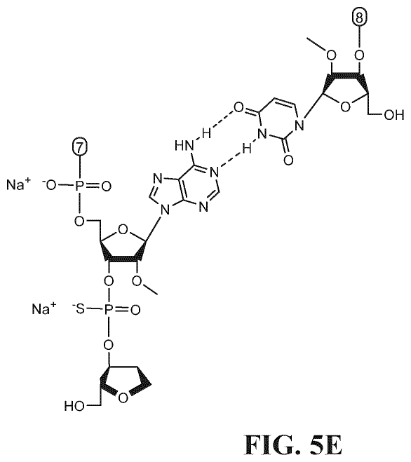

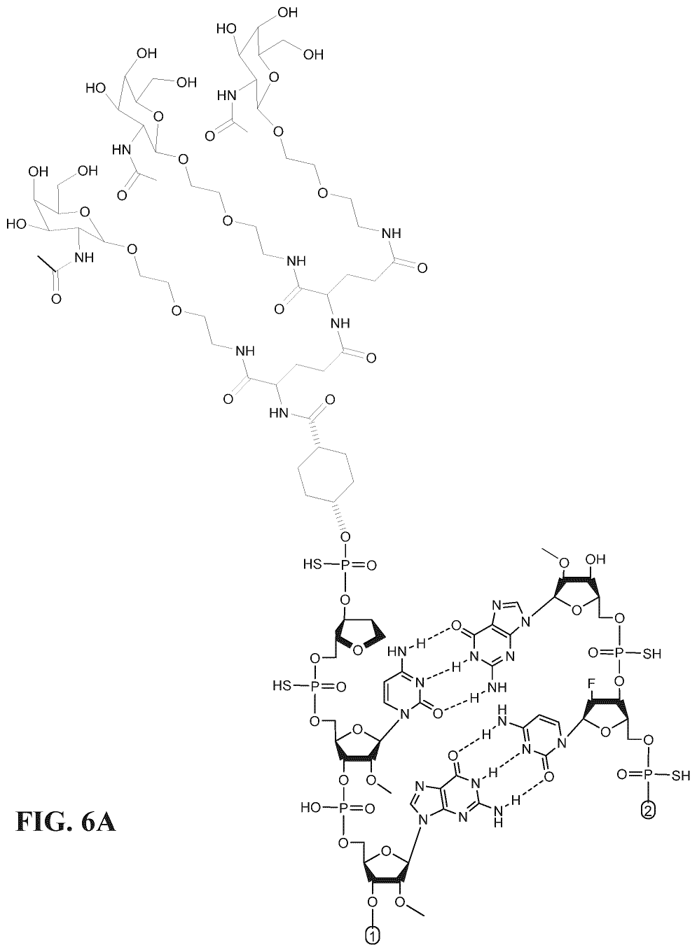

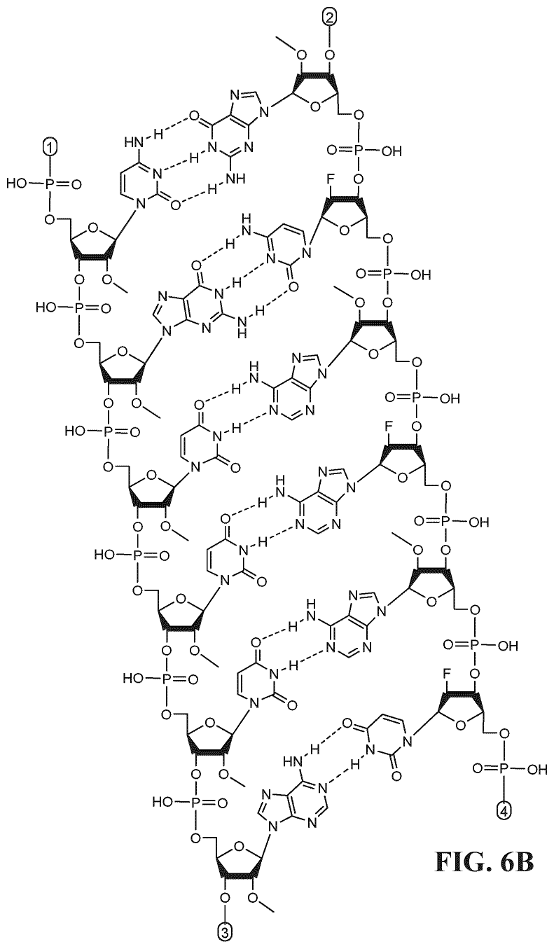

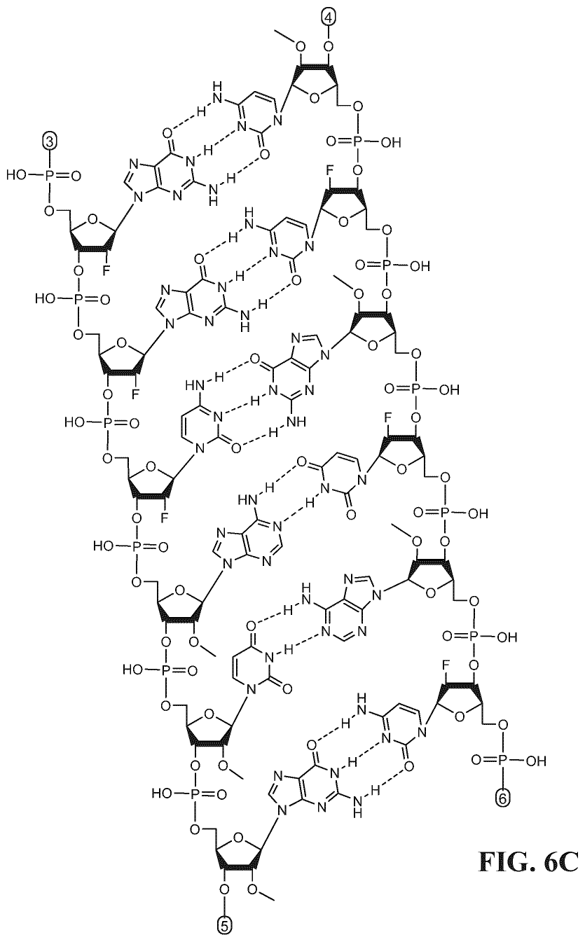

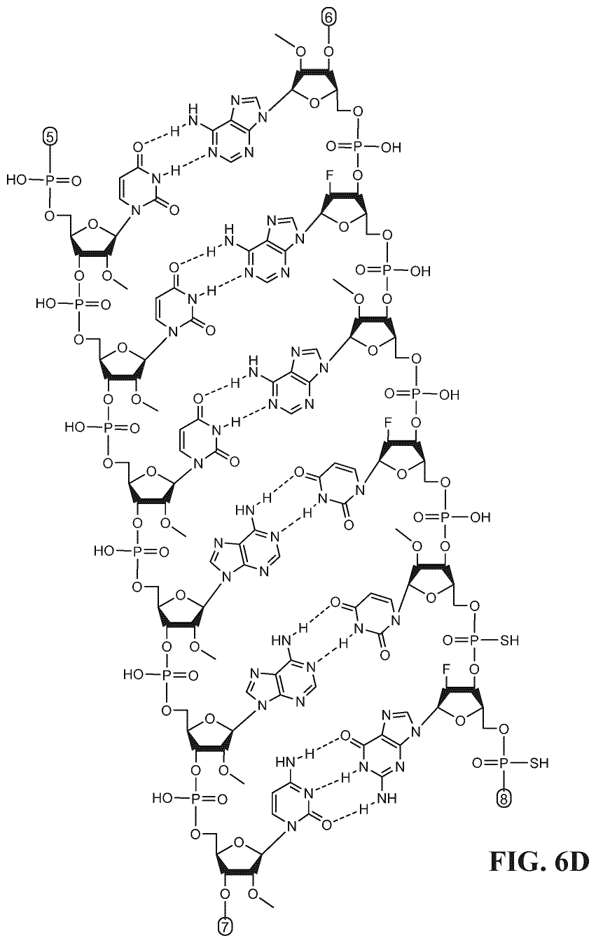

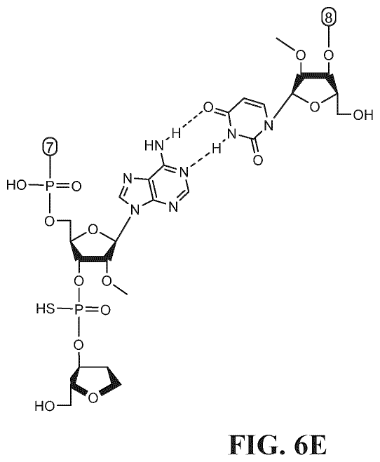

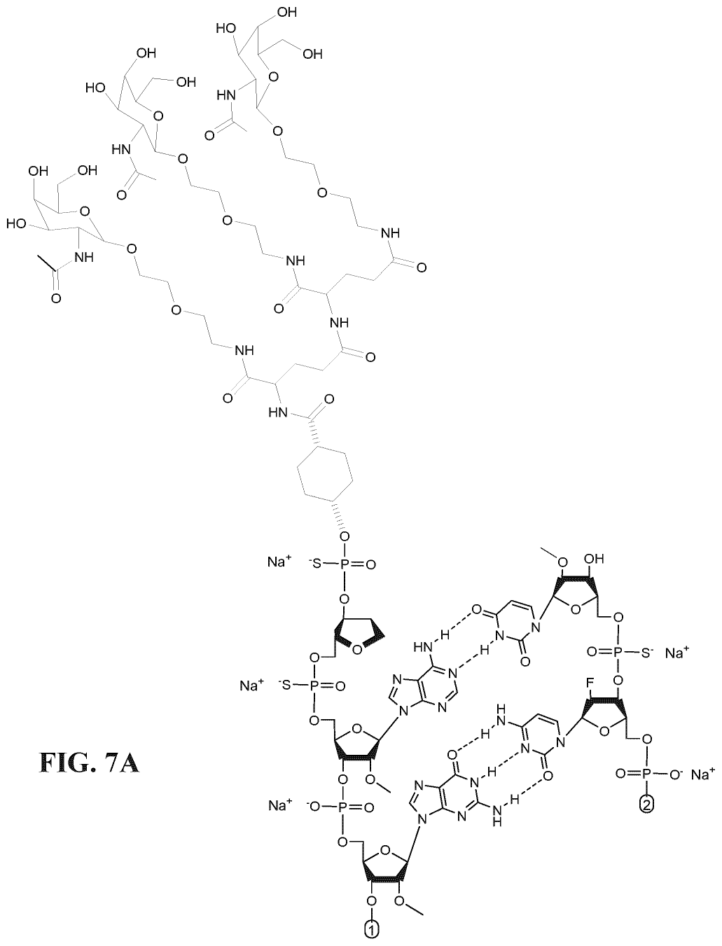

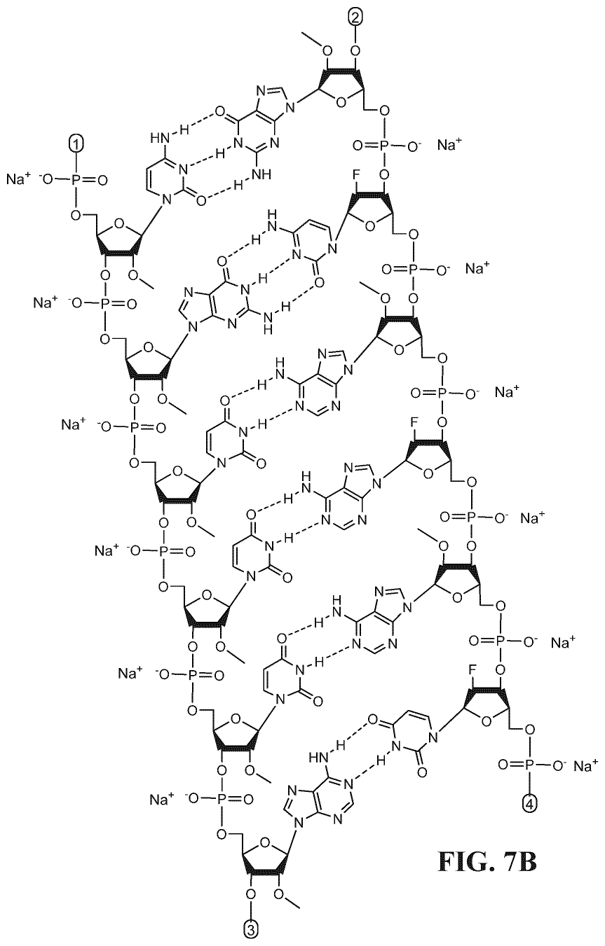

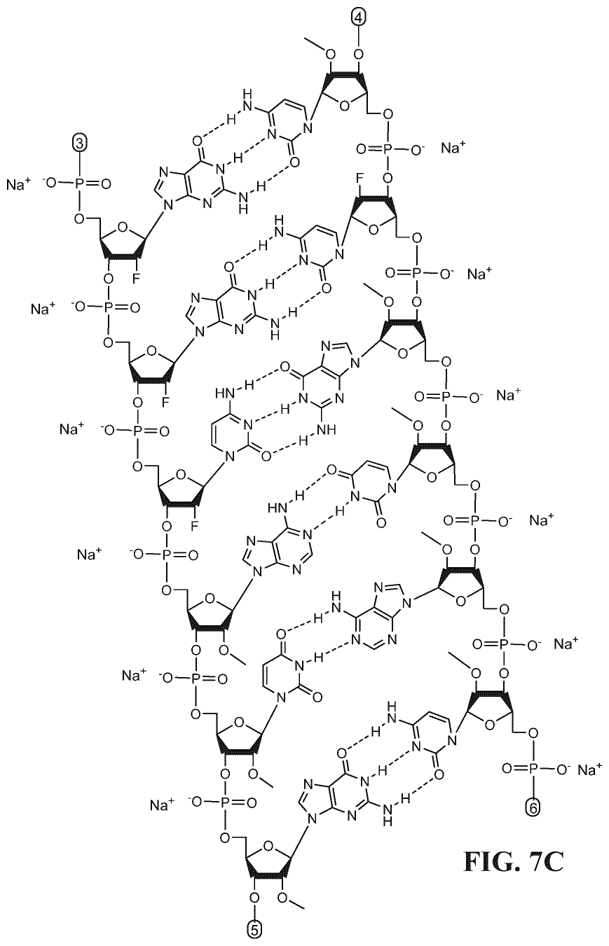

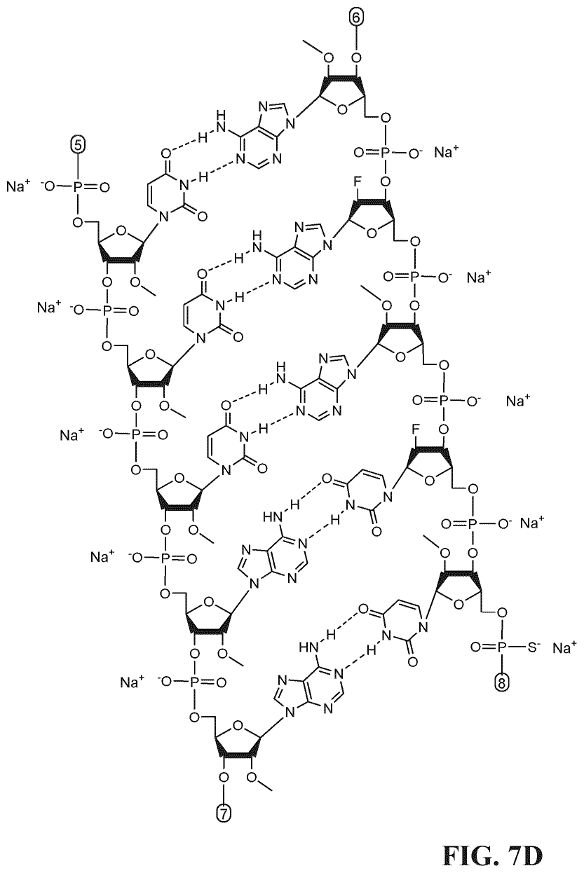

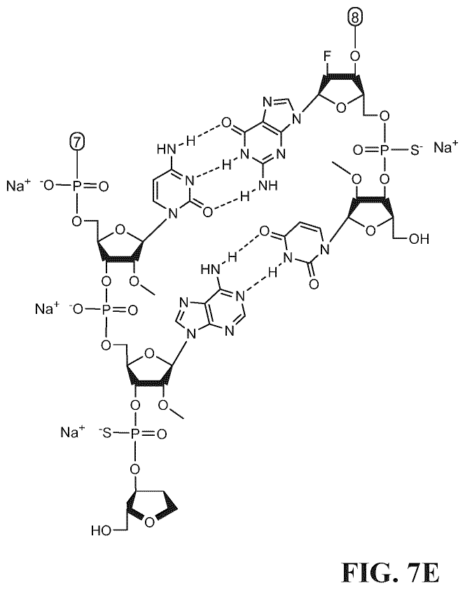

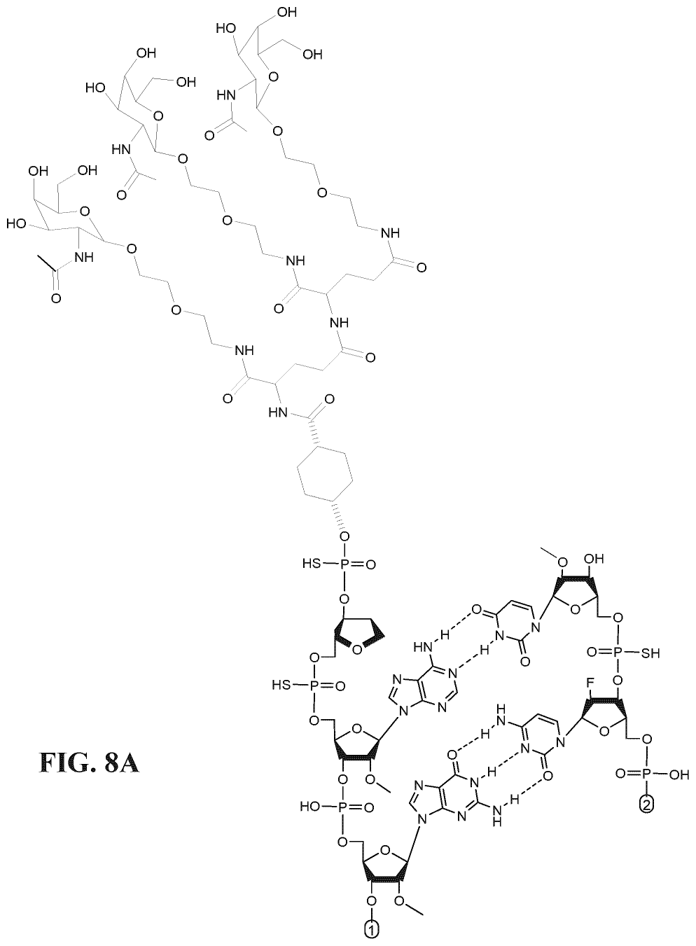

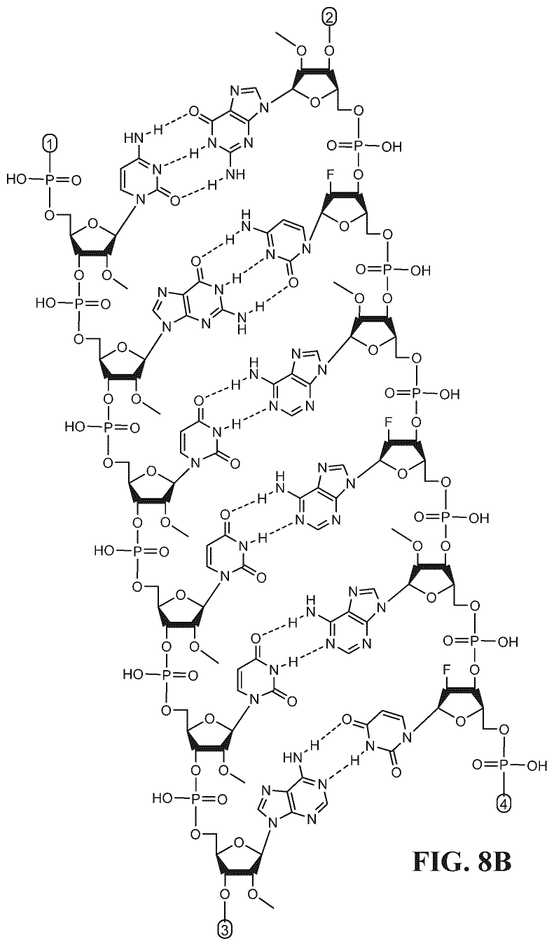

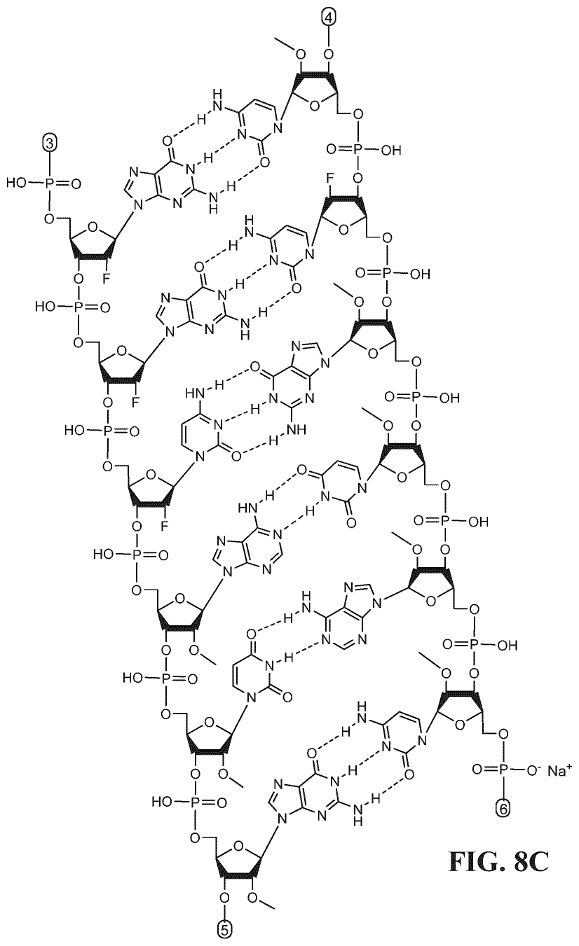

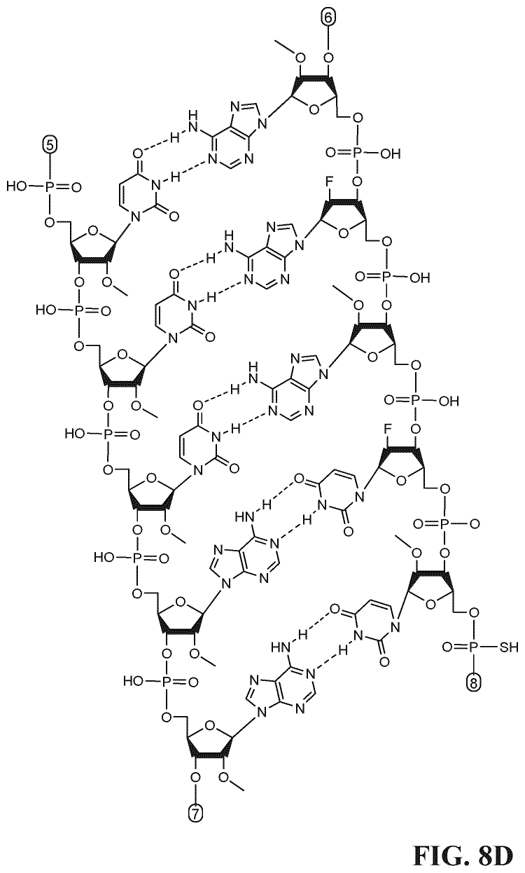

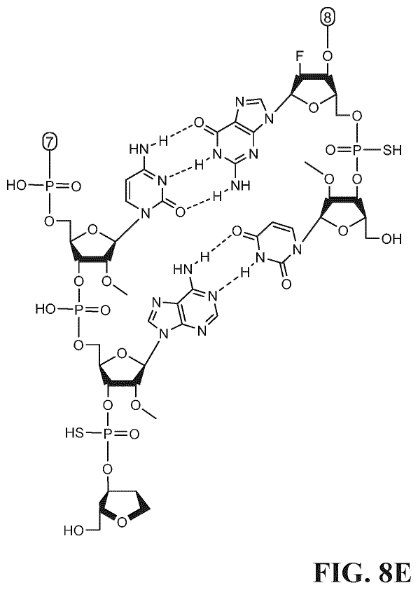

### DETAILED DESCRIPTION

RNAi Agents

Described herein are RNAi agents for inhibiting expression of an AAT gene (referred to herein as AAT RNAi agents or AAT RNAi triggers). Each AAT RNAi agent comprises a sense strand and an antisense strand. The sense strand and the antisense strand each can be 16 to 30 nucleotides in length. In some embodiments, the sense and antisense strands each can be 17 to 26 nucleotides in length. The sense and antisense strands can be either the same length or they can be different lengths. In some embodiments, the sense and antisense strands are each independently 17-21 nucleotides in length. In some embodiments, the sense and antisense strands are each 21-26 nucleotides in length. In some embodiments, the sense and antisense strands are each 21-24 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, a sense strand is 22 nucleotides in length and an antisense strand is 21 nucleotides in length. In some embodiments, a sense strand is 19 nucleotides in length and an antisense strand is 21 nucleotides in length. In some embodiments, the RNAi agent sense and antisense strands are each independently 17, 18, 19, 20, 21, 22, 23, 24, 25, or 26 nucleotides in length. In some embodiments, a double-stranded RNAi agent has a duplex length of about 16, 17, 18, 19, 20, 21, 22, 23 or 24 nucleotides.

In some embodiments, the region of perfect or substantial complementarity between the sense strand and the antisense strand is 16-26 (e.g., 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly or substantially complementary).

The sense strand and antisense strand each contain a core stretch sequence that is 16 to 23 nucleobases in length. An antisense strand core stretch sequence is 100% (perfectly) complementary or at least about 85% (substantially) complementary to a nucleotide sequence (sometimes referred to, e.g., as a target sequence) present in the AAT mRNA target. A sense strand core stretch sequence is 100% (perfectly) complementary or at least about 85% (substantially) complementary to a core stretch sequence in the antisense strand, and thus the sense strand core stretch sequence is perfectly identical or at least about 85% identical to a nucleotide sequence (target sequence) present in the AAT mRNA target. A sense strand core stretch sequence can be the same length as a corresponding antisense core sequence or it can be a different length. In some embodiments, the antisense strand core stretch sequence is 16, 17, 18, 19, 20, 21, 22, or 23 nucleotides in length. In some embodiments, the sense strand core stretch sequence is 16, 17, 18, 19, 20, 21, 22, or 23 nucleotides in length.

Examples of nucleotide sequences used in forming AAT RNAi agents are provided in Tables 2, 3, 4 and 5. Examples of AAT RNAi agent duplexes, that include the sense strand and antisense strand sequences in Tables 2, 3, 4, and 5, are shown in Table 6.

The AAT RNAi agent sense and antisense strands anneal to form a duplex. A sense strand and an antisense strand of an AAT RNAi agent may be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 16, at least 17, at least 18, at least 19, at least 20, at least 21, at least 22, or at least 23 nucleotides that is at least 85% or 100% complementary to a corresponding 16, 17, 18, 19, 20, 21, 22, or 23 nucleotide sequence of the antisense strand core stretch sequence (i.e., the sense and antisense core stretch sequences of an AAT RNAi agent have a region of at least 16, at least 17, at least 18, at least 19, at least 20, at least 21, at least 22, or at least 23 nucleotides that is at least 85% base paired or 100% base paired.)

In some embodiments, the antisense strand of an AAT RNAi agent disclosed herein differs by 0, 1, 2, or 3 nucleotides from any of the antisense strand sequences in Table 2, Table 3, or Table 4. In some embodiments, the sense strand of an AAT RNAi agent disclosed herein differs by 0, 1, 2, or 3 nucleotides from any of the sense strand sequences in Table 2, Table 3, or Table 5.

The sense strand and/or the antisense strand may optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in an AAT mRNA. The sense strand additional nucleotides, if present, may or may not be identical to the corresponding sequence in an AAT mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, an AAT RNAi agent has an antisense strand having a 3′ extension and a sense strand having a 5′ extension.

In some embodiments, an AAT RNAi agent comprises an antisense strand having a 3′ extension of 1, 2, 3, 4, 5, or 6 nucleotides in length. In other embodiments, an AAT RNAi agent comprises an antisense strand having a 3′ extension of 1, 2, or 3 nucleotides in length. In some embodiments, one or more of the antisense strand extension nucleotides comprise uracil or thymidine nucleotides or nucleotides that are complementary to the corresponding AAT mRNA sequence. In some embodiments, a 3′ antisense strand extension includes or consists of one of the following sequences, but is not limited to: AUA, UGCUU, CUG, UG, UGCC, CUGCC, CGU. CUU, UGCCUA, CUGCCU, UGCCU, UGAUU, GCCUAU, T, TT, U, UU (each listed 5′→3′).

In some embodiments, the 3′ end of the antisense strand can include additional abasic residues (Ab). An “abasic residue” or “abasic site” is a nucleotide or nucleoside that lacks a nucleobase at the 1′ position of the sugar. In some embodiments, Ab or AbAb can be added to the 3′ end of the antisense strand. In some embodiments, the abasic residue(s) can be added as inverted abasic residues (invAb) (see Table 7). (See. e.g., F. Czaudema, Nucleic Acids Res., 2003, 31(11), 2705-16).

In some embodiments, an AAT RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to nucleotides in the AAT mRNA sequence. In some embodiments, the 3′ sense strand extension includes or consists of one of the following sequences, but is not limited to: T. UT, TT, UU, UUT, TT, or TIT (each listed 5′ to 3′).

In some embodiments, the 3′ end of the sense strand may include additional abasic residues. In some embodiments, UUAb, UAb, or Ab are added to the 3′ end of the sense strand. In some embodiments, the one or more abasic residues added to the 3′ end of the sense strand are inverted (invAb). In some embodiments, one or more inverted abasic residues or abasic sites may be inserted between the targeting ligand and the nucleobase sequence of the sense strand of the RNAi agent. In some embodiments, the inclusion of one or more inverted abasic residues or abasic sites at or near the terminal end or terminal ends of the sense strand of an RNAi agent allows for enhanced activity or other desired properties of an RNAi agent.

In some embodiments, an AAT RNAi agent comprises a sense strand having a 5′ extension of 1, 2, 3, 4, 5, or 6 nucleotides in length. In some embodiments, one or more of the sense strand extension nucleotides comprise uracil or adenosine nucleotides or nucleotides that correspond to nucleotides in the AAT mRNA sequence. In some embodiments, the sense strand 5′ extension is one of the following sequences, but is not limited to: CA, AUAGGC, AUAGG, AUAG, AUA, A, AA, AC, GCA, GGCA, GGC, UAUCA, UAUC, UCA, UAU, U, UU (each listed 5′ to 3′). A sense strand can have a 3′ extension and/or a 5′ extension.

In some embodiments, the 5′ end of the sense strand can include one or more additional abasic residues (e.g., (Ab) or (AbAb)). In some embodiments, the one or more abasic residues added to the 5′ end of the sense strand can be inverted (e.g., invAb). In some embodiments, one or more inverted abasic residues can be inserted between the targeting ligand and the nucleobase sequence of the sense strand of the RNAi agent. In some embodiments, the inclusion of one or more inverted abasic residues at or near the terminal end or terminal ends of the sense strand of an RNAi agent may allow for enhanced activity or other desired properties of an RNAi agent. In some embodiments, an abasic (deoxyribose) residue can be replaced with a ribitol (abasic ribose) residue.

In some embodiments, the 3′ end of the antisense strand core stretch sequence, or the 3′ end of the antisense strand sequence, may include an inverted abasic residue (invAb (see Table 7)).

Examples of sequences used in forming AAT RNAi agents are provided in Tables 2, 3, 4, and 5. In some embodiments, an AAT RNAi agent antisense strand includes a sequence of any of the sequences in Tables 2, 3, or 4. In some embodiments, an AAT RNAi agent antisense strand includes the sequence of nucleotides (from 5′ end→3′ end) 1-17, 2-15, 2-17, 1-18, 2-18, 1-19, 2-19, 1-20, 2-20, 1-21, 2-21, 1-22, 2-22, 1-23, 2-23, 1-24, or 2-24, of any of the sequences in Table 2. Table 3, or Table 4. In certain embodiments, an AAT RNAi agent antisense strand comprises or consists of a modified sequence of any one of the modified sequences in Table 4. In some embodiments, an AAT RNAi agent sense strand includes the sequence of any of the sequences in Tables 2, 3, or 5. In some embodiments, an AAT RNAi agent sense strand includes the sequence of nucleotides (from 5′ end→3′ end) 1-18, 1-19, 1-20, 1-21, 1-22, 1-23, 1-24, 2-19, 2-20, 2-21, 2-22, 2-23, 2-24, 3-20, 3-21, 3-22, 3-23, 3-24, 4-21, 4-22, 4-23, 4-24, 5-22, 5-23, 5-24, 6-23, 6-24, 7-24, of any of the sequences in Tables 2, 3, or 5. In certain embodiments, an AAT RNAi agent sense strand comprises or consists of a modified sequence of any one of the modified sequences in Table 5.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a blunt end refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands from a pair (i.e., do not form an overhang) but are not complementary (i.e. form a non-complementary pair). As used herein, an overhang is a stretch of one or more unpaired nucleotides at the end of one strand of a double stranded RNAi agent. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends.

Modified nucleotides, when used in various polynucleotide or oligonucleotide constructs, can preserve activity of the compound in cells while at the same time increasing the serum stability of these compounds, and can also minimize the possibility of activating interferon activity in humans upon administering of the polynucleotide or oligonucleotide construct.

In some embodiments, an AAT RNAi agent is prepared or provided as a salt, mixed salt, or a free-acid. In some embodiments, an AAT RNAi agent is prepared as a sodium salt. Such forms are within the scope of the inventions disclosed herein.

Modified Nucleotides

In some embodiments, an AAT RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as N_{UNA or} NUNA), locked nucleotides (represented herein as N_{LNA} or NLNA), 3′-O-methoxy (2′ internucleoside linked) nucleotides (represented herein as 3′-OMen), 2′-F-Arabino nucleotides (represented herein as NfANA or NANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxyethyl (2′-O-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification can be incorporated in a single AAT RNAi agent or even in a single nucleotide thereof. The AAT RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4)nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides. In some embodiments, one or more nucleotides of an RNAi agent is a ribonucleotide.

Modified Internucleoside Linkages

In some embodiments, one or more nucleotides of an AAT RNAi agent are linked by non-standard linkages or backbones (i.e., modified internucleoside linkages or modified backbones). Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an AAT RNAi agent can contain 1, 2, 3, 4, 5, or 6 phosphorothioate linkages, an antisense strand of an AAT RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an AAT RNAi agent can contain 1, 2, 3, or 4 phosphorothioate linkages, an antisense strand of an AAT RNAi agent can contain 1, 2, 3, or 4 phosphorothioate linkages, or both the sense strand and the antisense strand independently can contain 1, 2, 3, or 4 phosphorothioate linkages.

In some embodiments, an AAT RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, an AAT RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, an AAT RNAi agent contains at least two phosphorothioate internucleoside linkages in the sense strand and three or four phosphorothioate internucleoside linkages in the antisense strand.

In some embodiments, an AAT RNAi agent contains one or more modified nucleotides and one or more modified internucleoside linkages. In some embodiments, a 2′-modified nucleoside is combined with modified internucleoside linkage.

AAT RNAi Agents

In some embodiments, the AAT RNAi agents disclosed herein target an AAT gene at or near the positions of the AAT gene shown in Table 1. In some embodiments, the antisense strand of an AAT RNAi agent disclosed herein includes a core stretch sequence that is fully, substantially, or at least partially complementary to a target AAT 19-mer sequence disclosed in Table 1.

TABLE 1
AAT 19-mer mRNA Target Sequences
(taken from human AAT cDNA, GenBank
NM_000295.4 (SEQ ID NO: 1))

| | | Corresponding |
|---|---|---|
| | | Gene |
| SEQ | AAT 19-mer | Position |
| ID | Target Sequences | (taken from |
| NO: | (5′ → 3′) | SEQ ID NO: 1) |
| 2 | CGUUUAGGCAUGUUUAACA | 1000-1018 |
| 3 | AACAGCACCAAUAUCUUCU | 469-487 |
| 4 | AUAUCAUCACCAAGUUCCU | 1142-1160 |
| 5 | AGAUGCUGCCCAGAAGACA | 348-366 |
| 6 | CUGGCACACCAGUCCAACA | 454-472 |
| 7 | UGGCACACCAGUCCAACAG | 455-473 |
| 8 | GCACACCAGUCCAACAGCA | 457-475 |
| 9 | CAGUCCAACAGCACCAAUA | 463-481 |
| 10 | AGUCCAACAGCACCAAUAU | 464-482 |
| 11 | GUCCAACAGCACCAAUAUC | 465-483 |
| 12 | CCAACAGCACCAAUAUCUU | 467-485 |
| 13 | CCCCAGUGAGCAUCGCUAC | 491-509 |
| 14 | GAGCAUCGCUACAGCCUUU | 498-516 |
| 15 | GCAUCGCUACAGCCUUUGC | 500-518 |
| 16 | CAUCGCUACAGCCUUUGCA | 501-519 |
| 17 | UCGCUACAGCCUUUGCAAU | 503-521 |
| 18 | CUACAGCCUUUGCAAUGCU | 506-524 |
| 19 | ACAGCCUUUGCAAUGCUCU | 508-526 |
| 20 | GAAGGCUUCCAGGAACUCC | 613-631 |
| 21 | UAGUGGAUAAGUUUUUGGA | 710-728 |
| 22 | UGUACCACUCAGAAGCCUU | 743-761 |
| 23 | GUACCACUCAGAAGCCUUC | 744-762 |
| 24 | ACACCGAAGAGGCCAAGAA | 779-797 |
| 25 | ACCGAAGAGGCCAAGAAAC | 781-799 |
| 26 | AGGCCAAGAAACAGAUCAA | 788-806 |
| 27 | GGCCAAGAAACAGAUCAAC | 789-807 |
| 28 | GCCAAGAAACAGAUCAACG | 790-808 |
| 29 | UACUCAAGGGAAAAUUGUG | 825-843 |
| 30 | CUCAAGGGAAAAUUGUGGA | 827-845 |
| 31 | UCAAGGGAAAAUUGUGGAU | 828-846 |
| 32 | UUGGUCAAGGAGCUUGACA | 847-865 |
| 33 | AGGAGCUUGACAGAGACAC | 854-872 |
| 34 | AGCUUGACAGAGACACAGU | 857-875 |
| 35 | UUUGCUCUGGUGAAUUACA | 877-895 |
| 36 | AGCGUUUAGGCAUGUUUAA | 998-1016 |
| 37 | GCGUUUAGGCAUGUUUAAC | 999-1017 |
| 38 | UUAGGCAUGUUUAACAUCC | 1003-1021 |
| 39 | UGGGUGCUGCUGAUGAAAU | 1045-1063 |
| 40 | UGCCACCGCCAUCUUCUUC | 1074-1092 |
| 41 | CCUGGAAAAUGAACUCACC | 1119-1137 |
| 42 | CGAUAUCAUCACCAAGUUC | 1140-1158 |
| 43 | ACCAAGUUCCUGGAAAAUG | 1150-1168 |
| 44 | UCCAUUACUGGAACCUAUG | 1207-1225 |
| 45 | CCAUUACUGGAACCUAUGA | 1208-1226 |
| 46 | ACUGGAACCUAUGAUCUGA | 1213-1231 |
| 47 | GGAACCUAUGAUCUGAAGA | 1216-1234 |
| 48 | GAACCUAUGAUCUGAAGAG | 1217-1235 |
| 49 | CAGCAAUGGGGCUGACCUC | 1269-1287 |
| 50 | GCAAUGGGGCUGACCUCUC | 1271-1289 |
| 51 | AGAGGAGGCACCCCUGAAG | 1299-1317 |
| 52 | AGGCACCCCUGAAGCUCUC | 1304-1322 |
| 53 | UCUCCAAGGCCGUGCAUAA | 1319-1337 |
| 54 | UCCAAGGCCGUGCAUAAGG | 1321-1339 |
| 55 | CCAAGGCCGUGCAUAAGGC | 1322-1340 |
| 56 | CAAGGCCGUGCAUAAGGCU | 1323-1341 |
| 57 | AAGGCUGUGCUGACCAUCG | 1336-1354 |
| 58 | GGCUGUGCUGACCAUCGAC | 1338-1356 |
| 59 | CUGCUGGGGCCAUGUUUUU | 1373-1391 |
| 60 | GCUGGGGCCAUGUUUUUAG | 1375-1393 |
| 61 | CUGGGGCCAUGUUUUUAGA | 1376-1394 |
| 62 | GGGGCCAUGUUUUUAGAGG | 1378-1396 |
| 63 | GGGCCAUGUUUUUAGAGGC | 1379-1397 |
| 64 | GAGGCCAUACCCAUGUCUA | 1393-1411 |
| 65 | GGCCAUACCCAUGUCUAUC | 1395-1413 |
| 66 | CCCGAGGUCAAGUUCAACA | 1417-1435 |
| 67 | AGGUCAAGUUCAACAAACC | 1421-1439 |
| 68 | CAAGUUCAACAAACCCUUU | 1425-1443 |
| 69 | AGUUCAACAAACCCUUUGU | 1427-1445 |
| 70 | GUUCAACAAACCCUUUGUC | 1428-1446 |
| 71 | UCAACAAACCCUUUGUCUU | 1430-1448 |
| 72 | ACCCUUUGUCUUCUUAAUG | 1437-1455 |
| 73 | CCUUUGUCUUCUUAAUGAU | 1439-1457 |
| 74 | UACCAAGUCUCCCCUCUUC | 1467-1485 |
| 75 | AAGUCUCCCCUCUUCAUGG | 1471-1489 |
| 76 | AGUCUCCCCUCUUCAUGGG | 1472-1490 |
| 77 | UCUCCCCUCUUCAUGGGAA | 1474-1492 |
| 78 | CUCCCCUCUUCAUGGGAAA | 1475-1493 |
| 79 | AUGACAUUAAAGAAGGGUU | 1569-1587 |

In some embodiments, an AAT RNAi agent includes an antisense strand wherein position 19 of the antisense strand (5′→3′) is capable of forming a base pair with position 1 of a 19-mer target sequence disclosed in Table 1. In some embodiments, an AAT RNAi agent includes an antisense strand wherein position 1 of the antisense strand (5′→3′) is capable of forming a base pair with position 19 of the 19-mer target sequence disclosed in Table 1.

In some embodiments, an AAT RNAi agent includes an antisense strand wherein position 2 of the antisense strand (5′→3′) is capable of forming a base pair with position 18 of the 19-mer target sequence disclosed in Table 1. In some embodiments, an AAT RNAi agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′end→3′ end) can be perfectly complementary to the AAT gene, or can be non-complementary to the AAT gene. In some embodiments, the nucleotide at position 1 of the antisense strand (from 5′end→3′ end) is a U, A, or dT. In some embodiments, the nucleotide at position 1 of the antisense strand (from 5′ end→3′ end) forms an A:U or U:A base pair with the sense strand.

In some embodiments, an AAT RNAi agent antisense strand comprises the sequence of nucleotides (from 5′ end→3′ end) 2-18 or 2-19 of any of the antisense strand sequences in Table 2, Table 3, or Table 4. In some embodiments, an AAT RNAi sense strand comprises the sequence of nucleotides (from 5′ end→3′ end) 1-17, 1-18, or 2-18 of any of the sense strand sequences in Table 2, Table 3, or Table 5.

In some embodiments, an AAT RNAi agent is comprised of (i) an antisense strand comprising the sequence of nucleotides (from 5′ end→3′ end) 2-18 or 2-19 of any of the antisense strand sequences in Table 2, Table 3, or Table 4, and (ii) a sense strand comprising the sequence of nucleotides (from 5′ end→3′ end) 1-17 or 1-18 of any of the sense strand sequences in Table 2, Table 3, or Table 5.

In some embodiments, the AAT RNAi agents include core 19-mer nucleotide sequences shown in the following Table 2.

TABLE 2
Example AAT RNAi Agent Antisense Strand and
Sense Strand Core Stretch Base Sequences
(N = any nucleobase)

| | Antisense | | Sense Base | Gene |
|---|---|---|---|---|
| | Base Sequence | | Sequence | Position |
| SEQ ID | (5′ → 3′) | SEQ ID | (5′ → 3′) | of SEQ ID |
| NO: | (19-mers) | NO: | (19-mers) | NO: 1 |
| 80 | UGUUAAACAUGCCUAAACG | 429 | CGUUUAGGCAUGUUUAACA | 1000-1018 |
| 81 | AGUUAAACAUGCCUAAACG | 430 | CGUUUAGGCAUGUUUAACU | 1000-1018 |
| 82 | NGUUAAACAUGCCUAAACG | 431 | CGUUUAGGCAUGUUUAACN | 1000-1018 |
| 83 | NGUUAAACAUGCCUAAACN | 432 | NGUUUAGGCAUGUUUAACN | 1000-1018 |
| 84 | AGAAGAUAUUGGUGCUGUU | 433 | AACAGCACCAAUAUCUUCU | 469-487 |
| 85 | UGAAGAUAUUGGUGCUGUU | 434 | AACAGCACCAAUAUCUUCA | 469-487 |
| 86 | NGAAGAUAUUGGUGCUGUU | 435 | AACAGCACCAAUAUCUUCN | 469-487 |
| 87 | NGAAGAUAUUGGUGCUGUN | 436 | NACAGCACCAAUAUCUUCN | 469-487 |
| 88 | AGGAACUUGGUGAUGAUAU | 437 | AUAUCAUCACCAAGUUCCU | 1142-1160 |
| 89 | UGGAACUUGGUGAUGAUAU | 438 | AUAUCAUCACCAAGUUCCA | 1142-1160 |
| 90 | NGGAACUUGGUGAUGAUAU | 439 | AUAUCAUCACCAAGUUCCN | 1142-1160 |
| 91 | NGGAACUUGGUGAUGAUAN | 440 | NUAUCAUCACCAAGUUCCN | 1142-1160 |
| 92 | UGUCUUCUGGGCAGCAUCU | 441 | AGAUGCUGCCCAGAAGACA | 348-366 |
| 93 | AGUCUUCUGGGCAGCAUCU | 442 | AGAUGCUGCCCAGAAGACU | 348-366 |
| 94 | NGUCUUCUGGGCAGCAUCU | 443 | AGAUGCUGCCCAGAAGACN | 348-366 |
| 95 | NGUCUUCUGGGCAGCAUCN | 444 | NGAUGCUGCCCAGAAGACN | 348-366 |
| 96 | UGUUGGACUGGUGUGCCAG | 445 | CUGGCACACCAGUCCAACA | 454-472 |
| 97 | AGUUGGACUGGUGUGCCAG | 446 | CUGGCACACCAGUCCAACU | 454-472 |
| 98 | NGUUGGACUGGUGUGCCAG | 447 | CUGGCACACCAGUCCAACN | 454-472 |
| 99 | NGUUGGACUGGUGUGCCAN | 448 | NUGGCACACCAGUCCAACN | 454-472 |
| 100 | CUGUUGGACUGGUGUGCCA | 449 | UGGCACACCAGUCCAACAG | 455-473 |
| 101 | UUGUUGGACUGGUGUGCCA | 450 | UGGCACACCAGUCCAACAA | 455-473 |
| 102 | AUGUUGGACUGGUGUGCCA | 451 | UGGCACACCAGUCCAACAU | 455-473 |
| 103 | NUGUUGGACUGGUGUGCCA | 452 | UGGCACACCAGUCCAACAN | 455-473 |
| 104 | NUGUUGGACUGGUGUGCCN | 453 | NGGCACACCAGUCCAACAN | 455-473 |
| 105 | UGCUGUUGGACUGGUGUGC | 454 | GCACACCAGUCCAACAGCA | 457-475 |
| 106 | AGCUGUUGGACUGGUGUGC | 455 | GCACACCAGUCCAACAGCU | 457-475 |
| 107 | NGCUGUUGGACUGGUGUGC | 456 | GCACACCAGUCCAACAGCN | 457-475 |
| 108 | NGCUGUUGGACUGGUGUGN | 457 | NCACACCAGUCCAACAGCN | 457-475 |
| 109 | UAUUGGUGCUGUUGGACUG | 458 | CAGUCCAACAGCACCAAUA | 463-481 |
| 110 | AAUUGGUGCUGUUGGACUG | 459 | CAGUCCAACAGCACCAAUU | 463-481 |
| 111 | NAUUGGUGCUGUUGGACUG | 460 | CAGUCCAACAGCACCAAUN | 463-481 |
| 112 | NAUUGGUGCUGUUGGACUN | 461 | NAGUCCAACAGCACCAAUN | 463-481 |
| 113 | AUAUUGGUGCUGUUGGACU | 462 | AGUCCAACAGCACCAAUAU | 464-482 |
| 114 | UUAUUGGUGCUGUUGGACU | 463 | AGUCCAACAGCACCAAUAA | 464-482 |
| 115 | NUAUUGGUGCUGUUGGACU | 464 | AGUCCAACAGCACCAAUAN | 464-482 |
| 116 | NUAUUGGUGCUGUUGGACN | 465 | NGUCCAACAGCACCAAUAN | 464-482 |
| 117 | GAUAUUGGUGCUGUUGGAC | 466 | GUCCAACAGCACCAAUAUC | 465-483 |
| 118 | UAUAUUGGUGCUGUUGGAC | 467 | GUCCAACAGCACCAAUAUA | 465-483 |
| 119 | AAUAUUGGUGCUGUUGGAC | 468 | GUCCAACAGCACCAAUAUU | 465-483 |
| 120 | NAUAUUGGUGCUGUUGGAC | 469 | GUCCAACAGCACCAAUAUN | 465-483 |
| 121 | NAUAUUGGUGCUGUUGGAN | 470 | NUCCAACAGCACCAAUAUN | 465-483 |
| 122 | AAGAUAUUGGUGCUGUUGG | 471 | CCAACAGCACCAAUAUCUU | 467-485 |
| 123 | UAGAUAUUGGUGCUGUUGG | 472 | CCAACAGCACCAAUAUCUA | 467-485 |
| 124 | NAGAUAUUGGUGCUGUUGG | 473 | CCAACAGCACCAAUAUCUN | 467-485 |
| 125 | NAGAUAUUGGUGCUGUUGN | 474 | NCAACAGCACCAAUAUCUN | 467-485 |
| 126 | GUAGCGAUGCUCACUGGGG | 475 | CCCCAGUGAGCAUCGCUAC | 491-509 |
| 127 | UUAGCGAUGCUCACUGGGG | 476 | CCCCAGUGAGCAUCGCUAA | 491-509 |
| 128 | AUAGCGAUGCUCACUGGGG | 477 | CCCCAGUGAGCAUCGCUAU | 491-509 |
| 129 | NUAGCGAUGCUCACUGGGG | 478 | CCCCAGUGAGCAUCGCUAN | 491-509 |
| 130 | NUAGCGAUGCUCACUGGGN | 479 | NCCCAGUGAGCAUCGCUAN | 491-509 |
| 131 | AAAGGCUGUAGCGAUGCUC | 480 | GAGCAUCGCUACAGCCUUU | 498-516 |
| 132 | UAAGGCUGUAGCGAUGCUC | 481 | GAGCAUCGCUACAGCCUUA | 498-516 |
| 133 | NAAGGCUGUAGCGAUGCUC | 482 | GAGCAUCGCUACAGCCUUN | 498-516 |
| 134 | NAAGGCUGUAGCGAUGCUN | 483 | NAGCAUCGCUACAGCCUUN | 498-516 |
| 135 | GCAAAGGCUGUAGCGAUGC | 484 | GCAUCGCUACAGCCUUUGC | 500-518 |
| 136 | UCAAAGGCUGUAGCGAUGC | 485 | GCAUCGCUACAGCCUUUGA | 500-518 |
| 137 | ACAAAGGCUGUAGCGAUGC | 486 | GCAUCGCUACAGCCUUUGU | 500-518 |
| 138 | NCAAAGGCUGUAGCGAUGC | 487 | GCAUCGCUACAGCCUUUGN | 500-518 |
| 139 | NCAAAGGCUGUAGCGAUGN | 488 | NCAUCGCUACAGCCUUUGN | 500-518 |
| 140 | UGCAAAGGCUGUAGCGAUG | 489 | CAUCGCUACAGCCUUUGCA | 501-519 |
| 141 | AGCAAAGGCUGUAGCGAUG | 490 | CAUCGCUACAGCCUUUGCU | 501-519 |
| 142 | NGCAAAGGCUGUAGCGAUG | 491 | NAUCGCUACAGCCUUUGCN | 501-519 |
| 143 | NGCAAAGGCUGUAGCGAUN | 492 | NAUCGCUACAGCCUUUGCN | 501-519 |
| 144 | AUUGCAAAGGCUGUAGCGA | 493 | UCGCUACAGCCUUUGCAAU | 503-521 |
| 145 | UUUGCAAAGGCUGUAGCGA | 494 | UCGCUACAGCCUUUGCAAA | 503-521 |
| 146 | NUUGCAAAGGCUGUAGCGA | 495 | UCGCUACAGCCUUUGCAAN | 503-521 |
| 147 | NUUGCAAAGGCUGUAGCGN | 496 | NCGCUACAGCCUUUGCAAN | 503-521 |
| 148 | AGCAUUGCAAAGGCUGUAG | 497 | CUACAGCCUUUGCAAUGCU | 506-524 |
| 149 | UGCAUUGCAAAGGCUGUAG | 498 | CUACAGCCUUUGCAAUGCA | 506-524 |
| 150 | NGCAUUGCAAAGGCUGUAG | 499 | CUACAGCCUUUGCAAUGCN | 506-524 |
| 151 | NGCAUUGCAAAGGCUGUAN | 500 | NUACAGCCUUUGCAAUGCN | 506-524 |
| 152 | AGAGCAUUGCAAAGGCUGU | 501 | ACAGCCUUUGCAAUGCUCU | 508-526 |
| 153 | UGAGCAUUGCAAAGGCUGU | 502 | ACAGCCUUUGCAAUGCUCA | 508-526 |
| 154 | NGAGCAUUGCAAAGGCUGU | 503 | ACAGCCUUUGCAAUGCUCN | 508-526 |
| 155 | NGAGCAUUGCAAAGGCUGU | 504 | NCAGCCUUUGCAAUGCUCN | 508-526 |
| 156 | GGAGUUCCUGGAAGCCUUC | 505 | GAAGGCUUCCAGGAACUCC | 613-631 |
| 157 | UGAGUUCCUGGAAGCCUUC | 506 | GAAGGCUUCCAGGAACUCA | 613-631 |
| 158 | AGAGUUCCUGGAAGCCUUC | 507 | GAAGGCUUCCAGGAACUCU | 613-631 |
| 159 | NGAGUUCCUGGAAGCCUUC | 508 | GAAGGCUUCCAGGAACUCN | 613-631 |
| 160 | NGAGUUCCUGGAAGCCUUN | 509 | NAAGGCUUCCAGGAACUCN | 613-631 |
| 161 | UCCAAAAACUUAUCCACUA | 510 | UAGUGGAUAAGUUUUUGGA | 710-728 |
| 162 | ACCAAAAACUUAUCCACUA | 511 | UAGUGGAUAAGUUUUUGGU | 710-728 |
| 163 | NCCAAAAACUUAUCCACUA | 512 | UAGUGGAUAAGUUUUUGGN | 710-728 |
| 164 | NCCAAAAACUUAUCCACUN | 513 | NAGUGGAUAAGUUUUUGGN | 710-728 |
| 165 | AAGGCUUCUGAGUGGUACA | 514 | UGUACCACUCAGAAGCCUU | 743-761 |
| 166 | UAGGCUUCUGAGUGGUACA | 515 | UGUACCACUCAGAAGCCUA | 743-761 |
| 167 | NAGGCUUCUGAGUGGUACA | 516 | UGUACCACUCAGAAGCCUN | 743-761 |
| 168 | NAGGCUUCUGAGUGGUACN | 517 | NGUACCACUCAGAAGCCUN | 743-761 |
| 169 | GAAGGCUUCUGAGUGGUAC | 518 | GUACCACUCAGAAGCCUUC | 744-762 |
| 170 | UAAGGCUUCUGAGUGGUAC | 519 | GUACCACUCAGAAGCCUUA | 744-762 |
| 171 | AAAGGCUUCUGAGUGGUAC | 520 | GUACCACUCAGAAGCCUUU | 744-762 |
| 172 | NAAGGCUUCUGAGUGGUAC | 521 | GUACCACUCAGAAGCCUUN | 744-762 |
| 173 | NAAGGCUUCUGAGUGGUAN | 522 | NUACCACUCAGAAGCCUUN | 744-762 |
| 174 | UUCUUGGCCUCUUCGGUGU | 523 | ACACCGAAGAGGCCAAGAA | 779-797 |
| 175 | AUCUUGGCCUCUUCGGUGU | 524 | ACACCGAAGAGGCCAAGAU | 779-797 |
| 176 | NUCUUGGCCUCUUCGGUGU | 525 | ACACCGAAGAGGCCAAGAN | 779-797 |
| 177 | NUCUUGGCCUCUUCGGUGN | 526 | NCACCGAAGAGGCCAAGAN | 779-797 |
| 178 | GUUUCUUGGCCUCUUCGGU | 527 | ACCGAAGAGGCCAAGAAAC | 781-799 |
| 179 | UUUUCUUGGCCUCUUCGGU | 528 | ACCGAAGAGGCCAAGAAAA | 781-799 |
| 180 | AUUUCUUGGCCUCUUCGGU | 529 | ACCGAAGAGGCCAAGAAAU | 781-799 |
| 181 | NUUUCUUGGCCUCUUCGGU | 530 | ACCGAAGAGGCCAAGAAAN | 781-799 |
| 182 | NUUUCUUGGCCUCUUCGGN | 531 | NCCGAAGAGGCCAAGAAAN | 781-799 |
| 183 | UUGAUCUGUUUCUUGGCCU | 532 | AGGCCAAGAAACAGAUCAA | 788-806 |
| 184 | AUGAUCUGUUUCUUGGCCU | 533 | AGGCCAAGAAACAGAUCAU | 788-806 |
| 185 | NUGAUCUGUUUCUUGGCCU | 534 | AGGCCAAGAAACAGAUCAN | 788-806 |
| 186 | NUGAUCUGUUUCUUGGCCN | 535 | NGGCCAAGAAACAGAUCAN | 788-806 |
| 187 | GUUGAUCUGUUUCUUGGCC | 536 | GGCCAAGAAACAGAUCAAC | 789-807 |
| 188 | UUUGAUCUGUUUCUUGGCC | 537 | GGCCAAGAAACAGAUCAAA | 789-807 |
| 189 | AUUGAUCUGUUUCUUGGCC | 538 | GGCCAAGAAACAGAUCAAU | 789-807 |
| 190 | NUUGAUCUGUUUCUUGGCC | 539 | GGCCAAGAAACAGAUCAAN | 789-807 |
| 191 | NUUGAUCUGUUUCUUGGCN | 540 | NGCCAAGAAACAGAUCAAN | 789-807 |
| 192 | CGUUGAUCUGUUUCUUGGC | 541 | GCCAAGAAACAGAUCAACG | 790-808 |
| 193 | UGUUGAUCUGUUUCUUGGC | 542 | GCCAAGAAACAGAUCAACA | 790-808 |
| 194 | AGUUGAUCUGUUUCUUGGC | 543 | GCCAAGAAACAGAUCAACU | 790-808 |
| 195 | NGUUGAUCUGUUUCUUGGC | 544 | GCCAAGAAACAGAUCAACN | 790-808 |
| 196 | NGUUGAUCUGUUUCUUGGN | 545 | NCCAAGAAACAGAUCAACN | 790-808 |
| 197 | CACAAUUUUCCCUUGAGUA | 546 | UACUCAAGGGAAAAUUGUG | 825-843 |
| 198 | UACAAUUUUCCCUUGAGUA | 547 | UACUCAAGGGAAAAUUGUA | 825-843 |
| 199 | AACAAUUUUCCCUUGAGUA | 548 | UACUCAAGGGAAAAUUGUU | 825-843 |
| 200 | NACAAUUUUCCCUUGAGUA | 549 | UACUCAAGGGAAAAUUGUN | 825-843 |
| 201 | NACAAUUUUCCCUUGAGUN | 550 | NACUCAAGGGAAAAUUGUN | 825-843 |
| 202 | UCCACAAUUUUCCCUUGAG | 551 | CUCAAGGGAAAAUUGUGGA | 827-845 |
| 203 | ACCACAAUUUUCCCUUGAG | 552 | CUCAAGGGAAAAUUGUGGU | 827-845 |
| 204 | NCCACAAUUUUCCCUUGAG | 553 | CUCAAGGGAAAAUUGUGGN | 827-845 |
| 205 | NCCACAAUUUUCCCUUGAN | 554 | NUCAAGGGAAAAUUGUGGN | 827-845 |
| 206 | AUCCACAAUUUUCCCUUGA | 555 | UCAAGGGAAAAUUGUGGAU | 828-846 |
| 207 | UUCCACAAUUUUCCCUUGA | 556 | UCAAGGGAAAAUUGUGGAA | 828-846 |
| 208 | NUCCACAAUUUUCCCUUGA | 557 | UCAAGGGAAAAUUGUGGAN | 828-846 |
| 209 | NUCCACAAUUUUCCCUUGN | 558 | NCAAGGGAAAAUUGUGGAN | 828-846 |
| 210 | UGUCAAGCUCCUUGACCAA | 559 | UUGGUCAAGGAGCUUGACA | 847-865 |
| 211 | AGUCAAGCUCCUUGACCAA | 560 | UUGGUCAAGGAGCUUGACU | 847-865 |
| 212 | NGUCAAGCUCCUUGACCAA | 561 | NUGGUCAAGGAGCUUGACA | 847-865 |
| 213 | NGUCAAGCUCCUUGACCAA | 562 | NUGGUCAAGGAGCUUGACN | 847-865 |
| 214 | GUGUCUCUGUCAAGCUCCU | 563 | AGGAGCUUGACAGAGACAC | 854-872 |
| 215 | UUGUCUCUGUCAAGCUCCU | 564 | AGGAGCUUGACAGAGACAA | 854-872 |
| 216 | AUGUCUCUGUCAAGCUCCU | 565 | AGGAGCUUGACAGAGACAU | 854-872 |
| 217 | NUGUCUCUGUCAAGCUCCU | 566 | AGGAGCUUGACAGAGACAN | 854-872 |
| 218 | NUGUCUCUGUCAAGCUCCN | 567 | NGGAGCUUGACAGAGACAN | 854-872 |
| 219 | ACUGUGUCUCUGUCAAGCU | 568 | AGCUUGACAGAGACACAGU | 857-875 |
| 220 | UCUGUGUCUCUGUCAAGCU | 569 | AGCUUGACAGAGACACAGA | 857-875 |
| 221 | NCUGUGUCUCUGUCAAGCU | 570 | AGCUUGACAGAGACACAGN | 857-875 |
| 222 | NCUGUGUCUCUGUCAAGCN | 571 | NGCUUGACAGAGACACAGN | 857-875 |
| 223 | UGUAAUUCACCAGAGCAAA | 572 | UUUGCUCUGGUGAAUUACA | 877-895 |
| 224 | AGUAAUUCACCAGAGCAAA | 573 | UUUGCUCUGGUGAAUUACU | 877-895 |
| 225 | NGUAAUUCACCAGAGCAAA | 574 | UUUGCUCUGGUGAAUUACN | 877-895 |
| 226 | NGUAAUUCACCAGAGCAAN | 575 | NUUGCUCUGGUGAAUUACN | 877-895 |
| 227 | UUAAACAUGCCUAAACGCU | 576 | AGCGUUUAGGCAUGUUUAA | 998-1016 |
| 228 | AUAAACAUGCCUAAACGCU | 577 | AGCGUUUAGGCAUGUUUAU | 998-1016 |
| 229 | NUAAACAUGCCUAAACGCU | 578 | AGCGUUUAGGCAUGUUUAN | 998-1016 |
| 230 | NUAAACAUGCCUAAACGCN | 579 | NGCGUUUAGGCAUGUUUAN | 998-1016 |
| 231 | GUUAAACAUGCCUAAACGC | 580 | GCGUUUAGGCAUGUUUAAC | 999-1017 |
| 232 | UUUAAACAUGCCUAAACGC | 581 | GCGUUUAGGCAUGUUUAAA | 999-1017 |
| 233 | AUUAAACAUGCCUAAACGC | 582 | GCGUUUAGGCAUGUUUAAU | 999-1017 |
| 234 | NUUAAACAUGCCUAAACGC | 583 | GCGUUUAGGCAUGUUUAAN | 999-1017 |
| 235 | NUUAAACAUGCCUAAACGN | 584 | NCGUUUAGGCAUGUUUAAN | 999-1017 |
| 236 | GGAUGUUAAACAUGCCUAA | 585 | UUAGGCAUGUUUAACAUCC | 1003-1021 |
| 237 | UGAUGUUAAACAUGCCUAA | 586 | UUAGGCAUGUUUAACAUCA | 1003-1021 |
| 238 | AGAUGUUAAACAUGCCUAA | 587 | UUAGGCAUGUUUAACAUCU | 1003-1021 |
| 239 | NGAUGUUAAACAUGCCUAA | 588 | UUAGGCAUGUUUAACAUCN | 1003-1021 |
| 240 | NGAUGUUAAACAUGCCUAN | 589 | NUAGGCAUGUUUAACAUCN | 1003-1021 |
| 241 | AUUUCAUCAGCAGCACCCA | 590 | UGGGUGCUGCUGAUGAAAU | 1045-1063 |
| 242 | UUUUCAUCAGCAGCACCCA | 591 | UGGGUGCUGCUGAUGAAAA | 1045-1063 |
| 243 | NUUUCAUCAGCAGCACCCA | 592 | UGGGUGCUGCUGAUGAAAN | 1045-1063 |
| 244 | NUUUCAUCAGCAGCACCCN | 593 | NGGGUGCUGCUGAUGAAAN | 1045-1063 |
| 245 | GAAGAAGAUGGCGGUGGCA | 594 | UGCCACCGCCAUCUUCUUC | 1074-1092 |
| 246 | UAAGAAGAUGGCGGUGGCA | 595 | UGCCACCGCCAUCUUCUUA | 1074-1092 |
| 247 | AAAGAAGAUGGCGGUGGCA | 596 | UGCCACCGCCAUCUUCUUU | 1074-1092 |
| 248 | NAAGAAGAUGGCGGUGGCA | 597 | UGCCACCGCCAUCUUCUUN | 1074-1092 |
| 249 | NAAGAAGAUGGCGGUGGCN | 598 | NGCCACCGCCAUCUUCUUN | 1074-1092 |
| 250 | GGUGAGUUCAUUUUCCAGG | 599 | CCUGGAAAAUGAACUCACC | 1119-1137 |
| 251 | UGUGAGUUCAUUUUCCAGG | 600 | CCUGGAAAAUGAACUCACA | 1119-1137 |
| 252 | AGUGAGUUCAUUUUCCAGG | 601 | CCUGGAAAAUGAACUCACU | 1119-1137 |
| 253 | NGUGAGUUCAUUUUCCAGG | 602 | CCUGGAAAAUGAACUCACN | 1119-1137 |
| 254 | NGUGAGUUCAUUUUCCAGN | 603 | NCUGGAAAAUGAACUCACN | 1119-1137 |
| 255 | GAACUUGGUGAUGAUAUCG | 604 | CGAUAUCAUCACCAAGUUC | 1140-1158 |
| 256 | UAACUUGGUGAUGAUAUCG | 605 | CGAUAUCAUCACCAAGUUA | 1140-1158 |
| 257 | AAACUUGGUGAUGAUAUCG | 606 | CGAUAUCAUCACCAAGUUU | 1140-1158 |
| 258 | NAACUUGGUGAUGAUAUCG | 607 | CGAUAUCAUCACCAAGUUN | 1140-1158 |
| 259 | NAACUUGGUGAUGAUAUCN | 608 | NGAUAUCAUCACCAAGUUN | 1140-1158 |
| 260 | CAUUUUCCAGGAACUUGGU | 609 | ACCAAGUUCCUGGAAAAUG | 1150-1168 |
| 261 | UAUUUUCCAGGAACUUGGU | 610 | ACCAAGUUCCUGGAAAAUA | 1150-1168 |
| 262 | AAUUUUCCAGGAACUUGGU | 611 | ACCAAGUUCCUGGAAAAUU | 1150-1168 |
| 263 | NAUUUUCCAGGAACUUGGU | 612 | ACCAAGUUCCUGGAAAAUN | 1150-1168 |
| 264 | NAUUUUCCAGGAACUUGGN | 613 | NCCAAGUUCCUGGAAAAUN | 1150-1168 |
| 265 | CAUAGGUUCCAGUAAUGGA | 614 | UCCAUUACUGGAACCUAUG | 1207-1225 |
| 266 | UAUAGGUUCCAGUAAUGGA | 615 | UCCAUUACUGGAACCUAUA | 1207-1225 |
| 267 | AAUAGGUUCCAGUAAUGGA | 616 | UCCAUUACUGGAACCUAUU | 1207-1225 |
| 268 | NAUAGGUUCCAGUAAUGGA | 617 | UCCAUUACUGGAACCUAUN | 1207-1225 |
| 269 | NAUAGGUUCCAGUAAUGGN | 618 | NCCAUUACUGGAACCUAUN | 1207-1225 |
| 270 | UCAUAGGUUCCAGUAAUGG | 619 | CCAUUACUGGAACCUAUGA | 1208-1226 |
| 271 | ACAUAGGUUCCAGUAAUGG | 620 | CCAUUACUGGAACCUAUGU | 1208-1226 |
| 272 | NCAUAGGUUCCAGUAAUGG | 621 | CCAUUACUGGAACCUAUGN | 1208-1226 |
| 273 | NCAUAGGUUCCAGUAAUGN | 622 | NCAUUACUGGAACCUAUGN | 1208-1226 |
| 274 | UCAGAUCAUAGGUUCCAGU | 623 | ACUGGAACCUAUGAUCUGA | 1213-1231 |
| 275 | ACAGAUCAUAGGUUCCAGU | 624 | ACUGGAACCUAUGAUCUGU | 1213-1231 |
| 276 | NCAGAUCAUAGGUUCCAGU | 625 | ACUGGAACCUAUGAUCUGN | 1213-1231 |
| 277 | NCAGAUCAUAGGUUCCAGN | 626 | NCUGGAACCUAUGAUCUGN | 1213-1231 |
| 278 | UCUUCAGAUCAUAGGUUCC | 627 | GGAACCUAUGAUCUGAAGA | 1216-1234 |
| 279 | ACUUCAGAUCAUAGGUUCC | 628 | GGAACCUAUGAUCUGAAGU | 1216-1234 |
| 280 | NCUUCAGAUCAUAGGUUCC | 629 | GGAACCUAUGAUCUGAAGN | 1216-1234 |
| 281 | NCUUCAGAUCAUAGGUUCN | 630 | NGAACCUAUGAUCUGAAGN | 1216-1234 |
| 282 | CUCUUCAGAUCAUAGGUUC | 631 | GAACCUAUGAUCUGAAGAG | 1217-1235 |
| 283 | UUCUUCAGAUCAUAGGUUC | 632 | GAACCUAUGAUCUGAAGAA | 1217-1235 |
| 284 | AUCUUCAGAUCAUAGGUUC | 633 | GAACCUAUGAUCUGAAGAU | 1217-1235 |
| 285 | NUCUUCAGAUCAUAGGUUC | 634 | GAACCUAUGAUCUGAAGAG | 1217-1235 |
| 286 | NUCUUCAGAUCAUAGGUUN | 635 | NAACCUAUGAUCUGAAGAN | 1217-1235 |
| 287 | GAGGUCAGCCCCAUUGCUG | 636 | CAGCAAUGGGGCUGACCUC | 1269-1287 |
| 288 | UAGGUCAGCCCCAUUGCUG | 637 | CAGCAAUGGGGCUGACCUA | 1269-1287 |
| 289 | AAGGUCAGCCCCAUUGCUG | 638 | CAGCAAUGGGGCUGACCUU | 1269-1287 |
| 290 | NAGGUCAGCCCCAUUGCUG | 639 | CAGCAAUGGGGCUGACCUN | 1269-1287 |
| 291 | NAGGUCAGCCCCAUUGCUN | 640 | NAGCAAUGGGGCUGACCUN | 1269-1287 |
| 292 | GAGAGGUCAGCCCCAUUGC | 641 | GCAAUGGGGCUGACCUCUC | 1271-1289 |
| 293 | UAGAGGUCAGCCCCAUUGC | 642 | GCAAUGGGGCUGACCUCUA | 1271-1289 |
| 294 | AAGAGGUCAGCCCCAUUGC | 643 | GCAAUGGGGCUGACCUCUU | 1271-1289 |
| 295 | NAGAGGUCAGCCCCAUUGC | 644 | GCAAUGGGGCUGACCUCUN | 1271-1289 |
| 296 | NAGAGGUCAGCCCCAUUGN | 645 | NCAAUGGGGCUGACCUCUN | 1271-1289 |
| 297 | CUUCAGGGGUGCCUCCUCU | 646 | AGAGGAGGCACCCCUGAAG | 1299-1317 |
| 298 | UUUCAGGGGUGCCUCCUCU | 647 | AGAGGAGGCACCCCUGAAA | 1299-1317 |
| 299 | AUUCAGGGGUGCCUCCUCU | 648 | AGAGGAGGCACCCCUGAAU | 1299-1317 |
| 300 | NUUCAGGGGUGCCUCCUCU | 649 | AGAGGAGGCACCCCUGAAN | 1299-1317 |
| 301 | NUUCAGGGGUGCCUCCUCN | 650 | NGAGGAGGCACCCCUGAAN | 1299-1317 |
| 302 | GAGAGCUUCAGGGGUGCCU | 651 | AGGCACCCCUGAAGCUCUC | 1304-1322 |
| 303 | UAGAGCUUCAGGGGUGCCU | 652 | AGGCACCCCUGAAGCUCUA | 1304-1322 |
| 304 | AAGAGCUUCAGGGGUGCCU | 653 | AGGCACCCCUGAAGCUCUU | 1304-1322 |
| 305 | NAGAGCUUCAGGGGUGCCU | 654 | AGGCACCCCUGAAGCUCUN | 1304-1322 |
| 306 | NAGAGCUUCAGGGGUGCCN | 655 | NGGCACCCCUGAAGCUCUN | 1304-1322 |
| 307 | UUAUGCACGGCCUUGGAGA | 656 | UCUCCAAGGCCGUGCAUAA | 1319-1337 |
| 308 | AUAUGCACGGCCUUGGAGA | 657 | UCUCCAAGGCCGUGCAUAU | 1319-1337 |
| 309 | NUAUGCACGGCCUUGGAGA | 658 | UCUCCAAGGCCGUGCAUAN | 1319-1337 |
| 310 | NUAUGCACGGCCUUGGAGN | 659 | NCUCCAAGGCCGUGCAUAN | 1319-1337 |
| 311 | CCUUAUGCACGGCCUUGGA | 660 | UCCAAGGCCGUGCAUAAGG | 1321-1339 |
| 312 | UCUUAUGCACGGCCUUGGA | 661 | UCCAAGGCCGUGCAUAAGA | 1321-1339 |
| 313 | ACUUAUGCACGGCCUUGGA | 662 | UCCAAGGCCGUGCAUAAGU | 1321-1339 |
| 314 | NCUUAUGCACGGCCUUGGA | 663 | UCCAAGGCCGUGCAUAAGN | 1321-1339 |
| 315 | NCUUAUGCACGGCCUUGGN | 664 | NCCAAGGCCGUGCAUAAGN | 1321-1339 |
| 316 | GCCUUAUGCACGGCCUUGG | 665 | CCAAGGCCGUGCAUAAGGC | 1322-1340 |
| 317 | UCCUUAUGCACGGCCUUGG | 666 | CCAAGGCCGUGCAUAAGGA | 1322-1340 |
| 318 | ACCUUAUGCACGGCCUUGG | 667 | CCAAGGCCGUGCAUAAGGU | 1322-1340 |
| 319 | NCCUUAUGCACGGCCUUGG | 668 | CCAAGGCCGUGCAUAAGGN | 1322-1340 |
| 320 | NCCUUAUGCACGGCCUUGN | 669 | NCAAGGCCGUGCAUAAGGN | 1322-1340 |
| 321 | AGCCUUAUGCACGGCCUUG | 670 | CAAGGCCGUGCAUAAGGCU | 1323-1341 |
| 322 | UGCCUUAUGCACGGCCUUG | 671 | CAAGGCCGUGCAUAAGGCA | 1323-1341 |
| 323 | NGCCUUAUGCACGGCCUUG | 672 | CAAGGCCGUGCAUAAGGCN | 1323-1341 |
| 324 | NGCCUUAUGCACGGCCUUN | 673 | NAAGGCCGUGCAUAAGGCN | 1323-1341 |
| 325 | CGAUGGUCAGCACAGCCUU | 674 | AAGGCUGUGCUGACCAUCG | 1336-1354 |
| 326 | UGAUGGUCAGCACAGCCUU | 675 | AAGGCUGUGCUGACCAUCA | 1336-1354 |
| 327 | AGAUGGUCAGCACAGCCUU | 676 | AAGGCUGUGCUGACCAUCU | 1336-1354 |
| 328 | NGAUGGUCAGCACAGCCUU | 677 | AAGGCUGUGCUGACCAUCN | 1336-1354 |
| 329 | NGAUGGUCAGCACAGCCUN | 678 | NAGGCUGUGCUGACCAUCN | 1336-1354 |
| 330 | GUCGAUGGUCAGCACAGCC | 679 | GGCUGUGCUGACCAUCGAC | 1338-1356 |
| 331 | UUCGAUGGUCAGCACAGCC | 680 | GGCUGUGCUGACCAUCGAA | 1338-1356 |
| 332 | AUCGAUGGUCAGCACAGCC | 681 | GGCUGUGCUGACCAUCGAU | 1338-1356 |
| 333 | NUCGAUGGUCAGCACAGCC | 682 | GGCUGUGCUGACCAUCGAN | 1338-1356 |
| 334 | NUCGAUGGUCAGCACAGCN | 683 | NGCUGUGCUGACCAUCGAN | 1338-1356 |
| 335 | AAAAACAUGGCCCCAGCAG | 684 | CUGCUGGGGCCAUGUUUUU | 1373-1391 |
| 336 | UAAAACAUGGCCCCAGCAG | 685 | CUGCUGGGGCCAUGUUUUA | 1373-1391 |
| 337 | NAAAACAUGGCCCCAGCAG | 686 | CUGCUGGGGCCAUGUUUUN | 1373-1391 |
| 338 | NAAAACAUGGCCCCAGCAN | 687 | NUGCUGGGGCCAUGUUUUN | 1373-1391 |
| 339 | CUAAAAACAUGGCCCCAGC | 688 | GCUGGGGCCAUGUUUUUAG | 1375-1393 |
| 340 | UUAAAAACAUGGCCCCAGC | 689 | GCUGGGGCCAUGUUUUUAA | 1375-1393 |
| 341 | AUAAAAACAUGGCCCCAGC | 690 | GCUGGGGCCAUGUUUUUAU | 1375-1393 |
| 342 | NUAAAAACAUGGCCCCAGC | 691 | GCUGGGGCCAUGUUUUUAN | 1375-1393 |
| 343 | NUAAAAACAUGGCCCCAGN | 692 | NCUGGGGCCAUGUUUUUAN | 1375-1393 |
| 344 | UCUAAAAACAUGGCCCCAG | 693 | CUGGGGCCAUGUUUUUAGA | 1376-1394 |
| 345 | ACUAAAAACAUGGCCCCAG | 694 | CUGGGGCCAUGUUUUUAGU | 1376-1394 |
| 346 | NCUAAAAACAUGGCCCCAG | 695 | CUGGGGCCAUGUUUUUAGN | 1376-1394 |
| 347 | NCUAAAAACAUGGCCCCAN | 696 | NUGGGGCCAUGUUUUUAGN | 1376-1394 |
| 348 | CCUCUAAAAACAUGGCCCC | 697 | GGGGCCAUGUUUUUAGAGG | 1378-1396 |
| 349 | UCUCUAAAAACAUGGCCCC | 698 | GGGGCCAUGUUUUUAGAGA | 1378-1396 |
| 350 | ACUCUAAAAACAUGGCCCC | 699 | GGGGCCAUGUUUUUAGAGU | 1378-1396 |
| 351 | NCUCUAAAAACAUGGCCCC | 700 | GGGGCCAUGUUUUUAGAGN | 1378-1396 |
| 352 | NCUCUAAAAACAUGGCCCN | 701 | NGGGCCAUGUUUUUAGAGN | 1378-1396 |
| 353 | GCCUCUAAAAACAUGGCCC | 702 | GGGCCAUGUUUUUAGAGGC | 1379-1397 |
| 354 | UCCUCUAAAAACAUGGCCC | 703 | GGGCCAUGUUUUUAGAGGA | 1379-1397 |
| 355 | ACCUCUAAAAACAUGGCCC | 704 | GGGCCAUGUUUUUAGAGGU | 1379-1397 |
| 356 | NCCUCUAAAAACAUGGCCC | 705 | GGGCCAUGUUUUUAGAGGN | 1379-1397 |
| 357 | NCCUCUAAAAACAUGGCCN | 706 | NGGCCAUGUUUUUAGAGGN | 1379-1397 |
| 358 | UAGACAUGGGUAUGGCCUC | 707 | GAGGCCAUACCCAUGUCUA | 1393-1411 |
| 359 | AAGACAUGGGUAUGGCCUC | 708 | GAGGCCAUACCCAUGUCUU | 1393-1411 |
| 360 | NAGACAUGGGUAUGGCCUC | 709 | GAGGCCAUACCCAUGUCUN | 1393-1411 |
| 361 | NAGACAUGGGUAUGGCCUN | 710 | NAGGCCAUACCCAUGUCUN | 1393-1411 |
| 362 | GAUAGACAUGGGUAUGGCC | 711 | GGCCAUACCCAUGUCUAUC | 1395-1413 |
| 363 | UAUAGACAUGGGUAUGGCC | 712 | GGCCAUACCCAUGUCUAUA | 1395-1413 |
| 364 | AAUAGACAUGGGUAUGGCC | 713 | GGCCAUACCCAUGUCUAUU | 1395-1413 |
| 365 | NAUAGACAUGGGUAUGGCC | 714 | GGCCAUACCCAUGUCUAUN | 1395-1413 |
| 366 | NAUAGACAUGGGUAUGGCN | 715 | NGCCAUACCCAUGUCUAUN | 1395-1413 |
| 367 | UGUUGAACUUGACCUCGGG | 716 | CCCGAGGUCAAGUUCAACA | 1417-1435 |
| 368 | AGUUGAACUUGACCUCGGG | 717 | CCCGAGGUCAAGUUCAACU | 1417-1435 |
| 369 | NGUUGAACUUGACCUCGGG | 718 | CCCGAGGUCAAGUUCAACN | 1417-1435 |
| 370 | NGUUGAACUUGACCUCGGN | 719 | NCCGAGGUCAAGUUCAACN | 1417-1435 |
| 371 | GGUUUGUUGAACUUGACCU | 720 | AGGUCAAGUUCAACAAACC | 1421-1439 |
| 372 | UGUUUGUUGAACUUGACCU | 721 | AGGUCAAGUUCAACAAACA | 1421-1439 |
| 373 | AGUUUGUUGAACUUGACCU | 722 | AGGUCAAGUUCAACAAACU | 1421-1439 |
| 374 | NGUUUGUUGAACUUGACCU | 723 | AGGUCAAGUUCAACAAACN | 1421-1439 |
| 375 | NGUUUGUUGAACUUGACCN | 724 | NGGUCAAGUUCAACAAACN | 1421-1439 |
| 376 | AAAGGGUUUGUUGAACUUG | 725 | CAAGUUCAACAAACCCUUU | 1425-1443 |
| 377 | UAAGGGUUUGUUGAACUUG | 726 | CAAGUUCAACAAACCCUUA | 1425-1443 |
| 378 | NAAGGGUUUGUUGAACUUG | 727 | CAAGUUCAACAAACCCUUN | 1425-1443 |
| 379 | NAAGGGUUUGUUGAACUUN | 728 | NAAGUUCAACAAACCCUUN | 1425-1443 |
| 380 | ACAAAGGGUUUGUUGAACU | 729 | AGUUCAACAAACCCUUUGU | 1427-1445 |
| 381 | UCAAAGGGUUUGUUGAACU | 730 | AGUUCAACAAACCCUUUGA | 1427-1445 |
| 382 | NCAAAGGGUUUGUUGAACU | 731 | AGUUCAACAAACCCUUUGN | 1427-1445 |
| 383 | NCAAAGGGUUUGUUGAACN | 732 | NGUUCAACAAACCCUUUGN | 1427-1445 |
| 384 | GACAAAGGGUUUGUUGAAC | 733 | GUUCAACAAACCCUUUGUC | 1428-1446 |
| 385 | UACAAAGGGUUUGUUGAAC | 734 | GUUCAACAAACCCUUUGUA | 1428-1446 |
| 386 | AACAAAGGGUUUGUUGAAC | 735 | GUUCAACAAACCCUUUGUU | 1428-1446 |
| 387 | NACAAAGGGUUUGUUGAAC | 736 | GUUCAACAAACCCUUUGUN | 1428-1446 |
| 388 | NACAAAGGGUUUGUUGAAN | 737 | NUUCAACAAACCCUUUGUN | 1428-1446 |
| 389 | AAGACAAAGGGUUUGUUGA | 738 | UCAACAAACCCUUUGUCUU | 1430-1448 |
| 390 | UAGACAAAGGGUUUGUUGA | 739 | UCAACAAACCCUUUGUCUA | 1430-1448 |
| 391 | NAGACAAAGGGUUUGUUGA | 740 | UCAACAAACCCUUUGUCUN | 1430-1448 |
| 392 | NAGACAAAGGGUUUGUUGN | 741 | NCAACAAACCCUUUGUCUN | 1430-1448 |
| 393 | CAUUAAGAAGACAAAGGGU | 742 | ACCCUUUGUCUUCUUAAUG | 1437-1455 |
| 394 | UAUUAAGAAGACAAAGGGU | 743 | ACCCUUUGUCUUCUUAAUA | 1437-1455 |
| 395 | AAUUAAGAAGACAAAGGGU | 744 | ACCCUUUGUCUUCUUAAUU | 1437-1455 |
| 396 | NAUUAAGAAGACAAAGGGU | 745 | ACCCUUUGUCUUCUUAAUN | 1437-1455 |
| 397 | NAUUAAGAAGACAAAGGGN | 746 | NCCCUUUGUCUUCUUAAUN | 1437-1455 |
| 398 | AUCAUUAAGAAGACAAAGG | 747 | CCUUUGUCUUCUUAAUGAU | 1439-1457 |
| 399 | UUCAUUAAGAAGACAAAGG | 748 | CCUUUGUCUUCUUAAUGAA | 1439-1457 |
| 400 | NUCAUUAAGAAGACAAAGG | 749 | CCUUUGUCUUCUUAAUGAN | 1439-1457 |
| 401 | NUCAUUAAGAAGACAAAGN | 750 | NCUUUGUCUUCUUAAUGAN | 1439-1457 |
| 402 | GAAGAGGGGAGACUUGGUA | 751 | UACCAAGUCUCCCCUCUUC | 1467-1485 |
| 403 | UAAGAGGGGAGACUUGGUA | 752 | UACCAAGUCUCCCCUCUUA | 1467-1485 |
| 404 | AAAGAGGGGAGACUUGGUA | 753 | UACCAAGUCUCCCCUCUUU | 1467-1485 |
| 405 | NAAGAGGGGAGACUUGGUA | 754 | UACCAAGUCUCCCCUCUUN | 1467-1485 |
| 406 | NAAGAGGGGAGACUUGGUN | 755 | NACCAAGUCUCCCCUCUUN | 1467-1485 |
| 407 | CCAUGAAGAGGGGAGACUU | 756 | AAGUCUCCCCUCUUCAUGG | 1471-1489 |
| 408 | UCAUGAAGAGGGGAGACUU | 757 | AAGUCUCCCCUCUUCAUGA | 1471-1489 |
| 409 | ACAUGAAGAGGGGAGACUU | 758 | AAGUCUCCCCUCUUCAUGU | 1471-1489 |
| 410 | NCAUGAAGAGGGGAGACUU | 759 | AAGUCUCCCCUCUUCAUGN | 1471-1489 |
| 411 | NCAUGAAGAGGGGAGACUN | 760 | NAGUCUCCCCUCUUCAUGN | 1471-1489 |
| 412 | CCCAUGAAGAGGGGAGACU | 761 | AGUCUCCCCUCUUCAUGGG | 1472-1490 |
| 413 | UCCAUGAAGAGGGGAGACU | 762 | AGUCUCCCCUCUUCAUGGA | 1472-1490 |
| 414 | ACCAUGAAGAGGGGAGACU | 763 | AGUCUCCCCUCUUCAUGGU | 1472-1490 |
| 415 | NCCAUGAAGAGGGGAGACU | 764 | AGUCUCCCCUCUUCAUGGN | 1472-1490 |
| 416 | NCCAUGAAGAGGGGAGACN | 765 | NGUCUCCCCUCUUCAUGGN | 1472-1490 |
| 417 | UUCCCAUGAAGAGGGGAGA | 766 | UCUCCCCUCUUCAUGGGAA | 1474-1492 |
| 418 | AUCCCAUGAAGAGGGGAGA | 767 | UCUCCCCUCUUCAUGGGAU | 1474-1492 |
| 419 | NUCCCAUGAAGAGGGGAGA | 768 | UCUCCCCUCUUCAUGGGAN | 1474-1492 |
| 420 | NUCCCAUGAAGAGGGGAGN | 769 | NCUCCCCUCUUCAUGGGAN | 1474-1492 |
| 421 | UUUCCCAUGAAGAGGGGAG | 770 | CUCCCCUCUUCAUGGGAAA | 1475-1493 |
| 422 | AUUCCCAUGAAGAGGGGAG | 771 | CUCCCCUCUUCAUGGGAAU | 1475-1493 |
| 423 | NUUCCCAUGAAGAGGGGAG | 772 | CUCCCCUCUUCAUGGGAAN | 1475-1493 |
| 424 | NUUCCCAUGAAGAGGGGAN | 773 | NUCCCCUCUUCAUGGGAAN | 1475-1493 |
| 425 | AACCCUUCUUUAAUGUCAU | 774 | AUGACAUUAAAGAAGGGUU | 1569-1587 |
| 426 | UACCCUUCUUUAAUGUCAU | 775 | AUGACAUUAAAGAAGGGUA | 1569-1587 |
| 427 | NACCCUUCUUUAAUGUCAU | 776 | AUGACAUUAAAGAAGGGUN | 1569-1587 |
| 428 | NACCCUUCUUUAAUGUCAN | 777 | NUGACAUUAAAGAAGGGUN | 1569-1587 |

TABLE 3
Example AAT RNAi Agent Antisense Strand and
Sense Strand Base Sequences

| SEQ ID | Antisense Base | SEQ ID | Sense Base |
|---|---|---|---|
| NO: | Sequence (5′ → 3′) | NO: | Sequence (5′ → 3′) |
| 778 | GGAACUUGGUGAUGAUAU | 840 | AUAUCAUCACCAAGUUCC |
| 779 | GAUCAUAGGUUCCAGUAA | 841 | UUACUGGAACCUAUGAUC |
| 780 | ACAGCCUUAUGCACGGCC | 842 | GGCCGUGCAUAAGGCUGU |
| 781 | UCGAUGGUCAGCACAGCC | 843 | GGCUGUGCUGACCAUCGA |
| 782 | CAAAGGGUUUGUUGAACU | 844 | AGUUCAACAAACCCUUUG |
| 783 | TGGAACUUGGUGAUGAUAUTT | 845 | UAUAUAUCAUCACCAAGUUCCAT |
| 783 | TGGAACUUGGUGAUGAUAUTT | 846 | AUAUCAUCACCAAGUUCCAT |
| 784 | TGGAACUUGGUGAUGAUAUCGUG | 847 | CGAUAUCAUCACCAAGUUCCA |
| 785 | ACUUGGUGAUGAUAUTT | 848 | UAUCAUCACCAAGUUCCAT |
| 786 | TGGAACTTGGTGATGATATTT | 849 | TATATATCATCACCAAGTTCCAT |
| 787 | UUUAAACAUGCCUAAACGCUU | 850 | GCGUUUAGGCAUGUUUAAAUU |
| 788 | UGCAUUGCCCAGGUAUUUCUU | 851 | GAAAUACCUGGGCAAUGCAUU |
| 789 | UGGAACUUGGUGAUGAUAUUU | 852 | AUAUCAUCACCAAGUUCCAUU |
| 790 | UGAUCAUAGGUUCCAGUAAUU | 853 | UUACUGGAACCUAUGAUCAUU |
| 791 | UACAGCCUUAUGCACGGCCUU | 854 | GGCCGUGCAUAAGGCUGUAUU |
| 792 | UUCGAUGGUCAGCACAGCCUU | 855 | GGCUGUGCUGACCAUCGAAUU |
| 793 | UCAAAGGGUUUGUUGAACUUU | 856 | AGUUCAACAAACCCUUUGAUU |
| 794 | UGUUAAACAUGCCUAAACGUU | 857 | CGUUUAGGCAUGUUUAACAUU |
| 795 | UUUAAACGUGCCUAAACGCUG | 858 | CAGCGUUUAGGCAUGUUUAAA |
| 796 | UGCAUUGCCCAGGUAUUUCAG | 859 | CUGAAAUACCUGGGCAAUGCA |
| 797 | UGGAACUUGGUGAUGAUAUCG | 847 | CGAUAUCAUCACCAAGUUCCA |
| 798 | UGAUCAUAGGUUCCAGUAAUG | 860 | CAUUACUGGAACCUAUGAUCA |
| 791 | UACAGCCUUAUGCACGGCCUU | 861 | AAGGCCGUGCAUAAGGCUGUA |
| 792 | UUCGAUGGUCAGCACAGCCUU | 862 | AAGGCUGUGCUGACCAUCGAA |
| 799 | UCAAAGGGUUUGUUGAACUUG | 863 | CAAGUUCAACAAACCCUUUGA |
| 800 | UGUUAAACAUGCCUAAACGCG | 864 | CGCGUUUAGGCAUGUUUAACA |
| 801 | UGUUAAACAUGCCUAAACGCU | 857 | CGUUUAGGCAUGUUUAACAUU |
| 794 | UGUUAAACAUGCCUAAACGUU | 1265 | CGUUUAGGCAUGUUUAACA |
| 801 | UGUUAAACAUGCCUAAACGCU | 1265 | CGUUUAGGCAUGUUUAACA |
| 794 | UGUUAAACAUGCCUAAACGUU | 865 | AACGUUUAGGCAUGUUUAACA |
| 801 | UGUUAAACAUGCCUAAACGCU | 866 | AGCGUUUAGGCAUGUUUAACA |
| 802 | UGUUAAACAUGCCUAAACGCUUC | 866 | AGCGUUUAGGCAUGUUUAACA |
| 803 | UGCUGUUGGACUGGUGUGCUU | 1266 | GCACACCAGUCCAACAGCA |
| 804 | UGCUGUUGGACUGGUGUGCCA | 1266 | GCACACCAGUCCAACAGCA |
| 804 | UGCUGUUGGACUGGUGUGCCA | 867 | UGGCACACCAGUCCAACAGCA |
| 803 | UGCUGUUGGACUGGUGUGCUU | 868 | AAGCACACCAGUCCAACAGCA |
| 805 | UGCUGUUGGACUGGUGUGCCAUU | 867 | UGGCACACCAGUCCAACAGCA |
| 806 | UGCUGUUGGACUGGUGUGCCAGC | 867 | UGGCACACCAGUCCAACAGCA |
| 807 | UAAGGCUUCUGAGUGGUACUU | 1267 | GUACCACUCAGAAGCCUUA |
| 808 | UAAGGCUUCUGAGUGGUACAA | 1267 | GUACCACUCAGAAGCCUUA |
| 808 | UAAGGCUUCUGAGUGGUACAA | 869 | UUGUACCACUCAGAAGCCUUA |
| 809 | UAAGGCUUCUGAGUGGUACAACU | 869 | UUGUACCACUCAGAAGCCUUA |
| 810 | GAAGGCUUCUGAGUGGUACUU | 1268 | GUACCACUCAGAAGCCUUC |
| 811 | AAGACAAAGGGUUUGUUGAUU | 1269 | UCAACAAACCCUUUGUCUU |
| 812 | AAGACAAAGGGUUUGUUGAAC | 1269 | UCAACAAACCCUUUGUCUU |
| 812 | AAGACAAAGGGUUUGUUGAAC | 870 | GUUCAACAAACCCUUUGUCUU |
| 813 | UAGACAAAGGGUUUGUUGAAC | 871 | GUUCAACAAACCCUUUGUCUA |
| 814 | AAGACAAAGGGUUUGUUGAACUU | 870 | GUUCAACAAACCCUUUGUCUU |
| 815 | UAGACAUGGGUAUGGCCUCUU | 1270 | GAGGCCAUACCCAUGUCUA |
| 816 | UAGACAUGGGUAUGGCCUCUA | 1270 | GAGGCCAUACCCAUGUCUA |
| 816 | UAGACAUGGGUAUGGCCUCUA | 872 | UAGAGGCCAUACCCAUGUCUA |
| 817 | UAGACAUGGGUAUGGCCUCUAAA | 872 | UAGAGGCCAUACCCAUGUCUA |
| 818 | UAGACAUGGGUAUGGCCUCUAUU | 872 | UAGAGGCCAUACCCAUGUCUA |
| 819 | UUUGAUCUGUUUCUUGGCCUU | 1271 | GGCCAAGAAACAGAUCAAA |
| 820 | UUUGAUCUGUUUCUUGGCCUC | 1271 | GGCCAAGAAACAGAUCAAA |
| 820 | UUUGAUCUGUUUCUUGGCCUC | 873 | GAGGCCAAGAAACAGAUCAAA |
| 821 | UUUGAUCUGUUUCUUGGCCUCUU | 873 | GAGGCCAAGAAACAGAUCAAA |
| 822 | UGUUGGACUGGUGUGCCAGUU | 1272 | CUGGCACACCAGUCCAACA |
| 823 | UGUUGGACUGGUGUGCCAGCU | 874 | AGCUGGCACACCAGUCCAACA |
| 824 | UGUUGGACUGGUGUGCCAGCUGG | 874 | AGCUGGCACACCAGUCCAACA |
| 825 | UGUUGGACUGGUGUGCCAGCUG | 875 | GCUGGCACACCAGUCCAACA |
| 826 | AAAGGGUUUGUUGAACUUGUU | 1273 | CAAGUUCAACAAACCCUUU |
| 827 | AAAGGGUUUGUUGAACUUGAC | 876 | GUCAAGUUCAACAAACCCUUU |
| 828 | UAAGGGUUUGUUGAACUUGACCU | 877 | GUCAAGUUCAACAAACCCUUA |
| 829 | UAAGGGUUUGUUGAACUUGAC | 877 | GUCAAGUUCAACAAACCCUUA |
| 830 | UAUUGGUGCUGUUGGACUGUU | 1274 | CAGUCCAACAGCACCAAUA |
| 831 | UAUUGGUGCUGUUGGACUGGU | 878 | ACCAGUCCAACAGCACCAAUA |
| 832 | UAUUGGUGCUGUUGGACUGGUU | 879 | CCAGUCCAACAGCACCAAUA |
| 833 | UUGUUGGACUGGUGUGCCAG | 880 | CUGGCACACCAGUCCAACAA |
| 834 | UUGUUGGACUGGUGUGCCAGCU | 880 | CUGGCACACCAGUCCAACAA |
| 835 | UAUAGACAUGGGUAUGGCCUC | 1275 | GGCCAUACCCAUGUCUAUA |
| 835 | UAUAGACAUGGGUAUGGCCUC | 881 | GAGGCCAUACCCAUGUCUAUA |
| 836 | UCAAAGGGUUUGUUGAACUUGAC | 882 | GUCAAGUUCAACAAACCCUUUGA |
| 836 | UCAAAGGGUUUGUUGAACUUGAC | 863 | CAAGUUCAACAAACCCUUUGA |
| 837 | UUAUUGGUGCUGUUGGACUGG | 883 | CCAGUCCAACAGCACCAAUAA |
| 838 | UGUUAAACAUGCCUAAACGC | 884 | GCGUUUAGGCAUGUUUAACA |
| 839 | UGUUAAACAUGCCUAAACGCUU | 884 | GCGUUUAGGCAUGUUUAACA |
| 839 | UGUUAAACAUGCCUAAACGCUU | 885 | GCGUUUAGGCAUGUUUAACAUU |
| 800 | UGUUAAACAUGCCUAAACGCG | 886 | CGCGUUUAGGCAUGUUUAACAUU |
| 801 | UGUUAAACAUGCCUAAACGCU | 887 | AGCGUUUAGGCAUGUUUAACAUU |
| 838 | UGUUAAACAUGCCUAAACGC | 885 | GCGUUUAGGCAUGUUUAACAUU |

The AAT RNAi agent sense strands and antisense strands that comprise or consist of the nucleotide sequences in Table 2 or Table 3 can be modified nucleotides or unmodified nucleotides. In some embodiments, the AAT RNAi agents having the sense and antisense strand sequences that comprise or consist of any of the nucleotide sequences in Table 2 or Table 3 are all or substantially all modified nucleotides.

In some embodiments, the antisense strand of an AAT RNAi agent disclosed herein differs by 0, 1, 2, or 3 nucleotides from any of the antisense strand sequences in Table 2 or Table 3. In some embodiments, the sense strand of an AAT RNAi agent disclosed herein differs by 0, 1, 2, or 3 nucleotides from any of the sense strand sequences in Table 2 or Table 3.

As used herein, each N listed in a sequence disclosed in Table 2 may be independently selected. In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified AAT RNAi agent sense and antisense strands are provided in Table 4 and Table 5. Modified AAT RNAi agent antisense strands, as well as their underlying unmodified nucleobase sequences, are provided in Table 4. Modified AAT RNAi agent sense strands, as well as their underlying unmodified sequences, are provided in Table 5. In forming AAT RNAi agents, each of the nucleotides in each of the unmodified sequences listed in Tables 4 and 5, as well as in Table 2 and Table 3, above, can be a modified nucleotide.

The AAT RNAi agents described herein are formed by annealing an antisense strand with a sense strand. A sense strand containing a sequence listed in Table 2, Table 3, or Table 5, can be hybridized to any antisense strand containing a sequence listed in Table 2. Table 3, or Table 4, provided the two sequences have a region of at least 85% complementarity over a contiguous 16, 17, 18, 19, 20, or 21 nucleotide sequence.

In some embodiments, an AAT RNAi agent antisense strand comprises a nucleotide sequence of any of the sequences in Table 2, Table 3, or Table 4.

In some embodiments, an AAT RNAi agent comprises or consists of a duplex having the nucleobase sequences of the sense strand and the antisense strand of any of the sequences in Table 2 or Table 3.

Examples of antisense strands containing modified nucleotides are provided in Table 4. Examples of sense strands containing modified nucleotides are provided in Table 5.

As used in Tables 4 and 5, the following notations are used to indicate modified nucleotides, targeting groups, and linking groups. As the person of ordinary skill in the art would readily understand, unless otherwise indicated by the sequence, that when present in an oligonucleotide, the monomers are mutually linked by 5′-3′-phosphodiester bonds;
A=adenosine-3′-phosphate;
C=cytidine-3′-phosphate:
G=guanosine-3′-phosphate;
U=uridine-3′-phosphate
n=any 2′-OMe modified nucleotide
a=2′-O-methyladenosine-3′-phosphate
as =2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Nf=any 2′-fluoro modified nucleotide
Af=2′-fluoroadenosine-3′-phosphate
As=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluoroundine-3′-phosphate
Ufs=2′-fluorouridine-3′-phosphorothioate
dN=any 2′-deoxvribonucleotide
dT=2′-deoxythymidine-3′-phosphate
N_{UNA}=2′,3′-seco nucleotide mimics (unlocked nucleobase analogs)-3′-Phosphate
N_{UNAS}=2′,3′-seco nucleotide mimics (unlocked nucleobase analogs)-3′-phosphorothioate
U_{UNA}=2′,3′-seco-uridine-3′-phosphate
U_{UNAS}=2′,3′-seco-uridine-3′-phosphorothioate
a_2N=see Table 7
a_2Ns=see Table 7
pu_2N=see Table 7
pu_2Ns=see Table 7
Npu=see Table 7
Nus=see Table 7
NLNA=locked nucleotide
NfANA=2′-F-Arabino nucleotide
NM=2′-methoxyethyl nucleotide
AM=2′-methoxyethyladenosine-3′-phosphate
AMs=2′-methoxyethyladenosine-3′-phosphorothioate
TM=2′-methoxyethylthymidine-3′-phosphate
TMs=2′-methoxyethylthymidine-3′-phosphorothioate
R=ribitol
(invdN)=any inverted deoxyribonucleotide (3′-3′ linked nucleotide)
(inv,Ab)=inverted (3′-3′ linked) abasic deoxyribonucleotide, see Table 7
(invAb)s=inverted (3′-3′ linked) abasic deoxyribonucleotide-5′-phosphorothioate, see Table 7
(invn)=any inverted 2′-OMe nucleotide (3′-3′ linked nucleotide)
s=phosphorothioate linkage
vpdN=vinyl phosphonate deoxyribonucleotide
(5Me-Nf)=5′-Me, 2′-fluoro nucleotide
cPrp=cyclopropyl phosphonate, see Table 7
epTcPr=see Table 7
epTM=see Table 7
(Chol-TEG)=see Table 7
(TEG-Biotin)=see Table 7
(PEG-C3-SS)=see Table 7
(Alk-SS-C6)=see Table 7
(C6-SS-Alk)=see Table 7
(C6-SS-Alk-Me)=see Table 7

The person or ordinary skill in the art would readily understand that the terminal nucleotide at the 3′ end of a given oligonucleotide sequence would typically have a hydroxyl (—OH) group at the respective 3′ position of the given monomer instead of a phosphate moiety ex viw). Unless expressly indicated otherwise herein, such understandings of the person of ordinary skill in the art are used when describing the AAT RNAi agents and compositions of AAT RNAi agents disclosed herein.

Targeting groups and linking groups include the following, for which their chemical structures are provided below in Table 7: (PAZ), (NAG13), (NAG13)s, (NAG18). (NAG18)s, (NAG24). (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), (NAG39)s. Each sense strand and/or antisense strand can have any targeting groups or linking groups listed above, as well as other targeting or linking groups, conjugated to the 5′ and/or 3′ end of the sequence.

TABLE 4
AAT RNAi Agent Antisense Strand Sequences

| Antisense | SEQ | | SEQ | Underlying |
|---|---|---|---|---|
| Strand | ID | Antisense Sequence (Modified) | ID | Base Sequence |
| ID: | NO. | (5′ → 3′) | NO. | (5′ → 3′) |
| AM00516-AS | 888 | dTGfgAfaCfUuNAUfgGfuGfaUfgAfuAfudTsdT | 783 | TGGAACUUGGUGAUGAUAUTT |
| AM02129-AS | 889 | dTsGfsgAfaCfUuNAUfgGfuGfaUfgAfuAfudTsdT | 783 | TGGAACUUGGUGAUGAUAUTT |
| AM02130-AS | 890 | dTsGfsgAfaCfUuNAUfgGfuGfaUfgAfuAfuCfgsu | 784 | TGGAACUUGGUGAUGAUAUCGUG |
| | | sg | | |
| AM04786-AS | 891 | aCfUuNAUfgGfuGfaUfgAfuAfudTsdT | 785 | ACUUGGUGAUGAUAUTT |
| AM05303-AS | 892 | dTdGdGdAdAdCdTdTdGdGdTdGdAdTdGdAdTdA | 786 | TGGAACTTGGTGATGATATTT |
| | | dTdTsdT | | |
| AM05643-AS | 893 | usUfsusAfaAfcAfUfGfcCfuAfaAfcGfcusu | 787 | UUUAAACAUGCCUAAACGCUU |
| AM05645-AS | 894 | usGfscsAfuUfgCfCfCfaGfgUfaUfuUfcusu | 788 | UGCAUUGCCCAGGUAUUUCUU |
| AM05647-AS | 895 | usGfsgsAfaCfuUfGfGfuGfaUfgAfuAfuusu | 789 | UGGAACUUGGUGAUGAUAUUU |
| AM05649-AS | 896 | usGfsasUfcAftiAfGfGfuucCfaGfuAfausu | 790 | UGAUCAUAGGUUCCAGUAAUU |
| AM05651-AS | 897 | usAfscsAfgCfcUfUfAfuGfcAfcGfgCfcusu | 791 | UACAGCCUUAUGCACGGCCUU |
| AM05653-AS | 898 | usUfscsGfaUfgGfUfCfaGfcAfcAfgCfcusu | 792 | UUCGAUGGUCAGCACAGCCUU |
| AM05655-AS | 899 | usCfsasAfaGfgGfUfUfuGfuUfgAfaCfuusu | 793 | UCAAAGGGUUUGUUGAACUUU |
| AM05657-AS | 900 | usGfsusUfaAfaCfAfUfgCfcUfaAfaCfgusu | 794 | UGUUAAACAUGCCUAAACGUU |
| AM05659-AS | 901 | usUfuAfaAfcgugcCfuAfaAfcGfcsUfsg | 795 | UUUAAACGUGCCUAAACGCUG |
| AM05661-AS | 902 | usGfscAfuUfgcccaGfgUfaUfuUfcsAfsg | 796 | UGCAUUGCCCAGGUAUUUCAG |
| AM05663-AS | 903 | usGfsgAfaCfuugguGfaUfgAfuAfusCfsg | 797 | UGGAACUUGGUGAUGAUAUCG |
| AM05665-AS | 904 | usGfsaUfcAfuagguUfcCfaGfuAfasUfsg | 798 | UGAUCAUAGGUUCCAGUAAUG |
| AM05667-AS | 905 | usAfscAfgCfcuuauGfcAfcGfgCfcsUfsu | 791 | UACAGCCUUAUGCACGGCCUU |
| AM05669-AS | 906 | usUfscGfaUfggucaGfcAfcAfgCfcsUfsu | 792 | UUCGAUGGUCAGCACAGCCUU |
| AM05671-AS | 907 | usCfsaAfaGfgguuuGfuUfgAfaCfusUfsg | 799 | UCAAAGGGUUUGUUGAACUUG |
| AM05673-AS | 908 | usGfsuUfaAfacaugCfcUfaAfaCfgsCfsg | 800 | UGUUAAACAUGCCUAAACGCG |
| AM05677-AS | 909 | usUfsuAfaAfcgugcCfuAfaAfcGfcsUfsg | 795 | UUUAAACGUGCCUAAACGCUG |
| AM05884-AS | 910 | vpusGfsusUfaAfaCfAfUfgCfcUfaAfaCfgusu | 794 | UGUUAAACAUGCCUAAACGUU |
| AM05885-AS | 911 | cPrpusGfsusUfaAfaCfAfUfgCfcUfaAfaCfgusu | 794 | UGUUAAACAUGCCUAAACGUU |
| AM05886-AS | 912 | usGfsusUfaAfaCfAfUfgCfcUfaAfaCfgcsu | 801 | UGUUAAACAUGCCUAAACGCU |
| AM05887-AS | 913 | usGfsusUfaAfaCfaUfgCfcUfaAfaCfgusu | 794 | UGUUAAACAUGCCUAAACGUU |
| AM05888-AS | 914 | usGfsusUfaAfaCfaUfgCfcUfaAfaCfgcsu | 801 | UGUUAAACAUGCCUAAACGCU |
| AM05889-AS | 915 | usGfsusUfaAfaCfAfUfgCfcUfaAfaCfgCfsu | 801 | UGUUAAACAUGCCUAAACGCU |
| AM05890-AS | 916 | usGfsusUfaAfaCfAfUfgCfcUfaAfaCfgCfuusc | 802 | UGUUAAACAUGCCUAAACGCUUC |
| AM05891-AS | 917 | usGfsusUfaAfaCfaUfgCfcUfaAfaCfgCfsu | 801 | UGUUAAACAUGCCUAAACGCU |
| AM05892-AS | 918 | usGfsusUfaAfaCfaUfgCfcUfaAfaCfgCfuusc | 802 | UGUUAAACAUGCCUAAACGCUUC |
| AM05900-AS | 919 | cPrpusGfsuUfaAfacaugCfcUfaAfaCfgsCfsg | 800 | UGUUAAACAUGCCUAAACGCG |
| AM05901-AS | 920 | usGfsgsAfaCfUuNAUfGfGfuGfaUfgAfuAfuusu | 789 | UGGAACUUGGUGAUGAUAUUU |
| AM05954-AS | 921 | usGfscsUfgUfuggacUfgGfuGfuGfcusu | 803 | UGCUGUUGGACUGGUGUGCUU |
| AM05955-AS | 922 | usGfscsUfgUfuggacUfgGfuGfuGfccsa | 804 | UGCUGUUGGACUGGUGUGCCA |
| AM05956-AS | 923 | usGfscsUfgUfuggacUfgGfuGfuGfccausu | 805 | UGCUGUUGGACUGGUGUGCCAUU |
| AM05957-AS | 924 | usGfscsUfgUfuggacUfgGfuGfuGfccagsc | 806 | UGCUGUUGGACUGGUGUGCCAGC |
| AM05961-AS | 925 | usAfsasGfgCfuUfcUfgAfgUfgGfuAfcusu | 807 | UAAGGCUUCUGAGUGGUACUU |
| AM05962-AS | 926 | usAfsasGfgCfuUfcUfgAfgUfgGfuAfcasa | 808 | UAAGGCUUCUGAGUGGUACAA |
| AM05963-AS | 927 | usAfsasGfgCfuUfcUfgAfgUfgGfuAfcaacsu | 809 | UAAGGCUUCUGAGUGGUACAACU |
| AM05964-AS | 928 | gsAfsasGfgCfuUfcUfgAfgUfgGfuAfcusu | 810 | GAAGGCUUCUGAGUGGUACUU |
| AM05969-AS | 929 | asAfsgsAfcAfaAfgGfgUfuUfgUfuGfausu | 811 | AAGACAAAGGGUUUGUUGAUU |
| AM05970-AS | 930 | asAfsgsAfcAfaAfgGfgUfuUfgUfuGfaasc | 812 | AAGACAAAGGGUUUGUUGAAC |
| AM05973-AS | 931 | usAfsgsAfcAfaAfgGfgUfuUfgUfuGfaasc | 813 | UAGACAAAGGGUUUGUUGAAC |
| AM05974-AS | 932 | asAfsgsAfcAfaAfgGfgUfuUfgUfuGfaacusu | 814 | AAGACAAAGGGUUUGUUGAACUU |
| AM05976-AS | 933 | usAfsgsAfcAfuGfgGfuAfuGfgCfcUfcusu | 815 | UAGACAUGGGUAUGGCCUCUU |
| AM05977-AS | 934 | usAfsgsAfcAfuGfgGfuAfuGfgCfcUfcusa | 816 | UAGACAUGGGUAUGGCCUCUA |
| AM05979-AS | 935 | usAfsgsAfcAfuGfgGfuAfuGfgCfcticuaasa | 817 | UAGACAUGGGUAUGGCCUCUAAA |
| AM05980-AS | 936 | usAfsgsAfcAfuGfgGfuAfuGfgCfcUfcuausu | 818 | UAGACAUGGGUAUGGCCUCUAUU |
| AM05982-AS | 937 | usUfsusGfaUfcUfgUfuUfcUfuGfgCfcusu | 819 | UUUGAUCUGUUUCUUGGCCUU |
| AM05983-AS | 938 | usUfsusGfaUfcUfgUfuUfcUfuGfgCfcusc | 820 | UUUGAUCUGUUUCUUGGCCUC |
| AM05985-AS | 939 | usUfsusGfaUfcUfgUfuUfcUfuGfgCfcucusu | 821 | UUUGAUCUGUUUCUUGGCCUCUU |
| AM05987-AS | 940 | usGfsusUfgGfacuggUfgUfgCfcAfgusu | 822 | UGUUGGACUGGUGUGCCAGUU |
| AM05989-AS | 941 | usGfsusUfgGfacuggUfgUfgCfcAfgcsu | 823 | UGUUGGACUGGUGUGCCAGCU |
| AM05990-AS | 942 | usGfsusUfgGfacuggUfgUfgCfcAfgcugsg | 824 | UGUUGGACUGGUGUGCCAGCUGG |
| AM05992-AS | 943 | usGfsusUfgGfacuggUfgUfgCfcAfgcusg | 825 | UGUUGGACUGGUGUGCCAGCUG |
| AM05994-AS | 944 | asAfsasGfgGfuUfuGfuUfgAfaCfuUfgusu | 826 | AAAGGGUUUGUUGAACUUGUU |
| AM05996-AS | 945 | asAfsasGfgGfuUfuGfuUfgAfaCfuUfgasc | 827 | AAAGGGUUUGUUGAACUUGAC |
| AM05998-AS | 946 | usAfsasGfgGfuUfuGfuUfgAfaCfuUfgaccsu | 828 | UAAGGGUUUGUUGAACUUGACCU |
| AM05999-AS | 947 | usAfsasGfgGfuUfuGfuUfgAfaCfuUfgasc | 829 | UAAGGGUUUGUUGAACUUGAC |
| AM06124-AS | 948 | usAfsusUfgGfuGfcUfgUfuGfgAfcUfgusu | 830 | UAUUGGUGCUGUUGGACUGUU |
| AM06125-AS | 949 | usAfsusUfgGfuGfcUfgUfuGfgAfcUfggsu | 831 | UAUUGGUGCUGUUGGACUGGU |
| AM06126-AS | 950 | usAfsusUfgGfuGfcUfgUfuGfgAfcUfggusu | 832 | UAUUGGUGCUGUUGGACUGGUU |
| AM06130-AS | 951 | usUfsgsUfuGfgacugGfuGfuGfcCfasg | 833 | UUGUUGGACUGGUGUGCCAG |
| AM06131-AS | 952 | usUfsgsUfuGfgacugGfuGfuGfcCfagcsu | 834 | UUGUUGGACUGGUGUGCCAGCU |
| AM06133-AS | 953 | usAfsusAfgAfcAfuGfgGfuAfuGfgCfcusc | 835 | UAUAGACAUGGGUAUGGCCUC |
| AM06134-AS | 954 | usAfsusAfgAfcauggGfuAfuGfgCfcusc | 835 | UAUAGACAUGGGUAUGGCCUC |
| AM06137-AS | 955 | usCfsasAfaGfgGfuUfuGfuUfgAfaCfuugasc | 836 | UCAAAGGGUUUGUUGAACUUGAC |
| AM06140-AS | 956 | usUfsasUfuGfgugcuGfuUfgGfaCfugsg | 837 | UUAUUGGUGCUGUUGGACUGG |
| AM06227-AS | 957 | usGfsusUfaAfaCfaUfgCfcUfaAfaCfgsc | 838 | UGUUAAACAUGCCUAAACGC |
| AM06228-AS | 958 | usGfsusUfaAfaCfaUfgCfcUfaAfaCfgcusu | 839 | UGUUAAACAUGCCUAAACGCUU |
| AM06234-AS | 959 | usGfsuUfaAfaCfaUfgCfcUfaAfaCfgsCfsg | 800 | UGUUAAACAUGCCUAAACGCG |
| AM06235-AS | 960 | usGfsuUfaAfacaugCfcUfaAfaCfgCfsu | 801 | UGUUAAACAUGCCUAAACGCU |
| AM06237-AS | 961 | usGfsuUfaAfaCfAfUfgCfcUfaAfaCfgsCfsg | 800 | UGUUAAACAUGCCUAAACGCG |
| AM06238-AS | 962 | NpusGfsusUfaAfaCfaUfgCfcUfaAfaCfgusu | 794 | UGUUAAACAUGCCUAAACGUU |
| AM06261-AS | 963 | NusGfsusUfaAfaCfaUfgCfcUfaAfaCfgusu | 794 | UGUUAAACAUGCCUAAACGUU |

TABLE 5
AAT RNAi Agent Sense Strand Sequences

| Sense | SEQ | | SEQ | Underlying |
|---|---|---|---|---|
| Strand | ID | Sense Sequence (Modified) | ID | Base Sequence |
| ID: | NO. | (5′ → 3′) | NO. | (5′ → 3′) |
| AM01887-SS | 964 | (Chol-TEG)uAuAfuAfuCfaUfcAfcCfaAfgUfuCfcAf(invdT)(TEG- | 845 | UAUAUAUCAUCACCAAGUUCCAT |
| | | Biotin) | | |
| AM01888-SS | 965 | (Chol-TEG)uAuAfuAfuCfaUfcAfcCfaAfgUthCfcAf(invdT)(PEG- | 845 | UAUAUAUCAUCACCAAGUUCCAT |
| | | C3-SS) | | |
| AM01855-SS | 966 | (Alk-SS-C6)AfuAfuCfaUfcAfcCfaAfgUfuCfcAf(invdT) | 846 | AUAUCAUCACCAAGUUCCAT |
| AM02132-SS | 967 | CfsgsAfuAfuCfaUfcAfcCfaAfgUfuCfcAf(C6-SS-Alk) | 847 | CGAUAUCAUCACCAAGUUCCA |
| AM02390-SS | 968 | CfsgsAfuAfuCfaUfcAfcCfaAfgUfuCfcAf(C6-SS-Alk-Me) | 847 | CGAUAUCAUCACCAAGUUCCA |
| AM04785-SS | 969 | uAfuCfaUfcAfcCfaAfgUfuCfcAf(invdT) | 848 | UAUCAUCACCAAGUUCCAT |
| AM05304-SS | 970 | (Chol-TEG)dTdAdTdAdTdAdTdCdAdTdCdAdCdCdAdAdGdTdTdCdCsd | 849 | TATATATCATCACCAAGTTCCAT |
| | | A(invdT)(TEG-Biotin) | | |
| AM05599-SS | 971 | (Chol-TEG)dTdAdTdAdTdAdTdCdAdTdCdAdCdCdAdAdGdTdTdCdCsd | 849 | TATATATCATCACCAAGTTCCAT |
| | | A(invdT) | | |
| AM05642-SS | 972 | (NAG25)(invAb)GfcGfuUfuAfGfGfcAfuGfuUfuAfaausu(invAb) | 850 | GCGUUUAGGCAUGUUUAAAUU |
| AM05644-SS | 973 | (NAG25)(invAb)GfaAfaUfaCfCfUfgGfgCfaAfuGfcausu(invAb) | 851 | GAAAUACCUGGGCAAUGCAUU |
| AM05646-SS | 974 | (NAG25)(invAb)AfuAfuCfaUfCfAfcCfaAfgUfuCfcausu(invAb) | 852 | AUAUCAUCACCAAGUUCCAUU |
| AM05648-SS | 975 | (NAG25)(invAb)UfuAfcUfgGfAfAfcCfuAfuGfaUfcausu(invAb) | 853 | UUACUGGAACCUAUGAUCAUU |
| AM05650-SS | 976 | (NAG25)(invAb)GfgCfcGfuGfCfAfuAfaGfgCfuGfuausu(invAb) | 854 | GGCCGUGCAUAAGGCUGUAUU |
| AM05652-SS | 977 | (NAG25)(invAb)GfgCfuGfuGfCfUfgAfcCfaUfcGfaausu(invAb) | 855 | GGCUGUGCUGACCAUCGAAUU |
| AM05654-SS | 978 | (NAG25)(invAb)AfgUfuCfaAfCfAfaAfcCfcUfuUfgausu(invAb) | 856 | AGUUCAACAAACCCUUUGAUU |
| AM05656-SS | 979 | (NAG25)(invAb)CfgUfuUfaGfGfCfaUfgUfuUfaAfcausu(invAb) | 857 | CGUUUAGGCAUGUUUAACAUU |
| AM05658-SS | 980 | (NAG25)scsagcguuuAfGfGfcauguuuaasa(invAb) | 858 | CAGCGUUUAGGCAUGUUUAAA |
| AM05660-SS | 981 | (NAG25)scsugaaauaCfCfUfgggcaaugcsa(invAb) | 859 | CUGAAAUACCUGGGCAAUGCA |
| AM05662-SS | 982 | (NAG25)scsgauaucaUfCfAfccaaguuccsa(invAb) | 847 | CGAUAUCAUCACCAAGUUCCA |
| AM05664-SS | 983 | (NAG25)scsauuacugGfAfAfccuaugaucsa(invAb) | 860 | CAUUACUGGAACCUAUGAUCA |
| AM05666-SS | 984 | (NAG25)sasaggccguGfCfAfuaaggcugusa(invAb) | 861 | AAGGCCGUGCAUAAGGCUGUA |
| AM05668-SS | 985 | (NAG25)sasaggcuguGfCfUfgaccaucgasa(invAb) | 862 | AAGGCUGUGCUGACCAUCGAA |
| AM05670-SS | 986 | (NAG25)scsaaguucaAfCfAfaacccuuugsa(invAb) | 863 | CAAGUUCAACAAACCCUUUGA |
| AM05672-SS | 987 | (NAG25)scsgcguuuaGfGfCfauguuuaacsa(invAb) | 864 | CGCGUUUAGGCAUGUUUAACA |
| AM05658-SS | 988 | (NAG25)scsagcguuuAfGfGfcauguuuaasa(invAb) | 858 | CAGCGUUUAGGCAUGUUUAAA |
| AM05893-SS | 989 | (NAG25)s(invAb)scguuuaGfGfCfauguuuaacausu(invAb) | 857 | CGUUUAGGCAUGUUUAACAUU |
| AM05894-SS | 990 | (NAG25)s(invAb)sCfgUfuUfaGfGfCfaUfgUfuUfaAfcas(invAb) | 429 | CGUUUAGGCAUGUUUAACA |
| AM05895-SS | 991 | (NAG25)s(invAb)scguuuaGfGfCfauguuuaacas(invAb) | 429 | CGUUUAGGCAUGUUUAACA |
| AM05896-SS | 992 | (NAG25)s(invAb)saaCfgUfuUfaGfGfCfaUfgUfuUfaAfcas(invAb) | 865 | AACGUUUAGGCAUGUUUAACA |
| AM05897-SS | 993 | (NAG25)s(invAb)sagCfgUfuUfaGfGfCfaUfgUfuUfaAfcas(invAb) | 866 | AGCGUUUAGGCAUGUUUAACA |
| AM05898-SS | 994 | (NAG25)s(invAb)saacguuuaGfGfCfauguuuaacas(invAb) | 865 | AACGUUUAGGCAUGUUUAACA |
| AM05899-SS | 995 | (NAG25)s(invAb)sagcguuuaGfGfCfauguuuaacas(invAb) | 866 | AGCGUUUAGGCAUGUUUAACA |
| AM05958-SS | 996 | (NAG37)s(invAb)sgcacacCfAfGfuccaacagcas(invAb) | 454 | GCACACCAGUCCAACAGCA |
| AM05959-SS | 997 | (NAG37)s(invAb)suggcacacCfAfGfuccaacagcas(invAb) | 867 | UGGCACACCAGUCCAACAGCA |
| AM05960-SS | 998 | (NAG37)s(invAb)saagcacacCfAfGfuccaacagcas(invAb) | 868 | AAGCACACCAGUCCAACAGCA |
| AM05965-SS | 999 | (NAG37)s(invAb)sguaccaCfUfCfagaagccuuas(invAb) | 519 | GUACCACUCAGAAGCCUUA |
| AM05966-SS | 1000 | (NAG37)s(invAb)suuguaccaCfUfCfagaagccuuas(invAb) | 869 | UUGUACCACUCAGAAGCCUUA |
| AM05967-SS | 1001 | (NAG37)s(invAb)sguaccaCfUfCfagaagccuucs(invAb) | 518 | GUACCACUCAGAAGCCUUC |
| AM05968-SS | 1002 | (NAG37)s(invAb)sucaacaAfAfCfccuuugucuus(invAb) | 738 | UCAACAAACCCUUUGUCUU |
| AM05971-SS | 1003 | (NAG37)s(invAb)sguucaacaAfAfCfccuuugucuus(invAb) | 870 | GUUCAACAAACCCUUUGUCUU |
| AM05972-SS | 1004 | (NAG37)s(invAb)sguucaacaAfAfCfccuuugucuas(invAb) | 871 | GUUCAACAAACCCUUUGUCUA |
| AM05975-SS | 1005 | (NAG37)s(invAb)sgaggccAfUfAfcccaugucuas(invAb) | 707 | GAGGCCAUACCCAUGUCUA |
| AM05978-SS | 1006 | (NAG37)s(invAb)suagaggccAfUfAfcccaugucuas(invAb) | 872 | UAGAGGCCAUACCCAUGUCUA |
| AM05981-SS | 1007 | (NAG37)s(invAb)sggccaaGfAfAfacagaucaaas(invAb) | 537 | GGCCAAGAAACAGAUCAAA |
| AM05984-SS | 1008 | (NAG37)s(invAb)sgaggccaaGfAfAfacagaucalas(invAb) | 873 | GAGGCCAAGAAACAGAUCAAA |
| AM05986-SS | 1009 | (NAG37)s(invAb)scuggcaCfAfCfcaguccaacas(invAb) | 445 | CUGGCACACCAGUCCAACA |
| AM05988-SS | 1010 | (NAG37)s(invAb)sagcuggcaCfAfCfcaguccaacas(invAb) | 874 | AGCUGGCACACCAGUCCAACA |
| AM05991-SS | 1011 | (NAG37)s(invAb)sgcuggcaCfAfCfcaguccaacas(invAb) | 875 | GCUGGCACACCAGUCCAACA |
| AM05993-SS | 1012 | (NAG37)s(invAb)scaaguuCfAfAfcaaacccuuus(invAb) | 725 | CAAGUUCAACAAACCCUUU |
| AM05995-SS | 1013 | (NAG37)s(invAb)sgucaaguuCfAfAfcaaacccuuus(invAb) | 876 | GUCAAGUUCAACAAACCCUUU |
| AM05997-SS | 1014 | (NAG37)s(invAb)sgucaaguuCfAfAfcaaacccuuas(invAb) | 877 | GUCAAGUUCAACAAACCCUUA |
| AM06127-SS | 1015 | (NAG37)s(invAb)scaguccAfAfCfagcaccaauas(invAb) | 458 | CAGUCCAACAGCACCAAUA |
| AM06128-SS | 1016 | (NAG37)s(invAb)saccaguccAfAfCfagcaccaauas(invAb) | 878 | ACCAGUCCAACAGCACCAAUA |
| AM06129-SS | 1017 | (NAG37)s(invAb)sccaguccAfAfCfagcaccaauas(invAb) | 879 | CCAGUCCAACAGCACCAAUA |
| AM06132-SS | 1018 | (NAG37)s(invAb)scuggcacAfCfCfaguccaacaas(invAb) | 880 | CUGGCACACCAGUCCAACAA |
| AM06135-SS | 1019 | (NAG37)s(invAb)sggccauAfCfCfcaugucuauas(invAb) | 712 | GGCCAUACCCAUGUCUAUA |
| AM06136-SS | 1020 | (NAG37)s(invAb)sgaggccauAfCfCfcaugucuauas(invAb) | 881 | GAGGCCAUACCCAUGUCUAUA |
| AM06138-SS | 1021 | (NAG37)s(invAb)sgucaaguucaAfCfAfaacccuuugas(invAb) | 882 | GUCAAGUUCAACAAACCCUUUGA |
| AM06139-SS | 1022 | (NAG37)s(invAb)scaaguucaAfCfAfaacccuuugas(invAb) | 863 | CAAGUUCAACAAACCCUUUGA |
| AM06141-SS | 1023 | (NAG37)s(invAb)sccaguccaAfCfAfgcaccaauaas(invAb) | 883 | CCAGUCCAACAGCACCAAUAA |
| AM06195-SS | 1024 | (NAG37)s(invAb)scgcguuuaGfGfCfauguuuaacas(invAb) | 864 | CGCGUUUAGGCAUGUUUAACA |
| AM06223-SS | 1025 | (NAG37)s(invAb)scguuuaGfGfCfauguuuaacas(invAb) | 429 | CGUUUAGGCAUGUUUAACA |
| AM06224-SS | 1026 | (NAG37)s(invAb)scsgcguuuaGfGfCfauguuuaacsa(invAb) | 864 | CGCGUUUAGGCAUGUUUAACA |
| AM06225-SS | 1027 | (NAG37)s(invAb)sagCfgUfuUfaGfGfCfaUfgUfuUfaAfcas(invAb) | 866 | AGCGUUUAGGCAUGUUUAACA |
| AM06226-SS | 1028 | (NAG37)s(invAb)scguuuaGfGfCfauguuuaacausu(invAb) | 857 | CGUUUAGGCAUGUUUAACAUU |
| AM06229-SS | 1029 | (NAG37)s(invAb)sgcguuuaGfGfCfauguuuaacas(invAb) | 884 | GCGUUUAGGCAUGUUUAACA |
| AM06230-SS | 1030 | (NAG37)s(invAb)sgcguuuaGfGfCfauguuuaacausu(invAb) | 885 | GCGUUUAGGCAUGUUUAACAUU |
| AM06231-SS | 1031 | (NAG37)s(invAb)scgcguuuaGfGfCfauguuuaacsausu(invAb) | 886 | CGCGUUUAGGCAUGUUUAACAUU |
| AM06232-SS | 1032 | (NAG37)s(invAb)sagCfgUfuUfaGfGfCfaUfgUfuUfaAfcausu | 887 | AGCGUUUAGGCAUGUUUAACAUU |
| | | (invAb) | | |
| AM06236-SS | 1033 | (NAG37)s(invAb)sagcguuuaGfGfCfauguuuaacas(invAb) | 866 | AGCGUUUAGGCAUGUUUAACA |
| AM06239-SS | 1034 | (NAG37)s(invAb)scgcguuuaGfGfCfauguuuaacsas(invAb) | 864 | CGCGUUUAGGCAUGUUUAACA |

The AAT RNAi agents described herein are formed by annealing an antisense strand with a sense strand. A sense strand containing a sequence listed in Table 2, Table 3, or Table 5 can be hybridized to any antisense strand containing a sequence listed in Table 2, Table 3, or Table 4, provided the two sequences have a region of at least 85% complementarity over a contiguous 16, 17, 18, 19, 20, or 21 nucleotide sequence.

In some embodiments, the antisense strand of an AAT RNAi agent disclosed herein differs by 0, 1, 2, or 3 nucleotides from any of the antisense strand sequences in Table 4. In some embodiments, the sense strand of an AAT RNAi agent disclosed herein differs by 0, 1, 2, or 3 nucleotides from any of the sense strand sequences in Table 5.

In some embodiments, an AAT RNAi agent antisense strand comprises a nucleotide sequence of any of the sequences in Table 2, Table 3, or Table 4. In some embodiments, an AAT RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24, of any of the sequences in Table 2, Table 3, or Table 4. In certain embodiments, an AAT RNAi agent antisense strand comprises or consists of a modified sequence of any one of the modified sequences in Table 4.

In some embodiments, an AAT RNAi agent sense strand comprises the nucleotide sequence of any of the sequences in Table 2, Table 3, or Table 5. In some embodiments, an AAT RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24 of any of the sequences in Table 2, Table 3, or Table 5. In certain embodiments, an AAT RNAi agent sense strand comprises or consists of a modified sequence of any one of the modified sequences in Table 5.

For the AAT RNAi agents disclosed herein, the nucleotide at position 1 of the antisense strand (from 5′ end→3′ end) can be perfectly complementary to an AAT gene, or can be non-complementary to an AAT gene. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, an AAT RNAi agent antisense strand comprises the sequence of nucleotides (from 5′ end→3′ end) 2-18 or 2-19 of any of the antisense strand sequences in Table 2, Table 3, or Table 4. In some embodiments, an AAT RNAi sense strand comprises the sequence of nucleotides (from 5′ end→3′ end) 1-17 or 1-18 of any of the sense strand sequences in Table 2, Table 3, or Table 5.

In some embodiments, an AAT RNAi agent includes (i) an antisense strand comprising the sequence of nucleotides (from 5′ end→3′ end) 2-18 or 2-19 of any of the antisense strand sequences in Table 2, Table 3, or Table 4, and (ii) a sense strand comprising the sequence of nucleotides (from 5′ end→3′ end) 1-17 or 1-18 of any of the sense strand sequences in Table 2, Table 3, or Table 5.

A sense strand containing a sequence listed in Table 2, Table 3, or Table 5 can be hybridized to any antisense strand containing a sequence listed in Table 2, Table 3, or Table 5, provided the two sequences have a region of at least 85% complementarity over a contiguous 16, 17, 18, 19, 20, or 21 nucleotide sequence. In some embodiments, the AAT RNAi agent has a sense strand consisting of the modified sequence of any of the modified sequences in Table 5, and an antisense strand consisting of the modified sequence of any of the modified sequences in Table 4. Representative sequence pairings are exemplified by the Duplex ID Nos. shown in Table 6.

In some embodiments, an AAT RNAi agent comprises any of the duplexes represented by any of the Duplex ID Nos. presented herein. In some embodiments, an AAT RNAi agent consists of any of the duplexes represented by any of the Duplex ID Nos. presented herein. In some embodiments, an AAT RNAi agent comprises the sense strand and antisense strand nucleotide sequences of any of the duplexes represented by any of the Duplex ID Nos. presented herein. In some embodiments, an AAT RNAi agent includes the sense strand and antisense strand nucleotide sequences of any of the duplexes represented by any of the Duplex ID Nos. presented herein and a targeting group and/or linking group, wherein the targeting group and/or linking group is covalently linked (i.e., conjugated) to the sense strand or the antisense strand. In some embodiments, an AAT RNAi agent includes the sense strand and antisense strand modified nucleotide sequences of any of the duplexes represented by any of the Duplex ID Nos. presented herein. In some embodiments, an AAT RNAi agent comprises the sense strand and antisense strand modified nucleotide sequences of any of the duplexes represented by any of the Duplex ID Nos. presented herein and a targeting group and/or linking group, wherein the targeting group and/or linking group is covalently linked to the sense strand or the antisense strand.

In some embodiments, an AAT RNAi agent comprises an antisense strand and a sense strand having the nucleotide sequences of any of the antisense strand/sense strand duplexes of Table 2, Table 3, or Table 6, and comprises an asialoglycoprotein receptor ligand targeting group.

In some embodiments, an AAT RNAi agent comprises an antisense strand and a sense strand having the nucleotide sequences of any of the antisense strand/sense strand duplexes of Table 2 or Table 5, and further comprises a targeting group selected from the group consisting of (PAZ), (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34). (NAG34)s, (NAG35), (NAG35)s, (NAG36), (NAG36)s, (NAG37). (NAG37)s, (NAG38), (NAG38)s. (NAG39), (NAG39)s. In some embodiments, the targeting group is (NAG25) or (NAG25)s as defined in Table 7. In other embodiments, the targeting group is (NAG37) or (NAG37)s as defined in Table 7.

In some embodiments, an AAT RNAi agent comprises an antisense strand and a sense strand having the modified nucleotide sequence of any of the antisense strand and/or sense strand nucleotide sequences of any of the duplexes of Table 6.

In some embodiments, an AAT RNAi agent comprises an antisense strand and a sense strand having a modified nucleotide sequence of any of the antisense strand and/or sense strand nucleotide sequences of any of the duplexes of Table 6, and comprises an asialoglycoprotein receptor ligand targeting group.

In some embodiments, an AAT RNAi agent comprises the duplex structure of any of the duplexes in Table 6.

In some embodiments, an AAT RNAi agent consists of the duplex structure of any of the duplexes in Table 6.

TABLE 6
AAT RNAi Agents Identified by Duplex ID No.
with Corresponding Sense and Antisense Strands

| Duplex | Antisense | Sense |
|---|---|---|
| ID | Strand ID | Strand ID |
| AD01131 | AM00516-AS | AM01887-SS |
| AD01132 | AM00516-AS | AM01888-SS |
| AD01174 | AM00516-AS | AM01855-SS |
| AD01286 | AM02129-AS | AM01855-SS |
| AD01287 | AM02130-AS | AM02132-SS |
| AD01442 | AM02130-AS | AM02390-SS |
| AD03752 | AM04786-AS | AM04785-SS |
| AD04156 | AM05303-AS | AM05304-SS |
| AD04406 | AM05303-AS | AM05599-SS |
| AD04444 | AM05643-AS | AM05642-SS |
| AD04445 | AM05645-AS | AM05644-SS |
| AD04446 | AM05647-AS | AM05646-SS |
| AD04447 | AM05649-AS | AM05648-SS |
| AD04448 | AM05651-AS | AM05650-SS |
| AD04449 | AM05653-AS | AM05652-SS |
| AD04450 | AM05655-AS | AM05654-SS |
| AD04451 | AM05657-AS | AM05656-SS |
| AD04452 | AM05659-AS | AM05658-SS |
| AD04453 | AM05661-AS | AM05660-SS |
| AD04454 | AM05663-AS | AM05662-SS |
| AD04455 | AM05665-AS | AM05664-SS |
| AD04456 | AM05667-AS | AM05666-SS |
| AD04457 | AM05669-AS | AM05668-SS |
| AD04458 | AM05671-AS | AM05670-SS |
| AD04459 | AM05673-AS | AM05672-SS |
| AD04464 | AM05677-AS | AM05658-SS |
| AD04601 | AM05884-AS | AM05656-SS |
| AD04602 | AM05885-AS | AM05656-SS |
| AD04603 | AM05886-AS | AM05656-SS |
| AD04604 | AM05887-AS | AM05893-SS |
| AD04605 | AM05888-AS | AM05893-SS |
| AD04606 | AM05657-AS | AM05894-SS |
| AD04607 | AM05886-AS | AM05894-SS |
| AD04608 | AM05887-AS | AM05895-SS |
| AD04609 | AM05888-AS | AM05895-SS |
| AD04610 | AM05657-AS | AM05896-SS |
| AD04611 | AM05889-AS | AM05897-SS |
| AD04612 | AM05890-AS | AM05897-SS |
| AD04613 | AM05887-AS | AM05898-SS |
| AD04614 | AM05891-AS | AM05899-SS |
| AD04615 | AM05892-AS | AM05899-SS |
| AD04616 | AM05900-AS | AM05672-SS |
| AD04617 | AM05901-AS | AM05646-SS |
| AD04652 | AM05954-AS | AM05958-SS |
| AD04653 | AM05955-AS | AM05958-SS |
| AD04654 | AM05955-AS | AM05959-SS |
| AD04655 | AM05954-AS | AM05960-SS |
| AD04656 | AM05956-AS | AM05959-SS |
| AD04657 | AM05957-AS | AM05959-SS |
| AD04658 | AM05961-AS | AM05965-SS |
| AD04659 | AM05962-AS | AM05965-SS |
| AD04660 | AM05962-AS | AM05966-SS |
| AD04661 | AM05963-AS | AM05966-SS |
| AD04662 | AM05964-AS | AM05967-SS |
| AD04663 | AM05969-AS | AM05968-SS |
| AD04664 | AM05970-AS | AM05968-SS |
| AD04665 | AM05970-AS | AM05971-SS |
| AD04666 | AM05973-AS | AM05972-SS |
| AD04667 | AM05974-AS | AM05971-SS |
| AD04668 | AM05976-AS | AM05975-SS |
| AD04669 | AM05977-AS | AM05975-SS |
| AD04670 | AM05977-AS | AM05978-SS |
| AD04671 | AM05979-AS | AM05978-SS |
| AD04672 | AM05980-AS | AM05978-SS |
| AD04673 | AM05982-AS | AM05981-SS |
| AD04674 | AM05983-AS | AM05981-SS |
| AD04675 | AM05983-AS | AM05984-SS |
| AD04676 | AM05985-AS | AM05984-SS |
| AD04677 | AM05987-AS | AM05986-SS |
| AD04678 | AM05989-AS | AM05988-SS |
| AD04679 | AM05990-AS | AM05988-SS |
| AD04680 | AM05992-AS | AM05991-SS |
| AD04681 | AM05994-AS | AM05993-SS |
| AD04682 | AM05996-AS | AM05995-SS |
| AD04683 | AM05998-AS | AM05997-SS |
| AD04684 | AM05999-AS | AM05997-SS |
| AD04761 | AM06124-AS | AM06127-SS |
| AD04762 | AM06125-AS | AM06128-SS |
| AD04763 | AM06126-AS | AM06129-SS |
| AD04764 | AM06130-AS | AM06132-SS |
| AD04765 | AM06131-AS | AM06132-SS |
| AD04766 | AM06133-AS | AM06135-SS |
| AD04767 | AM06134-AS | AM06136-SS |
| AD04768 | AM06137-AS | AM06138-SS |
| AD04769 | AM06137-AS | AM06139-SS |
| AD04770 | AM06140-AS | AM06141-SS |
| AD04805 | AM05673-AS | AM06195-SS |
| AD04824 | AM05887-AS | AM06223-SS |
| AD04825 | AM05900-AS | AM06224-SS |
| AD04826 | AM05889-AS | AM06225-SS |
| AD04827 | AM05888-AS | AM06223-SS |
| AD04828 | AM05887-AS | AM06226-SS |
| AD04829 | AM06227-AS | AM06229-SS |
| AD04830 | AM06228-AS | AM06229-SS |
| AD04831 | AM06228-AS | AM06230-SS |
| AD04832 | AM05673-AS | AM06231-SS |
| AD04833 | AM05889-AS | AM06232-SS |
| AD04834 | AM06227-AS | AM06230-SS |
| AD04836 | AM06234-AS | AM06195-SS |
| AD04837 | AM06235-AS | AM06236-SS |
| AD04838 | AM06237-AS | AM06239-SS |
| AD04839 | AM05673-AS | AM06239-SS |
| AD04840 | AM06238-AS | AM06223-SS |
| AD04857 | AM06261-AS | AM06223-SS |

In some embodiments, an AAT RNAi agent is prepared or provided as a salt, mixed salt, or a free-acid. The RNAi agents described herein, upon delivery to a cell expressing an AAT gene, inhibit or knockdown expression of one or more AAT genes in vivo.

Targeting Groups, Linking Groups, and Delivery Vehicles

In some embodiments, an AAT RNAi agent is conjugated to one or more non-nucleotide groups including, but not limited to, a targeting group, linking group, delivery polymer, or a delivery vehicle. The non-nucleotide group can enhance targeting, delivery or attachment of the RNAi agent. Examples of targeting groups and linking groups are provided in Table 7. The non-nucleotide group can be covalently linked to the 3′ and/or 5′ end of either the sense strand and/or the antisense strand. In some embodiments, an AAT RNAi agent contains a non-nucleotide group linked to the 3′ and/or 5′ end of the sense strand. In some embodiments, a non-nucleotide group is linked to the 5′ end of an AAT RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the RNAi agent or conjugate. In some embodiments, a non-nucleotide group enhances endocytosis of the RNAi agent.

Targeting groups or targeting moieties can enhance the pharmacokinetic or biodistribution properties of a conjugate or RNAi agent to which they are attached to improve cell-specific distribution and cell-specific uptake of the conjugate or RNAi agent. A targeting group can be monovalent, divalent, trivalent, tetravalent, or have higher valency for the target to which it is directed. Representative targeting groups include, without limitation, compounds with affinity to cell surface molecules, cell receptor ligands, haptens, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers. In some embodiments, a targeting group comprises a galactose derivative cluster.

The AAT RNAi agents described herein can be synthesized having a reactive group, such as an amine group, at the 5′-terminus. The reactive group can be used to subsequently attach a targeting group using methods typical in the art.

In some embodiments, a targeting group comprises an asialoglycoprotein receptor ligand. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formylgalactosamine, N-acetyl-galactosamine, N-propionyl-galactosamine, N-n-butanoyl-galactosamine, and N-iso-butanoylgalactos-amine (see for example: S. T. Iobst and K. Drickamer, J. B. C., 1996, 271, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945).

Galactose derivatives have been used to target molecules to hepatocytes in vivo through their binding to the asialoglycoprotein receptor expressed on the surface of hepatocytes. Binding of asialoglycoprotein receptor ligands to the asialoglycoprotein receptor(s) facilitates cell-specific targeting to hepatocytes and endocytosis of the molecule into hepatocytes. Asialoglycoprotein receptor ligands can be monomeric (e.g., having a single galactose derivative) or multimeric (e.g., having multiple galactose derivatives). The galactose derivative or galactose derivative cluster can be attached to the 3′ or 5′ end of the sense or antisense strand of the RNAi agent using methods known in the art. The preparation of targeting groups, such as galactose derivative clusters, is described in, for example, U.S. patent application Ser. No. 15/452,324 and U.S. Patent Publication No. US 2017/0253875, the contents of both of which are incorporated by reference herein in their entirety.

As used herein, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamines. In some embodiments, the galactose derivative cluster comprises three N-acetyl-galactosamines. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamines.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see, for example, U.S. Pat. No. 5,885,968; Biessen et al. J. Med. Chem. 1995 Vol. 39 p. 1538-1546). In some embodiments, the PEG spacer is a PEG3 spacer. The branch point can be any small molecule which permits attachment of three galactose derivatives and further permits attachment of the branch point to the RNAi agent. An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative cluster is comprised of a galactose derivative tetramer, which can be, for example, an N-acetyl-galactosamine tetramer.

Embodiments of the present disclosure include pharmaceutical compositions for delivering an AAT RNAi agent to a liver cell in vivo. Such pharmaceutical compositions can include, for example, an AAT RNAi agent conjugated to a galactose derivative cluster. In some embodiments, the galactose derivative cluster is comprised of a galactose derivative trimer, which can be, for example, an N-acetyl-galactosamine trimer, or galactose derivative tetramer, which can be, for example, an N-acetyl-galactosamine tetramer.

Targeting groups include, but are not limited to, (PAZ), (NAG13), (NAG13)s, (NAG18), (NAG18)s, (NAG24), (NAG24)s, (NAG25), (NAG25)s, (NAG26), (NAG26)s, (NAG27), (NAG27)s, (NAG28). (NAG28)s, (NAG29). (NAG29)s, (NAG30). (NAG30)s. (NAG31), (NAG31)s, (NAG32), (NAG32)s, (NAG33), (NAG33)s, (NAG34), (NAG34)s, (NAG35), (NAG35)s, (NAG36). (NAG36)s, (NAG37). (NAG37)s, (NAG38). (NAG38)s, (NAG39), and (NAG39)s as defined in Table 7. Other targeting groups, including galactose cluster targeting ligands, are known in the art.

In some embodiments, a linking group is conjugated to the RNAi agent. The linking group facilitates covalent linkage of the agent to a targeting group or delivery polymer or delivery vehicle. The linking group can be linked to the 3′ or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, can include, but are not limited to, reactive groups such a primary amines and alkynes, alkyl groups, abasic nucleotides, ribitol (abasic ribose), and/or PEG groups.

A linker or linking group is a connection between two atoms that links one chemical group (such as an RNAi agent) or segment of interest to another chemical group (such as a targeting group or delivery polymer) or segment of interest via one or more covalent bonds. A labile linkage contains a labile bond. A linkage may optionally include a spacer that increases the distance between the two joined atoms. A spacer can further add flexibility and/or length to the linkage. Spacers can include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

Any of the AAT RNAi agent nucleotide sequences listed in Tables 2, 3, 4, or 5, whether modified or unmodified, may contain 3′ or 5′ targeting groups or linking groups. Any of the AAT RNAi agent sequences listed in Tables 4 or 5 which contain a 3′ or 5′ targeting group or linking group, may alternatively contain no 3′ or 5′ targeting group or linking group, or may contain a different 3′ or 5′ targeting group or linking group including, but not limited to, those depicted in Table 7. Any of the AAT RNAi agent duplexes listed in Table 2, Table 3, or Table 6, whether modified or unmodified, may further comprise a targeting group or linking group, including, but not limited to, those depicted in Table 7, and the targeting group or linking group may be attached to the 3′ or 5′ terminus of either the sense strand or the antisense strand of the AAT RNAi agent duplex.

Examples of targeting groups and linking groups are provided in Table 7. Table 5 provides several embodiments of AAT RNAi agent sense strands having a targeting group or linking group linked to the 5′ or 3′ end.

TABLE 7
Structures Representing Various Modified Nucleotides, Targeting Groups, and Linking Groups

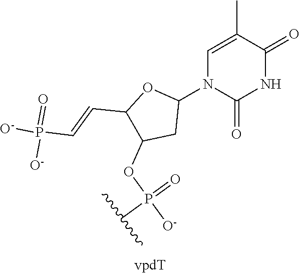 [SMILES: *P(=O)([O-])OC1CC(n2cc(C)c(=O)[nH]c2=O)OC1/C=C/P(=O)([O-])[O-]]

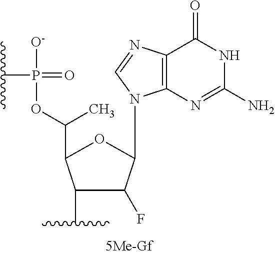 [SMILES: *C1C(F)C(n2cnc3c(=O)[nH]c(N)nc32)OC1C(C)OP(*)(=O)[O-]]

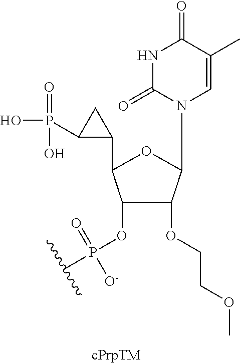 [SMILES: *P(=O)([O-])OC1C(C2CC2P(=O)(O)O)OC(n2cc(C)c(=O)[nH]c2=O)C1OCCOC]

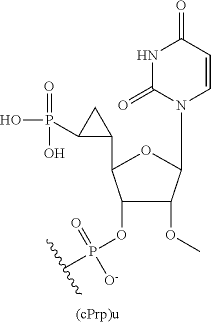 [SMILES: *P(=O)([O-])OC1C(C2CC2P(=O)(O)O)OC(n2ccc(=O)[nH]c2=O)C1OC]

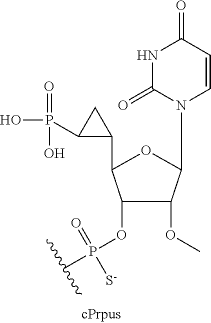 [SMILES: *P(=O)([S-])OC1C(C2CC2P(=O)(O)O)OC(n2ccc(=O)[nH]c2=O)C1OC]

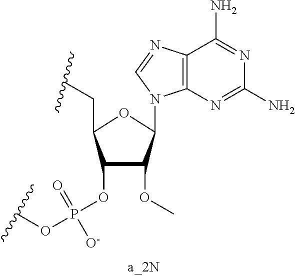

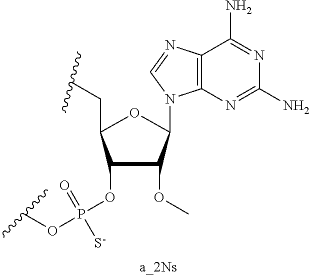

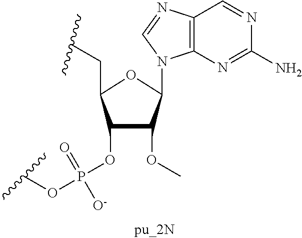

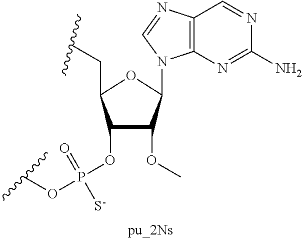

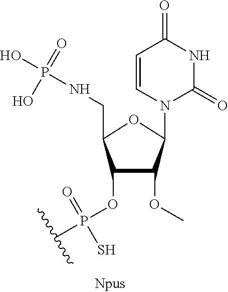

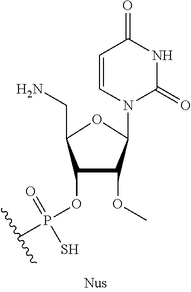

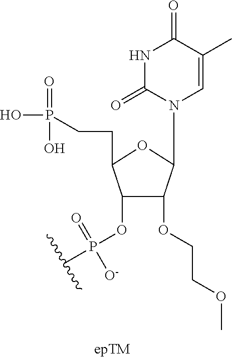 [SMILES: *P(=O)([O-])OC1C(CCP(=O)(O)O)OC(n2cc(C)c(=O)[nH]c2=O)C1OCCOC]

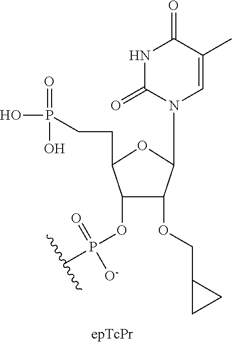 [SMILES: *P(=O)([O-])OC1C(CCP(=O)(O)O)OC(n2cc(C)c(=O)[nH]c2=O)C1OCC1CC1]

When positioned internally on oligonucleotide:

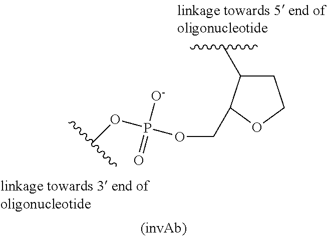 [SMILES: *OP(=O)([O-])OCC1OCCC1*]

When positioned internally on oligonucleotide:

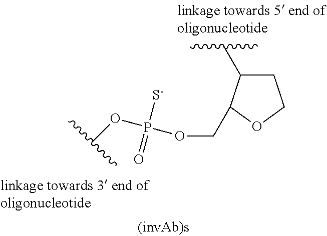 [SMILES: *OP(=O)([S-])OCC1OCCC1*]

When positioned at the 3′ terminal end of oligonucleotide:

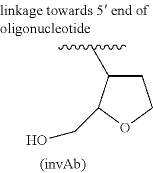 [SMILES: *C1CCOC1CO]

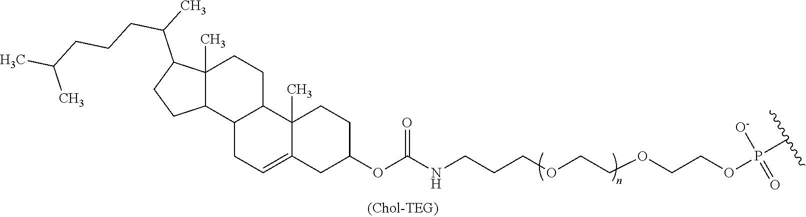

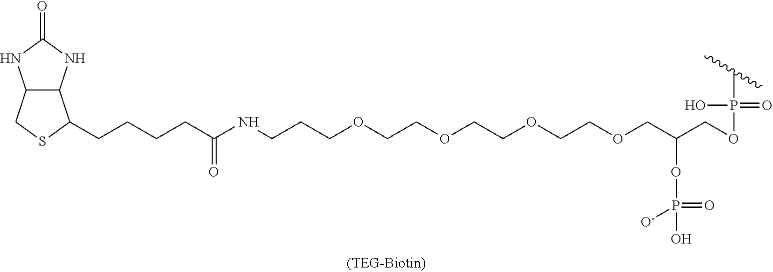 [SMILES: *P(=O)(O)OCC(COCCOCCOCCOCCCNC(=O)CCCCC1SCC2NC(=O)NC21)OP(=O)([O-])O]

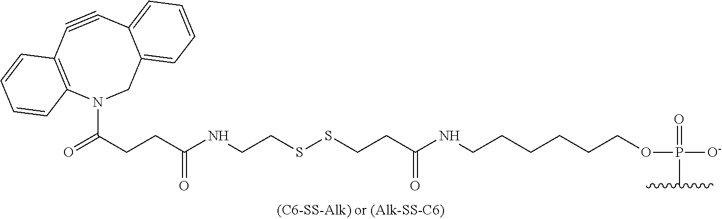 [SMILES: *P(=O)([O-])OCCCCCCNC(=O)CCSSCCNC(=O)CCC(=O)N1Cc2ccccc2C#Cc2ccccc21]

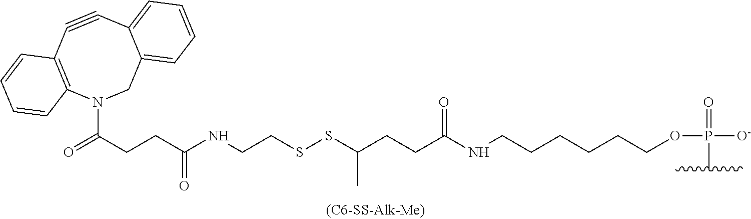 [SMILES: *P(=O)([O-])OCCCCCCNC(=O)CCC(C)SSCCNC(=O)CCC(=O)N1Cc2ccccc2C#Cc2ccccc21]

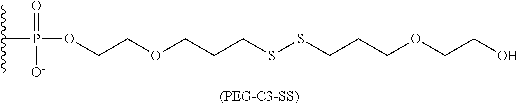 [SMILES: *P(=O)([O-])OCCOCCCSSCCCOCCO]

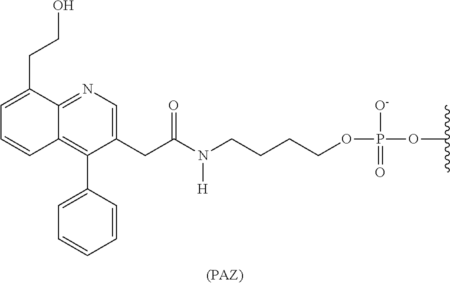 [SMILES: *OP(=O)([O-])OCCCCNC(=O)Cc1cnc2c(CCO)cccc2c1-c1ccccc1]

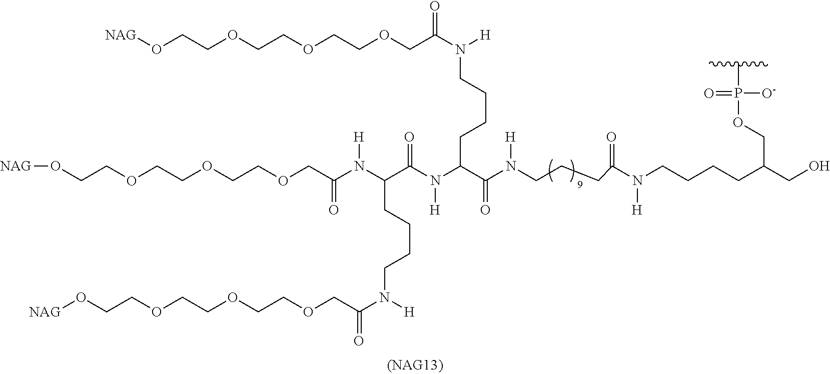

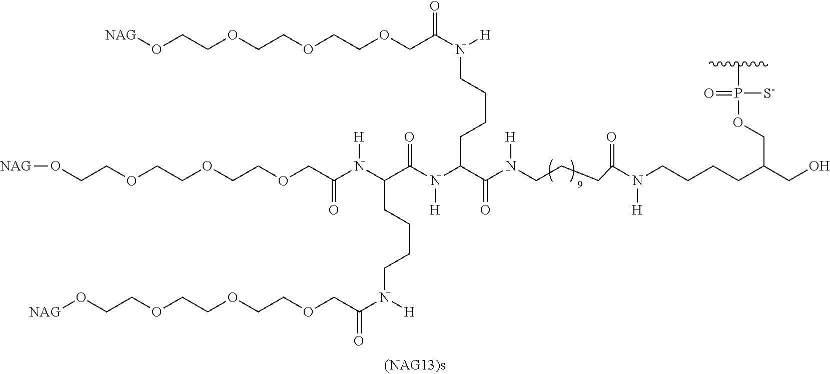

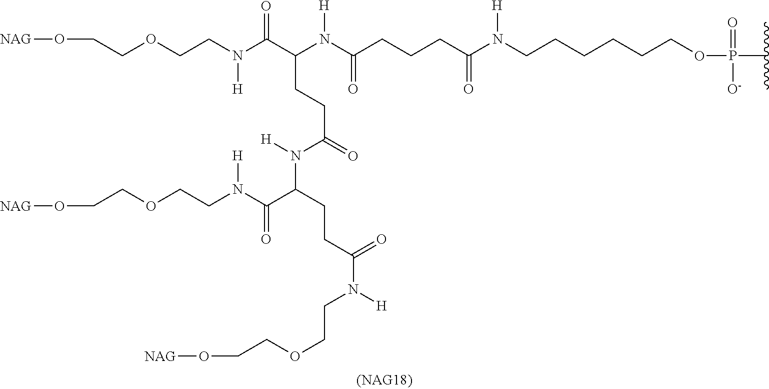

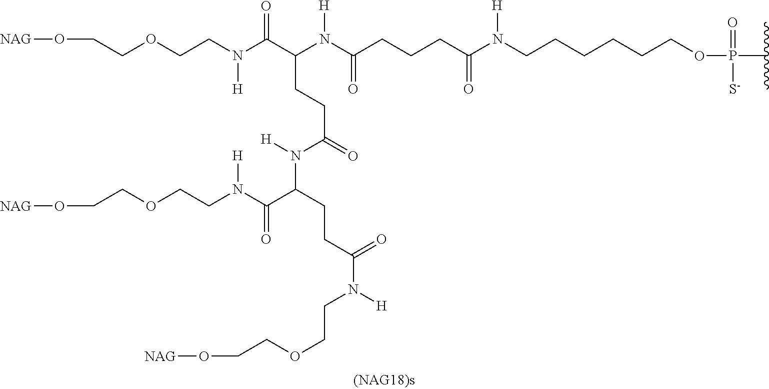

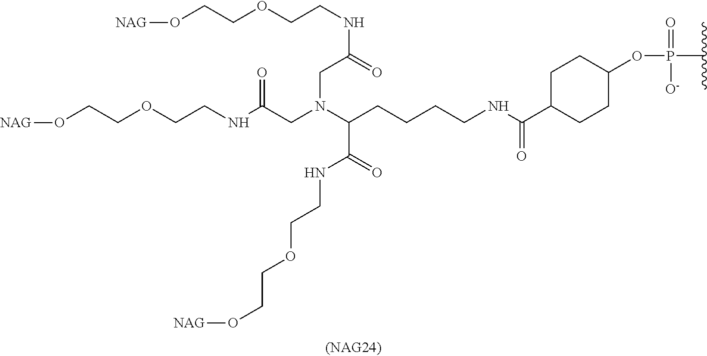

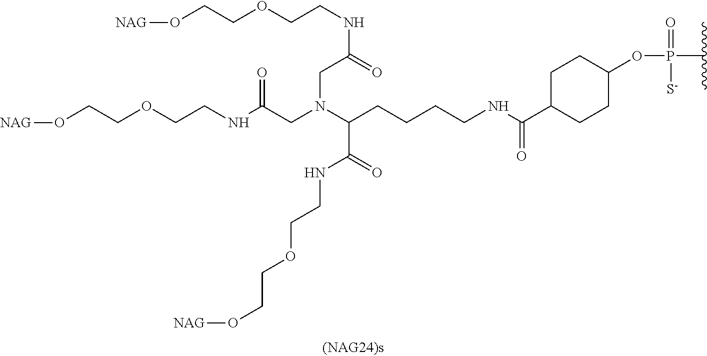

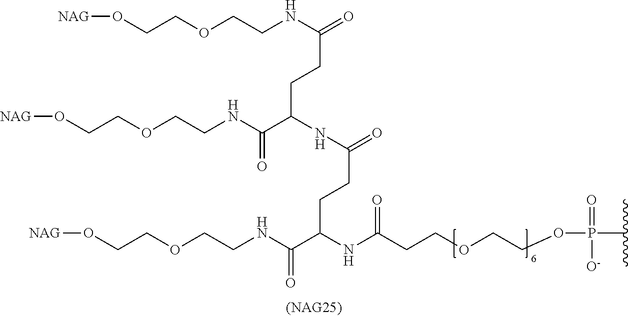

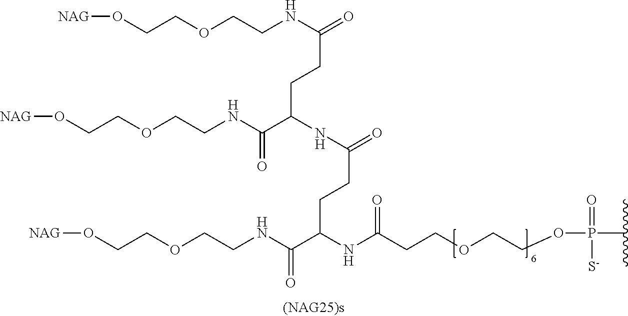

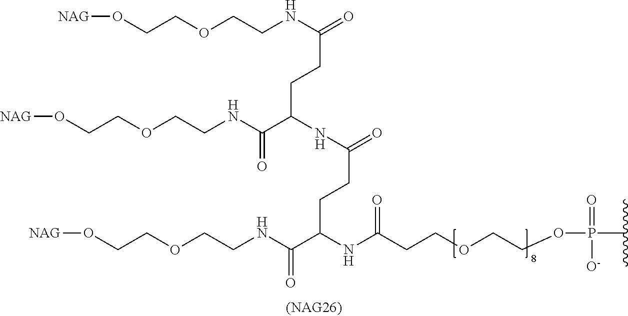

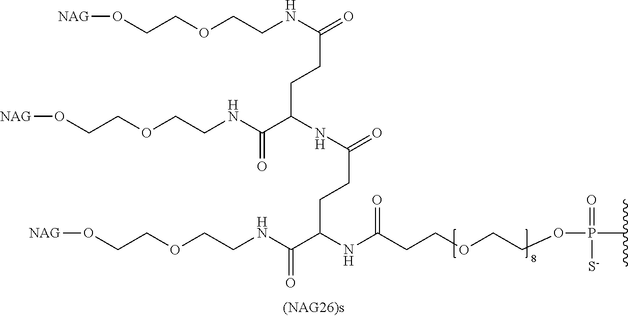

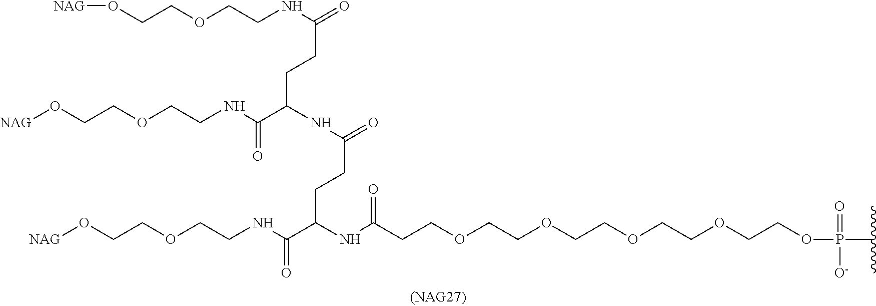

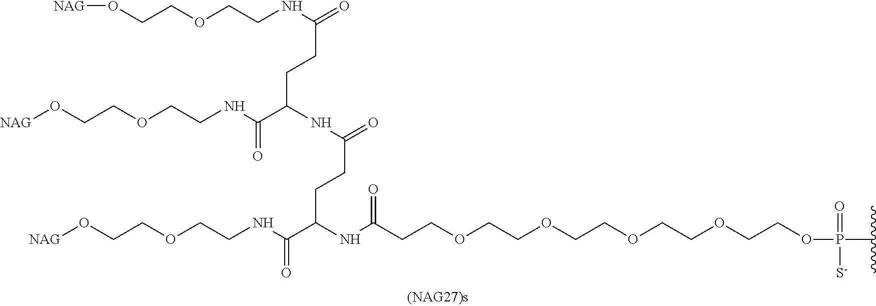

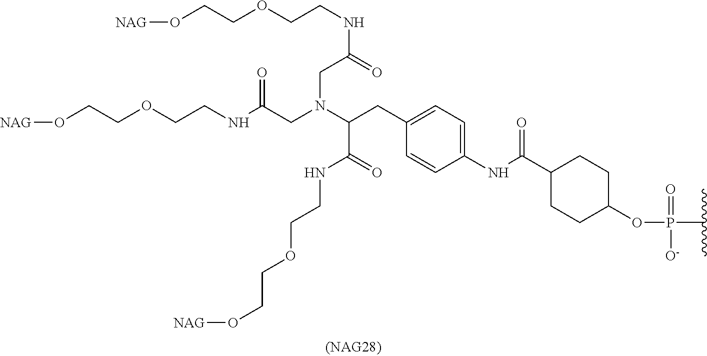

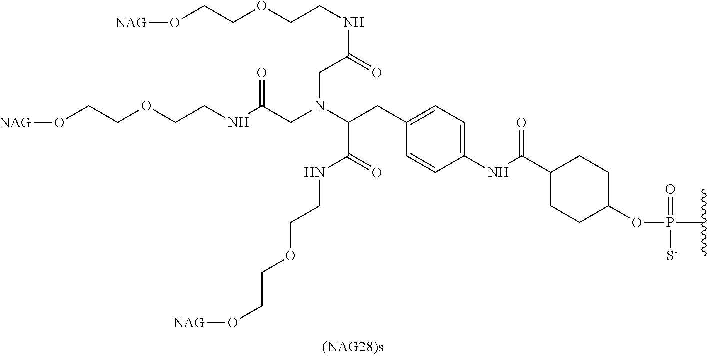

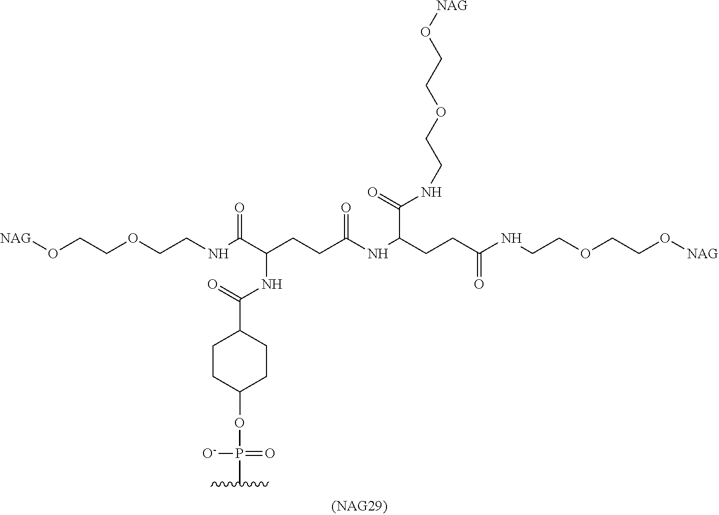

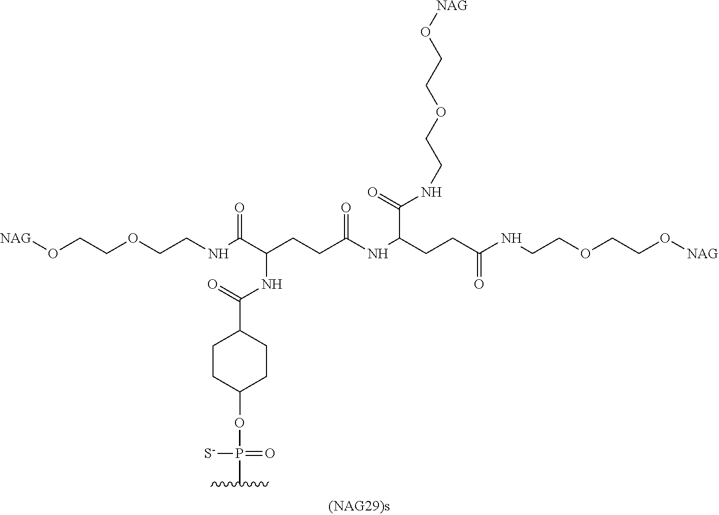

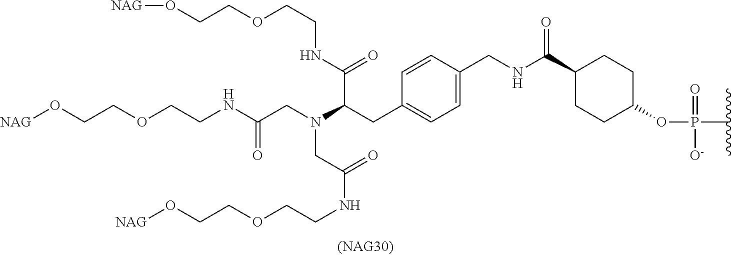

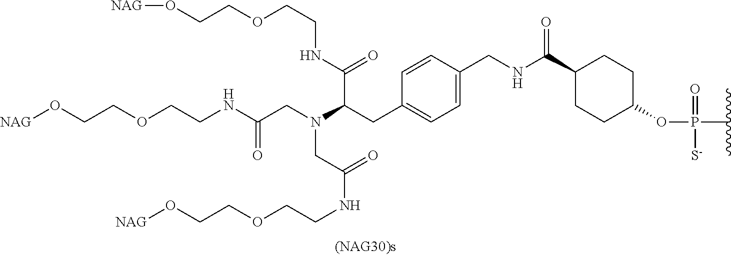

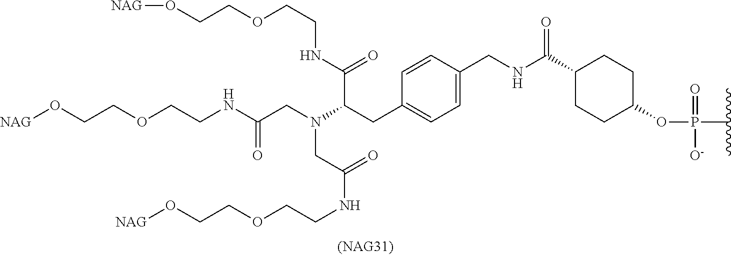

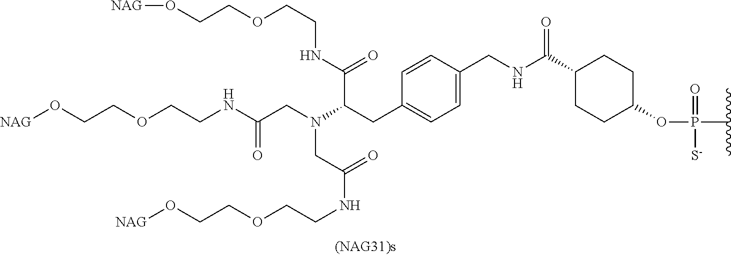

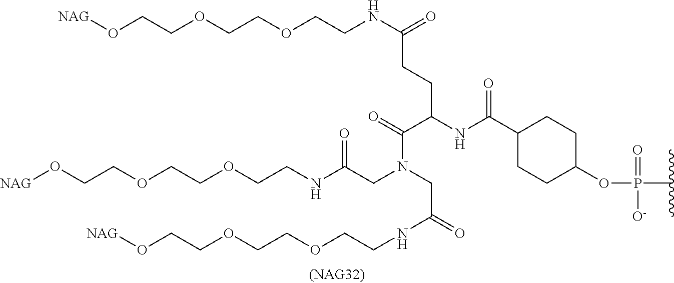

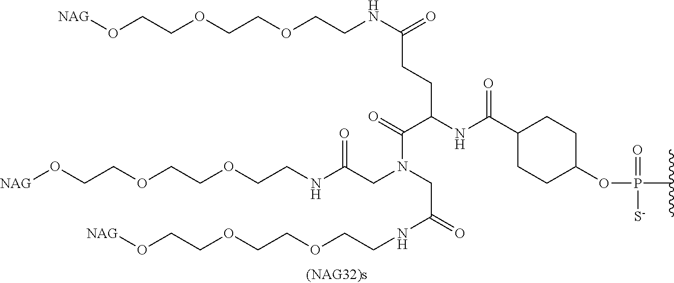

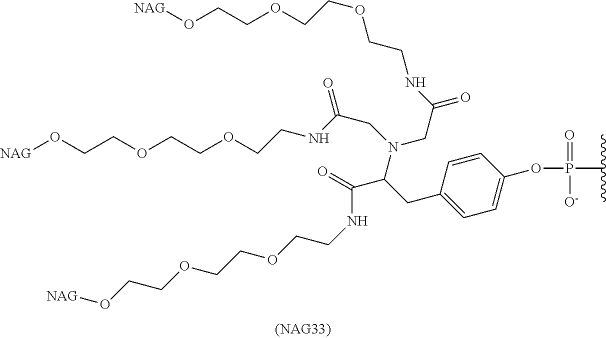

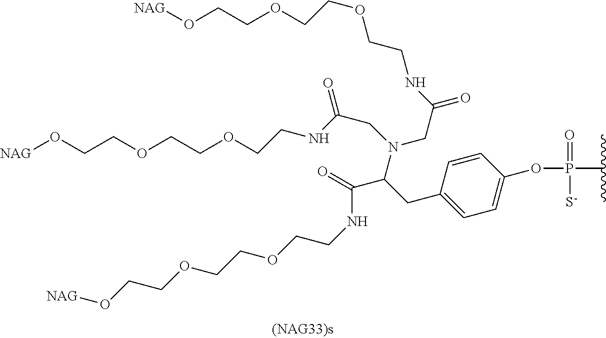

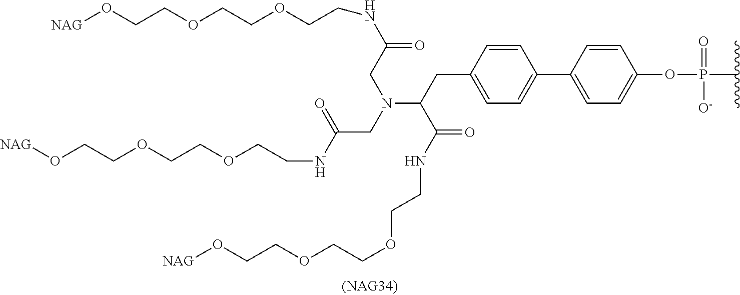

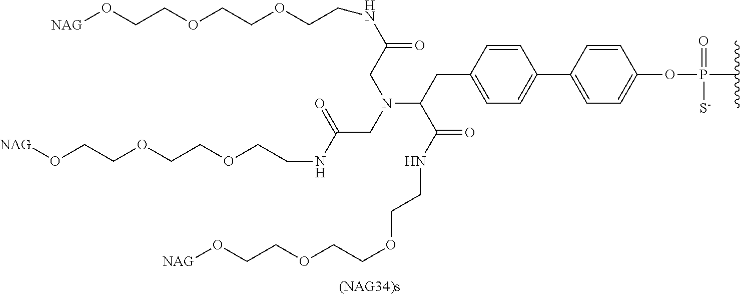

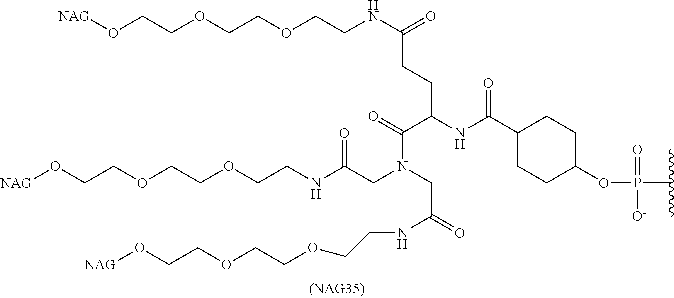

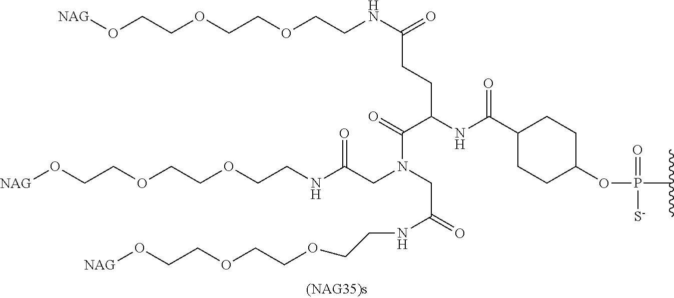

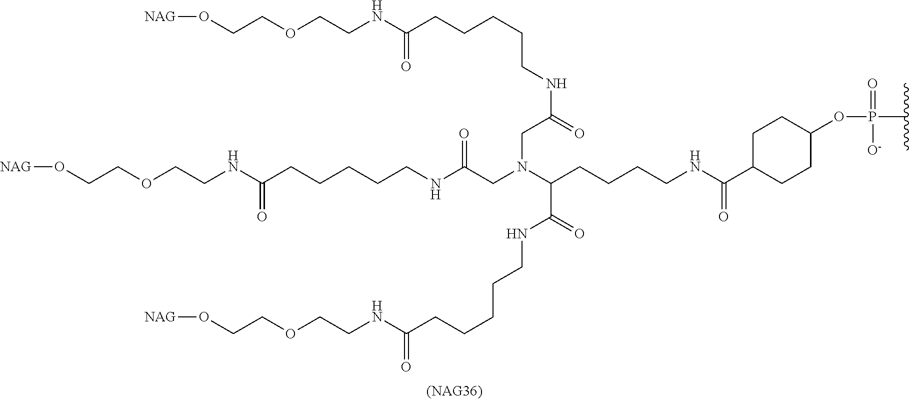

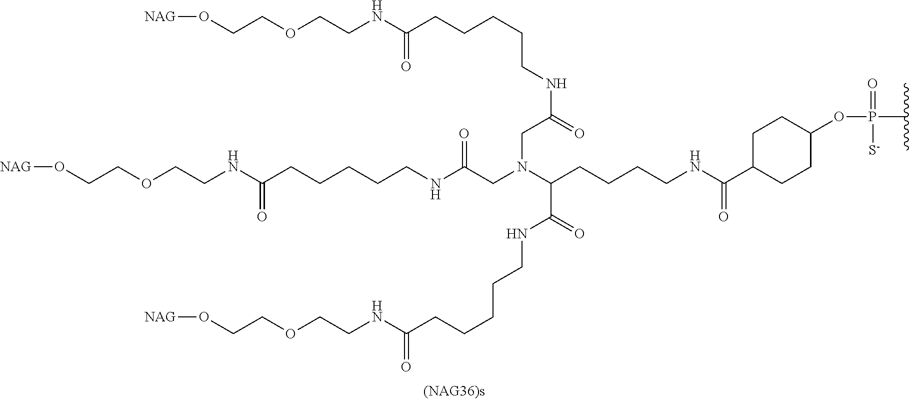

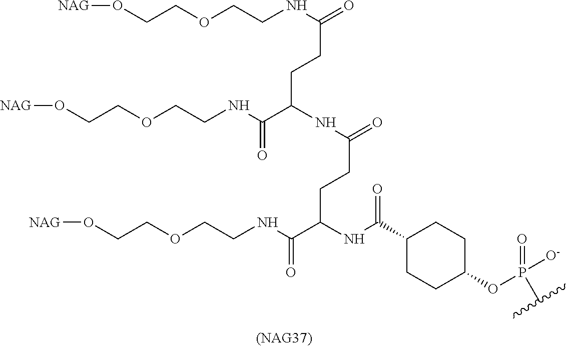

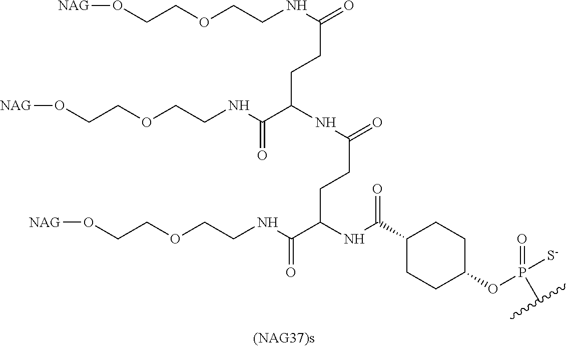

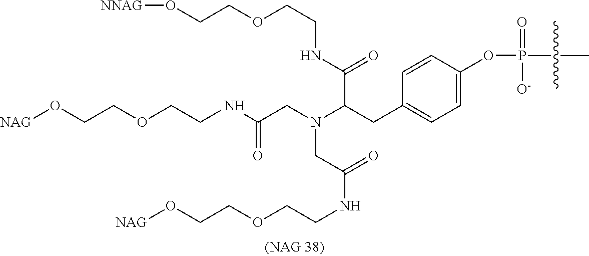

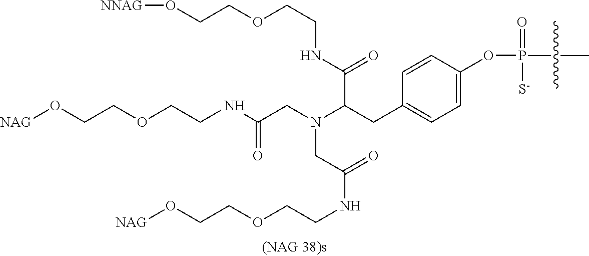

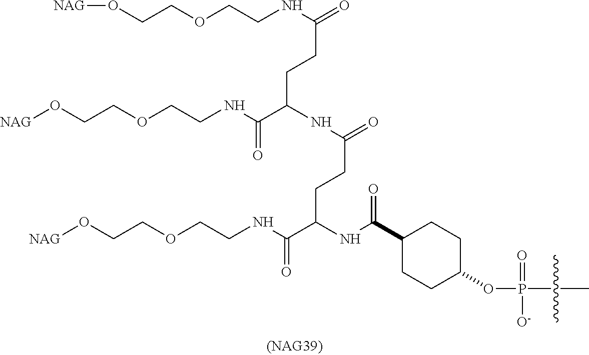

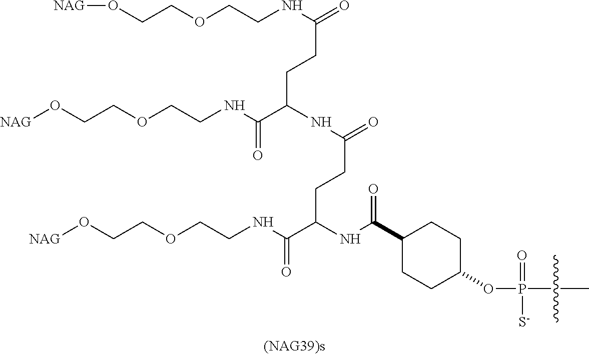

In each of the above structures in Table 7, NAG comprises an N-acetyl-galactosamine or another asialoglycoprotein receptor ligand, as would be understood by a person of ordinary skill in the art to be attached in view of the structures above and description provided herein. For example, in some embodiments, NAG in the structures provided in Table 7 is represented by the following structure:

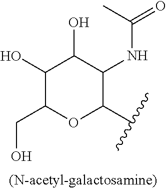 [SMILES: *C1OC(CO)C(O)C(O)C1NC(C)=O]

Each (NAGx) can be attached to an AAT RNAi agent via a phosphate group (as in (NAG25), (NAG30), and (NAG31)), or a phosphorothioate group, (as is (NAG25)s, (NAG29)s, (NAG30)s, (NAG31)s, or (NAG37)s), or another linking group.

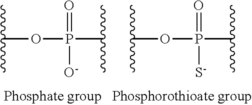 [SMILES: *OP(*)(=O)[O-] ; *OP(*)(=O)[S-]]

Other linking groups known in the art may be used.

In some embodiments, a delivery vehicle can be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine. In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/0022309, WO 2011/104169, and WO 2012/083185, WO 2013/032829, WO 2013/158141, each of which is incorporated herein by reference), or other delivery systems available in the art.

Pharmaceutical Compositions and Formulations

The AAT RNAi agents disclosed herein can be prepared as pharmaceutical compositions or formulations. In some embodiments, pharmaceutical compositions include at least one AAT RNAi agent. These pharmaceutical compositions are particularly useful in the inhibition of the expression of the target mRNA in a target cell, a group of cells, a tissue, or an organism. The pharmaceutical compositions can be used to treat a subject having a disease or disorder that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering an AAT RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions including an AAT RNAi agent, thereby forming a pharmaceutical formulation suitable for in vivo delivery to a subject, including a human.

The pharmaceutical compositions that include an AAT RNAi agent and methods disclosed herein decrease the level of the target mRNA in a cell, group of cells, group of cells, tissue, or subject, including: administering to the subject a therapeutically effective amount of a herein described AAT RNAi agent, thereby inhibiting the expression of AAT mRNA in the subject.

In some embodiments, the described pharmaceutical compositions including an AAT RNAi agent are used for treating or managing clinical presentations in a subject with AATD, such as chronic hepatitis, cirrhosis, hepatocellular carcinoma, transaminitis, cholestasis, fibrosis, and even fulminant hepatic failure. In some embodiments, a therapeutically or prophylactically effective amount of one or more of pharmaceutical compositions is administered to a subject in need of such treatment. In some embodiments, administration of any of the disclosed AAT RNAi agents can be used to decrease the number, severity, and/or frequency of symptoms of a disease in a subject.

The described pharmaceutical compositions including an AAT RNAi agent can be used to treat at least one symptom in a subject having a disease or disorder that would benefit from reduction or inhibition in expression of AAT mRNA. In some embodiments, the subject is administered a therapeutically effective amount of one or more pharmaceutical compositions including an AAT RNAi agent thereby treating the symptom. In other embodiments, the subject is administered a prophylactically effective amount of one or more AAT RNAi agents, thereby preventing the at least one symptom.

The route of administration is the path by which an AAT RNAi agent is brought into contact with the body. In general, methods of administering drugs and nucleic acids for treatment of a mammal are well known in the art and can be applied to administration of the compositions described herein. The AAT RNAi agents disclosed herein can be administered via any suitable route in a preparation appropriately tailored to the particular route. Thus, herein described pharmaceutical compositions can be administered by injection, for example, intravenously, intramuscularly, intracutaneously, subcutaneously, intraarticularly, or intraperitoneally. In some embodiments, the herein described pharmaceutical compositions are administered via subcutaneous injection.

The pharmaceutical compositions including an AAT RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

Accordingly, in some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described AAT RNAi agents and one or more pharmaceutically acceptable excipients. Pharmaceutically acceptable excipients (excipients) are substances other than the Active Pharmaceutical Ingredient (API, therapeutic product, e.g., AAT RNAi agent) that are intentionally included in the drug delivery system. Excipients do not exert or are not intended to exert a therapeutic effect at the intended dosage. Excipients can act to a) aid in processing of the drug delivery system during manufacture, b) protect, support, or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophort EL™ (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The AAT RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic or preventive result.

Generally, an effective amount of an active compound will be in the range of from about 0.1 to about 100 mg/kg of body weight/day, e.g., from about 1.0 to about 50 mg/kg of body weight/day. In some embodiments, an effective amount of an active compound will be in the range of from about 0.25 to about 5 mg/kg of body weight per dose. In some embodiments, an effective amount of an active ingredient will be in the range of from about 0.5 to about 4 mg/kg of body weight per dose. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can, in some instances, be increased beyond the above upper level to rapidly achieve the desired blood-level or tissue level, or the initial dosage can, in some instances, be smaller than the optimum.

For treatment of disease or for formation of a medicament or composition for treatment of a disease, the pharmaceutical compositions described herein including an AAT RNAi agent can be combined with an excipient or with a second therapeutic agent or treatment including, but not limited to: a second or other RNAi agent, a small molecule drug, an antibody, an antibody fragment, peptide and/or aptamer.

The described AAT RNAi agents, when added to pharmaceutically acceptable excipients or adjuvants, can be packaged into kits, containers, packs, or dispensers. The pharmaceutical compositions described herein can be packaged in pre-filled syringes or vials.

Methods of Treatment and Inhibition of Expression

The AAT RNAi agents disclosed herein can be used to treat a subject (e.g., a human or other mammal) having a disease or disorder that would benefit from administration of the compound. In some embodiments, the RNAi agents disclosed herein can be used to treat a subject (e.g., a human) having AATD, or symptoms, diseases, or disorders that would benefit from reduction or inhibition in expression of AAT mRNA, such as AATD liver disease. The subject is administered a therapeutically effective amount of any one or more of the AAT RNAi agents described herein. The subject can be a human, patient, or human patient. The subject may be an adult, adolescent, child, or infant. The described pharmaceutical compositions including an AAT RNAi agent can be used to provide methods for the therapeutic treatment of diseases, such as AATD. Such methods include administration of a pharmaceutical composition described herein to a human being or animal.

In some embodiments, the AAT RNAi agents described herein are used to treat a subject with AATD, including symptoms, diseases or disorders related to AATD. AATD liver diseases or disorders include, but are not limited to, chronic hepatitis, cirrhosis, hepatocellular carcinoma, transaminitis, cholestasis, fibrosis, and fulminant hepatic failure. In some embodiments, the described AAT RNAi agents are used to treat at least one symptom in a subject having AATD. The subject is administered a therapeutically effective amount of any one or more of the described RNAi agents.

In certain embodiments, the present invention provides methods for treatment of AATD in a patient in need thereof, comprising administering to the patient any of the AAT RNAi agents described herein.

In some embodiments, the AAT RNAi agents are used to treat or manage a clinical presentation of a subject with an AATD liver disease or disorder. The subject is administered a therapeutically effective amount of one or more of the AAT RNAi agents or AAT RNAi agent-containing compositions described herein. In some embodiments, the method comprises administering a composition comprising an AAT RNAi agent described herein to a subject to be treated.

In some embodiments, the gene expression level and/or mRNA level of an AAT gene in a subject to whom a described AAT RNAi agent is administered is reduced by at least about 5%, 10%, 15%, 20%, 25%, 30%, 35%, 40%, 45%, 50%, 55%, 60%, 65%, 70%, 75%, 80%, 85%, 95%, 96%, 97%, 98%, 99%, or greater than 99% relative to the subject prior to being administered the AAT RNAi agent or to a subject not receiving the AAT RNAi agent. The gene expression level and/or mRNA level in the subject is reduced in a cell, group of cells, and/or tissue of the subject.

In some embodiments, the protein level of AAT in a subject to whom a described AAT RNAi agent has been administered is reduced by at least about 5%, 10%, 15%, 20%, 25%, 30%, 35%, 40%, 45%, 50%, 55%, 60%, 65%, 70%, 75%, 80%, 85%, 90%/o, 95%, 96%, 97%, 98%, 99%, or greater than 99% relative to the subject prior to being administered the AAT RNAi agent or to a subject not receiving the AAT RNAi agent. The protein level in the subject is reduced in a cell, group of cells, tissue, blood, and/or other fluid of the subject.

In some embodiments, the Z-AAT polymer protein level in a subject having AATD to whom a described AAT RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the AAT RNAi agent or to a subject not receiving the AAT RNAi agent. In some embodiments, the Z-AAT polymer protein level in a subject to whom a described AAT RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 990, or greater than 99% relative to the subject prior to being administered the AAT RNAi agent or to a subject not receiving the AAT RNAi agent.

A reduction in AAT gene expression, AAT mRNA, or AAT protein levels can be assessed and quantified by general methods known in the art. The Examples disclosed herein forth generally known methods for assessing inhibition of AAT gene expression and reduction in AAT protein levels. The reduction or decrease in AAT mRNA level and/or protein level (including Z-AAT polymer and/or monomer) are collectively referred to herein as a reduction or decrease in AAT or inhibiting or reducing the expression of AAT.

Cells and Tissues and Non-Human Organisms

Cells, tissues, and non-human organisms that include at least one of the AAT RNAi agents described herein is contemplated. The cell, tissue, or non-human organism is made by delivering the RNAi agent to the cell, tissue, or non-human organism.

The above provided embodiments and items are now illustrated with the following, non-limiting examples.

### EXAMPLES

### Example 1. Identification of RNAi Agent Sequences and Synthesis of RNAi Agents

A selection process for identifying lead sequences for inhibiting expression of the AAT gene began with in silico methods to identify conserved sequences across variants of an AAT gene (SEQ ID NO: 1). The AAT sequence was initially screened using bioinformatics for 19-nucleotide sequences having a complementary sequence in known variants of human AAT. Sequences known to have manufacturing challenges and those predicted to have poor RNAi activity based on known parameters were eliminated. Sequences were then subjected to cross-species reactivity analysis to select candidates that would cross-react with cynomolgus monkey AAT. The sequences were also evaluated for specificity to avoid off-target effects against the human and cynomolgus monkey genomes. One-hundred fifteen (115) sequence families of 19-mers were selected as candidates.

The duplexes in Table 6 herein were synthesized according to the following procedures:

Synthesis

The sense and antisense strands of the AAT RNAi agents were synthesized according to phosphoramidite technology on solid phase used in oligonucleotide synthesis. Depending on the scale, either a MerMade96E® (Bioautomation) or a MerMade12® (Bioautomation) was used. Syntheses were performed on a solid support made of controlled pore glass (CPG, 500 Å or 600 Å, obtained from Prime Synthesis, Aston, PA, USA). All RNA and 2′-modified RNA phosphoramidites were purchased from Thermo Fisher Scientific (Milwaukee, WI, USA). Specifically, the following 2′-O-methyl phosphoramidites were used: (5′-O-dimethoxytrityl-N6-(benzoyl)-2′-O-methyl-adenosine-3′-O-(2-cyanoethyl-N,N-diisopropy-lamino) phosphoramidite, 5′-O-dimethoxy-trityl-N4-(acetyl)-2′-O-methyl-cytidine-3′-O-(2-cyanoethyl-N,N-diisopropyl-amino) phosphoramidite, (5′-O-dimethoxytrityl-N2-(isobutyryl)-2′-O-methyl-guanosine-3′-O-(2-cyano-ethyl-N,N-diisopropylamino)phosphoramidite, and 5′-O-dimethoxy-trityl-2′-O-methyl-uridine-3′-O-(2-cyanoethyl-N,N-diisopropylamino)phosphoramidite. The 2′-deoxy-2′-fluoro-phosphoramidites carried the same protecting groups as the 2′-O-methyl RNA amidites. The following UNA phosphoramidites were used: 5′-(4,4′-Dimethoxytrityl)-N-benzoyl-2′,3′-seco-adenosine, 2′-benzoyl-3′-[(2-cyanoethyl)-(N,N-diisopropyl)]-phosphor-amidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite.

Targeting ligand-containing phosphoramidites were dissolved in anhydrous dichloromethane or anhydrous acetonitrile (50 mM), while all other amidites were dissolved in anhydrous acetonitrile (50 mM) and molecular sieves (3 Å) were added, 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 15 min (targeting ligand), 90 sec (2′OMe), and 60 sec (2′F). In order to introduce phosphorothioate linkages, a 100 mM solution of 3-phenyl 1,2,4-dithiazoline-5-one (POS, obtained from PolyOrg, Inc., Leominster, MA, USA) in anhydrous Acetonitrile was employed.

Cleavage and Deprotection of Support Bound Oligomers.

After finalization of the solid phase synthesis, the dried solid support was treated with a 1:1 volume solution of 40 wt. % methylamine in water and 28% ammonium hydroxide solution (Aldrich) for two hours at 30° C. The solution was evaporated and the solid residue was reconstituted in water (see below).

Purification

Crude oligomers were purified by anionic exchange HPLC using a TKSgel SuperQ-5PW 13u column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 medium with a running buffer of 100 mM ammonium bicarbonate, pH 6.7 and 20% Acetonitrile.

Annealing

Complementary strands were mixed by combining equimolar RNA solutions (sense and antisense)in 0.2×PBS (Phosphate-Buffered Saline, 1×, Corning, Cellgro) to form the RNAi agents. This solution was placed into a thermomixer at 70° C., heated to 95° C., held at 95° C. for 5 min, and cooled to room temperature slowly. Some RNAi agents were lyophilized and stored at −15 to −25° C. Duplex concentration was determined by measuring the solution absorbance on a UV-Vis spectrometer in 0.2×PBS. The solution absorbance at 260 nm was then multiplied by a conversion factor and the dilution factor to determine the duplex concentration. Unless otherwise stated, all conversion factor was 0.037 mg/(mL·cm). For some experiments, a conversion factor was calculated from an experimentally determined extinction coefficient.

### Example 2. In Vitro Testing of AAT RNAi Agents

Candidate sequence duplexes were tested in vitro. The antisense strand sequences and sense strand sequences were annealed to form duplexes of 21-mer strands (having 19 base pairs and a di-nucleotide UU overhang on each 3′ end) for in vitro testing, as shown in the following Table 8:

TABLE 8
Sequences of AAT RNAi Agents in Example 2

| SEQ ID | Antisense Sequence | SEQ ID | Sense Sequence | Duplex |
|---|---|---|---|---|
| NO: | (5′ → 3′) | NO: | (5′ → 3′) | ID No. |
| 1035 | AGAAGAUAUUGGUGCUGUUUU | 1150 | AACAGCACCAAUAUCUUCUUU | D1 |
| 1036 | AGGAACUUGGUGAUGAUAUUU | 1151 | AUAUCAUCACCAAGUUCCUUU | D2 |
| 1037 | UGUCUUCUGGGCAGCAUCUUU | 1152 | AGAUGCUGCCCAGAAGACAUU | D3 |
| 1038 | UGUUGGACUGGUGUGCCAGUU | 1153 | CUGGCACACCAGUCCAACAUU | D4 |
| 1039 | CUGUUGGACUGGUGUGCCAUU | 1154 | UGGCACACCAGUCCAACAGUU | D5 |
| 1040 | UGCUGUUGGACUGGUGUGCUU | 1155 | GCACACCAGUCCAACAGCAUU | D6 |
| 1041 | UAUUGGUGCUGUUGGACUGUU | 1156 | CAGUCCAACAGCACCAAUAUU | D7 |
| 1042 | AUAUUGGUGCUGUUGGACUUU | 1157 | AGUCCAACAGCACCAAUAUUU | D8 |
| 1043 | GAUAUUGGUGCUGUUGGACUU | 1158 | GUCCAACAGCACCAAUAUCUU | D9 |
| 1044 | AAGAUAUUGGUGCUGUUGGUU | 1159 | CCAACAGCACCAAUAUCUUUU | D10 |
| 1045 | GUAGCGAUGCUCACUGGGGUU | 1160 | CCCCAGUGAGCAUCGCUACUU | D11 |
| 1046 | AAAGGCUGUAGCGAUGCUCUU | 1161 | GAGCAUCGCUACAGCCUUUUU | D12 |
| 1047 | GCAAAGGCUGUAGCGAUGCUU | 1162 | GCAUCGCUACAGCCUUUGCUU | D13 |
| 1048 | UGCAAAGGCUGUAGCGAUGUU | 1163 | CAUCGCUACAGCCUUUGCAUU | D14 |
| 1049 | AUUGCAAAGGCUGUAGCGAUU | 1164 | UCGCUACAGCCUUUGCAAUUU | D15 |
| 1050 | AGCAUUGCAAAGGCUGUAGUU | 1165 | CUACAGCCUUUGCAAUGCUUU | D16 |
| 1051 | AGAGCAUUGCAAAGGCUGUUU | 1166 | ACAGCCUUUGCAAUGCUCUUU | D17 |
| 1052 | GGAGUUCCUGGAAGCCUUCUU | 1167 | GAAGGCUUCCAGGAACUCCUU | D18 |
| 1053 | UCCAAAAACUUAUCCACUAUU | 1168 | UAGUGGAUAAGUUUUUGGAUU | D19 |
| 1054 | AAGGCUUCUGAGUGGUACAUU | 1169 | UGUACCACUCAGAAGCCUUUU | D20 |
| 1055 | GAAGGCUUCUGAGUGGUACUU | 1170 | GUACCACUCAGAAGCCUUCUU | D21 |
| 1056 | UUCUUGGCCUCUUCGGUGUUU | 1171 | ACACCGAAGAGGCCAAGAAUU | D22 |
| 1057 | GUUUCUUGGCCUCUUCGGUUU | 1172 | ACCGAAGAGGCCAAGAAACUU | D23 |
| 1058 | UUGAUCUGUUUCUUGGCCUUU | 1173 | AGGCCAAGAAACAGAUCAAUU | D24 |
| 1059 | GUUGAUCUGUUUCUUGGCCUU | 1174 | GGCCAAGAAACAGAUCAACUU | D25 |
| 1060 | CGUUGAUCUGUUUCUUGGCUU | 1175 | GCCAAGAAACAGAUCAACGUU | D26 |
| 1061 | CACAAUUUUCCCUUGAGUAUU | 1176 | UACUCAAGGGAAAAUUGUGUU | D27 |
| 1062 | UCCACAAUUUUCCCUUGAGUU | 1177 | CUCAAGGGAAAAUUGUGGAUU | D28 |
| 1063 | AUCCACAAUUUUCCCUUGAUU | 1178 | UCAAGGGAAAAUUGUGGAUUU | D29 |
| 1064 | UGUCAAGCUCCUUGACCAAUU | 1179 | UUGGUCAAGGAGCUUGACAUU | D30 |
| 1065 | GUGUCUCUGUCAAGCUCCUUU | 1180 | AGGAGCUUGACAGAGACACUU | D31 |
| 1066 | ACUGUGUCUCUGUCAAGCUUU | 1181 | AGCUUGACAGAGACACAGUUU | D32 |
| 1067 | UGUAAUUCACCAGAGCAAAUU | 1182 | UUUGCUCUGGUGAAUUACAUU | D33 |
| 1068 | UUAAACAUGCCUAAACGCUUU | 1183 | AGCGUUUAGGCAUGUUUAAUU | D34 |
| 1069 | GUUAAACAUGCCUAAACGCUU | 1184 | GCGUUUAGGCAUGUUUAACUU | D35 |
| 1070 | UGUUAAACAUGCCUAAACGUU | 1185 | CGUUUAGGCAUGUUUAACAUU | D36 |
| 1071 | GGAUGUUAAACAUGCCUAAUU | 1186 | UUAGGCAUGUUUAACAUCCUU | D37 |
| 1072 | AUUUCAUCAGCAGCACCCAUU | 1187 | UGGGUGCUGCUGAUGAAAUUU | D38 |
| 1073 | GAAGAAGAUGGCGGUGGCAUU | 1188 | UGCCACCGCCAUCUUCUUCUU | D39 |
| 1074 | GGUGAGUUCAUUUUCCAGGUU | 1189 | CCUGGAAAAUGAACUCACCUU | D40 |
| 1075 | GAACUUGGUGAUGAUAUCGUU | 1190 | CGAUAUCAUCACCAAGUUCUU | D41 |
| 1076 | CAUUUUCCAGGAACUUGGUUU | 1191 | ACCAAGUUCCUGGAAAAUGUU | D42 |
| 1077 | CAUAGGUUCCAGUAAUGGAUU | 1192 | UCCAUUACUGGAACCUAUGUU | D43 |
| 1078 | UCAUAGGUUCCAGUAAUGGUU | 1193 | CCAUUACUGGAACCUAUGAUU | D44 |
| 1079 | UCAGAUCAUAGGUUCCAGUUU | 1194 | ACUGGAACCUAUGAUCUGAUU | D45 |
| 1080 | UCUUCAGAUCAUAGGUUCCUU | 1195 | GGAACCUAUGAUCUGAAGAUU | D46 |
| 1081 | CUCUUCAGAUCAUAGGUUCUU | 1196 | GAACCUAUGAUCUGAAGAGUU | D47 |
| 1082 | GAGGUCAGCCCCAUUGCUGUU | 1197 | CAGCAAUGGGGCUGACCUCUU | D48 |
| 1083 | GAGAGGUCAGCCCCAUUGCUU | 1198 | GCAAUGGGGCUGACCUCUCUU | D49 |
| 1084 | CUUCAGGGGUGCCUCCUCUUU | 1199 | AGAGGAGGCACCCCUGAAGUU | D50 |
| 1085 | GAGAGCUUCAGGGGUGCCUUU | 1200 | AGGCACCCCUGAAGCUCUCUU | D51 |
| 1086 | UUAUGCACGGCCUUGGAGAUU | 1201 | UCUCCAAGGCCGUGCAUAAUU | D52 |
| 1087 | CCUUAUGCACGGCCUUGGAUU | 1202 | UCCAAGGCCGUGCAUAAGGUU | D53 |
| 1088 | GCCUUAUGCACGGCCUUGGUU | 1203 | CCAAGGCCGUGCAUAAGGCUU | D54 |
| 1089 | AGCCUUAUGCACGGCCUUGUU | 1204 | CAAGGCCGUGCAUAAGGCUUU | D55 |
| 1090 | CGAUGGUCAGCACAGCCUUUU | 1205 | AAGGCUGUGCUGACCAUCGUU | D56 |
| 1091 | GUCGAUGGUCAGCACAGCCUU | 1206 | GGCUGUGCUGACCAUCGACUU | D57 |
| 1092 | AAAAACAUGGCCCCAGCAGUU | 1207 | CUGCUGGGGCCAUGUUUUUUU | D58 |
| 1093 | CUAAAAACAUGGCCCCAGCUU | 1208 | GCUGGGGCCAUGUUUUUAGUU | D59 |
| 1094 | UCUAAAAACAUGGCCCCAGUU | 1209 | CUGGGGCCAUGUUUUUAGAUU | D60 |
| 1095 | CCUCUAAAAACAUGGCCCCUU | 1210 | GGGGCCAUGUUUUUAGAGGUU | D61 |
| 1096 | GCCUCUAAAAACAUGGCCCUU | 1211 | GGGCCAUGUUUUUAGAGGCUU | D62 |
| 1097 | UAGACAUGGGUAUGGCCUCUU | 1212 | GAGGCCAUACCCAUGUCUAUU | D63 |
| 1098 | GAUAGACAUGGGUAUGGCCUU | 1213 | GGCCAUACCCAUGUCUAUCUU | D64 |
| 1099 | UGUUGAACUUGACCUCGGGUU | 1214 | CCCGAGGUCAAGUUCAACAUU | D65 |
| 1100 | GGUUUGUUGAACUUGACCUUU | 1215 | AGGUCAAGUUCAACAAACCUU | D66 |
| 1101 | AAAGGGUUUGUUGAACUUGUU | 1216 | CAAGUUCAACAAACCCUUUUU | D67 |
| 1102 | ACAAAGGGUUUGUUGAACUUU | 1217 | AGUUCAACAAACCCUUUGUUU | D68 |
| 1103 | GACAAAGGGUUUGUUGAACUU | 1218 | GUUCAACAAACCCUUUGUCUU | D69 |
| 1104 | AAGACAAAGGGUUUGUUGAUU | 1219 | UCAACAAACCCUUUGUCUUUU | D70 |
| 1105 | CAUUAAGAAGACAAAGGGUUU | 1220 | ACCCUUUGUCUUCUUAAUGUU | D71 |
| 1106 | AUCAUUAAGAAGACAAAGGUU | 1221 | CCUUUGUCUUCUUAAUGAUUU | D72 |
| 1107 | GAAGAGGGGAGACUUGGUAUU | 1222 | UACCAAGUCUCCCCUCUUCUU | D73 |
| 1108 | CCAUGAAGAGGGGAGACUUUU | 1223 | AAGUCUCCCCUCUUCAUGGUU | D74 |
| 1109 | CCCAUGAAGAGGGGAGACUUU | 1224 | AGUCUCCCCUCUUCAUGGGUU | D75 |
| 1110 | UUCCCAUGAAGAGGGGAGAUU | 1225 | UCUCCCCUCUUCAUGGGAAUU | D76 |
| 1111 | UUUCCCAUGAAGAGGGGAGUU | 1226 | CUCCCCUCUUCAUGGGAAAUU | D77 |
| 1112 | AACCCUUCUUUAAUGUCAUUU | 1227 | AUGACAUUAAAGAAGGGUUUU | D78 |
| 1113 | UUGUUGGACUGGUGUGCCAUU | 1228 | UGGCACACCAGUCCAACAAUU | D79 |
| 1114 | UAUAUUGGUGCUGUUGGACUU | 1229 | GUCCAACAGCACCAAUAUAUU | D80 |
| 1115 | UUAGCGAUGCUCACUGGGGUU | 1230 | CCCCAGUGAGCAUCGCUAAUU | D81 |
| 1116 | UCAAAGGCUGUAGCGAUGCUU | 1231 | GCAUCGCUACAGCCUUUGAUU | D82 |
| 1117 | UGAGUUCCUGGAAGCCUUCUU | 1232 | GAAGGCUUCCAGGAACUCAUU | D83 |
| 1118 | UAAGGCUUCUGAGUGGUACUU | 1233 | GUACCACUCAGAAGCCUUAUU | D84 |
| 1119 | UUUUCUUGGCCUCUUCGGUUU | 1234 | ACCGAAGAGGCCAAGAAAAUU | D85 |
| 1120 | UUUGAUCUGUUUCUUGGCCUU | 1235 | GGCCAAGAAACAGAUCAAAUU | D86 |
| 1121 | UGUUGAUCUGUUUCUUGGCUU | 1236 | GCCAAGAAACAGAUCAACAUU | D87 |
| 1122 | UACAAUUUUCCCUUGAGUAUU | 1237 | UACUCAAGGGAAAAUUGUAUU | D88 |
| 1123 | UUGUCUCUGUCAAGCUCCUUU | 1238 | AGGAGCUUGACAGAGACAAUU | D89 |
| 1124 | UUUAAACAUGCCUAAACGCUU | 1239 | GCGUUUAGGCAUGUUUAAAUU | D90 |
| 1125 | UGAUGUUAAACAUGCCUAAUU | 1240 | UUAGGCAUGUUUAACAUCAUU | D91 |
| 1126 | UAAGAAGAUGGCGGUGGCAUU | 1241 | UGCCACCGCCAUCUUCUUAUU | D92 |
| 1127 | UGUGAGUUCAUUUUCCAGGUU | 1242 | CCUGGAAAAUGAACUCACAUU | D93 |
| 1128 | UAACUUGGUGAUGAUAUCGUU | 1243 | CGAUAUCAUCACCAAGUUAUU | D94 |
| 1129 | UAUUUUCCAGGAACUUGGUUU | 1244 | ACCAAGUUCCUGGAAAAUAUU | D95 |
| 1130 | UAUAGGUUCCAGUAAUGGAUU | 1245 | UCCAUUACUGGAACCUAUAUU | D96 |
| 1131 | UUCUUCAGAUCAUAGGUUCUU | 1246 | GAACCUAUGAUCUGAAGAAUU | D97 |
| 1132 | UAGGUCAGCCCCAUUGCUGUU | 1247 | CAGCAAUGGGGCUGACCUAUU | D98 |
| 1133 | UAGAGGUCAGCCCCAUUGCUU | 1248 | GCAAUGGGGCUGACCUCUAUU | D99 |
| 1134 | UUUCAGGGGUGCCUCCUCUUU | 1249 | AGAGGAGGCACCCCUGAAAUU | D100 |
| 1135 | UAGAGCUUCAGGGGUGCCUUU | 1250 | AGGCACCCCUGAAGCUCUAUU | D101 |
| 1136 | UCUUAUGCACGGCCUUGGAUU | 1251 | UCCAAGGCCGUGCAUAAGAUU | D102 |
| 1137 | UCCUUAUGCACGGCCUUGGUU | 1252 | CCAAGGCCGUGCAUAAGGAUU | D103 |
| 1138 | UGAUGGUCAGCACAGCCUUUU | 1253 | AAGGCUGUGCUGACCAUCAUU | D104 |
| 1139 | UUCGAUGGUCAGCACAGCCUU | 1254 | GGCUGUGCUGACCAUCGAAUU | D105 |
| 1140 | UUAAAAACAUGGCCCCAGCUU | 1255 | GCUGGGGCCAUGUUUUUAAUU | D106 |
| 1141 | UCUCUAAAAACAUGGCCCCUU | 1256 | GGGGCCAUGUUUUUAGAGAUU | D107 |
| 1142 | UCCUCUAAAAACAUGGCCCUU | 1257 | GGGCCAUGUUUUUAGAGGAUU | D108 |
| 1143 | UAUAGACAUGGGUAUGGCCUU | 1258 | GGCCAUACCCAUGUCUAUAUU | D109 |
| 1144 | UGUUUGUUGAACUUGACCUUU | 1259 | AGGUCAAGUUCAACAAACAUU | D110 |
| 1145 | UACAAAGGGUUUGUUGAACUU | 1260 | GUUCAACAAACCCUUUGUAUU | D111 |
| 1146 | UAUUAAGAAGACAAAGGGUUU | 1261 | ACCCUUUGUCUUCUUAAUAUU | D112 |
| 1147 | UAAGAGGGGAGACUUGGUAUU | 1262 | UACCAAGUCUCCCCUCUUAUU | D113 |
| 1148 | UCAUGAAGAGGGGAGACUUUU | 1263 | AAGUCUCCCCUCUUCAUGAUU | D114 |
| 1149 | UCCAUGAAGAGGGGAGACUUU | 1264 | AGUCUCCCCUCUUCAUGGAUU | D115 |

AAT RNAi agents were evaluated by transfection of Hep3B cells, a human hepatocellular carcinoma line. Cells were plated at ˜10,000 cells per well in 96-well format, and each of the 115 AAT RNAi agent duplexes was transfected at three concentrations (10 nM, 1 nM, and 0.1 nM), using LipoFectamine RNAiMax (Thermo Fisher) transfection reagent. Relative expression of each of the 115 AAT RNAi agents was determined by qRT-PCR by comparing the expression levels of AAT mRNA to an endogenous control, and normalized to untreated Hep3B cells (ΔΔ_{CT} analysis), as shown in Table 9.

TABLE 9
In Vitro Data from Duplexes of Example 2

| Duplex ID No. | Avg. Rel. | Avg. Rel. | Avg. Rel. |
|---|---|---|---|
| (From Table 8) | Exp. 10 nM | Exp. 1 nM | Exp. 0.1 nM |
| D1 | 1.037 | 0.896 | 0.709 |
| D2 | 0.068 | 0.089 | 0.381 |
| D3 | 0.046 | 0.064 | 0.403 |
| D4 | 0.075 | 0.090 | 0.391 |
| D5 | 0.408 | 0.424 | 0.743 |
| D6 | 0.018 | 0.032 | 0.347 |
| D7 | 0.069 | 0.125 | 0.666 |
| D8 | 0.092 | 0.193 | 0.794 |
| D9 | 0.206 | 0.228 | 0.839 |
| D10 | 0.023 | 0.032 | 0.235 |
| D11 | 0.309 | 0.522 | 0.894 |
| D12 | 0.049 | 0.092 | 0.732 |
| D13 | 0.549 | 0.665 | 0.955 |
| D14 | 0.531 | 0.654 | 0.934 |
| D15 | 0.108 | 0.197 | 0.820 |
| D16 | 0.558 | 0.516 | 0.834 |
| D17 | 0.626 | 0.606 | 0.841 |
| D18 | 0.668 | 0.703 | 0.778 |
| D19 | 0.624 | 0.803 | 0.785 |
| D20 | 0.071 | 0.080 | 0.506 |
| D21 | 0.022 | 0.037 | 0.345 |
| D22 | 0.086 | 0.127 | 0.588 |
| D23 | 0.175 | 0.238 | 0.893 |
| D24 | 0.134 | 0.078 | 0.368 |
| D25 | 0.056 | 0.075 | 0.687 |
| D26 | 0.122 | 0.196 | 0.756 |
| D27 | 0.517 | 0.560 | 0.846 |
| D28 | 0.801 | 0.838 | 0.884 |
| D29 | 0.820 | 0.870 | 0.903 |
| D30 | 0.558 | 0.632 | 0.879 |
| D31 | 1.112 | 1.110 | 0.922 |
| D32 | 0.246 | 0.359 | 1.041 |
| D33 | 0.107 | 0.355 | 0.967 |
| D34 | 0.096 | 0.170 | 0.962 |
| D35 | 0.317 | 0.552 | 0.949 |
| D36 | 0.064 | 0.134 | 0.873 |
| D37 | 0.463 | 1.005 | 1.006 |
| D38 | 0.428 | 0.688 | 0.486 |
| D39 | 0.730 | 0.918 | 1.258 |
| D40 | 0.059 | 0.067 | 0.912 |
| D41 | 0.093 | 0.095 | 0.952 |
| D42 | 0.582 | 0.665 | 0.944 |
| D43 | 0.196 | 0.283 | 1.004 |
| D44 | 0.195 | 0.278 | 0.860 |
| D45 | 0.053 | 0.103 | 0.817 |
| D46 | 0.082 | 0.127 | 1.034 |
| D47 | 0.089 | 0.156 | 0.821 |
| D48 | 0.735 | 0.695 | 0.838 |
| D49 | 0.604 | 0.610 | 0.838 |
| D50 | 0.543 | 0.633 | 0.806 |
| D51 | 0.114 | 0.144 | 0.775 |
| D52 | 0.108 | 0.203 | 0.836 |
| D53 | 1.062 | 0.836 | 0.931 |
| D54 | 0.091 | 0.274 | 1.081 |
| D55 | 0.526 | 0.623 | 0.914 |
| D56 | 0.500 | 0.588 | 0.884 |
| D57 | 0.049 | 0.126 | 0.797 |
| D58 | 0.198 | 0.302 | 0.917 |
| D59 | 0.732 | 0.745 | 0.953 |
| D60 | 0.389 | 0.580 | 0.897 |
| D61 | 0.585 | 0.624 | 1.802 |
| D62 | 0.174 | 0.215 | 1.115 |
| D63 | 0.093 | 0.074 | 0.917 |
| D64 | 0.133 | 0.133 | 1.055 |
| D65 | 0.395 | 0.362 | 0.986 |
| D66 | 0.054 | 0.055 | 1.083 |
| D67 | 0.105 | 0.118 | 1.018 |
| D68 | 0.106 | 0.122 | 1.290 |
| D69 | 0.201 | 0.194 | 1.062 |
| D70 | 0.050 | 0.048 | 0.709 |
| D71 | 0.231 | 0.216 | 0.767 |
| D72 | 0.046 | 0.030 | 0.737 |
| D73 | 0.521 | 0.423 | 0.782 |
| D74 | 0.479 | 0.467 | 0.694 |
| D75 | 0.531 | 0.583 | 0.794 |
| D76 | 0.210 | 0.285 | 0.924 |
| D77 | 0.152 | 0.181 | 0.803 |
| D78 | 0.425 | 0.485 | 0.703 |
| D79 | 0.120 | 0.127 | 0.711 |
| D80 | 0.203 | 0.167 | 0.672 |
| D81 | 0.477 | 0.402 | 0.611 |
| D82 | 0.540 | 0.489 | 0.661 |
| D83 | 0.315 | 0.316 | 0.838 |
| D84 | 0.135 | 0.118 | 0.375 |
| D85 | 0.209 | 0.270 | 1.050 |
| D86 | 0.120 | 0.136 | 0.928 |
| D87 | 0.172 | 0.207 | 1.056 |
| D88 | 0.218 | 0.308 | 1.006 |
| D89 | 0.605 | 0.643 | 0.925 |
| D90 | 0.205 | 0.259 | 0.927 |
| D91 | 0.594 | 1.097 | 1.052 |
| D92 | 0.337 | 0.887 | 1.015 |
| D93 | 0.068 | 0.503 | 0.864 |
| D94 | 0.067 | 0.475 | 0.811 |
| D95 | 0.186 | 0.770 | 0.931 |
| D96 | 0.062 | 0.389 | 0.550 |
| D97 | 0.066 | 0.470 | 0.896 |
| D98 | 0.567 | 0.998 | 1.044 |
| D99 | 0.451 | 1.092 | 1.359 |
| D100 | 0.292 | 0.745 | 0.875 |
| D101 | 0.049 | 0.320 | 0.659 |
| D102 | 0.313 | 0.799 | 0.732 |
| D103 | 0.068 | 0.541 | 0.630 |
| D104 | 0.077 | 0.552 | 0.682 |
| D105 | 0.071 | 0.355 | 0.459 |
| D106 | 1.179 | 1.117 | 1.076 |
| D107 | 0.328 | 0.597 | 0.876 |
| D108 | 0.125 | 0.467 | 0.573 |
| D109 | 0.141 | 0.545 | 0.753 |
| D110 | 0.076 | 0.497 | 0.778 |
| D111 | 0.132 | 0.511 | 0.634 |
| D112 | 0.216 | 0.586 | 0.784 |
| D113 | 0.462 | 0.687 | 1.021 |
| D114 | 0.507 | 0.792 | 1.170 |
| D115 | 0.259 | 0.797 | 1.027 |

### Example 3. In Vivo Testing of NAG-Conjugated AAT RNAI Agents in P Mice

A transgenic PiZ mouse model (PiZ mice) was used to evaluate AAT RNAi agents in vivo. PiZ mice harbor the human PiZ AAT mutant allele and model human AATD (Carlson et al., Journal of Clinical Investigation 1989).

NAG-conjugated AAT RNAi agents were prepared in a pharmaceutically acceptable saline buffer and administered to PiZ mice to evaluate knockdown of AAT gene expression. On day 1, each mouse received a single subcutaneous (SQ) dose into the loose skin on the back between the shoulders of 5.0 mg/kg (mpk) of either AD04446, AD04447. AD04448, AD04449, AD04450, AD04451, AD04454, AD04455, AD04456, AD04457, AD04458, or AD04459. (See Tables 4-7 for the modified AAT RNAi agents and NAG ligand structures). AAT RNAi agents AD04451 and ADU4459 included a modified nucleotide antisense strand sequence designed to target an AAT gene (SEQ ID NO: 1) at position 1000; AAT RNAi agents AD04446 and AD04454 included a modified nucleotide antisense strand sequence designed to target an AAT gene (SEQ ID NO: 1) at position 1142; AAT RNAi agents AD04447 and AD04455 included a modified nucleotide antisense strand sequence designed to target an AAT gene (SEQ ID NO: 1) at position 1211; AAT RNAi agents AD04448 and AD04456 included a modified nucleotide antisense strand sequence designed to target an AAT gene (SEQ ID NO: 1) at position 1326; AAT RNAi agents AD04449 and AD04457 included a modified nucleotide antisense strand sequence designed to target an AAT gene (SEQ ID NO: 1) at position 1338; and AAT RNAi agents AD04450 and AD04458 included a modified nucleotide antisense strand sequence designed to target an AAT gene (SEQ ID NO: 1) at position 1427. (Se also Tables 1 and 2). Three mice were dosed with each AAT RNAi agent (n=3).

Plasma samples were drawn and analyzed for AAT (Z-AAT) protein levels on day 1 (pre-dose), day 8, day 15, day 22, day 29, and day 36. AAT levels were normalized to day 1 (pre-dose) AAT plasma levels. Protein levels were measured by quantifying circulating human Z-AAT levels in plasma by a commercially available ELISA kit according to the manufacturer's recommendations. The average normalized AAT (Z-AAT) levels for each RNAi agent are reported in the following Table 10:

TABLE 10
Average Normalized AAT Protein (Normalized to Pre-Treatment) from Example 3

| | Day 8 | | Day 15 | | Day 22 | | Day 29 | | Day 36 | |
|---|---|---|---|---|---|---|---|---|---|---|
| | Avg | Std Dev | Avg | Std Dev | Avg | Std Dev | Avg | Std Dev | Avg | Std Dev |
| Group ID | AAT | (+/−) | AAT | (+/−) | AAT | (+/−) | AAT | (+/−) | AAT | (+/−) |
| Group 1 (5.0 | 1.010 | 0.256 | 1.050 | 0.108 | 1.451 | 0.137 | 1.145 | 0.154 | 1.117 | 0.080 |
| mg/kg AD04447) | | | | | | | | | | |
| Group 2 (5.0 | 0.884 | 0.262 | 0.866 | 0.276 | 1.306 | 0.112 | 1.147 | 0.119 | 1.076 | 0.172 |
| mg/kg AD04448) | | | | | | | | | | |
| Group 3 (5.0 | 0.909 | 0.060 | 0.969 | 0.152 | 1.290 | 0.185 | 1.290 | 0.201 | 1.245 | 0.106 |
| mg/kg AD04449) | | | | | | | | | | |
| Group 4 (5.0 | 0.595 | 0.083 | 0.799 | 0.131 | 1.099 | 0.256 | 1.090 | 0.346 | 1.229 | 0.444 |
| mg/kg AD04450) | | | | | | | | | | |
| Group 5 (5.0 | 0.282 | 0.006 | 0.525 | 0.020 | 1.358 | 0.188 | 1.767 | 0.325 | 1.586 | 0.297 |
| mg/kg AD04451) | | | | | | | | | | |
| Group 6 (5.0 | 0.656 | 0.126 | 0.639 | 0.039 | 0.741 | 0.089 | 0.738 | 0.235 | 0.819 | 0.156 |
| mg/kg AD04455) | | | | | | | | | | |
| Group 7 (5.0 | 0.605 | 0.129 | 0.469 | 0.036 | 0.717 | 0.105 | 0.662 | 0.097 | 0.875 | 0.195 |
| mg/kg AD04456) | | | | | | | | | | |
| Group 8 (5.0 | 0.501 | 0.108 | 0.663 | 0.091 | 1.031 | 0.324 | 1.176 | 0.368 | 1.603 | 0.597 |
| mg/kg AD04457) | | | | | | | | | | |
| Group 9 (5.0 | 0.308 | 0.081 | 0.174 | 0.031 | 0.177 | 0.010 | 0.211 | 0.010 | 0.345 | 0.041 |
| mg/kg AD04458) | | | | | | | | | | |
| Group 10 (5.0 | 0.256 | 0.021 | 0.134 | 0.045 | 0.174 | 0.084 | 0.234 | 0.174 | 0.315 | 0.336 |
| mg/kg AD04459) | | | | | | | | | | |
| Group 11 (5.0 | 0.686 | 0.178 | 0.739 | 0.130 | 0.973 | 0.263 | 0.955 | 0.107 | 0.885 | 0.119 |
| mg/kg AD04446) | | | | | | | | | | |
| Group 12 (5.0 | 0.338 | 0.014 | 0.361 | 0.105 | 0.602 | 0.252 | 0.729 | 0.266 | 0.970 | 0.245 |
| mg/kg AD04454) | | | | | | | | | | |

As shown from the data in Table 10, above, while AAT RNAi agent AD04447 showed essentially no reduction in AAT protein, AAT RNAi agents AD04458 (which included a modified nucleotide sequence designed to target an AAT gene (SEQ ID NO: 1) at position 1427) and AD04459 (which included a modified nucleotide sequence designed to target an AAT gene (SEQ ID NO: 1) at position 1000) showed a substantial reduction in AAT protein across all timepoints. For example, AD04458 showed knockdown of approximately 69% at day 8 (0.308); approximately 83% at day 15 (0.174), and approximately 82% at day 22 (0.177). Additionally, for example, AD04459 showed a knockdown of approximately 74% at day 8 (0.256), approximately 87% at day 15 (0.134), and approximately 83% at day 22 (0.174).

### Example 4. In Vivo Testing of NAG-Conjugated AAT RNAI Agents in Cynomolgus Monkeys

NAG-conjugated AAT RNAi agents were made and combined in a pharmaceutically acceptable saline buffer as known in the art for subcutaneous (SC) injection. On day 1, cynomolgus macaque (Macaca fascicularis) primates (referred to herein as “cynos” or “monkeys”) were injected subcutaneously with 3 mg/kg of either AD04824, AD04825, AD04826, or AD04827 (see Tables 4-7 for the modified AAT RNAi agents and NAG ligand structures). Each of these AAT RNAi agents included a modified nucleotide sequence designed to target an AAT gene (SEQ ID NO: 1) at position 1000, and was cross-reactive with cynos. Three monkeys in each group were tested (n=3).

Serum samples from treated cynomolgus monkeys were taken on day −7 and day 1 (pre-dose), and on days 8, 15, 22, and 29 to monitor knockdown. Day 36 was also measured for cynos that were injected with AD04825 and AD04826. At the indicated time points, blood samples were drawn and analyzed for cynomolgus monkey AAT (cAAT). Blood was collected from the femoral vein. cAAT levels were determined on a Cobas Integra 400 Plus (Roche Diagnostics) according to the manufacturer's recommendations. AAT levels for each animal at a respective time point was divided by the pre-treatment level (average of day −7 and day 1 (pre-dose)) of expression in that animal to determine the ratio of expression “normalized to pre-dose.”

Normalized cynomolgus monkey AAT (cAAT) protein levels after treatment with each respective AAT RNAi agent are reported in the following Table 11:

TABLE 11
Normalized cAAT Protein (Normalized to Pre-Treatment)
from Example 4 in Cynomolgus Monkeys.

| | Day 8 | Day 15 | Day 22 | Day 29 | Day 36 |
|---|---|---|---|---|---|
| Group ID | cAAT | cAAT | cAAT | cAAT | cAAT |
| Group 1, Cyno A (3.0 mg/kg AD04824) | 0.62 | 0.52 | 0.45 | 0.52 | |
| Group 1, Cyno B (3.0 mg/kg AD04824) | 0.60 | 0.36 | 0.32 | 0.32 | |
| Group 1, Cyno C (3.0 mg/kg AD04824) | 0.62 | 0.44 | 0.41 | 0.41 | |
| Group 2, Cyno A (3.0 mg/kg AD04825) | 0.58 | 0.33 | 0.24 | 0.24 | 0.22 |
| Group 2, Cyno B (3.0 mg/kg AD04825) | 0.58 | 0.38 | 0.27 | 0.25 | 0.27 |
| Group 2, Cyno C (3.0 mg/kg AD04825) | 0.79 | 0.58 | 0.43 | 0,43 | 0.44 |
| Group 3, Cyno A (3.0 mg/kg AD04826) | 0.75 | 0.59 | 0.44 | 0.42 | 0.38 |
| Group 3, Cyno B (3.0 mg/kg AD04826) | 0.66 | 0.43 | 0.30 | 0.26 | 0.24 |
| Group 3, Cyno C (3.0 mg/kg AD04826) | 0.62 | 0.36 | 0.27 | 0.25 | 0.25 |
| Group 4, Cyno A (3.0 mg/kg AD04827) | 0.57 | 0.38 | 0.26 | 0.26 | |
| Group 4, Cyno B (3.0 mg/kg AD04827) | 0.61 | 0.37 | 0.34 | 0.34 | |
| Group 4, Cyno C (3.0 mg/kg AD04827) | 0.66 | 0.43 | 0.41 | 0.39 | |

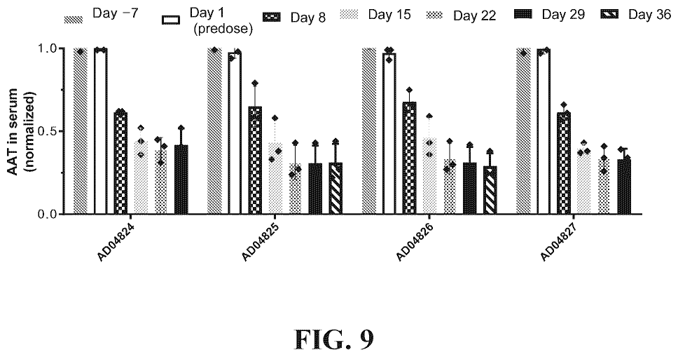

Average normalized cAAT levels for each of the respective treatment groups is shown in the bar graph of FIG. 9. As illustrated in Table 11, above, and in FIG. 9, each of the AAT RNAi agents tested showed substantial knockdown of cAAT in cynomolgus monkeys across all time points measured.

### Example 5. In Vivo Testing of NAG-Conjugated AAT RNAi Agents in Cynomolgus Monkeys

NAG-conjugated AAT RNAi agents were made and combined in a pharmaceutically acceptable saline buffer as known in the art for subcutaneous (SC) injection. On day 1, cynomolgus macaque (Macaca fascicularis) primates were injected subcutaneously with 3 mg/kg of either AD04828, AD04831, AD04836, or AD04837 (see Tables 4-7 for the modified AAT RNAi agents and NAG ligand structures). Each of these AAT RNAi agents included a modified nucleotide sequence designed to target an AAT gene (SEQ ID NO: 1) at position 1000, and was cross-reactive with cynos. Three monkeys in each group were tested (n=3) for AD04828 and AD04831, and two monkeys in each group were tested (n=2) for AD04836 and AD04837.

Serum samples from treated cynos were taken on day −35 and day 1 (pre-dose), and on days 8, 15, 21, and 29 to monitor knockdown. At the indicated time points, blood samples were drawn and analyzed for cAAT. Blood was collected from the femoral vein. cAAT levels were determined on a Cobas Integra 400 Plus (Roche Diagnostics) according to the manufacturer's recommendations. cAAT levels for each animal at a respective time point was divided by the pre-treatment level (average of day −35 and day 1 (pre-dose)) of expression in that animal to determine the ratio of expression “normalized to pre-treatment”.

Normalized cynomolgus monkey AAT (cAAT) protein levels after treatment with each respective AAT RNAi agent are reported in the following Table 12:

TABLE 12
Normalized AAT Protein (Normalized to Pre-Treatment)
from Example 5 in Cynomolgus Monkeys

| | Day 8 | Day 15 | Day 22 | Day 29 |
|---|---|---|---|---|
| Group ID | cAAT | cAAT | cAAT | cAAT |
| Group 1, Cyno A (3,0 mg/kg AD04828) | 0.60 | 0.32 | 0.25 | 0.23 |
| Group 1, Cyno B (3,0 mg/kg AD04828) | 0.67 | 0.59 | 0.61 | 0.76 |
| Group 1, Cyno C (3.0 mg/kg AD04828) | 0.51 | 0.35 | 0.29 | 0.29 |
| Group 2, Cyno A (3.0 mg/kg AD04831) | 0.68 | 0.43 | 0.32 | 0.28 |
| Group 2, Cyno B (3.0 mg/kg AD04831) | 0.71 | 0.49 | 0.47 | 0.44 |
| Group 2, Cyno C (3.0 mg/kg AD04831) | 0.61 | 0.43 | 0.34 | 0.30 |
| Group 3, Cyno A (3.0 mg/kg AD04836) | 0.61 | 0.37 | 0.27 | 0.23 |
| Group 3, Cyno B (3.0 mg/kg AD04836) | 0.67 | 0.43 | 0.32 | 0.27 |
| Group 4, Cyno A (3.0 mg/kg AD04837) | 0.65 | 0.40 | 0.28 | 0.24 |
| Group 4, Cyno B (3.0 mg/kg AD04837) | 0.55 | 0.29 | 0.20 | 0.17 |

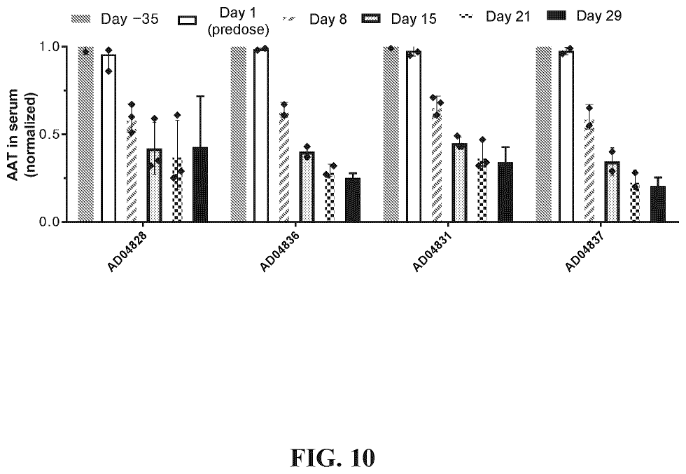

Average normalized cAAT levels for each of the respective treatment groups is shown in the bar graph of FIG. 10. As shown above in Table 12, as well as in the bar graph of FIG. 10, each of the AAT RNAi agents tested showed substantial knockdown of cAAT in cynomolgus monkeys across all time points measured.

### Example 6. In Vivo Testing of NAG-Conjugated AAT RNAi Agents in PiZ Mice

The transgenic PiZ mouse model (PiZ mice) as set forth in Example 3 was used to evaluate AAT RNAi agents in vivo. NAG-conjugated AAT RNAi agents were prepared in a pharmaceutically acceptable saline buffer and administered to PiZ mice to evaluate knockdown of AAT gene expression. On day 1, each mouse received a single subcutaneous (SQ) dose into the loose skin on the back between the shoulders of 2.0 mg/kg (mpk) of either AD04824, AD04828, AD04829, AD04830, AD04831, AD04832, AD04833, AD04834, AD04836, AD04837, AD04838, AD04839, or AD04857. (See Tables 4-7 for the modified AAT RNAi agents and NAG ligand structures). Each of the AAT RNAi agents in this study included a modified nucleotide antisense strand sequence designed to target an AAT gene (SEQ ID NO: 1) at position 1000. (See also Tables 1 and 2). Three mice were dosed with each AAT RNAi agent (n=3).

Plasma samples were drawn and analyzed for AAT (Z-AAT) protein levels on days −2, day 1 (pre-dose), day 8, day 15, day 22, day 29, and day 36. AAT levels were normalized to day 1 (pre-dose) AAT plasma levels. Protein levels were measured by quantifying circulating human Z-AAT levels in plasma by a commercially available ELISA kit according to the manufacturer's recommendations. The average normalized AAT (Z-AAT) levels for each RNAi agent are reported in the following Table 13:

TABLE 13
Average Normalized AAT Protein (Normalized to Pre-Treatment) from Example 6

| | Day 8 | | Day 15 | | Day 22 | | Day 29 | | Day 36 | |
|---|---|---|---|---|---|---|---|---|---|---|
| | Avg | Std Dev | Avg | Std Dev | Avg | Std Dev | Avg | Std Dev | Avg | Std Dev |
| Group ID | AAT | (+/−) | AAT | (+/−) | AAT | (+/−) | AAT | (+/−) | AAT | (+/−) |
| Group 1 (2.0 | 0.105 | 0.036 | 0.140 | 0.067 | 0.204 | 0.108 | 0.313 | 0.104 | 0.437 | 0.229 |
| mg/kg AD04824) | | | | | | | | | | |
| Group 2 (2.0 | 0.141 | 0.055 | 0.236 | 0.111 | 0.304 | 0.138 | 0.624 | 0.289 | 0.814 | 0.139 |
| mg/kg AD04828) | | | | | | | | | | |
| Group 3 (2.0 | 0.109 | 0.072 | 0.119 | 0.102 | 0.140 | 0.119 | 0.141 | 0.116 | 0.179 | 0.145 |
| mg/kg AD04829) | | | | | | | | | | |
| Group 4 (2.0 | 0.147 | 0.095 | 0.190 | 0.148 | 0.307 | 0.192 | 0.521 | 0.424 | 0.547 | 0.202 |
| mg/kg AD04830) | | | | | | | | | | |
| Group 5 (2.0 | 0.154 | 0.104 | 0.215 | 0.171 | 0.449 | 0.375 | 0.701 | 0.519 | 0.584 | 0.418 |
| mg/kg AD04831) | | | | | | | | | | |
| Group 6 (2.0 | 0.088 | 0.032 | 0.089 | 0.048 | 0.090 | 0.046 | 0.117 | 0.071 | 0.193 | 0.131 |
| mg/kg AD04832) | | | | | | | | | | |
| Group 7 (2.0 | 0.168 | 0.029 | 0.282 | 0.047 | 0.448 | 0.048 | 0.748 | 0.223 | 1.361 | 0.346 |
| mg/kg AD04833) | | | | | | | | | | |
| Group 8 (2.0 | 0.159 | 0.037 | 0.255 | 0.159 | 0.470 | 0.315 | 0.662 | 0.346 | 0.728 | 0.141 |
| mg/kg AD04834) | | | | | | | | | | |
| Group 9 (2.0 | 0.108 | 0.035 | 0.070 | 0.024 | 0.083 | 0.032 | 0.090 | 0.035 | 0.168 | 0.078 |
| mg/kg AD04836) | | | | | | | | | | |
| Group 10 (2.0 | 0.157 | 0.071 | 0.209 | 0.104 | 0.242 | 0.097 | 0.417 | 0.198 | 0.550 | 0.193 |
| mg/kg AD04837) | | | | | | | | | | |
| Group 11 (2.0 | 0.106 | 0.017 | 0.099 | 0.022 | 0.108 | 0.039 | 0.158 | 0.072 | 0.188 | 0.050 |
| mg/kg AD04838) | | | | | | | | | | |
| Group 12 (2.0 | 0.096 | 0.026 | 0.069 | 0.019 | 0.089 | 0.036 | 0.120 | 0.038 | 0.186 | 0.083 |
| mg/kg AD04839) | | | | | | | | | | |
| Group 12 (2.0 | 0.272 | 0.130 | 0.302 | 0.145 | 0.478 | 0.187 | 0.815 | 0.436 | 1.772 | 1.412 |
| mg/kg AD04857) | | | | | | | | | | |

As shown from the data in Table 13, above, each of the AAT RNAi agent showed a substantial reduction in AAT protein through at least day 29. For example, at day 15, each of the AAT RNAi agents tested achieved at least approximately 70% knockdown of protein compared to pre-treatment levels, with multiple groups achieving 90% or better knockdown.

### Example 7. In Vivo Testing of NAG-Conjugated AAT RNAi Agents in PiZ Mice

The transgenic PiZ mouse model described in Example 3 was used. Each mouse was 5 weeks old at the beginning of the study. NAG-conjugated AAT RNAi agents were prepared in a pharmaceutically acceptable saline buffer and administered to PiZ mice to evaluate knockdown of AAT gene expression. Starting on day 1, each mouse received a subcutaneous (SQ) dose q2w (i.e., one injection every two weeks, for a total of 4 injections) into the loose skin on the back between the shoulders of 4.0 mg/kg (mpk) of either: (1) saline vehicle; (2) AD04837 (see Tables 4-7 for the modified AAT RNAi agent and NAG ligand structures), which as noted previously included a modified nucleotide antisense strand sequence designed to target an AAT gene (SEQ ID NO: 1) at position 1000; or (3) a NAG-conjugated RNAi agent that included a nucleotide sequence targeting the HBV gene, to be used as a negative control. Single subcutaneous injections for the saline vehicle group, AAT RNAi agent group, and HBV RNAi agent group were performed on days 1, 15, 29, and 43. Seven (7) mice were dosed q2w with the saline vehicle (Group 1); nine (9) mice were dosed q2w with the AAT RNAi agent (Group 2); and six (6) mice were dosed with the HBV RNAi agent (Group 3). The mice in the three treatment groups were sacrificed on day 57 (13 weeks old). In addition to the treatment groups, seven (7) mice were sacrificed at week 1 of the study (i.e., 5-week old mice) to serve as a baseline control.

Plasma samples were drawn and analyzed for AAT (Z-AAT) protein levels on day 1 (pre-dose), day 8, day 15, day 22, day 29, and day 36 for all groups. Additional samples for the AAT RNAi agent group and the saline vehicle group were drawn on day 43, day 50, and day 57. AAT levels were normalized to day 1 (pre-dose) AAT plasma levels. Protein levels were measured by quantifying circulating human Z-AAT levels in plasma by a commercially available ELISA kit according to the manufacturer's recommendations. The average normalized AAT (Z-AAT) levels for the saline vehicle and each RNAi agent are reported in the following Table 14:

TABLE 14
Average Normalized AAT Protein (Normalized to Pre-Treatment) from Example 7

| | Day 8 | | Day 15 | | Day 22 | | Day 29 | |
|---|---|---|---|---|---|---|---|---|
| | Avg | Std Dev | Avg | Std Dev | Avg | Std Dev | Avg | Std Dev |
| Group ID | AAT | (+/−) | AAT | (+/−) | AAT | (+/−) | AAT | (+/−) |
| Group 1 (saline vehicle) (n = 7) | 0.876 | 0.172 | 1.264 | 0.386 | 1.234 | 0.457 | 1.319 | 0.453 |
| Group 2 (AD04837) (n = 9) | 0.139 | 0.050 | 0.146 | 0.064 | 0.067 | 0.029 | 0.072 | 0.038 |
| Group 3 (HBV RNAi agent − negative | 1.212 | 0.360 | 1.019 | 0.201 | 1.540 | 0.155 | 1.585 | 0.640 |
| control) (n = 6) | | | | | | | | |
| | Day 36 | | Day 43 | | Day 50 | | Day 57 | |
| | Avg | Std Dev | Avg | Std Dev | Avg | Std Dev | Avg | Std Dev |
| Group ID | AAT | (+/−) | AAT | (+/−) | AAT | (+/−) | AAT | (+/−) |
| Group 1 (saline vehicle) (n = 7) | 1.267 | 0.491 | 1.441 | 0.416 | 1.172 | 0.340 | 1.058 | 0.299 |
| Group 2 (AD04837) (n = 9) | 0.040 | 0.011 | 0.051 | 0.020 | 0.034 | 0.007 | 0.038 | 0.009 |
| Group 3 (HBV RNAi agent − negative | 1.665 | 0.476 | 1.943 | 0.221 | 1.580 | 0.491 | 2.001 | 0.770 |
| control) (n = 6) | | | | | | | | |

As shown from the data in Table 14, above, the HBV RNAi agent performed successfully as a negative control showing essentially no AAT inhibition. Further, the NAG-conjugated AAT RNAi agent (AD04837) achieved significant knockdown of expression compared to saline and the HBV RNAi agent negative control across all timepoints. In dosing q2w, the AAT RNAi agent in Example 7 showed a knockdown of approximately 96% of AAT protein at day 36 (0.040) and maintained a similar level of knockdown through day 57.

In addition to monitoring serum AAT levels, homogenized liver tissue from PiZ mice treated with NAG-conjugated AAT RNAi agent (AD04837) was further analyzed to determine if both soluble Z-AAT (which is expected to be predominantly monomeric protein), and insoluble polymers of Z-AAT (which is expected to be polymeric protein) were effectively reduced. A modified western blot protocol was used to separate the soluble and insoluble Z-AAT fractions under non-denaturing conditions as previously described and known in the art (see. e.g., Mueller et al., Molecular Therapy, March 2012, 20(3): 590-600).

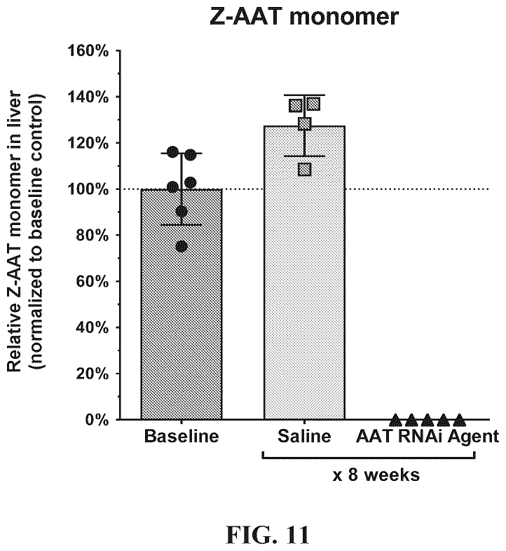

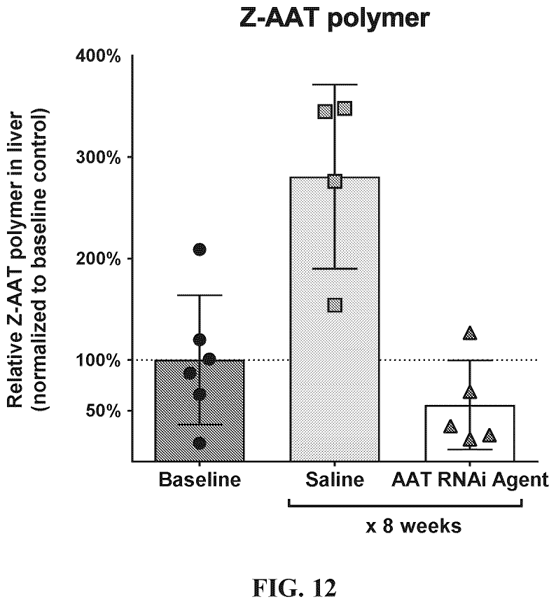

A western blot was prepared to examine certain livers of the sacrificed mice. Specifically, livers were examined of (i) 6 baseline mice; (ii) 5 AAT RNAi agent mice; and (iii) 4 saline mice. (The gels used for the western blot analysis included 15 wells). The samples for the animals used for this western blot were randomly selected from the various groups. FIGS. 11 and 12 show bar graphs reflecting the Z-AAT polymer and Z-AAT monomer levels quantified from the western blot analysis.

As seen from the bar graph in FIG. 11, which reports the monomeric protein levels, when compared to baseline each of the mice dosed with AAT RNAi agent shown a significant reduction in AAT monomeric protein across all time points, indicating significant inhibition of the gene. Further, as shown in FIG. 12, which reports the polymeric protein levels, the animals treated with the saline vehicle continued to have increased polymeric AAT burden after 8 weeks. Conversely, the animals treated with AAT RNAi agent showed a reduction in polymeric burden of approximately 50% over the course of 8 weeks as compared to the baseline (5-week-old) mice, indicating that the administration of NAG-conjugated AAT RNAi agent (AD04837) is capable of preventing and potentially reversing the production of polymeric AAT protein.

### Example 8. In Vivo Testing of NAG-Conjugated AAT RNAI Agents in PZ Mice

The transgenic PiZ mouse model described in Example 3 was used to evaluate RNAi agents in vivo. Each mouse received a single subcutaneous (SQ) dose on day 1 into the loose skin on the back between the shoulders of either: (1) saline: (2) 1.0 mg/kg of the NAG-conjugated AAT RNAi agent of AD04837 (which includes a modified nucleotide antisense strand sequence designed to target an AAT gene (SEQ ID NO: 1) at position 1000); (3) 2.0 mg/kg of AD04837; (4) 4.0 mg/kg of AD04837; or (5) 8.0 mg/kg of AD04837. Four animals were dosed in group 1 (saline), and all four were sacrificed on day 43. Fifteen (15) animals were dosed in each of groups 2, 3, 4, and 5, and 3 animals from each group were sacrificed on day 8, day 15, day 22, day 29, and day 43, respectively.

Plasma samples were drawn and analyzed for AAT (Z-AAT) protein levels on day 1 (pre-dose), day 8, day 15, day 22, day 29, day 36, and day 43 for all groups. For the sacrificed mice, cardiac sticks were performed for serum isolation for Z-AAT protein level assessment (200 μl plasma). AAT levels were normalized to day 1 (pre-dose) AAT plasma levels. Protein levels were measured by quantifying circulating human Z-AAT levels in plasma by a commercially available ELISA kit according to the manufacturer's recommendations. The average normalized AAT (Z-AAT) levels for the saline vehicle and each RNAi agent dosing group are reported in the following Table 15:

TABLE 15
Average Normalized Plasma AAT Protein (Normalized to Pre-Treatment) from Example 8.

| | Day 8 | | Day 15 | | Day 22 | | Day 29 | |
|---|---|---|---|---|---|---|---|---|
| | Avg | Std Dev | Avg | Std Dev | Avg | Std Dev | Avg | Std Dev |
| Group ID | AAT | (+/−) | AAT | (+/−) | AAT | (+/−) | AAT | (+/−) |
| Group 1 (saline vehicle) | 1.240 | 0.633 | 1.037 | 0.256 | 0.884 | 0.229 | 0.857 | 0.286 |
| Group 2 (1.0 mg/kg AD04837) | 0.266 | 0.100 | 0.250 | 0.107 | 0.259 | 0.060 | 0.412 | 0.191 |
| Group 3 (2.0 mg/kg AD04837) | 0.170 | 0.102 | 0.162 | 0.132 | 0.199 | 0.161 | 0.511 | 0.514 |
| Group 4 (4.0 mg/kg AD04837) | 0.051 | 0.015 | 0.038 | 0.010 | 0.051 | 0.021 | 0.110 | 0.045 |
| Group 5 (8.0 mg/kg AD04837) | 0.030 | 0.011 | 0.025 | 0.010 | 0.040 | 0.024 | 0.063 | 0.030 |

| | Day 36 | | Day 43 | |
|---|---|---|---|---|
| Group 1 (saline vehicle) | 1.485 | 0.431 | 0.932 | 0.243 |
| Group 2 (1.0 mg/kg AD04837) | 0.791 | 0.207 | 0.560 | 0.111 |
| Group 3 (2.0 mg/kg AD04837) | 0.600 | 0.140 | 0.595 | 0.217 |
| Group 4 (4.0 mg/kg AD04837) | 0.156 | 0.008 | 0.148 | 0.022 |
| Group 5 (8.0 mg/kg AD04837) | 0.239 | 0.183 | 0.202 | 0.119 |

As shown from the data in Table 15, above, the NAG-conjugated AAT RNAi agent achieved significant knockdown of expression compared to saline across all timepoints measured at all dosing levels tested.

In addition, AAT mRNA levels were also assessed for the sacrificed mice at each respective timepoint. As described above, for Groups 2 through 5 (i.e., the RNAi agent groups), 3 mice were sacrificed on each of days 8, 15, 22, 29 and 43, and for Group 1, all 4 mice were sacrificed on day 43. Half of the left lateral liver lobe was collected and snap-frozen in liquid nitrogen for RNA isolation.

TABLE 16
Relative AAT mRNA Levels in PiZ Mice Following Administration of a Single SQ
Injection of Saline or AAT RNAi Agent

| | | | Average Relative | Low | High |
|---|---|---|---|---|---|
| Treatment Group | Day | Animals | mRNA Expression | Variance | Variance |
| Group 1 (saline vehicle) | 43 | n = 4 | 1.000 | 0.071 | 0.076 |
| Group 2 (1.0 mg/kg | 8 | n = 3 | 0.412 | 0.080 | 0.099 |
| AD04837) | 15 | n = 3 | 0.419 | 0.037 | 0.040 |
| | 22 | n = 3 | 0.483 | 0.066 | 0.076 |
| | 29 | n = 3 | 0.696 | 0.069 | 0.076 |
| | 43 | n = 3 | 0.813 | 0.103 | 0.118 |
| Group 3 (2.0 mg/kg | 8 | n = 3 | 0.272 | 0.101 | 0.160 |
| AD04837) | 15 | n = 3 | 0.235 | 0.039 | 0.046 |
| | 22 | n = 3 | 0.327 | 0.099 | 0.141 |
| | 29 | n = 3 | 0.587 | 0.155 | 0.210 |
| | 43 | n = 3 | 0.845 | 0.123 | 0.145 |
| Group 4 (4.0 mg/kg | 8 | n = 3 | 0.129 | 0.025 | 0.031 |
| AD04837) | 15 | n = 3 | 0.161 | 0.017 | 0.020 |
| | 22 | n = 3 | 0.222 | 0.048 | 0.061 |
| | 29 | n = 3 | 0.247 | 0.067 | 0.093 |
| | 43 | n = 3 | 0.454 | 0.051 | 0.057 |
| Group 5 (8.0 mg/kg | 8 | n = 3 | 0.078 | 0.013 | 0.015 |
| AD04837) | 15 | n = 3 | 0.055 | 0.014 | 0.019 |
| | 22 | n = 3 | 0.077 | 0.009 | 0.010 |
| | 29 | n = 3 | 0.116 | 0.038 | 0.056 |
| | 43 | n = 3 | 0.332 | 0.122 | 0.193 |

As shown in Table 16, above, relative AAT mRNA expression levels were significantly reduced across all timepoints measured compared to saline vehicle. For example, on day 15, Group 2 (1.0 mg/kg AAT RNAi agent) showed approximately 58% reduction of AAT mRNA levels (0.419): Group 3 (2.0 mg/kg AAT RNAi agent) showed approximately 67% reduction of AAT mRNA levels (0.327): Group 4 (4.0 mg/kg AAT RNAi agent) showed approximately 84% reduction of AAT mRNA levels (0.161): and Group 5 (8.0 mg/kg AAT RNAi agent) showed approximately 94% reduction of Z-AAT mRNA levels (0.055) upon a single SQ dose at day 1.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting the expression of an alpha-1 antitrypsin (AAT) gene, wherein the RNAi agent comprises a sense strand and an antisense strand, wherein the antisense strand comprises nucleotides 2-18 of any of the antisense strand sequences in Table 2, Table 3, or Table 4, and wherein the sense strand is at least substantially complementary to the antisense strand.

2. An RNAi agent for inhibiting the expression of an alpha-1 antitrypsin (AAT) gene, wherein the RNAi agent comprises a sense strand and an antisense strand, wherein the sense strand comprises nucleotides 2-18 of any of the sense strand sequences in Table 2, Table 3, or Table 5, and wherein the antisense strand is at least substantially complementary to the sense strand.

3. The RNAi agent of claim 1, wherein antisense strand comprises the nucleotide sequence of any of the antisense strand sequences in Table 2, Table 3, or Table 4.

4. The RNAi agent of claim 2, wherein sense strand comprises the nucleotide sequence of any of the antisense strand sequences in Table 2, Table 3, or Table 5.

5. The RNAi agent of claim 1, wherein the sense strand comprises the nucleotide sequence of any of the sense strand sequences in Table 4, and the antisense strand comprises the nucleotide sequence of any of the antisense strand sequences in Table 5.

6. The RNAi agent of claim 1, wherein the RNAi agent comprises at least one modified nucleotide.

7. The RNAi agent of any of claim 6, wherein the at least one modified nucleotide is selected from the group consisting of: 2′-O-methyl nucleotide, 2′-Fluoro nucleotide, 2′-deoxy nucleotide, 2′,3′-seco nucleotide mimic, locked nucleotide, 2′-F-Arabino nucleotide, 2′-methoxyethyl nucleotide, abasic nucleotide, ribitol, inverted nucleotide, inverted abasic nucleotide, inverted 2′-OMe nucleotide, inverted 2′-deoxy nucleotide, 2′-amino-modified nucleotide, 2′-alkyl-modified nucleotide, morpholino nucleotide, vinyl phosphonate deoxyribonucleotide, and 3′-OMe nucleotide.

8. The RNAi agent of claim 1, wherein the RNAi agent comprises at least one phosphorothioate internucleoside linkage.

9. The RNAi agent of claim 8, wherein the sense strand comprises at least one phosphorothioate internucleoside linkage.

10. The RNAi agent of claim 8, wherein the antisense strand contains one, two, three, or four phosphorothioate internucleoside linkages.

11. The RNAi agent of claim 1, wherein all or substantially all of the nucleotides of the sense strand and the antisense strand are modified nucleotides.

12. The RNAi agent of claim 1, wherein the sense strand is no more than 30 nucleotides in length, and the antisense strand is no more than 30 nucleotides in length.

13. The RNAi agent of claim 12, wherein the sense strand is no more than 24 nucleotides in length, and the antisense strand is no more than 24 nucleotides in length.

14. The RNAi agent of claim 13, wherein the sense strand and the antisense strand are each between 18 and 24 nucleotides in length.

15. The RNAi agent of claim 14, wherein the sense strand and the antisense strand are each 21 nucleotides in length.

16. The RNAi agent of claim 1, wherein the RNAi agent has two blunt ends.

17. The RNAi agent of claim 1, wherein the RNAi agent comprises at least one modified nucleotide and further comprises one or more targeting groups or linking groups.

18. The RNAi agent of claim 17, wherein the targeting groups or linking groups have the structure of any of the structures represented in Table 7.

19. The RNAi agent of claim 18, wherein the one or more targeting groups or linking groups are conjugated to the sense strand.

20. The RNAi agent of claim 1, wherein the RNAi agent comprises a targeting group that includes an asialoglycoprotein receptor ligand.

21. The RNAi agent of claim 20, wherein the asialoglycoprotein receptor ligand comprises N-acetyl-galactosamine.

22. The RNAi agent of claim 21, wherein the asialoglycoprotein receptor ligand comprises an N-acetyl-galactosamine trimer.

23. The RNAi agent of claim 1, wherein the RNAi agent comprises at least one modified nucleotide and further comprises one or more targeting groups, wherein the targeting group has a structure selected from the group consisting of: (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), (NAG39)s.

24. The RNAi agent of claim 19, wherein the RNAi agent comprises a targeting group that is conjugated to the 5′ terminal end of the sense strand.

25. The RNAi agent of claim 1, wherein the RNAi agent is comprised of a sense strand and antisense strand forming a duplex having the structure of any of the duplexes in Table 6.

26. The RNAi agent of claim 1, wherein the RNAi agent has the duplex structure selected from the group consisting of: AD04824, AD04825, AD04826, AD04827, AD04828, AD04829, AD04830, AD04831, AD04832, AD04833, AD04834, AD04835, AD04836, and AD04837.

27. The RNAi agent of claim 1, wherein the antisense strand comprises the sequence (5′→3′) UGUUAAACAUGCCUAAACGCU (SEQ ID NO: 801), wherein all or substantially all of the nucleotides of the antisense strand are modified nucleotides, and wherein the sense strand comprises the sequence (5′→3′) AGCGUUUAGGCAUGUUUAACA (SEQ ID NO: 866), wherein all or substantially all of the nucleotides of the sense strand are modified nucleotides.

28. The RNAi agent of claim 1, wherein the antisense strand comprises the sequence (5′→3′) UGUUAAACAUGCCUAAACGUU (SEQ ID NO: 794), wherein all or substantially all of the nucleotides of the antisense strand are modified nucleotides, and wherein the sense strand comprises the sequence (5′→3′) CGUUUAGGCAUGUUUAACAUU (SEQ ID NO: 857), wherein all or substantially all of the nucleotides of the sense strand are modified nucleotides.

29. The RNAi agent of claim 1, wherein the antisense strand comprises the sequence (5′→3′) UGUUAAACAUGCCUAAACGCUU (SEQ ID NO: 839), wherein all or substantially all of the nucleotides of the antisense strand are modified nucleotides, and wherein the sense strand comprises the sequence (5′→3′) GCGUUUAGGCAUGUUUAACAUU (SEQ ID NO: 885), wherein all or substantially all of the nucleotides of the sense strand are modified nucleotides.

30. The RNAi agent of claim 1, wherein the antisense strand comprises the sequence (5′ 4 3′) UGUUAAACAUGCCUAAACGCG (SEQ ID NO: 800), wherein all or substantially all of the nucleotides of the antisense strand are modified nucleotides, and wherein the sense strand comprises the sequence (5′→3′) CGCGUUUAGGCAUGUUUAACA (SEQ ID NO: 864), wherein all or substantially all of the nucleotides of the sense strand are modified nucleotides.

31. The RNAi agent of claim 1, wherein the antisense strand comprises the sequence (5′→3′) UGUUAAACAUGCCUAAACGCU (SEQ ID NO: 801), wherein all or substantially all of the nucleotides are modified nucleotides, and wherein SEQ ID NO: 801 is located at positions 1 to 21 (5′→3′) of the antisense strand.

32. The RNAi agent of claim 31, wherein the antisense strand comprises the nucleotide sequence (5′→3′) usGfsuUfaAfacaugCfcUfaAfaCfgCfsu (SEQ ID NO: 960), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; and s is a phosphorothioate linkage.

33. The RNAi agent of claim 32, wherein the sense strand comprises the sequence (5′→3′) agcguuuaGfGfCfauguuuaaca (SEQ ID NO: 1279), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively, s is a phosphorothioate linkage: wherein optionally present on the sense strand is one or two inverted abasic deoxyribose residues (invAb) and/or one, two, three, or four phosphorothioate internucleoside linkages, and wherein optionally linked to the 5′ terminal end of the sense strand is a targeting ligand that includes N-acetyl-galactosamine.

34. The RNAi agent of claim 33, wherein the RNAi agent has the duplex structure of AD04837 (SEQ ID PAIR NOs: 960/1033).

35. The RNAi agent of claim 1, wherein the antisense strand comprises the sequence (5′→3′) UGUUAAACAUGCCUAAACGUU (SEQ ID NO: 794), wherein all or substantially all of the nucleotides are modified nucleotides, and wherein SEQ ID NO: 794 is located at positions 1 to 21 (5′→3′) of the antisense strand.

36. The RNAi agent of claim 35, wherein the antisense strand of the RNAi agent comprises the sequence (5′→3′) usGfsusUfaAfaCfaUfgCfcUfaAfaCfgusu (SEQ ID NO: 913), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; and s is a phosphorothioate linkage.

37. The RNAi agent of claim 36, wherein the sense strand comprises the sequence (5′→3′) cguuuaGfGfCfauguuuaacausu (SEQ ID NO: 1276), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively: s is a phosphorothioate linkage: wherein optionally present on the sense strand is one or two inverted abasic deoxyribose residues (invAb) and/or one, two, three, or four phosphorothioate internucleoside linkages; and wherein optionally linked to the 5′ terminal end of the sense strand is a targeting ligand that includes N-acetyl-galactosamine.

38. The RNAi agent of claim 37, wherein the RNAi agent has the duplex structure of AD04828 (SEQ ID PAIR NOs: 913/1028).

39. The RNAi agent of claim 1, wherein the antisense strand comprises the sequence (5′→3′) UGUUAAACAUGCCUAAACGCUU (SEQ ID NO: 839), wherein all or substantially all of the nucleotides are modified nucleotides, and wherein SEQ ID NO: 839 is located at positions 1 to 22 (5′→3′) of the antisense strand.

40. The RNAi agent of claim 39, wherein the antisense strand of the RNAi agent comprises the sequence (5′→3′) usGfsusUfaAfaCfaUfgCfcUfaAfaCfgcusu (SEQ ID NO: 958), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; and s is a phosphorothioate linkage.

41. The RNAi agent of claim 40, wherein the sense strand comprises the sequence (5′→3′) gcguuuaGfGfCfauguuuaacausu (SEQ ID NO: 1277), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively; s is a phosphorothioate linkage; wherein optionally present on the sense strand is one or two inverted abasic deoxyribose residues (invAb) and/or one, two, three, or four phosphorothioate internucleoside linkages; and wherein optionally linked to the 5′ terminal end of the sense strand is a targeting ligand that includes N-acetyl-galactosamine.

42. The RNAi agent of claim 41, wherein the RNAi agent has the duplex structure of AD04831 (SEQ ID PAIR NOs: 958/1030).

43. The RNAi agent of claim 1, wherein the antisense strand comprises the sequence (5′→3′) UGUUAAACAUGCCUAAACGCG (SEQ ID NO: 800), wherein all or substantially all of the nucleotides are modified nucleotides, and wherein SEQ ID NO: 800 is located at positions 1 to 21 (5′→3′) of the antisense strand.

44. The RNAi agent of claim 43, wherein the antisense strand of the RNAi agent comprises the sequence (5′→3′) usGfsuUfaAfaCfaUfgCfcUfaAfaCfgsCfsg (SEQ ID NO: 959), wherein a, c, g, and u are 2′-O-methyl adenosine, cytidine, guanosine, or uridine, respectively: Af, Cf, Gf, and Uf are 2′-fluoro adenosine, cytidine, guanosine, or uridine, respectively; and s is a phosphorothioate linkage.

45. The RNAi agent of claim 44, wherein the sense strand comprises the sequence (5′→3′) cgcguuuaGfGfCfauguuuaaca (SEQ ID NO: 1278), wherein a, c, g, and u are 2′-O-methyl adenosine, cytidine, guanosine, or uridine, respectively; Af, Cf, Gf, and Uf are 2′-fluoro adenosine, cytidine, guanosine, or uridine, respectively: s is a phosphorothioate linkage: wherein optionally present on the sense strand is one or two inverted abasic deoxyribose residues (invAb) and/or one, two, three, or four phosphorothioate internucleoside linkages; and wherein optionally linked to the 5′ terminal end of the sense strand is a targeting ligand that includes N-acetyl-galactosamine.

46. The RNAi agent of claim 45, wherein the RNAi agent has the duplex structure of AD04836 (SEQ ID PAIR NOs: 959/1024).

47. A composition comprising the RNAi agent of claim 1, and at least one pharmaceutically acceptable excipient.

48. The composition of claim 47, further comprising one or more additional therapeutics or treatments.

49. The composition of claim 47, wherein the composition is packaged in a kit, container, pack, dispenser, pre-filled syringes, or vials.

50. The composition of claim 47, wherein the composition is formulated for administration by subcutaneous injection.

51. The composition of claim 47, wherein the composition includes one or more duplexes having the structure selected from the group consisting of: AD04824, AD04825, AD04826, AD(4827, AD04828, AD04829, AD04830, AD04831, AD04832, AD04833, AD04834, AD04835, AD04836, and AD04837.

52. A method for inhibiting the expression of an AAT gene in a cell, the method comprising administering an effective amount of an RNAi agent of claim 1.

53. A method for inhibiting the expression of an AAT gene in a subject, the method comprising administering to the subject an effective amount of an RNAi agent of claim 1.

54. A method for the treatment of alpha-1 antitrypsin deficiency (AATD), the method comprising administering to a subject in need thereof a therapeutically effective amount of an RNAi agent of claim 1.

55. A method for the treatment of a condition or disease caused by alpha-1 antitrypsin deficiency (AATD), the method comprising administering to a subject in need thereof a therapeutically effective amount of an RNAi agent of claim 1.

56. The method of claim 55, wherein the condition or disease caused by AAT is a liver disease.

57. The method of claim 56, wherein the liver disease is chronic hepatitis, cirrhosis, hepatocellular carcinoma, transaminitis, cholestasis, fibrosis, or fulminant hepatic failure.

58. A method of decreasing or reducing the level of insoluble AAT protein in a subject compared to baseline pre-treatment levels, the method comprising administering to the subject an effective amount of an RNAi agent of claim 1.

59. The method of claim 53, wherein the RNAi agent is administered to the subject subcutaneously.

60. The method of any of claim 53, further comprising measuring serum AAT protein levels (soluble and/or insoluble) or AAT mRNA levels in the subject.

61. The method of any of claim 53, wherein the serum AAT protein levels (soluble and/or insoluble) and/or the AAT mRNA levels in the subject are decreased.

62. A method for inhibiting the expression of an AAT gene in a subject, the method comprising administering to the subject an effective amount of a composition of claim 47.

63. A method for the treatment of alpha-1 antitrypsin deficiency (AATD), the method comprising administering to a subject in need thereof a therapeutically effective amount of a composition of claim 47.

64. A method for the treatment of a condition or disease caused by alpha-1 antitrypsin deficiency (AATD), the method comprising administering to a subject in need thereof a therapeutically effective amount of a composition of claim 47.

65. A method of decreasing or reducing the level of insoluble AAT protein in a subject compared to baseline pre-treatment levels, the method comprising administering to the subject an effective amount of a composition of claims 47.


SEQUENCE LISTING (1279 sequences)

SEQ ID NO: 1 - DNA, 3220 bp, Homo sapiens
  misc_feature 1..3220: note = Serpin family A member 1 (SERPINA1), transcript variant 1, complete gene (NM_000295.4)
  source 1..3220: mol_type = genomic DNA
acaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 2 - RNA, 19 nt, synthetic construct
  misc_feature 1..19: note = AAT mRNA target sequence
  source 1..19: mol_type = other RNA
cgtttaggcatgtttaaca

SEQ ID NO: 3 - RNA, 19 nt, synthetic construct
  misc_feature 1..19: note = AAT mRNA target sequence
  source 1..19: mol_type = other RNA
aacagcaccaatatcttct

SEQ ID NO: 4 - RNA, 19 nt, synthetic construct
  misc_feature 1..19: note = AAT mRNA target sequence
  source 1..19: mol_type = other RNA
atatcatcaccaagttcct

SEQ ID NO: 5 - RNA, 19 nt, synthetic construct
  misc_feature 1..19: note = AAT mRNA target sequence
  source 1..19: mol_type = other RNA
agatgctgcccagaagaca

SEQ ID NO: 6 - RNA, 19 nt, synthetic construct
  misc_feature 1..19: note = AAT mRNA target sequence
  source 1..19: mol_type = other RNA
ctggcacaccagtccaaca

SEQ ID NO: 7 - RNA, 19 nt, synthetic construct
  misc_feature 1..19: note = AAT mRNA target sequence
  source 1..19: mol_type = other RNA
tggcacaccagtccaacag

SEQ ID NO: 8 - RNA, 19 nt, synthetic construct
  misc_feature 1..19: note = AAT mRNA target sequence
  source 1..19: mol_type = other RNA
gcacaccagtccaacagca

SEQ ID NO: 9 - RNA, 19 nt, synthetic construct
  misc_feature 1..19: note = AAT mRNA target sequence
  source 1..19: mol_type = other RNA
cagtccaacagcaccaata

SEQ ID NO: 10 - RNA, 19 nt, synthetic construct
  misc_feature 1..19: note = AAT mRNA target sequence
  source 1..19: mol_type = other RNA
agtccaacagcaccaatat

SEQ ID NO: 11 - RNA, 19 nt, synthetic construct
  misc_feature 1..19: note = AAT mRNA target sequence
  source 1..19: mol_type = other RNA
gtccaacagcaccaatatc

SEQ ID NO: 12 - RNA, 19 nt, synthetic construct
  misc_feature 1..19: note = AAT mRNA target sequence
  source 1..19: mol_type = other RNA
ccaacagcaccaatatctt

SEQ ID NO: 13 - RNA, 19 nt, synthetic construct
  misc_feature 1..19: note = AAT mRNA target sequence
  source 1..19: mol_type = other RNA
ccccagtgagcatcgctac

SEQ ID NO: 14 - RNA, 19 nt, synthetic construct
  misc_feature 1..19: note = AAT mRNA target sequence
  source 1..19: mol_type = other RNA
gagcatcgctacagccttt

SEQ ID NO: 15 - RNA, 19 nt, synthetic construct
  misc_feature 1..19: note = AAT mRNA target sequence
  source 1..19: mol_type = other RNA
gcatcgctacagcctttgc

SEQ ID NO: 16 - RNA, 19 nt, synthetic construct
  misc_feature 1..19: note = AAT mRNA target sequence
  source 1..19: mol_type = other RNA
catcgctacagcctttgca

SEQ ID NO: 17 - RNA, 19 nt, synthetic construct
  misc_feature 1..19: note = AAT mRNA target sequence
  source 1..19: mol_type = other RNA
tcgctacagcctttgcaat

SEQ ID NO: 18 - RNA, 19 nt, synthetic construct
  misc_feature 1..19: note = AAT mRNA target sequence
  source 1..19: mol_type = other RNA
ctacagcctttgcaatgct

SEQ ID NO: 19 - RNA, 19 nt, synthetic construct
  misc_feature 1..19: note = AAT mRNA target sequence
  source 1..19: mol_type = other RNA
acagcctttgcaatgctct

SEQ ID NO: 20 - RNA, 19 nt, synthetic construct
  misc_feature 1..19: note = AAT mRNA target sequence
  source 1..19: mol_type = other RNA
gaaggcttccaggaactcc

SEQ ID NO: 21 - RNA, 19 nt, synthetic construct
  misc_feature 1..19: note = AAT mRNA target sequence
  source 1..19: mol_type = other RNA
tagtggataagtttttgga

SEQ ID NO: 22 - RNA, 19 nt, synthetic construct
  misc_feature 1..19: note = AAT mRNA target sequence
  source 1..19: mol_type = other RNA
tgtaccactcagaagcctt

SEQ ID NO: 23 - RNA, 19 nt, synthetic construct
  misc_feature 1..19: note = AAT mRNA target sequence
  source 1..19: mol_type = other RNA
gtaccactcagaagccttc

SEQ ID NO: 24 - RNA, 19 nt, synthetic construct
  misc_feature 1..19: note = AAT mRNA target sequence
  source 1..19: mol_type = other RNA
acaccgaagaggccaagaa

SEQ ID NO: 25 - RNA, 19 nt, synthetic construct
  misc_feature 1..19: note = AAT mRNA target sequence
  source 1..19: mol_type = other RNA
accgaagaggccaagaaac

SEQ ID NO: 26 - RNA, 19 nt, synthetic construct
  misc_feature 1..19: note = AAT mRNA target sequence
  source 1..19: mol_type = other RNA
aggccaagaaacagatcaa

SEQ ID NO: 27 - RNA, 19 nt, synthetic construct
  misc_feature 1..19: note = AAT mRNA target sequence
  source 1..19: mol_type = other RNA
ggccaagaaacagatcaac

SEQ ID NO: 28 - RNA, 19 nt, synthetic construct
  misc_feature 1..19: note = AAT mRNA target sequence
  source 1..19: mol_type = other RNA
gccaagaaacagatcaacg

SEQ ID NO: 29 - RNA, 19 nt, synthetic construct
  misc_feature 1..19: note = AAT mRNA target sequence
  source 1..19: mol_type = other RNA
tactcaagggaaaattgtg

SEQ ID NO: 30 - RNA, 19 nt, synthetic construct
  misc_feature 1..19: note = AAT mRNA target sequence
  source 1..19: mol_type = other RNA
ctcaagggaaaattgtgga

SEQ ID NO: 31 - RNA, 19 nt, synthetic construct
  misc_feature 1..19: note = AAT mRNA target sequence
  source 1..19: mol_type = other RNA
tcaagggaaaattgtggat

SEQ ID NO: 32 - RNA, 19 nt, synthetic construct
  misc_feature 1..19: note = AAT mRNA target sequence
  source 1..19: mol_type = other RNA
ttggtcaaggagcttgaca

SEQ ID NO: 33 - RNA, 19 nt, synthetic construct
  misc_feature 1..19: note = AAT mRNA target sequence
  source 1..19: mol_type = other RNA
aggagcttgacagagacac

SEQ ID NO: 34 - RNA, 19 nt, synthetic construct
  misc_feature 1..19: note = AAT mRNA target sequence
  source 1..19: mol_type = other RNA
agcttgacagagacacagt

SEQ ID NO: 35 - RNA, 19 nt, synthetic construct
  misc_feature 1..19: note = AAT mRNA target sequence
  source 1..19: mol_type = other RNA
tttgctctggtgaattaca

SEQ ID NO: 36 - RNA, 19 nt, synthetic construct
  misc_feature 1..19: note = AAT mRNA target sequence
  source 1..19: mol_type = other RNA
agcgtttaggcatgtttaa

SEQ ID NO: 37 - RNA, 19 nt, synthetic construct
  misc_feature 1..19: note = AAT mRNA target sequence
  source 1..19: mol_type = other RNA
gcgtttaggcatgtttaac

SEQ ID NO: 38 - RNA, 19 nt, synthetic construct
  misc_feature 1..19: note = AAT mRNA target sequence
  source 1..19: mol_type = other RNA
ttaggcatgtttaacatcc

SEQ ID NO: 39 - RNA, 19 nt, synthetic construct
  misc_feature 1..19: note = AAT mRNA target sequence
  source 1..19: mol_type = other RNA
tgggtgctgctgatgaaat

SEQ ID NO: 40 - RNA, 19 nt, synthetic construct
  misc_feature 1..19: note = AAT mRNA target sequence
  source 1..19: mol_type = other RNA
tgccaccgccatcttcttc

SEQ ID NO: 41 - RNA, 19 nt, synthetic construct
  misc_feature 1..19: note = AAT mRNA target sequence
  source 1..19: mol_type = other RNA
cctggaaaatgaactcacc

SEQ ID NO: 42 - RNA, 19 nt, synthetic construct
  misc_feature 1..19: note = AAT mRNA target sequence
  source 1..19: mol_type = other RNA
cgatatcatcaccaagttc

SEQ ID NO: 43 - RNA, 19 nt, synthetic construct
  misc_feature 1..19: note = AAT mRNA target sequence
  source 1..19: mol_type = other RNA
accaagttcctggaaaatg

SEQ ID NO: 44 - RNA, 19 nt, synthetic construct
  misc_feature 1..19: note = AAT mRNA target sequence
  source 1..19: mol_type = other RNA
tccattactggaacctatg

SEQ ID NO: 45 - RNA, 19 nt, synthetic construct
  misc_feature 1..19: note = AAT mRNA target sequence
  source 1..19: mol_type = other RNA
ccattactggaacctatga

SEQ ID NO: 46 - RNA, 19 nt, synthetic construct
  misc_feature 1..19: note = AAT mRNA target sequence
  source 1..19: mol_type = other RNA
actggaacctatgatctga

SEQ ID NO: 47 - RNA, 19 nt, synthetic construct
  misc_feature 1..19: note = AAT mRNA target sequence
  source 1..19: mol_type = other RNA
ggaacctatgatctgaaga

SEQ ID NO: 48 - RNA, 19 nt, synthetic construct
  misc_feature 1..19: note = AAT mRNA target sequence
  source 1..19: mol_type = other RNA
gaacctatgatctgaagag

SEQ ID NO: 49 - RNA, 19 nt, synthetic construct
  misc_feature 1..19: note = AAT mRNA target sequence
  source 1..19: mol_type = other RNA
cagcaatggggctgacctc

SEQ ID NO: 50 - RNA, 19 nt, synthetic construct
  misc_feature 1..19: note = AAT mRNA target sequence
  source 1..19: mol_type = other RNA
gcaatggggctgacctctc

SEQ ID NO: 51 - RNA, 19 nt, synthetic construct
  misc_feature 1..19: note = AAT mRNA target sequence
  source 1..19: mol_type = other RNA
agaggaggcacccctgaag

SEQ ID NO: 52 - RNA, 19 nt, synthetic construct
  misc_feature 1..19: note = AAT mRNA target sequence
  source 1..19: mol_type = other RNA
aggcacccctgaagctctc

SEQ ID NO: 53 - RNA, 19 nt, synthetic construct
  misc_feature 1..19: note = AAT mRNA target sequence
  source 1..19: mol_type = other RNA
tctccaaggccgtgcataa

SEQ ID NO: 54 - RNA, 19 nt, synthetic construct
  misc_feature 1..19: note = AAT mRNA target sequence
  source 1..19: mol_type = other RNA
tccaaggccgtgcataagg

SEQ ID NO: 55 - RNA, 19 nt, synthetic construct
  misc_feature 1..19: note = AAT mRNA target sequence
  source 1..19: mol_type = other RNA
ccaaggccgtgcataaggc

SEQ ID NO: 56 - RNA, 19 nt, synthetic construct
  misc_feature 1..19: note = AAT mRNA target sequence
  source 1..19: mol_type = other RNA
caaggccgtgcataaggct

SEQ ID NO: 57 - RNA, 19 nt, synthetic construct
  misc_feature 1..19: note = AAT mRNA target sequence
  source 1..19: mol_type = other RNA
aaggctgtgctgaccatcg

SEQ ID NO: 58 - RNA, 19 nt, synthetic construct
  misc_feature 1..19: note = AAT mRNA target sequence
  source 1..19: mol_type = other RNA
ggctgtgctgaccatcgac

SEQ ID NO: 59 - RNA, 19 nt, synthetic construct
  misc_feature 1..19: note = AAT mRNA target sequence
  source 1..19: mol_type = other RNA
ctgctggggccatgttttt

SEQ ID NO: 60 - RNA, 19 nt, synthetic construct
  misc_feature 1..19: note = AAT mRNA target sequence
  source 1..19: mol_type = other RNA
gctggggccatgtttttag

SEQ ID NO: 61 - RNA, 19 nt, synthetic construct
  misc_feature 1..19: note = AAT mRNA target sequence
  source 1..19: mol_type = other RNA
ctggggccatgtttttaga

SEQ ID NO: 62 - RNA, 19 nt, synthetic construct
  misc_feature 1..19: note = AAT mRNA target sequence
  source 1..19: mol_type = other RNA
ggggccatgtttttagagg

SEQ ID NO: 63 - RNA, 19 nt, synthetic construct
  misc_feature 1..19: note = AAT mRNA target sequence
  source 1..19: mol_type = other RNA
gggccatgtttttagaggc

SEQ ID NO: 64 - RNA, 19 nt, synthetic construct
  misc_feature 1..19: note = AAT mRNA target sequence
  source 1..19: mol_type = other RNA
gaggccatacccatgtcta

SEQ ID NO: 65 - RNA, 19 nt, synthetic construct
  misc_feature 1..19: note = AAT mRNA target sequence
  source 1..19: mol_type = other RNA
ggccatacccatgtctatc

SEQ ID NO: 66 - RNA, 19 nt, synthetic construct
  misc_feature 1..19: note = AAT mRNA target sequence
  source 1..19: mol_type = other RNA
cccgaggtcaagttcaaca

SEQ ID NO: 67 - RNA, 19 nt, synthetic construct
  misc_feature 1..19: note = AAT mRNA target sequence
  source 1..19: mol_type = other RNA
aggtcaagttcaacaaacc

SEQ ID NO: 68 - RNA, 19 nt, synthetic construct
  misc_feature 1..19: note = AAT mRNA target sequence
  source 1..19: mol_type = other RNA
caagttcaacaaacccttt

SEQ ID NO: 69 - RNA, 19 nt, synthetic construct
  misc_feature 1..19: note = AAT mRNA target sequence
  source 1..19: mol_type = other RNA
agttcaacaaaccctttgt

SEQ ID NO: 70 - RNA, 19 nt, synthetic construct
  misc_feature 1..19: note = AAT mRNA target sequence
  source 1..19: mol_type = other RNA
gttcaacaaaccctttgtc

SEQ ID NO: 71 - RNA, 19 nt, synthetic construct
  misc_feature 1..19: note = AAT mRNA target sequence
  source 1..19: mol_type = other RNA
tcaacaaaccctttgtctt

SEQ ID NO: 72 - RNA, 19 nt, synthetic construct
  misc_feature 1..19: note = AAT mRNA target sequence
  source 1..19: mol_type = other RNA
accctttgtcttcttaatg

SEQ ID NO: 73 - RNA, 19 nt, synthetic construct
  misc_feature 1..19: note = AAT mRNA target sequence
  source 1..19: mol_type = other RNA
cctttgtcttcttaatgat

SEQ ID NO: 74 - RNA, 19 nt, synthetic construct
  misc_feature 1..19: note = AAT mRNA target sequence
  source 1..19: mol_type = other RNA
taccaagtctcccctcttc

SEQ ID NO: 75 - RNA, 19 nt, synthetic construct
  misc_feature 1..19: note = AAT mRNA target sequence
  source 1..19: mol_type = other RNA
aagtctcccctcttcatgg

SEQ ID NO: 76 - RNA, 19 nt, synthetic construct
  misc_feature 1..19: note = AAT mRNA target sequence
  source 1..19: mol_type = other RNA
agtctcccctcttcatggg

SEQ ID NO: 77 - RNA, 19 nt, synthetic construct
  misc_feature 1..19: note = AAT mRNA target sequence
  source 1..19: mol_type = other RNA
tctcccctcttcatgggaa

SEQ ID NO: 78 - RNA, 19 nt, synthetic construct
  misc_feature 1..19: note = AAT mRNA target sequence
  source 1..19: mol_type = other RNA
ctcccctcttcatgggaaa

SEQ ID NO: 79 - RNA, 19 nt, synthetic construct
  misc_feature 1..19: note = AAT mRNA target sequence
  source 1..19: mol_type = other RNA
atgacattaaagaagggtt

SEQ ID NO: 80 - RNA, 19 nt, synthetic construct
  misc_feature 1..19: note = AAT RNAi agent antisense strand core stretch base sequence
  source 1..19: mol_type = other RNA
tgttaaacatgcctaaacg

SEQ ID NO: 81 - RNA, 19 nt, synthetic construct
  misc_feature 1..19: note = AAT RNAi agent antisense strand core stretch base sequence
  source 1..19: mol_type = other RNA
agttaaacatgcctaaacg

SEQ ID NO: 82 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngttaaacatgcctaaacg

SEQ ID NO: 83 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngttaaacatgcctaaacn

SEQ ID NO: 84 - RNA, 19 nt, synthetic construct
  misc_feature 1..19: note = AAT RNAi agent antisense strand core stretch base sequence
  source 1..19: mol_type = other RNA
agaagatattggtgctgtt

SEQ ID NO: 85 - RNA, 19 nt, synthetic construct
  misc_feature 1..19: note = AAT RNAi agent antisense strand core stretch base sequence
  source 1..19: mol_type = other RNA
tgaagatattggtgctgtt

SEQ ID NO: 86 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngaagatattggtgctgtt

SEQ ID NO: 87 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngaagatattggtgctgtn

SEQ ID NO: 88 - RNA, 19 nt, synthetic construct
  misc_feature 1..19: note = AAT RNAi agent antisense strand core stretch base sequence
  source 1..19: mol_type = other RNA
aggaacttggtgatgatat

SEQ ID NO: 89 - RNA, 19 nt, synthetic construct
  misc_feature 1..19: note = AAT RNAi agent antisense strand core stretch base sequence
  source 1..19: mol_type = other RNA
tggaacttggtgatgatat

SEQ ID NO: 90 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nggaacttggtgatgatat

SEQ ID NO: 91 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nggaacttggtgatgatan

SEQ ID NO: 92 - RNA, 19 nt, synthetic construct
  misc_feature 1..19: note = AAT RNAi agent antisense strand core stretch base sequence
  source 1..19: mol_type = other RNA
tgtcttctgggcagcatct

SEQ ID NO: 93 - RNA, 19 nt, synthetic construct
  misc_feature 1..19: note = AAT RNAi agent antisense strand core stretch base sequence
  source 1..19: mol_type = other RNA
agtcttctgggcagcatct

SEQ ID NO: 94 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngtcttctgggcagcatct

SEQ ID NO: 95 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngtcttctgggcagcatcn

SEQ ID NO: 96 - RNA, 19 nt, synthetic construct
  misc_feature 1..19: note = AAT RNAi agent antisense strand core stretch base sequence
  source 1..19: mol_type = other RNA
tgttggactggtgtgccag

SEQ ID NO: 97 - RNA, 19 nt, synthetic construct
  misc_feature 1..19: note = AAT RNAi agent antisense strand core stretch base sequence
  source 1..19: mol_type = other RNA
agttggactggtgtgccag

SEQ ID NO: 98 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngttggactggtgtgccag

SEQ ID NO: 99 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngttggactggtgtgccan

SEQ ID NO: 100 - RNA, 19 nt, synthetic construct
  misc_feature 1..19: note = AAT RNAi agent antisense strand core stretch base sequence
  source 1..19: mol_type = other RNA
ctgttggactggtgtgcca

SEQ ID NO: 101 - RNA, 19 nt, synthetic construct
  misc_feature 1..19: note = AAT RNAi agent antisense strand core stretch base sequence
  source 1..19: mol_type = other RNA
ttgttggactggtgtgcca

SEQ ID NO: 102 - RNA, 19 nt, synthetic construct
  misc_feature 1..19: note = AAT RNAi agent antisense strand core stretch base sequence
  source 1..19: mol_type = other RNA
atgttggactggtgtgcca

SEQ ID NO: 103 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntgttggactggtgtgcca

SEQ ID NO: 104 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntgttggactggtgtgccn

SEQ ID NO: 105 - RNA, 19 nt, synthetic construct
  misc_feature 1..19: note = AAT RNAi agent antisense strand core stretch base sequence
  source 1..19: mol_type = other RNA
tgctgttggactggtgtgc

SEQ ID NO: 106 - RNA, 19 nt, synthetic construct
  misc_feature 1..19: note = AAT RNAi agent antisense strand core stretch base sequence
  source 1..19: mol_type = other RNA
agctgttggactggtgtgc

SEQ ID NO: 107 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngctgttggactggtgtgc

SEQ ID NO: 108 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngctgttggactggtgtgn

SEQ ID NO: 109 - RNA, 19 nt, synthetic construct
  misc_feature 1..19: note = AAT RNAi agent antisense strand core stretch base sequence
  source 1..19: mol_type = other RNA
tattggtgctgttggactg

SEQ ID NO: 110 - RNA, 19 nt, synthetic construct
  misc_feature 1..19: note = AAT RNAi agent antisense strand core stretch base sequence
  source 1..19: mol_type = other RNA
aattggtgctgttggactg

SEQ ID NO: 111 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nattggtgctgttggactg

SEQ ID NO: 112 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nattggtgctgttggactn

SEQ ID NO: 113 - RNA, 19 nt, synthetic construct
  misc_feature 1..19: note = AAT RNAi agent antisense strand core stretch base sequence
  source 1..19: mol_type = other RNA
atattggtgctgttggact

SEQ ID NO: 114 - RNA, 19 nt, synthetic construct
  misc_feature 1..19: note = AAT RNAi agent antisense strand core stretch base sequence
  source 1..19: mol_type = other RNA
ttattggtgctgttggact

SEQ ID NO: 115 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntattggtgctgttggact

SEQ ID NO: 116 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntattggtgctgttggacn

SEQ ID NO: 117 - RNA, 19 nt, synthetic construct
  misc_feature 1..19: note = AAT RNAi agent antisense strand core stretch base sequence
  source 1..19: mol_type = other RNA
gatattggtgctgttggac

SEQ ID NO: 118 - RNA, 19 nt, synthetic construct
  misc_feature 1..19: note = AAT RNAi agent antisense strand core stretch base sequence
  source 1..19: mol_type = other RNA
tatattggtgctgttggac

SEQ ID NO: 119 - RNA, 19 nt, synthetic construct
  misc_feature 1..19: note = AAT RNAi agent antisense strand core stretch base sequence
  source 1..19: mol_type = other RNA
aatattggtgctgttggac

SEQ ID NO: 120 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
natattggtgctgttggac

SEQ ID NO: 121 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
natattggtgctgttggan

SEQ ID NO: 122 - RNA, 19 nt, synthetic construct
  misc_feature 1..19: note = AAT RNAi agent antisense strand core stretch base sequence
  source 1..19: mol_type = other RNA
aagatattggtgctgttgg

SEQ ID NO: 123 - RNA, 19 nt, synthetic construct
  misc_feature 1..19: note = AAT RNAi agent antisense strand core stretch base sequence
  source 1..19: mol_type = other RNA
tagatattggtgctgttgg

SEQ ID NO: 124 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nagatattggtgctgttgg

SEQ ID NO: 125 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nagatattggtgctgttgn

SEQ ID NO: 126 - RNA, 19 nt, synthetic construct
  misc_feature 1..19: note = AAT RNAi agent antisense strand core stretch base sequence
  source 1..19: mol_type = other RNA
gtagcgatgctcactgggg

SEQ ID NO: 127 - RNA, 19 nt, synthetic construct
  misc_feature 1..19: note = AAT RNAi agent antisense strand core stretch base sequence
  source 1..19: mol_type = other RNA
ttagcgatgctcactgggg

SEQ ID NO: 128 - RNA, 19 nt, synthetic construct
  misc_feature 1..19: note = AAT RNAi agent antisense strand core stretch base sequence
  source 1..19: mol_type = other RNA
atagcgatgctcactgggg

SEQ ID NO: 129 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntagcgatgctcactgggg

SEQ ID NO: 130 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntagcgatgctcactgggn

SEQ ID NO: 131 - RNA, 19 nt, synthetic construct
  misc_feature 1..19: note = AAT RNAi agent antisense strand core stretch base sequence
  source 1..19: mol_type = other RNA
aaaggctgtagcgatgctc

SEQ ID NO: 132 - RNA, 19 nt, synthetic construct
  misc_feature 1..19: note = AAT RNAi agent antisense strand core stretch base sequence
  source 1..19: mol_type = other RNA
taaggctgtagcgatgctc

SEQ ID NO: 133 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naaggctgtagcgatgctc

SEQ ID NO: 134 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naaggctgtagcgatgctn

SEQ ID NO: 135 - RNA, 19 nt, synthetic construct
  misc_feature 1..19: note = AAT RNAi agent antisense strand core stretch base sequence
  source 1..19: mol_type = other RNA
gcaaaggctgtagcgatgc

SEQ ID NO: 136 - RNA, 19 nt, synthetic construct
  misc_feature 1..19: note = AAT RNAi agent antisense strand core stretch base sequence
  source 1..19: mol_type = other RNA
tcaaaggctgtagcgatgc

SEQ ID NO: 137 - RNA, 19 nt, synthetic construct
  misc_feature 1..19: note = AAT RNAi agent antisense strand core stretch base sequence
  source 1..19: mol_type = other RNA
acaaaggctgtagcgatgc

SEQ ID NO: 138 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncaaaggctgtagcgatgc

SEQ ID NO: 139 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncaaaggctgtagcgatgn

SEQ ID NO: 140 - RNA, 19 nt, synthetic construct
  misc_feature 1..19: note = AAT RNAi agent antisense strand core stretch base sequence
  source 1..19: mol_type = other RNA
tgcaaaggctgtagcgatg

SEQ ID NO: 141 - RNA, 19 nt, synthetic construct
  misc_feature 1..19: note = AAT RNAi agent antisense strand core stretch base sequence
  source 1..19: mol_type = other RNA
agcaaaggctgtagcgatg

SEQ ID NO: 142 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngcaaaggctgtagcgatg

SEQ ID NO: 143 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngcaaaggctgtagcgatn

SEQ ID NO: 144 - RNA, 19 nt, synthetic construct
  misc_feature 1..19: note = AAT RNAi agent antisense strand core stretch base sequence
  source 1..19: mol_type = other RNA
attgcaaaggctgtagcga

SEQ ID NO: 145 - RNA, 19 nt, synthetic construct
  misc_feature 1..19: note = AAT RNAi agent antisense strand core stretch base sequence
  source 1..19: mol_type = other RNA
tttgcaaaggctgtagcga

SEQ ID NO: 146 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nttgcaaaggctgtagcga

SEQ ID NO: 147 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nttgcaaaggctgtagcgn

SEQ ID NO: 148 - RNA, 19 nt, synthetic construct
  misc_feature 1..19: note = AAT RNAi agent antisense strand core stretch base sequence
  source 1..19: mol_type = other RNA
agcattgcaaaggctgtag

SEQ ID NO: 149 - RNA, 19 nt, synthetic construct
  misc_feature 1..19: note = AAT RNAi agent antisense strand core stretch base sequence
  source 1..19: mol_type = other RNA
tgcattgcaaaggctgtag

SEQ ID NO: 150 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngcattgcaaaggctgtag

SEQ ID NO: 151 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngcattgcaaaggctgtan

SEQ ID NO: 152 - RNA, 19 nt, synthetic construct
  misc_feature 1..19: note = AAT RNAi agent antisense strand core stretch base sequence
  source 1..19: mol_type = other RNA
agagcattgcaaaggctgt

SEQ ID NO: 153 - RNA, 19 nt, synthetic construct
  misc_feature 1..19: note = AAT RNAi agent antisense strand core stretch base sequence
  source 1..19: mol_type = other RNA
tgagcattgcaaaggctgt

SEQ ID NO: 154 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngagcattgcaaaggctgt

SEQ ID NO: 155 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngagcattgcaaaggctgt

SEQ ID NO: 156 - RNA, 19 nt, synthetic construct
  misc_feature 1..19: note = AAT RNAi agent antisense strand core stretch base sequence
  source 1..19: mol_type = other RNA
ggagttcctggaagccttc

SEQ ID NO: 157 - RNA, 19 nt, synthetic construct
  misc_feature 1..19: note = AAT RNAi agent antisense strand core stretch base sequence
  source 1..19: mol_type = other RNA
tgagttcctggaagccttc

SEQ ID NO: 158 - RNA, 19 nt, synthetic construct
  misc_feature 1..19: note = AAT RNAi agent antisense strand core stretch base sequence
  source 1..19: mol_type = other RNA
agagttcctggaagccttc

SEQ ID NO: 159 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngagttcctggaagccttc

SEQ ID NO: 160 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngagttcctggaagccttn

SEQ ID NO: 161 - RNA, 19 nt, synthetic construct
  misc_feature 1..19: note = AAT RNAi agent antisense strand core stretch base sequence
  source 1..19: mol_type = other RNA
tccaaaaacttatccacta

SEQ ID NO: 162 - RNA, 19 nt, synthetic construct
  misc_feature 1..19: note = AAT RNAi agent antisense strand core stretch base sequence
  source 1..19: mol_type = other RNA
accaaaaacttatccacta

SEQ ID NO: 163 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nccaaaaacttatccacta

SEQ ID NO: 164 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nccaaaaacttatccactn

SEQ ID NO: 165 - RNA, 19 nt, synthetic construct
  misc_feature 1..19: note = AAT RNAi agent antisense strand core stretch base sequence
  source 1..19: mol_type = other RNA
aaggcttctgagtggtaca

SEQ ID NO: 166 - RNA, 19 nt, synthetic construct
  misc_feature 1..19: note = AAT RNAi agent antisense strand core stretch base sequence
  source 1..19: mol_type = other RNA
taggcttctgagtggtaca

SEQ ID NO: 167 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naggcttctgagtggtaca

SEQ ID NO: 168 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naggcttctgagtggtacn

SEQ ID NO: 169 - RNA, 19 nt, synthetic construct
  misc_feature 1..19: note = AAT RNAi agent antisense strand core stretch base sequence
  source 1..19: mol_type = other RNA
gaaggcttctgagtggtac

SEQ ID NO: 170 - RNA, 19 nt, synthetic construct
  misc_feature 1..19: note = AAT RNAi agent antisense strand core stretch base sequence
  source 1..19: mol_type = other RNA
taaggcttctgagtggtac

SEQ ID NO: 171 - RNA, 19 nt, synthetic construct
  misc_feature 1..19: note = AAT RNAi agent antisense strand core stretch base sequence
  source 1..19: mol_type = other RNA
aaaggcttctgagtggtac

SEQ ID NO: 172 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naaggcttctgagtggtac

SEQ ID NO: 173 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naaggcttctgagtggtan

SEQ ID NO: 174 - RNA, 19 nt, synthetic construct
  misc_feature 1..19: note = AAT RNAi agent antisense strand core stretch base sequence
  source 1..19: mol_type = other RNA
ttcttggcctcttcggtgt

SEQ ID NO: 175 - RNA, 19 nt, synthetic construct
  misc_feature 1..19: note = AAT RNAi agent antisense strand core stretch base sequence
  source 1..19: mol_type = other RNA
atcttggcctcttcggtgt

SEQ ID NO: 176 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntcttggcctcttcggtgt

SEQ ID NO: 177 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntcttggcctcttcggtgn

SEQ ID NO: 178 - RNA, 19 nt, synthetic construct
  misc_feature 1..19: note = AAT RNAi agent antisense strand core stretch base sequence
  source 1..19: mol_type = other RNA
gtttcttggcctcttcggt

SEQ ID NO: 179 - RNA, 19 nt, synthetic construct
  misc_feature 1..19: note = AAT RNAi agent antisense strand core stretch base sequence
  source 1..19: mol_type = other RNA
ttttcttggcctcttcggt

SEQ ID NO: 180 - RNA, 19 nt, synthetic construct
  misc_feature 1..19: note = AAT RNAi agent antisense strand core stretch base sequence
  source 1..19: mol_type = other RNA
atttcttggcctcttcggt

SEQ ID NO: 181 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntttcttggcctcttcggt

SEQ ID NO: 182 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntttcttggcctcttcggn

SEQ ID NO: 183 - RNA, 19 nt, synthetic construct
  misc_feature 1..19: note = AAT RNAi agent antisense strand core stretch base sequence
  source 1..19: mol_type = other RNA
ttgatctgtttcttggcct

SEQ ID NO: 184 - RNA, 19 nt, synthetic construct
  misc_feature 1..19: note = AAT RNAi agent antisense strand core stretch base sequence
  source 1..19: mol_type = other RNA
atgatctgtttcttggcct

SEQ ID NO: 185 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntgatctgtttcttggcct

SEQ ID NO: 186 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntgatctgtttcttggccn

SEQ ID NO: 187 - RNA, 19 nt, synthetic construct
  misc_feature 1..19: note = AAT RNAi agent antisense strand core stretch base sequence
  source 1..19: mol_type = other RNA
gttgatctgtttcttggcc

SEQ ID NO: 188 - RNA, 19 nt, synthetic construct
  misc_feature 1..19: note = AAT RNAi agent antisense strand core stretch base sequence
  source 1..19: mol_type = other RNA
tttgatctgtttcttggcc

SEQ ID NO: 189 - RNA, 19 nt, synthetic construct
  misc_feature 1..19: note = AAT RNAi agent antisense strand core stretch base sequence
  source 1..19: mol_type = other RNA
attgatctgtttcttggcc

SEQ ID NO: 190 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nttgatctgtttcttggcc

SEQ ID NO: 191 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nttgatctgtttcttggcn

SEQ ID NO: 192 - RNA, 19 nt, synthetic construct
  misc_feature 1..19: note = AAT RNAi agent antisense strand core stretch base sequence
  source 1..19: mol_type = other RNA
cgttgatctgtttcttggc

SEQ ID NO: 193 - RNA, 19 nt, synthetic construct
  misc_feature 1..19: note = AAT RNAi agent antisense strand core stretch base sequence
  source 1..19: mol_type = other RNA
tgttgatctgtttcttggc

SEQ ID NO: 194 - RNA, 19 nt, synthetic construct
  misc_feature 1..19: note = AAT RNAi agent antisense strand core stretch base sequence
  source 1..19: mol_type = other RNA
agttgatctgtttcttggc

SEQ ID NO: 195 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngttgatctgtttcttggc

SEQ ID NO: 196 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngttgatctgtttcttggn

SEQ ID NO: 197 - RNA, 19 nt, synthetic construct
  misc_feature 1..19: note = AAT RNAi agent antisense strand core stretch base sequence
  source 1..19: mol_type = other RNA
cacaattttcccttgagta

SEQ ID NO: 198 - RNA, 19 nt, synthetic construct
  misc_feature 1..19: note = AAT RNAi agent antisense strand core stretch base sequence
  source 1..19: mol_type = other RNA
tacaattttcccttgagta

SEQ ID NO: 199 - RNA, 19 nt, synthetic construct
  misc_feature 1..19: note = AAT RNAi agent antisense strand core stretch base sequence
  source 1..19: mol_type = other RNA
aacaattttcccttgagta

SEQ ID NO: 200 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nacaattttcccttgagta

SEQ ID NO: 201 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nacaattttcccttgagtn

SEQ ID NO: 202 - RNA, 19 nt, synthetic construct
  misc_feature 1..19: note = AAT RNAi agent antisense strand core stretch base sequence
  source 1..19: mol_type = other RNA
tccacaattttcccttgag

SEQ ID NO: 203 - RNA, 19 nt, synthetic construct
  misc_feature 1..19: note = AAT RNAi agent antisense strand core stretch base sequence
  source 1..19: mol_type = other RNA
accacaattttcccttgag

SEQ ID NO: 204 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nccacaattttcccttgag

SEQ ID NO: 205 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nccacaattttcccttgan

SEQ ID NO: 206 - RNA, 19 nt, synthetic construct
  misc_feature 1..19: note = AAT RNAi agent antisense strand core stretch base sequence
  source 1..19: mol_type = other RNA
atccacaattttcccttga

SEQ ID NO: 207 - RNA, 19 nt, synthetic construct
  misc_feature 1..19: note = AAT RNAi agent antisense strand core stretch base sequence
  source 1..19: mol_type = other RNA
ttccacaattttcccttga

SEQ ID NO: 208 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntccacaattttcccttga

SEQ ID NO: 209 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntccacaattttcccttgn

SEQ ID NO: 210 - RNA, 19 nt, synthetic construct
  misc_feature 1..19: note = AAT RNAi agent antisense strand core stretch base sequence
  source 1..19: mol_type = other RNA
tgtcaagctccttgaccaa

SEQ ID NO: 211 - RNA, 19 nt, synthetic construct
  misc_feature 1..19: note = AAT RNAi agent antisense strand core stretch base sequence
  source 1..19: mol_type = other RNA
agtcaagctccttgaccaa

SEQ ID NO: 212 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngtcaagctccttgaccaa

SEQ ID NO: 213 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngtcaagctccttgaccaa

SEQ ID NO: 214 - RNA, 19 nt, synthetic construct
  misc_feature 1..19: note = AAT RNAi agent antisense strand core stretch base sequence
  source 1..19: mol_type = other RNA
gtgtctctgtcaagctcct

SEQ ID NO: 215 - RNA, 19 nt, synthetic construct
  misc_feature 1..19: note = AAT RNAi agent antisense strand core stretch base sequence
  source 1..19: mol_type = other RNA
ttgtctctgtcaagctcct

SEQ ID NO: 216 - RNA, 19 nt, synthetic construct
  misc_feature 1..19: note = AAT RNAi agent antisense strand core stretch base sequence
  source 1..19: mol_type = other RNA
atgtctctgtcaagctcct

SEQ ID NO: 217 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntgtctctgtcaagctcct

SEQ ID NO: 218 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntgtctctgtcaagctccn

SEQ ID NO: 219 - RNA, 19 nt, synthetic construct
  misc_feature 1..19: note = AAT RNAi agent antisense strand core stretch base sequence
  source 1..19: mol_type = other RNA
actgtgtctctgtcaagct

SEQ ID NO: 220 - RNA, 19 nt, synthetic construct
  misc_feature 1..19: note = AAT RNAi agent antisense strand core stretch base sequence
  source 1..19: mol_type = other RNA
tctgtgtctctgtcaagct

SEQ ID NO: 221 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nctgtgtctctgtcaagct

SEQ ID NO: 222 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nctgtgtctctgtcaagcn

SEQ ID NO: 223 - RNA, 19 nt, synthetic construct
  misc_feature 1..19: note = AAT RNAi agent antisense strand core stretch base sequence
  source 1..19: mol_type = other RNA
tgtaattcaccagagcaaa

SEQ ID NO: 224 - RNA, 19 nt, synthetic construct
  misc_feature 1..19: note = AAT RNAi agent antisense strand core stretch base sequence
  source 1..19: mol_type = other RNA
agtaattcaccagagcaaa

SEQ ID NO: 225 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngtaattcaccagagcaaa

SEQ ID NO: 226 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngtaattcaccagagcaan

SEQ ID NO: 227 - RNA, 19 nt, synthetic construct
  misc_feature 1..19: note = AAT RNAi agent antisense strand core stretch base sequence
  source 1..19: mol_type = other RNA
ttaaacatgcctaaacgct

SEQ ID NO: 228 - RNA, 19 nt, synthetic construct
  misc_feature 1..19: note = AAT RNAi agent antisense strand core stretch base sequence
  source 1..19: mol_type = other RNA
ataaacatgcctaaacgct

SEQ ID NO: 229 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntaaacatgcctaaacgct

SEQ ID NO: 230 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntaaacatgcctaaacgcn

SEQ ID NO: 231 - RNA, 19 nt, synthetic construct
  misc_feature 1..19: note = AAT RNAi agent antisense strand core stretch base sequence
  source 1..19: mol_type = other RNA
gttaaacatgcctaaacgc

SEQ ID NO: 232 - RNA, 19 nt, synthetic construct
  misc_feature 1..19: note = AAT RNAi agent antisense strand core stretch base sequence
  source 1..19: mol_type = other RNA
tttaaacatgcctaaacgc

SEQ ID NO: 233 - RNA, 19 nt, synthetic construct
  misc_feature 1..19: note = AAT RNAi agent antisense strand core stretch base sequence
  source 1..19: mol_type = other RNA
attaaacatgcctaaacgc

SEQ ID NO: 234 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nttaaacatgcctaaacgc

SEQ ID NO: 235 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nttaaacatgcctaaacgn

SEQ ID NO: 236 - RNA, 19 nt, synthetic construct
  misc_feature 1..19: note = AAT RNAi agent antisense strand core stretch base sequence
  source 1..19: mol_type = other RNA
ggatgttaaacatgcctaa

SEQ ID NO: 237 - RNA, 19 nt, synthetic construct
  misc_feature 1..19: note = AAT RNAi agent antisense strand core stretch base sequence
  source 1..19: mol_type = other RNA
tgatgttaaacatgcctaa

SEQ ID NO: 238 - RNA, 19 nt, synthetic construct
  misc_feature 1..19: note = AAT RNAi agent antisense strand core stretch base sequence
  source 1..19: mol_type = other RNA
agatgttaaacatgcctaa

SEQ ID NO: 239 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngatgttaaacatgcctaa

SEQ ID NO: 240 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngatgttaaacatgcctan

SEQ ID NO: 241 - RNA, 19 nt, synthetic construct
  misc_feature 1..19: note = AAT RNAi agent antisense strand core stretch base sequence
  source 1..19: mol_type = other RNA
atttcatcagcagcaccca

SEQ ID NO: 242 - RNA, 19 nt, synthetic construct
  misc_feature 1..19: note = AAT RNAi agent antisense strand core stretch base sequence
  source 1..19: mol_type = other RNA
ttttcatcagcagcaccca

SEQ ID NO: 243 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntttcatcagcagcaccca

SEQ ID NO: 244 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntttcatcagcagcacccn

SEQ ID NO: 245 - RNA, 19 nt, synthetic construct
  misc_feature 1..19: note = AAT RNAi agent antisense strand core stretch base sequence
  source 1..19: mol_type = other RNA
gaagaagatggcggtggca

SEQ ID NO: 246 - RNA, 19 nt, synthetic construct
  misc_feature 1..19: note = AAT RNAi agent antisense strand core stretch base sequence
  source 1..19: mol_type = other RNA
taagaagatggcggtggca

SEQ ID NO: 247 - RNA, 19 nt, synthetic construct
  misc_feature 1..19: note = AAT RNAi agent antisense strand core stretch base sequence
  source 1..19: mol_type = other RNA
aaagaagatggcggtggca

SEQ ID NO: 248 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naagaagatggcggtggca

SEQ ID NO: 249 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naagaagatggcggtggcn

SEQ ID NO: 250 - RNA, 19 nt, synthetic construct
  misc_feature 1..19: note = AAT RNAi agent antisense strand core stretch base sequence
  source 1..19: mol_type = other RNA
ggtgagttcattttccagg

SEQ ID NO: 251 - RNA, 19 nt, synthetic construct
  misc_feature 1..19: note = AAT RNAi agent antisense strand core stretch base sequence
  source 1..19: mol_type = other RNA
tgtgagttcattttccagg

SEQ ID NO: 252 - RNA, 19 nt, synthetic construct
  misc_feature 1..19: note = AAT RNAi agent antisense strand core stretch base sequence
  source 1..19: mol_type = other RNA
agtgagttcattttccagg

SEQ ID NO: 253 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngtgagttcattttccagg

SEQ ID NO: 254 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngtgagttcattttccagn

SEQ ID NO: 255 - RNA, 19 nt, synthetic construct
  misc_feature 1..19: note = AAT RNAi agent antisense strand core stretch base sequence
  source 1..19: mol_type = other RNA
gaacttggtgatgatatcg

SEQ ID NO: 256 - RNA, 19 nt, synthetic construct
  misc_feature 1..19: note = AAT RNAi agent antisense strand core stretch base sequence
  source 1..19: mol_type = other RNA
taacttggtgatgatatcg

SEQ ID NO: 257 - RNA, 19 nt, synthetic construct
  misc_feature 1..19: note = AAT RNAi agent antisense strand core stretch base sequence
  source 1..19: mol_type = other RNA
aaacttggtgatgatatcg

SEQ ID NO: 258 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naacttggtgatgatatcg

SEQ ID NO: 259 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naacttggtgatgatatcn

SEQ ID NO: 260 - RNA, 19 nt, synthetic construct
  misc_feature 1..19: note = AAT RNAi agent antisense strand core stretch base sequence
  source 1..19: mol_type = other RNA
cattttccaggaacttggt

SEQ ID NO: 261 - RNA, 19 nt, synthetic construct
  misc_feature 1..19: note = AAT RNAi agent antisense strand core stretch base sequence
  source 1..19: mol_type = other RNA
tattttccaggaacttggt

SEQ ID NO: 262 - RNA, 19 nt, synthetic construct
  misc_feature 1..19: note = AAT RNAi agent antisense strand core stretch base sequence
  source 1..19: mol_type = other RNA
aattttccaggaacttggt

SEQ ID NO: 263 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nattttccaggaacttggt

SEQ ID NO: 264 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nattttccaggaacttggn

SEQ ID NO: 265 - RNA, 19 nt, synthetic construct
  misc_feature 1..19: note = AAT RNAi agent antisense strand core stretch base sequence
  source 1..19: mol_type = other RNA
cataggttccagtaatgga

SEQ ID NO: 266 - RNA, 19 nt, synthetic construct
  misc_feature 1..19: note = AAT RNAi agent antisense strand core stretch base sequence
  source 1..19: mol_type = other RNA
tataggttccagtaatgga

SEQ ID NO: 267 - RNA, 19 nt, synthetic construct
  misc_feature 1..19: note = AAT RNAi agent antisense strand core stretch base sequence
  source 1..19: mol_type = other RNA
aataggttccagtaatgga

SEQ ID NO: 268 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nataggttccagtaatgga

SEQ ID NO: 269 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nataggttccagtaatggn

SEQ ID NO: 270 - RNA, 19 nt, synthetic construct
  misc_feature 1..19: note = AAT RNAi agent antisense strand core stretch base sequence
  source 1..19: mol_type = other RNA
tcataggttccagtaatgg

SEQ ID NO: 271 - RNA, 19 nt, synthetic construct
  misc_feature 1..19: note = AAT RNAi agent antisense strand core stretch base sequence
  source 1..19: mol_type = other RNA
acataggttccagtaatgg

SEQ ID NO: 272 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncataggttccagtaatgg

SEQ ID NO: 273 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncataggttccagtaatgn

SEQ ID NO: 274 - RNA, 19 nt, synthetic construct
  misc_feature 1..19: note = AAT RNAi agent antisense strand core stretch base sequence
  source 1..19: mol_type = other RNA
tcagatcataggttccagt

SEQ ID NO: 275 - RNA, 19 nt, synthetic construct
  misc_feature 1..19: note = AAT RNAi agent antisense strand core stretch base sequence
  source 1..19: mol_type = other RNA
acagatcataggttccagt

SEQ ID NO: 276 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncagatcataggttccagt

SEQ ID NO: 277 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncagatcataggttccagn

SEQ ID NO: 278 - RNA, 19 nt, synthetic construct
  misc_feature 1..19: note = AAT RNAi agent antisense strand core stretch base sequence
  source 1..19: mol_type = other RNA
tcttcagatcataggttcc

SEQ ID NO: 279 - RNA, 19 nt, synthetic construct
  misc_feature 1..19: note = AAT RNAi agent antisense strand core stretch base sequence
  source 1..19: mol_type = other RNA
acttcagatcataggttcc

SEQ ID NO: 280 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncttcagatcataggttcc

SEQ ID NO: 281 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncttcagatcataggttcn

SEQ ID NO: 282 - RNA, 19 nt, synthetic construct
  misc_feature 1..19: note = AAT RNAi agent antisense strand core stretch base sequence
  source 1..19: mol_type = other RNA
ctcttcagatcataggttc

SEQ ID NO: 283 - RNA, 19 nt, synthetic construct
  misc_feature 1..19: note = AAT RNAi agent antisense strand core stretch base sequence
  source 1..19: mol_type = other RNA
ttcttcagatcataggttc

SEQ ID NO: 284 - RNA, 19 nt, synthetic construct
  misc_feature 1..19: note = AAT RNAi agent antisense strand core stretch base sequence
  source 1..19: mol_type = other RNA
atcttcagatcataggttc

SEQ ID NO: 285 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntcttcagatcataggttc

SEQ ID NO: 286 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntcttcagatcataggttn

SEQ ID NO: 287 - RNA, 19 nt, synthetic construct
  misc_feature 1..19: note = AAT RNAi agent antisense strand core stretch base sequence
  source 1..19: mol_type = other RNA
gaggtcagccccattgctg

SEQ ID NO: 288 - RNA, 19 nt, synthetic construct
  misc_feature 1..19: note = AAT RNAi agent antisense strand core stretch base sequence
  source 1..19: mol_type = other RNA
taggtcagccccattgctg

SEQ ID NO: 289 - RNA, 19 nt, synthetic construct
  misc_feature 1..19: note = AAT RNAi agent antisense strand core stretch base sequence
  source 1..19: mol_type = other RNA
aaggtcagccccattgctg

SEQ ID NO: 290 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naggtcagccccattgctg

SEQ ID NO: 291 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naggtcagccccattgctn

SEQ ID NO: 292 - RNA, 19 nt, synthetic construct
  misc_feature 1..19: note = AAT RNAi agent antisense strand core stretch base sequence
  source 1..19: mol_type = other RNA
gagaggtcagccccattgc

SEQ ID NO: 293 - RNA, 19 nt, synthetic construct
  misc_feature 1..19: note = AAT RNAi agent antisense strand core stretch base sequence
  source 1..19: mol_type = other RNA
tagaggtcagccccattgc

SEQ ID NO: 294 - RNA, 19 nt, synthetic construct
  misc_feature 1..19: note = AAT RNAi agent antisense strand core stretch base sequence
  source 1..19: mol_type = other RNA
aagaggtcagccccattgc

SEQ ID NO: 295 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nagaggtcagccccattgc

SEQ ID NO: 296 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nagaggtcagccccattgn

SEQ ID NO: 297 - RNA, 19 nt, synthetic construct
  misc_feature 1..19: note = AAT RNAi agent antisense strand core stretch base sequence
  source 1..19: mol_type = other RNA
cttcaggggtgcctcctct

SEQ ID NO: 298 - RNA, 19 nt, synthetic construct
  misc_feature 1..19: note = AAT RNAi agent antisense strand core stretch base sequence
  source 1..19: mol_type = other RNA
tttcaggggtgcctcctct

SEQ ID NO: 299 - RNA, 19 nt, synthetic construct
  misc_feature 1..19: note = AAT RNAi agent antisense strand core stretch base sequence
  source 1..19: mol_type = other RNA
attcaggggtgcctcctct

SEQ ID NO: 300 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nttcaggggtgcctcctct

SEQ ID NO: 301 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nttcaggggtgcctcctcn

SEQ ID NO: 302 - RNA, 19 nt, synthetic construct
  misc_feature 1..19: note = AAT RNAi agent antisense strand core stretch base sequence
  source 1..19: mol_type = other RNA
gagagcttcaggggtgcct

SEQ ID NO: 303 - RNA, 19 nt, synthetic construct
  misc_feature 1..19: note = AAT RNAi agent antisense strand core stretch base sequence
  source 1..19: mol_type = other RNA
tagagcttcaggggtgcct

SEQ ID NO: 304 - RNA, 19 nt, synthetic construct
  misc_feature 1..19: note = AAT RNAi agent antisense strand core stretch base sequence
  source 1..19: mol_type = other RNA
aagagcttcaggggtgcct

SEQ ID NO: 305 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nagagcttcaggggtgcct

SEQ ID NO: 306 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nagagcttcaggggtgccn

SEQ ID NO: 307 - RNA, 19 nt, synthetic construct
  misc_feature 1..19: note = AAT RNAi agent antisense strand core stretch base sequence
  source 1..19: mol_type = other RNA
ttatgcacggccttggaga

SEQ ID NO: 308 - RNA, 19 nt, synthetic construct
  misc_feature 1..19: note = AAT RNAi agent antisense strand core stretch base sequence
  source 1..19: mol_type = other RNA
atatgcacggccttggaga

SEQ ID NO: 309 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntatgcacggccttggaga

SEQ ID NO: 310 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntatgcacggccttggagn

SEQ ID NO: 311 - RNA, 19 nt, synthetic construct
  misc_feature 1..19: note = AAT RNAi agent antisense strand core stretch base sequence
  source 1..19: mol_type = other RNA
ccttatgcacggccttgga

SEQ ID NO: 312 - RNA, 19 nt, synthetic construct
  misc_feature 1..19: note = AAT RNAi agent antisense strand core stretch base sequence
  source 1..19: mol_type = other RNA
tcttatgcacggccttgga

SEQ ID NO: 313 - RNA, 19 nt, synthetic construct
  misc_feature 1..19: note = AAT RNAi agent antisense strand core stretch base sequence
  source 1..19: mol_type = other RNA
acttatgcacggccttgga

SEQ ID NO: 314 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncttatgcacggccttgga

SEQ ID NO: 315 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncttatgcacggccttggn

SEQ ID NO: 316 - RNA, 19 nt, synthetic construct
  misc_feature 1..19: note = AAT RNAi agent antisense strand core stretch base sequence
  source 1..19: mol_type = other RNA
gccttatgcacggccttgg

SEQ ID NO: 317 - RNA, 19 nt, synthetic construct
  misc_feature 1..19: note = AAT RNAi agent antisense strand core stretch base sequence
  source 1..19: mol_type = other RNA
tccttatgcacggccttgg

SEQ ID NO: 318 - RNA, 19 nt, synthetic construct
  misc_feature 1..19: note = AAT RNAi agent antisense strand core stretch base sequence
  source 1..19: mol_type = other RNA
accttatgcacggccttgg

SEQ ID NO: 319 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nccttatgcacggccttgg

SEQ ID NO: 320 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nccttatgcacggccttgn

SEQ ID NO: 321 - RNA, 19 nt, synthetic construct
  misc_feature 1..19: note = AAT RNAi agent antisense strand core stretch base sequence
  source 1..19: mol_type = other RNA
agccttatgcacggccttg

SEQ ID NO: 322 - RNA, 19 nt, synthetic construct
  misc_feature 1..19: note = AAT RNAi agent antisense strand core stretch base sequence
  source 1..19: mol_type = other RNA
tgccttatgcacggccttg

SEQ ID NO: 323 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngccttatgcacggccttg

SEQ ID NO: 324 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngccttatgcacggccttn

SEQ ID NO: 325 - RNA, 19 nt, synthetic construct
  misc_feature 1..19: note = AAT RNAi agent antisense strand core stretch base sequence
  source 1..19: mol_type = other RNA
cgatggtcagcacagcctt

SEQ ID NO: 326 - RNA, 19 nt, synthetic construct
  misc_feature 1..19: note = AAT RNAi agent antisense strand core stretch base sequence
  source 1..19: mol_type = other RNA
tgatggtcagcacagcctt

SEQ ID NO: 327 - RNA, 19 nt, synthetic construct
  misc_feature 1..19: note = AAT RNAi agent antisense strand core stretch base sequence
  source 1..19: mol_type = other RNA
agatggtcagcacagcctt

SEQ ID NO: 328 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngatggtcagcacagcctt

SEQ ID NO: 329 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngatggtcagcacagcctn

SEQ ID NO: 330 - RNA, 19 nt, synthetic construct
  misc_feature 1..19: note = AAT RNAi agent antisense strand core stretch base sequence
  source 1..19: mol_type = other RNA
gtcgatggtcagcacagcc

SEQ ID NO: 331 - RNA, 19 nt, synthetic construct
  misc_feature 1..19: note = AAT RNAi agent antisense strand core stretch base sequence
  source 1..19: mol_type = other RNA
ttcgatggtcagcacagcc

SEQ ID NO: 332 - RNA, 19 nt, synthetic construct
  misc_feature 1..19: note = AAT RNAi agent antisense strand core stretch base sequence
  source 1..19: mol_type = other RNA
atcgatggtcagcacagcc

SEQ ID NO: 333 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntcgatggtcagcacagcc

SEQ ID NO: 334 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntcgatggtcagcacagcn

SEQ ID NO: 335 - RNA, 19 nt, synthetic construct
  misc_feature 1..19: note = AAT RNAi agent antisense strand core stretch base sequence
  source 1..19: mol_type = other RNA
aaaaacatggccccagcag

SEQ ID NO: 336 - RNA, 19 nt, synthetic construct
  misc_feature 1..19: note = AAT RNAi agent antisense strand core stretch base sequence
  source 1..19: mol_type = other RNA
taaaacatggccccagcag

SEQ ID NO: 337 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naaaacatggccccagcag

SEQ ID NO: 338 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naaaacatggccccagcan

SEQ ID NO: 339 - RNA, 19 nt, synthetic construct
  misc_feature 1..19: note = AAT RNAi agent antisense strand core stretch base sequence
  source 1..19: mol_type = other RNA
ctaaaaacatggccccagc

SEQ ID NO: 340 - RNA, 19 nt, synthetic construct
  misc_feature 1..19: note = AAT RNAi agent antisense strand core stretch base sequence
  source 1..19: mol_type = other RNA
ttaaaaacatggccccagc

SEQ ID NO: 341 - RNA, 19 nt, synthetic construct
  misc_feature 1..19: note = AAT RNAi agent antisense strand core stretch base sequence
  source 1..19: mol_type = other RNA
ataaaaacatggccccagc

SEQ ID NO: 342 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntaaaaacatggccccagc

SEQ ID NO: 343 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntaaaaacatggccccagn

SEQ ID NO: 344 - RNA, 19 nt, synthetic construct
  misc_feature 1..19: note = AAT RNAi agent antisense strand core stretch base sequence
  source 1..19: mol_type = other RNA
tctaaaaacatggccccag

SEQ ID NO: 345 - RNA, 19 nt, synthetic construct
  misc_feature 1..19: note = AAT RNAi agent antisense strand core stretch base sequence
  source 1..19: mol_type = other RNA
actaaaaacatggccccag

SEQ ID NO: 346 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nctaaaaacatggccccag

SEQ ID NO: 347 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nctaaaaacatggccccan

SEQ ID NO: 348 - RNA, 19 nt, synthetic construct
  misc_feature 1..19: note = AAT RNAi agent antisense strand core stretch base sequence
  source 1..19: mol_type = other RNA
cctctaaaaacatggcccc

SEQ ID NO: 349 - RNA, 19 nt, synthetic construct
  misc_feature 1..19: note = AAT RNAi agent antisense strand core stretch base sequence
  source 1..19: mol_type = other RNA
tctctaaaaacatggcccc

SEQ ID NO: 350 - RNA, 19 nt, synthetic construct
  misc_feature 1..19: note = AAT RNAi agent antisense strand core stretch base sequence
  source 1..19: mol_type = other RNA
actctaaaaacatggcccc

SEQ ID NO: 351 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nctctaaaaacatggcccc

SEQ ID NO: 352 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nctctaaaaacatggcccn

SEQ ID NO: 353 - RNA, 19 nt, synthetic construct
  misc_feature 1..19: note = AAT RNAi agent antisense strand core stretch base sequence
  source 1..19: mol_type = other RNA
gcctctaaaaacatggccc

SEQ ID NO: 354 - RNA, 19 nt, synthetic construct
  misc_feature 1..19: note = AAT RNAi agent antisense strand core stretch base sequence
  source 1..19: mol_type = other RNA
tcctctaaaaacatggccc

SEQ ID NO: 355 - RNA, 19 nt, synthetic construct
  misc_feature 1..19: note = AAT RNAi agent antisense strand core stretch base sequence
  source 1..19: mol_type = other RNA
acctctaaaaacatggccc

SEQ ID NO: 356 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncctctaaaaacatggccc

SEQ ID NO: 357 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncctctaaaaacatggccn

SEQ ID NO: 358 - RNA, 19 nt, synthetic construct
  misc_feature 1..19: note = AAT RNAi agent antisense strand core stretch base sequence
  source 1..19: mol_type = other RNA
tagacatgggtatggcctc

SEQ ID NO: 359 - RNA, 19 nt, synthetic construct
  misc_feature 1..19: note = AAT RNAi agent antisense strand core stretch base sequence
  source 1..19: mol_type = other RNA
aagacatgggtatggcctc

SEQ ID NO: 360 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nagacatgggtatggcctc

SEQ ID NO: 361 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nagacatgggtatggcctn

SEQ ID NO: 362 - RNA, 19 nt, synthetic construct
  misc_feature 1..19: note = AAT RNAi agent antisense strand core stretch base sequence
  source 1..19: mol_type = other RNA
gatagacatgggtatggcc

SEQ ID NO: 363 - RNA, 19 nt, synthetic construct
  misc_feature 1..19: note = AAT RNAi agent antisense strand core stretch base sequence
  source 1..19: mol_type = other RNA
tatagacatgggtatggcc

SEQ ID NO: 364 - RNA, 19 nt, synthetic construct
  misc_feature 1..19: note = AAT RNAi agent antisense strand core stretch base sequence
  source 1..19: mol_type = other RNA
aatagacatgggtatggcc

SEQ ID NO: 365 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
natagacatgggtatggcc

SEQ ID NO: 366 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
natagacatgggtatggcn

SEQ ID NO: 367 - RNA, 19 nt, synthetic construct
  misc_feature 1..19: note = AAT RNAi agent antisense strand core stretch base sequence
  source 1..19: mol_type = other RNA
tgttgaacttgacctcggg

SEQ ID NO: 368 - RNA, 19 nt, synthetic construct
  misc_feature 1..19: note = AAT RNAi agent antisense strand core stretch base sequence
  source 1..19: mol_type = other RNA
agttgaacttgacctcggg

SEQ ID NO: 369 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngttgaacttgacctcggg

SEQ ID NO: 370 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngttgaacttgacctcggn

SEQ ID NO: 371 - RNA, 19 nt, synthetic construct
  misc_feature 1..19: note = AAT RNAi agent antisense strand core stretch base sequence
  source 1..19: mol_type = other RNA
ggtttgttgaacttgacct

SEQ ID NO: 372 - RNA, 19 nt, synthetic construct
  misc_feature 1..19: note = AAT RNAi agent antisense strand core stretch base sequence
  source 1..19: mol_type = other RNA
tgtttgttgaacttgacct

SEQ ID NO: 373 - RNA, 19 nt, synthetic construct
  misc_feature 1..19: note = AAT RNAi agent antisense strand core stretch base sequence
  source 1..19: mol_type = other RNA
agtttgttgaacttgacct

SEQ ID NO: 374 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ngtttgttgaacttgacct

SEQ ID NO: 375 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ngtttgttgaacttgaccn

SEQ ID NO: 376 - RNA, 19 nt, synthetic construct
  misc_feature 1..19: note = AAT RNAi agent antisense strand core stretch base sequence
  source 1..19: mol_type = other RNA
aaagggtttgttgaacttg

SEQ ID NO: 377 - RNA, 19 nt, synthetic construct
  misc_feature 1..19: note = AAT RNAi agent antisense strand core stretch base sequence
  source 1..19: mol_type = other RNA
taagggtttgttgaacttg

SEQ ID NO: 378 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naagggtttgttgaacttg

SEQ ID NO: 379 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naagggtttgttgaacttn

SEQ ID NO: 380 - RNA, 19 nt, synthetic construct
  misc_feature 1..19: note = AAT RNAi agent antisense strand core stretch base sequence
  source 1..19: mol_type = other RNA
acaaagggtttgttgaact

SEQ ID NO: 381 - RNA, 19 nt, synthetic construct
  misc_feature 1..19: note = AAT RNAi agent antisense strand core stretch base sequence
  source 1..19: mol_type = other RNA
tcaaagggtttgttgaact

SEQ ID NO: 382 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncaaagggtttgttgaact

SEQ ID NO: 383 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncaaagggtttgttgaacn

SEQ ID NO: 384 - RNA, 19 nt, synthetic construct
  misc_feature 1..19: note = AAT RNAi agent antisense strand core stretch base sequence
  source 1..19: mol_type = other RNA
gacaaagggtttgttgaac

SEQ ID NO: 385 - RNA, 19 nt, synthetic construct
  misc_feature 1..19: note = AAT RNAi agent antisense strand core stretch base sequence
  source 1..19: mol_type = other RNA
tacaaagggtttgttgaac

SEQ ID NO: 386 - RNA, 19 nt, synthetic construct
  misc_feature 1..19: note = AAT RNAi agent antisense strand core stretch base sequence
  source 1..19: mol_type = other RNA
aacaaagggtttgttgaac

SEQ ID NO: 387 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nacaaagggtttgttgaac

SEQ ID NO: 388 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nacaaagggtttgttgaan

SEQ ID NO: 389 - RNA, 19 nt, synthetic construct
  misc_feature 1..19: note = AAT RNAi agent antisense strand core stretch base sequence
  source 1..19: mol_type = other RNA
aagacaaagggtttgttga

SEQ ID NO: 390 - RNA, 19 nt, synthetic construct
  misc_feature 1..19: note = AAT RNAi agent antisense strand core stretch base sequence
  source 1..19: mol_type = other RNA
tagacaaagggtttgttga

SEQ ID NO: 391 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nagacaaagggtttgttga

SEQ ID NO: 392 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nagacaaagggtttgttgn

SEQ ID NO: 393 - RNA, 19 nt, synthetic construct
  misc_feature 1..19: note = AAT RNAi agent antisense strand core stretch base sequence
  source 1..19: mol_type = other RNA
cattaagaagacaaagggt

SEQ ID NO: 394 - RNA, 19 nt, synthetic construct
  misc_feature 1..19: note = AAT RNAi agent antisense strand core stretch base sequence
  source 1..19: mol_type = other RNA
tattaagaagacaaagggt

SEQ ID NO: 395 - RNA, 19 nt, synthetic construct
  misc_feature 1..19: note = AAT RNAi agent antisense strand core stretch base sequence
  source 1..19: mol_type = other RNA
aattaagaagacaaagggt

SEQ ID NO: 396 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nattaagaagacaaagggt

SEQ ID NO: 397 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nattaagaagacaaagggn

SEQ ID NO: 398 - RNA, 19 nt, synthetic construct
  misc_feature 1..19: note = AAT RNAi agent antisense strand core stretch base sequence
  source 1..19: mol_type = other RNA
atcattaagaagacaaagg

SEQ ID NO: 399 - RNA, 19 nt, synthetic construct
  misc_feature 1..19: note = AAT RNAi agent antisense strand core stretch base sequence
  source 1..19: mol_type = other RNA
ttcattaagaagacaaagg

SEQ ID NO: 400 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntcattaagaagacaaagg

SEQ ID NO: 401 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntcattaagaagacaaagn

SEQ ID NO: 402 - RNA, 19 nt, synthetic construct
  misc_feature 1..19: note = AAT RNAi agent antisense strand core stretch base sequence
  source 1..19: mol_type = other RNA
gaagaggggagacttggta

SEQ ID NO: 403 - RNA, 19 nt, synthetic construct
  misc_feature 1..19: note = AAT RNAi agent antisense strand core stretch base sequence
  source 1..19: mol_type = other RNA
taagaggggagacttggta

SEQ ID NO: 404 - RNA, 19 nt, synthetic construct
  misc_feature 1..19: note = AAT RNAi agent antisense strand core stretch base sequence
  source 1..19: mol_type = other RNA
aaagaggggagacttggta

SEQ ID NO: 405 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
naagaggggagacttggta

SEQ ID NO: 406 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
naagaggggagacttggtn

SEQ ID NO: 407 - RNA, 19 nt, synthetic construct
  misc_feature 1..19: note = AAT RNAi agent antisense strand core stretch base sequence
  source 1..19: mol_type = other RNA
ccatgaagaggggagactt

SEQ ID NO: 408 - RNA, 19 nt, synthetic construct
  misc_feature 1..19: note = AAT RNAi agent antisense strand core stretch base sequence
  source 1..19: mol_type = other RNA
tcatgaagaggggagactt

SEQ ID NO: 409 - RNA, 19 nt, synthetic construct
  misc_feature 1..19: note = AAT RNAi agent antisense strand core stretch base sequence
  source 1..19: mol_type = other RNA
acatgaagaggggagactt

SEQ ID NO: 410 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ncatgaagaggggagactt

SEQ ID NO: 411 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ncatgaagaggggagactn

SEQ ID NO: 412 - RNA, 19 nt, synthetic construct
  misc_feature 1..19: note = AAT RNAi agent antisense strand core stretch base sequence
  source 1..19: mol_type = other RNA
cccatgaagaggggagact

SEQ ID NO: 413 - RNA, 19 nt, synthetic construct
  misc_feature 1..19: note = AAT RNAi agent antisense strand core stretch base sequence
  source 1..19: mol_type = other RNA
tccatgaagaggggagact

SEQ ID NO: 414 - RNA, 19 nt, synthetic construct
  misc_feature 1..19: note = AAT RNAi agent antisense strand core stretch base sequence
  source 1..19: mol_type = other RNA
accatgaagaggggagact

SEQ ID NO: 415 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nccatgaagaggggagact

SEQ ID NO: 416 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nccatgaagaggggagacn

SEQ ID NO: 417 - RNA, 19 nt, synthetic construct
  misc_feature 1..19: note = AAT RNAi agent antisense strand core stretch base sequence
  source 1..19: mol_type = other RNA
ttcccatgaagaggggaga

SEQ ID NO: 418 - RNA, 19 nt, synthetic construct
  misc_feature 1..19: note = AAT RNAi agent antisense strand core stretch base sequence
  source 1..19: mol_type = other RNA
atcccatgaagaggggaga

SEQ ID NO: 419 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
ntcccatgaagaggggaga

SEQ ID NO: 420 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
ntcccatgaagaggggagn

SEQ ID NO: 421 - RNA, 19 nt, synthetic construct
  misc_feature 1..19: note = AAT RNAi agent antisense strand core stretch base sequence
  source 1..19: mol_type = other RNA
tttcccatgaagaggggag

SEQ ID NO: 422 - RNA, 19 nt, synthetic construct
  misc_feature 1..19: note = AAT RNAi agent antisense strand core stretch base sequence
  source 1..19: mol_type = other RNA
attcccatgaagaggggag

SEQ ID NO: 423 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nttcccatgaagaggggag

SEQ ID NO: 424 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nttcccatgaagaggggan

SEQ ID NO: 425 - RNA, 19 nt, synthetic construct
  misc_feature 1..19: note = AAT RNAi agent antisense strand core stretch base sequence
  source 1..19: mol_type = other RNA
aacccttctttaatgtcat

SEQ ID NO: 426 - RNA, 19 nt, synthetic construct
  misc_feature 1..19: note = AAT RNAi agent antisense strand core stretch base sequence
  source 1..19: mol_type = other RNA
tacccttctttaatgtcat

SEQ ID NO: 427 - RNA, 19 nt, synthetic construct
  misc_feature 1..19: note = AAT RNAi agent antisense strand core stretch base sequence
  misc_difference 1: note = n = any nucleotide
  source 1..19: mol_type = other RNA
nacccttctttaatgtcat

SEQ ID NO: 428 - RNA, 19 nt, synthetic construct
  misc_feature 1..19: note = AAT RNAi agent antisense strand core stretch base sequence
  misc_difference 1: note = n = any nucleotide
  misc_difference 19: note = n = any nucleotide
  source 1..19: mol_type = other RNA
nacccttctttaatgtcan

SEQ ID NO: 429 - RNA, 19 nt, synthetic construct
  misc_feature 1..19: note = AAT RNAi agent sense strand core stretch base sequence
  source 1..19: mol_type = other RNA
cgtttaggcatgtttaaca

SEQ ID NO: 430 - RNA, 19 nt, synthetic construct
  misc_feature 1..19: note = AAT RNAi agent sense strand core stretch base sequence
  source 1..19: mol_type = other RNA
cgtttaggcatgtttaact

SEQ ID NO: 431 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
cgtttaggcatgtttaacn

SEQ ID NO: 432 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngtttaggcatgtttaacn

SEQ ID NO: 433 - RNA, 19 nt, synthetic construct
  misc_feature 1..19: note = AAT RNAi agent sense strand core stretch base sequence
  source 1..19: mol_type = other RNA
aacagcaccaatatcttct

SEQ ID NO: 434 - RNA, 19 nt, synthetic construct
  misc_feature 1..19: note = AAT RNAi agent sense strand core stretch base sequence
  source 1..19: mol_type = other RNA
aacagcaccaatatcttca

SEQ ID NO: 435 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
aacagcaccaatatcttcn

SEQ ID NO: 436 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nacagcaccaatatcttcn

SEQ ID NO: 437 - RNA, 19 nt, synthetic construct
  misc_feature 1..19: note = AAT RNAi agent sense strand core stretch base sequence
  source 1..19: mol_type = other RNA
atatcatcaccaagttcct

SEQ ID NO: 438 - RNA, 19 nt, synthetic construct
  misc_feature 1..19: note = AAT RNAi agent sense strand core stretch base sequence
  source 1..19: mol_type = other RNA
atatcatcaccaagttcca

SEQ ID NO: 439 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
atatcatcaccaagttccn

SEQ ID NO: 440 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntatcatcaccaagttccn

SEQ ID NO: 441 - RNA, 19 nt, synthetic construct
  misc_feature 1..19: note = AAT RNAi agent sense strand core stretch base sequence
  source 1..19: mol_type = other RNA
agatgctgcccagaagaca

SEQ ID NO: 442 - RNA, 19 nt, synthetic construct
  misc_feature 1..19: note = AAT RNAi agent sense strand core stretch base sequence
  source 1..19: mol_type = other RNA
agatgctgcccagaagact

SEQ ID NO: 443 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
agatgctgcccagaagacn

SEQ ID NO: 444 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngatgctgcccagaagacn

SEQ ID NO: 445 - RNA, 19 nt, synthetic construct
  misc_feature 1..19: note = AAT RNAi agent sense strand core stretch base sequence
  source 1..19: mol_type = other RNA
ctggcacaccagtccaaca

SEQ ID NO: 446 - RNA, 19 nt, synthetic construct
  misc_feature 1..19: note = AAT RNAi agent sense strand core stretch base sequence
  source 1..19: mol_type = other RNA
ctggcacaccagtccaact

SEQ ID NO: 447 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ctggcacaccagtccaacn

SEQ ID NO: 448 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntggcacaccagtccaacn

SEQ ID NO: 449 - RNA, 19 nt, synthetic construct
  misc_feature 1..19: note = AAT RNAi agent sense strand core stretch base sequence
  source 1..19: mol_type = other RNA
tggcacaccagtccaacag

SEQ ID NO: 450 - RNA, 19 nt, synthetic construct
  misc_feature 1..19: note = AAT RNAi agent sense strand core stretch base sequence
  source 1..19: mol_type = other RNA
tggcacaccagtccaacaa

SEQ ID NO: 451 - RNA, 19 nt, synthetic construct
  misc_feature 1..19: note = AAT RNAi agent sense strand core stretch base sequence
  source 1..19: mol_type = other RNA
tggcacaccagtccaacat

SEQ ID NO: 452 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tggcacaccagtccaacan

SEQ ID NO: 453 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nggcacaccagtccaacan

SEQ ID NO: 454 - RNA, 19 nt, synthetic construct
  misc_feature 1..19: note = AAT RNAi agent sense strand core stretch base sequence
  source 1..19: mol_type = other RNA
gcacaccagtccaacagca

SEQ ID NO: 455 - RNA, 19 nt, synthetic construct
  misc_feature 1..19: note = AAT RNAi agent sense strand core stretch base sequence
  source 1..19: mol_type = other RNA
gcacaccagtccaacagct

SEQ ID NO: 456 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gcacaccagtccaacagcn

SEQ ID NO: 457 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncacaccagtccaacagcn

SEQ ID NO: 458 - RNA, 19 nt, synthetic construct
  misc_feature 1..19: note = AAT RNAi agent sense strand core stretch base sequence
  source 1..19: mol_type = other RNA
cagtccaacagcaccaata

SEQ ID NO: 459 - RNA, 19 nt, synthetic construct
  misc_feature 1..19: note = AAT RNAi agent sense strand core stretch base sequence
  source 1..19: mol_type = other RNA
cagtccaacagcaccaatt

SEQ ID NO: 460 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
cagtccaacagcaccaatn

SEQ ID NO: 461 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nagtccaacagcaccaatn

SEQ ID NO: 462 - RNA, 19 nt, synthetic construct
  misc_feature 1..19: note = AAT RNAi agent sense strand core stretch base sequence
  source 1..19: mol_type = other RNA
agtccaacagcaccaatat

SEQ ID NO: 463 - RNA, 19 nt, synthetic construct
  misc_feature 1..19: note = AAT RNAi agent sense strand core stretch base sequence
  source 1..19: mol_type = other RNA
agtccaacagcaccaataa

SEQ ID NO: 464 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
agtccaacagcaccaatan

SEQ ID NO: 465 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngtccaacagcaccaatan

SEQ ID NO: 466 - RNA, 19 nt, synthetic construct
  misc_feature 1..19: note = AAT RNAi agent sense strand core stretch base sequence
  source 1..19: mol_type = other RNA
gtccaacagcaccaatatc

SEQ ID NO: 467 - RNA, 19 nt, synthetic construct
  misc_feature 1..19: note = AAT RNAi agent sense strand core stretch base sequence
  source 1..19: mol_type = other RNA
gtccaacagcaccaatata

SEQ ID NO: 468 - RNA, 19 nt, synthetic construct
  misc_feature 1..19: note = AAT RNAi agent sense strand core stretch base sequence
  source 1..19: mol_type = other RNA
gtccaacagcaccaatatt

SEQ ID NO: 469 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gtccaacagcaccaatatn

SEQ ID NO: 470 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntccaacagcaccaatatn

SEQ ID NO: 471 - RNA, 19 nt, synthetic construct
  misc_feature 1..19: note = AAT RNAi agent sense strand core stretch base sequence
  source 1..19: mol_type = other RNA
ccaacagcaccaatatctt

SEQ ID NO: 472 - RNA, 19 nt, synthetic construct
  misc_feature 1..19: note = AAT RNAi agent sense strand core stretch base sequence
  source 1..19: mol_type = other RNA
ccaacagcaccaatatcta

SEQ ID NO: 473 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ccaacagcaccaatatctn

SEQ ID NO: 474 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncaacagcaccaatatctn

SEQ ID NO: 475 - RNA, 19 nt, synthetic construct
  misc_feature 1..19: note = AAT RNAi agent sense strand core stretch base sequence
  source 1..19: mol_type = other RNA
ccccagtgagcatcgctac

SEQ ID NO: 476 - RNA, 19 nt, synthetic construct
  misc_feature 1..19: note = AAT RNAi agent sense strand core stretch base sequence
  source 1..19: mol_type = other RNA
ccccagtgagcatcgctaa

SEQ ID NO: 477 - RNA, 19 nt, synthetic construct
  misc_feature 1..19: note = AAT RNAi agent sense strand core stretch base sequence
  source 1..19: mol_type = other RNA
ccccagtgagcatcgctat

SEQ ID NO: 478 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ccccagtgagcatcgctan

SEQ ID NO: 479 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncccagtgagcatcgctan

SEQ ID NO: 480 - RNA, 19 nt, synthetic construct
  misc_feature 1..19: note = AAT RNAi agent sense strand core stretch base sequence
  source 1..19: mol_type = other RNA
gagcatcgctacagccttt

SEQ ID NO: 481 - RNA, 19 nt, synthetic construct
  misc_feature 1..19: note = AAT RNAi agent sense strand core stretch base sequence
  source 1..19: mol_type = other RNA
gagcatcgctacagcctta

SEQ ID NO: 482 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gagcatcgctacagccttn

SEQ ID NO: 483 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nagcatcgctacagccttn

SEQ ID NO: 484 - RNA, 19 nt, synthetic construct
  misc_feature 1..19: note = AAT RNAi agent sense strand core stretch base sequence
  source 1..19: mol_type = other RNA
gcatcgctacagcctttgc

SEQ ID NO: 485 - RNA, 19 nt, synthetic construct
  misc_feature 1..19: note = AAT RNAi agent sense strand core stretch base sequence
  source 1..19: mol_type = other RNA
gcatcgctacagcctttga

SEQ ID NO: 486 - RNA, 19 nt, synthetic construct
  misc_feature 1..19: note = AAT RNAi agent sense strand core stretch base sequence
  source 1..19: mol_type = other RNA
gcatcgctacagcctttgt

SEQ ID NO: 487 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gcatcgctacagcctttgn

SEQ ID NO: 488 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncatcgctacagcctttgn

SEQ ID NO: 489 - RNA, 19 nt, synthetic construct
  misc_feature 1..19: note = AAT RNAi agent sense strand core stretch base sequence
  source 1..19: mol_type = other RNA
catcgctacagcctttgca

SEQ ID NO: 490 - RNA, 19 nt, synthetic construct
  misc_feature 1..19: note = AAT RNAi agent sense strand core stretch base sequence
  source 1..19: mol_type = other RNA
catcgctacagcctttgct

SEQ ID NO: 491 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
natcgctacagcctttgcn

SEQ ID NO: 492 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
natcgctacagcctttgcn

SEQ ID NO: 493 - RNA, 19 nt, synthetic construct
  misc_feature 1..19: note = AAT RNAi agent sense strand core stretch base sequence
  source 1..19: mol_type = other RNA
tcgctacagcctttgcaat

SEQ ID NO: 494 - RNA, 19 nt, synthetic construct
  misc_feature 1..19: note = AAT RNAi agent sense strand core stretch base sequence
  source 1..19: mol_type = other RNA
tcgctacagcctttgcaaa

SEQ ID NO: 495 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tcgctacagcctttgcaan

SEQ ID NO: 496 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncgctacagcctttgcaan

SEQ ID NO: 497 - RNA, 19 nt, synthetic construct
  misc_feature 1..19: note = AAT RNAi agent sense strand core stretch base sequence
  source 1..19: mol_type = other RNA
ctacagcctttgcaatgct

SEQ ID NO: 498 - RNA, 19 nt, synthetic construct
  misc_feature 1..19: note = AAT RNAi agent sense strand core stretch base sequence
  source 1..19: mol_type = other RNA
ctacagcctttgcaatgca

SEQ ID NO: 499 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ctacagcctttgcaatgcn

SEQ ID NO: 500 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntacagcctttgcaatgcn

SEQ ID NO: 501 - RNA, 19 nt, synthetic construct
  misc_feature 1..19: note = AAT RNAi agent sense strand core stretch base sequence
  source 1..19: mol_type = other RNA
acagcctttgcaatgctct

SEQ ID NO: 502 - RNA, 19 nt, synthetic construct
  misc_feature 1..19: note = AAT RNAi agent sense strand core stretch base sequence
  source 1..19: mol_type = other RNA
acagcctttgcaatgctca

SEQ ID NO: 503 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
acagcctttgcaatgctcn

SEQ ID NO: 504 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncagcctttgcaatgctcn

SEQ ID NO: 505 - RNA, 19 nt, synthetic construct
  misc_feature 1..19: note = AAT RNAi agent sense strand core stretch base sequence
  source 1..19: mol_type = other RNA
gaaggcttccaggaactcc

SEQ ID NO: 506 - RNA, 19 nt, synthetic construct
  misc_feature 1..19: note = AAT RNAi agent sense strand core stretch base sequence
  source 1..19: mol_type = other RNA
gaaggcttccaggaactca

SEQ ID NO: 507 - RNA, 19 nt, synthetic construct
  misc_feature 1..19: note = AAT RNAi agent sense strand core stretch base sequence
  source 1..19: mol_type = other RNA
gaaggcttccaggaactct

SEQ ID NO: 508 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gaaggcttccaggaactcn

SEQ ID NO: 509 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
naaggcttccaggaactcn

SEQ ID NO: 510 - RNA, 19 nt, synthetic construct
  misc_feature 1..19: note = AAT RNAi agent sense strand core stretch base sequence
  source 1..19: mol_type = other RNA
tagtggataagtttttgga

SEQ ID NO: 511 - RNA, 19 nt, synthetic construct
  misc_feature 1..19: note = AAT RNAi agent sense strand core stretch base sequence
  source 1..19: mol_type = other RNA
tagtggataagtttttggt

SEQ ID NO: 512 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tagtggataagtttttggn

SEQ ID NO: 513 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nagtggataagtttttggn

SEQ ID NO: 514 - RNA, 19 nt, synthetic construct
  misc_feature 1..19: note = AAT RNAi agent sense strand core stretch base sequence
  source 1..19: mol_type = other RNA
tgtaccactcagaagcctt

SEQ ID NO: 515 - RNA, 19 nt, synthetic construct
  misc_feature 1..19: note = AAT RNAi agent sense strand core stretch base sequence
  source 1..19: mol_type = other RNA
tgtaccactcagaagccta

SEQ ID NO: 516 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tgtaccactcagaagcctn

SEQ ID NO: 517 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngtaccactcagaagcctn

SEQ ID NO: 518 - RNA, 19 nt, synthetic construct
  misc_feature 1..19: note = AAT RNAi agent sense strand core stretch base sequence
  source 1..19: mol_type = other RNA
gtaccactcagaagccttc

SEQ ID NO: 519 - RNA, 19 nt, synthetic construct
  misc_feature 1..19: note = AAT RNAi agent sense strand core stretch base sequence
  source 1..19: mol_type = other RNA
gtaccactcagaagcctta

SEQ ID NO: 520 - RNA, 19 nt, synthetic construct
  misc_feature 1..19: note = AAT RNAi agent sense strand core stretch base sequence
  source 1..19: mol_type = other RNA
gtaccactcagaagccttt

SEQ ID NO: 521 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gtaccactcagaagccttn

SEQ ID NO: 522 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntaccactcagaagccttn

SEQ ID NO: 523 - RNA, 19 nt, synthetic construct
  misc_feature 1..19: note = AAT RNAi agent sense strand core stretch base sequence
  source 1..19: mol_type = other RNA
acaccgaagaggccaagaa

SEQ ID NO: 524 - RNA, 19 nt, synthetic construct
  misc_feature 1..19: note = AAT RNAi agent sense strand core stretch base sequence
  source 1..19: mol_type = other RNA
acaccgaagaggccaagat

SEQ ID NO: 525 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
acaccgaagaggccaagan

SEQ ID NO: 526 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncaccgaagaggccaagan

SEQ ID NO: 527 - RNA, 19 nt, synthetic construct
  misc_feature 1..19: note = AAT RNAi agent sense strand core stretch base sequence
  source 1..19: mol_type = other RNA
accgaagaggccaagaaac

SEQ ID NO: 528 - RNA, 19 nt, synthetic construct
  misc_feature 1..19: note = AAT RNAi agent sense strand core stretch base sequence
  source 1..19: mol_type = other RNA
accgaagaggccaagaaaa

SEQ ID NO: 529 - RNA, 19 nt, synthetic construct
  misc_feature 1..19: note = AAT RNAi agent sense strand core stretch base sequence
  source 1..19: mol_type = other RNA
accgaagaggccaagaaat

SEQ ID NO: 530 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
accgaagaggccaagaaan

SEQ ID NO: 531 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nccgaagaggccaagaaan

SEQ ID NO: 532 - RNA, 19 nt, synthetic construct
  misc_feature 1..19: note = AAT RNAi agent sense strand core stretch base sequence
  source 1..19: mol_type = other RNA
aggccaagaaacagatcaa

SEQ ID NO: 533 - RNA, 19 nt, synthetic construct
  misc_feature 1..19: note = AAT RNAi agent sense strand core stretch base sequence
  source 1..19: mol_type = other RNA
aggccaagaaacagatcat

SEQ ID NO: 534 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
aggccaagaaacagatcan

SEQ ID NO: 535 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nggccaagaaacagatcan

SEQ ID NO: 536 - RNA, 19 nt, synthetic construct
  misc_feature 1..19: note = AAT RNAi agent sense strand core stretch base sequence
  source 1..19: mol_type = other RNA
ggccaagaaacagatcaac

SEQ ID NO: 537 - RNA, 19 nt, synthetic construct
  misc_feature 1..19: note = AAT RNAi agent sense strand core stretch base sequence
  source 1..19: mol_type = other RNA
ggccaagaaacagatcaaa

SEQ ID NO: 538 - RNA, 19 nt, synthetic construct
  misc_feature 1..19: note = AAT RNAi agent sense strand core stretch base sequence
  source 1..19: mol_type = other RNA
ggccaagaaacagatcaat

SEQ ID NO: 539 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ggccaagaaacagatcaan

SEQ ID NO: 540 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngccaagaaacagatcaan

SEQ ID NO: 541 - RNA, 19 nt, synthetic construct
  misc_feature 1..19: note = AAT RNAi agent sense strand core stretch base sequence
  source 1..19: mol_type = other RNA
gccaagaaacagatcaacg

SEQ ID NO: 542 - RNA, 19 nt, synthetic construct
  misc_feature 1..19: note = AAT RNAi agent sense strand core stretch base sequence
  source 1..19: mol_type = other RNA
gccaagaaacagatcaaca

SEQ ID NO: 543 - RNA, 19 nt, synthetic construct
  misc_feature 1..19: note = AAT RNAi agent sense strand core stretch base sequence
  source 1..19: mol_type = other RNA
gccaagaaacagatcaact

SEQ ID NO: 544 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gccaagaaacagatcaacn

SEQ ID NO: 545 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nccaagaaacagatcaacn

SEQ ID NO: 546 - RNA, 19 nt, synthetic construct
  misc_feature 1..19: note = AAT RNAi agent sense strand core stretch base sequence
  source 1..19: mol_type = other RNA
tactcaagggaaaattgtg

SEQ ID NO: 547 - RNA, 19 nt, synthetic construct
  misc_feature 1..19: note = AAT RNAi agent sense strand core stretch base sequence
  source 1..19: mol_type = other RNA
tactcaagggaaaattgta

SEQ ID NO: 548 - RNA, 19 nt, synthetic construct
  misc_feature 1..19: note = AAT RNAi agent sense strand core stretch base sequence
  source 1..19: mol_type = other RNA
tactcaagggaaaattgtt

SEQ ID NO: 549 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tactcaagggaaaattgtn

SEQ ID NO: 550 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nactcaagggaaaattgtn

SEQ ID NO: 551 - RNA, 19 nt, synthetic construct
  misc_feature 1..19: note = AAT RNAi agent sense strand core stretch base sequence
  source 1..19: mol_type = other RNA
ctcaagggaaaattgtgga

SEQ ID NO: 552 - RNA, 19 nt, synthetic construct
  misc_feature 1..19: note = AAT RNAi agent sense strand core stretch base sequence
  source 1..19: mol_type = other RNA
ctcaagggaaaattgtggt

SEQ ID NO: 553 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ctcaagggaaaattgtggn

SEQ ID NO: 554 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntcaagggaaaattgtggn

SEQ ID NO: 555 - RNA, 19 nt, synthetic construct
  misc_feature 1..19: note = AAT RNAi agent sense strand core stretch base sequence
  source 1..19: mol_type = other RNA
tcaagggaaaattgtggat

SEQ ID NO: 556 - RNA, 19 nt, synthetic construct
  misc_feature 1..19: note = AAT RNAi agent sense strand core stretch base sequence
  source 1..19: mol_type = other RNA
tcaagggaaaattgtggaa

SEQ ID NO: 557 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tcaagggaaaattgtggan

SEQ ID NO: 558 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncaagggaaaattgtggan

SEQ ID NO: 559 - RNA, 19 nt, synthetic construct
  misc_feature 1..19: note = AAT RNAi agent sense strand core stretch base sequence
  source 1..19: mol_type = other RNA
ttggtcaaggagcttgaca

SEQ ID NO: 560 - RNA, 19 nt, synthetic construct
  misc_feature 1..19: note = AAT RNAi agent sense strand core stretch base sequence
  source 1..19: mol_type = other RNA
ttggtcaaggagcttgact

SEQ ID NO: 561 - RNA, 19 nt, synthetic construct
  misc_feature 1..19: note = AAT RNAi agent sense strand core stretch base sequence
  misc_difference 1: note = n = any nucleotide
  source 1..19: mol_type = other RNA
ntggtcaaggagcttgaca

SEQ ID NO: 562 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntggtcaaggagcttgacn

SEQ ID NO: 563 - RNA, 19 nt, synthetic construct
  misc_feature 1..19: note = AAT RNAi agent sense strand core stretch base sequence
  source 1..19: mol_type = other RNA
aggagcttgacagagacac

SEQ ID NO: 564 - RNA, 19 nt, synthetic construct
  misc_feature 1..19: note = AAT RNAi agent sense strand core stretch base sequence
  source 1..19: mol_type = other RNA
aggagcttgacagagacaa

SEQ ID NO: 565 - RNA, 19 nt, synthetic construct
  misc_feature 1..19: note = AAT RNAi agent sense strand core stretch base sequence
  source 1..19: mol_type = other RNA
aggagcttgacagagacat

SEQ ID NO: 566 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
aggagcttgacagagacan

SEQ ID NO: 567 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nggagcttgacagagacan

SEQ ID NO: 568 - RNA, 19 nt, synthetic construct
  misc_feature 1..19: note = AAT RNAi agent sense strand core stretch base sequence
  source 1..19: mol_type = other RNA
agcttgacagagacacagt

SEQ ID NO: 569 - RNA, 19 nt, synthetic construct
  misc_feature 1..19: note = AAT RNAi agent sense strand core stretch base sequence
  source 1..19: mol_type = other RNA
agcttgacagagacacaga

SEQ ID NO: 570 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
agcttgacagagacacagn

SEQ ID NO: 571 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngcttgacagagacacagn

SEQ ID NO: 572 - RNA, 19 nt, synthetic construct
  misc_feature 1..19: note = AAT RNAi agent sense strand core stretch base sequence
  source 1..19: mol_type = other RNA
tttgctctggtgaattaca

SEQ ID NO: 573 - RNA, 19 nt, synthetic construct
  misc_feature 1..19: note = AAT RNAi agent sense strand core stretch base sequence
  source 1..19: mol_type = other RNA
tttgctctggtgaattact

SEQ ID NO: 574 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tttgctctggtgaattacn

SEQ ID NO: 575 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nttgctctggtgaattacn

SEQ ID NO: 576 - RNA, 19 nt, synthetic construct
  misc_feature 1..19: note = AAT RNAi agent sense strand core stretch base sequence
  source 1..19: mol_type = other RNA
agcgtttaggcatgtttaa

SEQ ID NO: 577 - RNA, 19 nt, synthetic construct
  misc_feature 1..19: note = AAT RNAi agent sense strand core stretch base sequence
  source 1..19: mol_type = other RNA
agcgtttaggcatgtttat

SEQ ID NO: 578 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
agcgtttaggcatgtttan

SEQ ID NO: 579 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngcgtttaggcatgtttan

SEQ ID NO: 580 - RNA, 19 nt, synthetic construct
  misc_feature 1..19: note = AAT RNAi agent sense strand core stretch base sequence
  source 1..19: mol_type = other RNA
gcgtttaggcatgtttaac

SEQ ID NO: 581 - RNA, 19 nt, synthetic construct
  misc_feature 1..19: note = AAT RNAi agent sense strand core stretch base sequence
  source 1..19: mol_type = other RNA
gcgtttaggcatgtttaaa

SEQ ID NO: 582 - RNA, 19 nt, synthetic construct
  misc_feature 1..19: note = AAT RNAi agent sense strand core stretch base sequence
  source 1..19: mol_type = other RNA
gcgtttaggcatgtttaat

SEQ ID NO: 583 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gcgtttaggcatgtttaan

SEQ ID NO: 584 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncgtttaggcatgtttaan

SEQ ID NO: 585 - RNA, 19 nt, synthetic construct
  misc_feature 1..19: note = AAT RNAi agent sense strand core stretch base sequence
  source 1..19: mol_type = other RNA
ttaggcatgtttaacatcc

SEQ ID NO: 586 - RNA, 19 nt, synthetic construct
  misc_feature 1..19: note = AAT RNAi agent sense strand core stretch base sequence
  source 1..19: mol_type = other RNA
ttaggcatgtttaacatca

SEQ ID NO: 587 - RNA, 19 nt, synthetic construct
  misc_feature 1..19: note = AAT RNAi agent sense strand core stretch base sequence
  source 1..19: mol_type = other RNA
ttaggcatgtttaacatct

SEQ ID NO: 588 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ttaggcatgtttaacatcn

SEQ ID NO: 589 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntaggcatgtttaacatcn

SEQ ID NO: 590 - RNA, 19 nt, synthetic construct
  misc_feature 1..19: note = AAT RNAi agent sense strand core stretch base sequence
  source 1..19: mol_type = other RNA
tgggtgctgctgatgaaat

SEQ ID NO: 591 - RNA, 19 nt, synthetic construct
  misc_feature 1..19: note = AAT RNAi agent sense strand core stretch base sequence
  source 1..19: mol_type = other RNA
tgggtgctgctgatgaaaa

SEQ ID NO: 592 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tgggtgctgctgatgaaan

SEQ ID NO: 593 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngggtgctgctgatgaaan

SEQ ID NO: 594 - RNA, 19 nt, synthetic construct
  misc_feature 1..19: note = AAT RNAi agent sense strand core stretch base sequence
  source 1..19: mol_type = other RNA
tgccaccgccatcttcttc

SEQ ID NO: 595 - RNA, 19 nt, synthetic construct
  misc_feature 1..19: note = AAT RNAi agent sense strand core stretch base sequence
  source 1..19: mol_type = other RNA
tgccaccgccatcttctta

SEQ ID NO: 596 - RNA, 19 nt, synthetic construct
  misc_feature 1..19: note = AAT RNAi agent sense strand core stretch base sequence
  source 1..19: mol_type = other RNA
tgccaccgccatcttcttt

SEQ ID NO: 597 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tgccaccgccatcttcttn

SEQ ID NO: 598 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngccaccgccatcttcttn

SEQ ID NO: 599 - RNA, 19 nt, synthetic construct
  misc_feature 1..19: note = AAT RNAi agent sense strand core stretch base sequence
  source 1..19: mol_type = other RNA
cctggaaaatgaactcacc

SEQ ID NO: 600 - RNA, 19 nt, synthetic construct
  misc_feature 1..19: note = AAT RNAi agent sense strand core stretch base sequence
  source 1..19: mol_type = other RNA
cctggaaaatgaactcaca

SEQ ID NO: 601 - RNA, 19 nt, synthetic construct
  misc_feature 1..19: note = AAT RNAi agent sense strand core stretch base sequence
  source 1..19: mol_type = other RNA
cctggaaaatgaactcact

SEQ ID NO: 602 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
cctggaaaatgaactcacn

SEQ ID NO: 603 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nctggaaaatgaactcacn

SEQ ID NO: 604 - RNA, 19 nt, synthetic construct
  misc_feature 1..19: note = AAT RNAi agent sense strand core stretch base sequence
  source 1..19: mol_type = other RNA
cgatatcatcaccaagttc

SEQ ID NO: 605 - RNA, 19 nt, synthetic construct
  misc_feature 1..19: note = AAT RNAi agent sense strand core stretch base sequence
  source 1..19: mol_type = other RNA
cgatatcatcaccaagtta

SEQ ID NO: 606 - RNA, 19 nt, synthetic construct
  misc_feature 1..19: note = AAT RNAi agent sense strand core stretch base sequence
  source 1..19: mol_type = other RNA
cgatatcatcaccaagttt

SEQ ID NO: 607 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
cgatatcatcaccaagttn

SEQ ID NO: 608 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngatatcatcaccaagttn

SEQ ID NO: 609 - RNA, 19 nt, synthetic construct
  misc_feature 1..19: note = AAT RNAi agent sense strand core stretch base sequence
  source 1..19: mol_type = other RNA
accaagttcctggaaaatg

SEQ ID NO: 610 - RNA, 19 nt, synthetic construct
  misc_feature 1..19: note = AAT RNAi agent sense strand core stretch base sequence
  source 1..19: mol_type = other RNA
accaagttcctggaaaata

SEQ ID NO: 611 - RNA, 19 nt, synthetic construct
  misc_feature 1..19: note = AAT RNAi agent sense strand core stretch base sequence
  source 1..19: mol_type = other RNA
accaagttcctggaaaatt

SEQ ID NO: 612 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
accaagttcctggaaaatn

SEQ ID NO: 613 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nccaagttcctggaaaatn

SEQ ID NO: 614 - RNA, 19 nt, synthetic construct
  misc_feature 1..19: note = AAT RNAi agent sense strand core stretch base sequence
  source 1..19: mol_type = other RNA
tccattactggaacctatg

SEQ ID NO: 615 - RNA, 19 nt, synthetic construct
  misc_feature 1..19: note = AAT RNAi agent sense strand core stretch base sequence
  source 1..19: mol_type = other RNA
tccattactggaacctata

SEQ ID NO: 616 - RNA, 19 nt, synthetic construct
  misc_feature 1..19: note = AAT RNAi agent sense strand core stretch base sequence
  source 1..19: mol_type = other RNA
tccattactggaacctatt

SEQ ID NO: 617 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tccattactggaacctatn

SEQ ID NO: 618 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nccattactggaacctatn

SEQ ID NO: 619 - RNA, 19 nt, synthetic construct
  misc_feature 1..19: note = AAT RNAi agent sense strand core stretch base sequence
  source 1..19: mol_type = other RNA
ccattactggaacctatga

SEQ ID NO: 620 - RNA, 19 nt, synthetic construct
  misc_feature 1..19: note = AAT RNAi agent sense strand core stretch base sequence
  source 1..19: mol_type = other RNA
ccattactggaacctatgt

SEQ ID NO: 621 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ccattactggaacctatgn

SEQ ID NO: 622 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncattactggaacctatgn

SEQ ID NO: 623 - RNA, 19 nt, synthetic construct
  misc_feature 1..19: note = AAT RNAi agent sense strand core stretch base sequence
  source 1..19: mol_type = other RNA
actggaacctatgatctga

SEQ ID NO: 624 - RNA, 19 nt, synthetic construct
  misc_feature 1..19: note = AAT RNAi agent sense strand core stretch base sequence
  source 1..19: mol_type = other RNA
actggaacctatgatctgt

SEQ ID NO: 625 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
actggaacctatgatctgn

SEQ ID NO: 626 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nctggaacctatgatctgn

SEQ ID NO: 627 - RNA, 19 nt, synthetic construct
  misc_feature 1..19: note = AAT RNAi agent sense strand core stretch base sequence
  source 1..19: mol_type = other RNA
ggaacctatgatctgaaga

SEQ ID NO: 628 - RNA, 19 nt, synthetic construct
  misc_feature 1..19: note = AAT RNAi agent sense strand core stretch base sequence
  source 1..19: mol_type = other RNA
ggaacctatgatctgaagt

SEQ ID NO: 629 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ggaacctatgatctgaagn

SEQ ID NO: 630 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngaacctatgatctgaagn

SEQ ID NO: 631 - RNA, 19 nt, synthetic construct
  misc_feature 1..19: note = AAT RNAi agent sense strand core stretch base sequence
  source 1..19: mol_type = other RNA
gaacctatgatctgaagag

SEQ ID NO: 632 - RNA, 19 nt, synthetic construct
  misc_feature 1..19: note = AAT RNAi agent sense strand core stretch base sequence
  source 1..19: mol_type = other RNA
gaacctatgatctgaagaa

SEQ ID NO: 633 - RNA, 19 nt, synthetic construct
  misc_feature 1..19: note = AAT RNAi agent sense strand core stretch base sequence
  source 1..19: mol_type = other RNA
gaacctatgatctgaagat

SEQ ID NO: 634 - RNA, 19 nt, synthetic construct
  misc_feature 1..19: note = AAT RNAi agent sense strand core stretch base sequence
  source 1..19: mol_type = other RNA
gaacctatgatctgaagag

SEQ ID NO: 635 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
naacctatgatctgaagan

SEQ ID NO: 636 - RNA, 19 nt, synthetic construct
  misc_feature 1..19: note = AAT RNAi agent sense strand core stretch base sequence
  source 1..19: mol_type = other RNA
cagcaatggggctgacctc

SEQ ID NO: 637 - RNA, 19 nt, synthetic construct
  misc_feature 1..19: note = AAT RNAi agent sense strand core stretch base sequence
  source 1..19: mol_type = other RNA
cagcaatggggctgaccta

SEQ ID NO: 638 - RNA, 19 nt, synthetic construct
  misc_feature 1..19: note = AAT RNAi agent sense strand core stretch base sequence
  source 1..19: mol_type = other RNA
cagcaatggggctgacctt

SEQ ID NO: 639 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
cagcaatggggctgacctn

SEQ ID NO: 640 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nagcaatggggctgacctn

SEQ ID NO: 641 - RNA, 19 nt, synthetic construct
  misc_feature 1..19: note = AAT RNAi agent sense strand core stretch base sequence
  source 1..19: mol_type = other RNA
gcaatggggctgacctctc

SEQ ID NO: 642 - RNA, 19 nt, synthetic construct
  misc_feature 1..19: note = AAT RNAi agent sense strand core stretch base sequence
  source 1..19: mol_type = other RNA
gcaatggggctgacctcta

SEQ ID NO: 643 - RNA, 19 nt, synthetic construct
  misc_feature 1..19: note = AAT RNAi agent sense strand core stretch base sequence
  misc_difference 19: note = n = any nuleotide
  source 1..19: mol_type = other RNA
gcaatggggctgacctctt

SEQ ID NO: 644 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gcaatggggctgacctctn

SEQ ID NO: 645 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncaatggggctgacctctn

SEQ ID NO: 646 - RNA, 19 nt, synthetic construct
  misc_feature 1..19: note = AAT RNAi agent sense strand core stretch base sequence
  source 1..19: mol_type = other RNA
agaggaggcacccctgaag

SEQ ID NO: 647 - RNA, 19 nt, synthetic construct
  misc_feature 1..19: note = AAT RNAi agent sense strand core stretch base sequence
  source 1..19: mol_type = other RNA
agaggaggcacccctgaaa

SEQ ID NO: 648 - RNA, 19 nt, synthetic construct
  misc_feature 1..19: note = AAT RNAi agent sense strand core stretch base sequence
  source 1..19: mol_type = other RNA
agaggaggcacccctgaat

SEQ ID NO: 649 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
agaggaggcacccctgaan

SEQ ID NO: 650 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngaggaggcacccctgaan

SEQ ID NO: 651 - RNA, 19 nt, synthetic construct
  misc_feature 1..19: note = AAT RNAi agent sense strand core stretch base sequence
  source 1..19: mol_type = other RNA
aggcacccctgaagctctc

SEQ ID NO: 652 - RNA, 19 nt, synthetic construct
  misc_feature 1..19: note = AAT RNAi agent sense strand core stretch base sequence
  source 1..19: mol_type = other RNA
aggcacccctgaagctcta

SEQ ID NO: 653 - RNA, 19 nt, synthetic construct
  misc_feature 1..19: note = AAT RNAi agent sense strand core stretch base sequence
  source 1..19: mol_type = other RNA
aggcacccctgaagctctt

SEQ ID NO: 654 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
aggcacccctgaagctctn

SEQ ID NO: 655 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nggcacccctgaagctctn

SEQ ID NO: 656 - RNA, 19 nt, synthetic construct
  misc_feature 1..19: note = AAT RNAi agent sense strand core stretch base sequence
  source 1..19: mol_type = other RNA
tctccaaggccgtgcataa

SEQ ID NO: 657 - RNA, 19 nt, synthetic construct
  misc_feature 1..19: note = AAT RNAi agent sense strand core stretch base sequence
  source 1..19: mol_type = other RNA
tctccaaggccgtgcatat

SEQ ID NO: 658 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tctccaaggccgtgcatan

SEQ ID NO: 659 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nctccaaggccgtgcatan

SEQ ID NO: 660 - RNA, 19 nt, synthetic construct
  misc_feature 1..19: note = AAT RNAi agent sense strand core stretch base sequence
  source 1..19: mol_type = other RNA
tccaaggccgtgcataagg

SEQ ID NO: 661 - RNA, 19 nt, synthetic construct
  misc_feature 1..19: note = AAT RNAi agent sense strand core stretch base sequence
  source 1..19: mol_type = other RNA
tccaaggccgtgcataaga

SEQ ID NO: 662 - RNA, 19 nt, synthetic construct
  misc_feature 1..19: note = AAT RNAi agent sense strand core stretch base sequence
  source 1..19: mol_type = other RNA
tccaaggccgtgcataagt

SEQ ID NO: 663 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tccaaggccgtgcataagn

SEQ ID NO: 664 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nccaaggccgtgcataagn

SEQ ID NO: 665 - RNA, 19 nt, synthetic construct
  misc_feature 1..19: note = AAT RNAi agent sense strand core stretch base sequence
  source 1..19: mol_type = other RNA
ccaaggccgtgcataaggc

SEQ ID NO: 666 - RNA, 19 nt, synthetic construct
  misc_feature 1..19: note = AAT RNAi agent sense strand core stretch base sequence
  source 1..19: mol_type = other RNA
ccaaggccgtgcataagga

SEQ ID NO: 667 - RNA, 19 nt, synthetic construct
  misc_feature 1..19: note = AAT RNAi agent sense strand core stretch base sequence
  source 1..19: mol_type = other RNA
ccaaggccgtgcataaggt

SEQ ID NO: 668 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ccaaggccgtgcataaggn

SEQ ID NO: 669 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncaaggccgtgcataaggn

SEQ ID NO: 670 - RNA, 19 nt, synthetic construct
  misc_feature 1..19: note = AAT RNAi agent sense strand core stretch base sequence
  source 1..19: mol_type = other RNA
caaggccgtgcataaggct

SEQ ID NO: 671 - RNA, 19 nt, synthetic construct
  misc_feature 1..19: note = AAT RNAi agent sense strand core stretch base sequence
  source 1..19: mol_type = other RNA
caaggccgtgcataaggca

SEQ ID NO: 672 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
caaggccgtgcataaggcn

SEQ ID NO: 673 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
naaggccgtgcataaggcn

SEQ ID NO: 674 - RNA, 19 nt, synthetic construct
  misc_feature 1..19: note = AAT RNAi agent sense strand core stretch base sequence
  source 1..19: mol_type = other RNA
aaggctgtgctgaccatcg

SEQ ID NO: 675 - RNA, 19 nt, synthetic construct
  misc_feature 1..19: note = AAT RNAi agent sense strand core stretch base sequence
  source 1..19: mol_type = other RNA
aaggctgtgctgaccatca

SEQ ID NO: 676 - RNA, 19 nt, synthetic construct
  misc_feature 1..19: note = AAT RNAi agent sense strand core stretch base sequence
  source 1..19: mol_type = other RNA
aaggctgtgctgaccatct

SEQ ID NO: 677 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
aaggctgtgctgaccatcn

SEQ ID NO: 678 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
naggctgtgctgaccatcn

SEQ ID NO: 679 - RNA, 19 nt, synthetic construct
  misc_feature 1..19: note = AAT RNAi agent sense strand core stretch base sequence
  source 1..19: mol_type = other RNA
ggctgtgctgaccatcgac

SEQ ID NO: 680 - RNA, 19 nt, synthetic construct
  misc_feature 1..19: note = AAT RNAi agent sense strand core stretch base sequence
  source 1..19: mol_type = other RNA
ggctgtgctgaccatcgaa

SEQ ID NO: 681 - RNA, 19 nt, synthetic construct
  misc_feature 1..19: note = AAT RNAi agent sense strand core stretch base sequence
  source 1..19: mol_type = other RNA
ggctgtgctgaccatcgat

SEQ ID NO: 682 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ggctgtgctgaccatcgan

SEQ ID NO: 683 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngctgtgctgaccatcgan

SEQ ID NO: 684 - RNA, 19 nt, synthetic construct
  misc_feature 1..19: note = AAT RNAi agent sense strand core stretch base sequence
  source 1..19: mol_type = other RNA
ctgctggggccatgttttt

SEQ ID NO: 685 - RNA, 19 nt, synthetic construct
  misc_feature 1..19: note = AAT RNAi agent sense strand core stretch base sequence
  source 1..19: mol_type = other RNA
ctgctggggccatgtttta

SEQ ID NO: 686 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ctgctggggccatgttttt

SEQ ID NO: 687 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntgctggggccatgttttn

SEQ ID NO: 688 - RNA, 19 nt, synthetic construct
  misc_feature 1..19: note = AAT RNAi agent sense strand core stretch base sequence
  source 1..19: mol_type = other RNA
gctggggccatgtttttag

SEQ ID NO: 689 - RNA, 19 nt, synthetic construct
  misc_feature 1..19: note = AAT RNAi agent sense strand core stretch base sequence
  source 1..19: mol_type = other RNA
gctggggccatgtttttaa

SEQ ID NO: 690 - RNA, 19 nt, synthetic construct
  misc_feature 1..19: note = AAT RNAi agent sense strand core stretch base sequence
  source 1..19: mol_type = other RNA
gctggggccatgtttttat

SEQ ID NO: 691 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gctggggccatgtttttan

SEQ ID NO: 692 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nctggggccatgtttttan

SEQ ID NO: 693 - RNA, 19 nt, synthetic construct
  misc_feature 1..19: note = AAT RNAi agent sense strand core stretch base sequence
  source 1..19: mol_type = other RNA
ctggggccatgtttttaga

SEQ ID NO: 694 - RNA, 19 nt, synthetic construct
  misc_feature 1..19: note = AAT RNAi agent sense strand core stretch base sequence
  source 1..19: mol_type = other RNA
ctggggccatgtttttagt

SEQ ID NO: 695 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ctggggccatgtttttagn

SEQ ID NO: 696 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntggggccatgtttttagn

SEQ ID NO: 697 - RNA, 19 nt, synthetic construct
  misc_feature 1..19: note = AAT RNAi agent sense strand core stretch base sequence
  source 1..19: mol_type = other RNA
ggggccatgtttttagagg

SEQ ID NO: 698 - RNA, 19 nt, synthetic construct
  misc_feature 1..19: note = AAT RNAi agent sense strand core stretch base sequence
  source 1..19: mol_type = other RNA
ggggccatgtttttagaga

SEQ ID NO: 699 - RNA, 19 nt, synthetic construct
  misc_feature 1..19: note = AAT RNAi agent sense strand core stretch base sequence
  source 1..19: mol_type = other RNA
ggggccatgtttttagagt

SEQ ID NO: 700 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ggggccatgtttttagagn

SEQ ID NO: 701 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngggccatgtttttagagn

SEQ ID NO: 702 - RNA, 19 nt, synthetic construct
  misc_feature 1..19: note = AAT RNAi agent sense strand core stretch base sequence
  source 1..19: mol_type = other RNA
gggccatgtttttagaggc

SEQ ID NO: 703 - RNA, 19 nt, synthetic construct
  misc_feature 1..19: note = AAT RNAi agent sense strand core stretch base sequence
  source 1..19: mol_type = other RNA
gggccatgtttttagagga

SEQ ID NO: 704 - RNA, 19 nt, synthetic construct
  misc_feature 1..19: note = AAT RNAi agent sense strand core stretch base sequence
  source 1..19: mol_type = other RNA
gggccatgtttttagaggt

SEQ ID NO: 705 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gggccatgtttttagaggn

SEQ ID NO: 706 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nggccatgtttttagaggn

SEQ ID NO: 707 - RNA, 19 nt, synthetic construct
  misc_feature 1..19: note = AAT RNAi agent sense strand core stretch base sequence
  source 1..19: mol_type = other RNA
gaggccatacccatgtcta

SEQ ID NO: 708 - RNA, 19 nt, synthetic construct
  misc_feature 1..19: note = AAT RNAi agent sense strand core stretch base sequence
  source 1..19: mol_type = other RNA
gaggccatacccatgtctt

SEQ ID NO: 709 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gaggccatacccatgtctn

SEQ ID NO: 710 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
naggccatacccatgtctn

SEQ ID NO: 711 - RNA, 19 nt, synthetic construct
  misc_feature 1..19: note = AAT RNAi agent sense strand core stretch base sequence
  source 1..19: mol_type = other RNA
ggccatacccatgtctatc

SEQ ID NO: 712 - RNA, 19 nt, synthetic construct
  misc_feature 1..19: note = AAT RNAi agent sense strand core stretch base sequence
  source 1..19: mol_type = other RNA
ggccatacccatgtctata

SEQ ID NO: 713 - RNA, 19 nt, synthetic construct
  misc_feature 1..19: note = AAT RNAi agent sense strand core stretch base sequence
  source 1..19: mol_type = other RNA
ggccatacccatgtctatt

SEQ ID NO: 714 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ggccatacccatgtctatn

SEQ ID NO: 715 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngccatacccatgtctatn

SEQ ID NO: 716 - RNA, 19 nt, synthetic construct
  misc_feature 1..19: note = AAT RNAi agent sense strand core stretch base sequence
  source 1..19: mol_type = other RNA
cccgaggtcaagttcaaca

SEQ ID NO: 717 - RNA, 19 nt, synthetic construct
  misc_feature 1..19: note = AAT RNAi agent sense strand core stretch base sequence
  source 1..19: mol_type = other RNA
cccgaggtcaagttcaact

SEQ ID NO: 718 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
cccgaggtcaagttcaacn

SEQ ID NO: 719 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nccgaggtcaagttcaacn

SEQ ID NO: 720 - RNA, 19 nt, synthetic construct
  misc_feature 1..19: note = AAT RNAi agent sense strand core stretch base sequence
  source 1..19: mol_type = other RNA
aggtcaagttcaacaaacc

SEQ ID NO: 721 - RNA, 19 nt, synthetic construct
  misc_feature 1..19: note = AAT RNAi agent sense strand core stretch base sequence
  source 1..19: mol_type = other RNA
aggtcaagttcaacaaaca

SEQ ID NO: 722 - RNA, 19 nt, synthetic construct
  misc_feature 1..19: note = AAT RNAi agent sense strand core stretch base sequence
  source 1..19: mol_type = other RNA
aggtcaagttcaacaaact

SEQ ID NO: 723 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
aggtcaagttcaacaaacn

SEQ ID NO: 724 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nggtcaagttcaacaaacn

SEQ ID NO: 725 - RNA, 19 nt, synthetic construct
  misc_feature 1..19: note = AAT RNAi agent sense strand core stretch base sequence
  source 1..19: mol_type = other RNA
caagttcaacaaacccttt

SEQ ID NO: 726 - RNA, 19 nt, synthetic construct
  misc_feature 1..19: note = AAT RNAi agent sense strand core stretch base sequence
  source 1..19: mol_type = other RNA
caagttcaacaaaccctta

SEQ ID NO: 727 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
caagttcaacaaacccttn

SEQ ID NO: 728 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
naagttcaacaaacccttn

SEQ ID NO: 729 - RNA, 19 nt, synthetic construct
  misc_feature 1..19: note = AAT RNAi agent sense strand core stretch base sequence
  source 1..19: mol_type = other RNA
agttcaacaaaccctttgt

SEQ ID NO: 730 - RNA, 19 nt, synthetic construct
  misc_feature 1..19: note = AAT RNAi agent sense strand core stretch base sequence
  source 1..19: mol_type = other RNA
agttcaacaaaccctttga

SEQ ID NO: 731 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
agttcaacaaaccctttgn

SEQ ID NO: 732 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngttcaacaaaccctttgn

SEQ ID NO: 733 - RNA, 19 nt, synthetic construct
  misc_feature 1..19: note = AAT RNAi agent sense strand core stretch base sequence
  source 1..19: mol_type = other RNA
gttcaacaaaccctttgtc

SEQ ID NO: 734 - RNA, 19 nt, synthetic construct
  misc_feature 1..19: note = AAT RNAi agent sense strand core stretch base sequence
  source 1..19: mol_type = other RNA
gttcaacaaaccctttgta

SEQ ID NO: 735 - RNA, 19 nt, synthetic construct
  misc_feature 1..19: note = AAT RNAi agent sense strand core stretch base sequence
  source 1..19: mol_type = other RNA
gttcaacaaaccctttgtt

SEQ ID NO: 736 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
gttcaacaaaccctttgtn

SEQ ID NO: 737 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nttcaacaaaccctttgtn

SEQ ID NO: 738 - RNA, 19 nt, synthetic construct
  misc_feature 1..19: note = AAT RNAi agent sense strand core stretch base sequence
  source 1..19: mol_type = other RNA
tcaacaaaccctttgtctt

SEQ ID NO: 739 - RNA, 19 nt, synthetic construct
  misc_feature 1..19: note = AAT RNAi agent sense strand core stretch base sequence
  source 1..19: mol_type = other RNA
tcaacaaaccctttgtcta

SEQ ID NO: 740 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tcaacaaaccctttgtctn

SEQ ID NO: 741 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ncaacaaaccctttgtctn

SEQ ID NO: 742 - RNA, 19 nt, synthetic construct
  misc_feature 1..19: note = AAT RNAi agent sense strand core stretch base sequence
  source 1..19: mol_type = other RNA
accctttgtcttcttaatg

SEQ ID NO: 743 - RNA, 19 nt, synthetic construct
  misc_feature 1..19: note = AAT RNAi agent sense strand core stretch base sequence
  source 1..19: mol_type = other RNA
accctttgtcttcttaata

SEQ ID NO: 744 - RNA, 19 nt, synthetic construct
  misc_feature 1..19: note = AAT RNAi agent sense strand core stretch base sequence
  source 1..19: mol_type = other RNA
accctttgtcttcttaatt

SEQ ID NO: 745 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
accctttgtcttcttaatn

SEQ ID NO: 746 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nccctttgtcttcttaatn

SEQ ID NO: 747 - RNA, 19 nt, synthetic construct
  misc_feature 1..19: note = AAT RNAi agent sense strand core stretch base sequence
  source 1..19: mol_type = other RNA
cctttgtcttcttaatgat

SEQ ID NO: 748 - RNA, 19 nt, synthetic construct
  misc_feature 1..19: note = AAT RNAi agent sense strand core stretch base sequence
  source 1..19: mol_type = other RNA
cctttgtcttcttaatgaa

SEQ ID NO: 749 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
cctttgtcttcttaatgan

SEQ ID NO: 750 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nctttgtcttcttaatgan

SEQ ID NO: 751 - RNA, 19 nt, synthetic construct
  misc_feature 1..19: note = AAT RNAi agent sense strand core stretch base sequence
  source 1..19: mol_type = other RNA
taccaagtctcccctcttc

SEQ ID NO: 752 - RNA, 19 nt, synthetic construct
  misc_feature 1..19: note = AAT RNAi agent sense strand core stretch base sequence
  source 1..19: mol_type = other RNA
taccaagtctcccctctta

SEQ ID NO: 753 - RNA, 19 nt, synthetic construct
  misc_feature 1..19: note = AAT RNAi agent sense strand core stretch base sequence
  source 1..19: mol_type = other RNA
taccaagtctcccctcttt

SEQ ID NO: 754 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
taccaagtctcccctcttn

SEQ ID NO: 755 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
naccaagtctcccctcttn

SEQ ID NO: 756 - RNA, 19 nt, synthetic construct
  misc_feature 1..19: note = AAT RNAi agent sense strand core stretch base sequence
  source 1..19: mol_type = other RNA
aagtctcccctcttcatgg

SEQ ID NO: 757 - RNA, 19 nt, synthetic construct
  misc_feature 1..19: note = AAT RNAi agent sense strand core stretch base sequence
  source 1..19: mol_type = other RNA
aagtctcccctcttcatga

SEQ ID NO: 758 - RNA, 19 nt, synthetic construct
  misc_feature 1..19: note = AAT RNAi agent sense strand core stretch base sequence
  source 1..19: mol_type = other RNA
aagtctcccctcttcatgt

SEQ ID NO: 759 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
aagtctcccctcttcatgn

SEQ ID NO: 760 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nagtctcccctcttcatgn

SEQ ID NO: 761 - RNA, 19 nt, synthetic construct
  misc_feature 1..19: note = AAT RNAi agent sense strand core stretch base sequence
  source 1..19: mol_type = other RNA
agtctcccctcttcatggg

SEQ ID NO: 762 - RNA, 19 nt, synthetic construct
  misc_feature 1..19: note = AAT RNAi agent sense strand core stretch base sequence
  source 1..19: mol_type = other RNA
agtctcccctcttcatgga

SEQ ID NO: 763 - RNA, 19 nt, synthetic construct
  misc_feature 1..19: note = AAT RNAi agent sense strand core stretch base sequence
  source 1..19: mol_type = other RNA
agtctcccctcttcatggt

SEQ ID NO: 764 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
agtctcccctcttcatggn

SEQ ID NO: 765 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ngtctcccctcttcatggn

SEQ ID NO: 766 - RNA, 19 nt, synthetic construct
  misc_feature 1..19: note = AAT RNAi agent sense strand core stretch base sequence
  source 1..19: mol_type = other RNA
tctcccctcttcatgggaa

SEQ ID NO: 767 - RNA, 19 nt, synthetic construct
  misc_feature 1..19: note = AAT RNAi agent sense strand core stretch base sequence
  source 1..19: mol_type = other RNA
tctcccctcttcatgggat

SEQ ID NO: 768 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
tctcccctcttcatgggan

SEQ ID NO: 769 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
nctcccctcttcatgggan

SEQ ID NO: 770 - RNA, 19 nt, synthetic construct
  misc_feature 1..19: note = AAT RNAi agent sense strand core stretch base sequence
  source 1..19: mol_type = other RNA
ctcccctcttcatgggaaa

SEQ ID NO: 771 - RNA, 19 nt, synthetic construct
  misc_feature 1..19: note = AAT RNAi agent sense strand core stretch base sequence
  source 1..19: mol_type = other RNA
ctcccctcttcatgggaat

SEQ ID NO: 772 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
ctcccctcttcatgggaan

SEQ ID NO: 773 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntcccctcttcatgggaan

SEQ ID NO: 774 - RNA, 19 nt, synthetic construct
  misc_feature 1..19: note = AAT RNAi agent sense strand core stretch base sequence
  source 1..19: mol_type = other RNA
atgacattaaagaagggtt

SEQ ID NO: 775 - RNA, 19 nt, synthetic construct
  misc_feature 1..19: note = AAT RNAi agent sense strand core stretch base sequence
  source 1..19: mol_type = other RNA
atgacattaaagaagggta

SEQ ID NO: 776 - RNA, 19 nt, synthetic construct
  misc_feature 1..19: note = AAT RNAi agent sense strand core stretch base sequence
  misc_difference 19: note = n = any nucleotide
  source 1..19: mol_type = other RNA
atgacattaaagaagggtn

SEQ ID NO: 777 - RNA, 19 nt, synthetic construct
  misc_feature 1..19: note = AAT RNAi agent sense strand core stretch base sequence
  misc_difference 1: note = n = any nucleotide
  misc_difference 19: note = n = any nucleotide
  source 1..19: mol_type = other RNA
ntgacattaaagaagggtn

SEQ ID NO: 778 - RNA, 18 nt, synthetic construct
  misc_feature 1..18: note = AAT RNAi agent antisense strand base sequence
  source 1..18: mol_type = other RNA
ggaacttggtgatgatat

SEQ ID NO: 779 - RNA, 18 nt, synthetic construct
  misc_feature 1..18: note = AAT RNAi agent antisense strand base sequence
  source 1..18: mol_type = other RNA
gatcataggttccagtaa

SEQ ID NO: 780 - RNA, 18 nt, synthetic construct
  misc_feature 1..18: note = AAT RNAi agent antisense strand base sequence
  source 1..18: mol_type = other RNA
acagccttatgcacggcc

SEQ ID NO: 781 - RNA, 18 nt, synthetic construct
  misc_feature 1..18: note = AAT RNAi agent antisense strand base sequence
  source 1..18: mol_type = other RNA
tcgatggtcagcacagcc

SEQ ID NO: 782 - RNA, 18 nt, synthetic construct
  misc_feature 1..18: note = AAT RNAi agent antisense strand base sequence
  source 1..18: mol_type = other RNA
caaagggtttgttgaact

SEQ ID NO: 783 - DNA, 21 bp, synthetic construct
  misc_feature 1..21: note = AAT RNAi agent antisense strand base sequence
  source 1..21: mol_type = other DNA
  modified_base 7..8: mod_base = OTHER; note = uracil
  modified_base 11: mod_base = OTHER; note = uracil
  modified_base 14: mod_base = OTHER; note = uracil
  modified_base 17: mod_base = OTHER; note = uracil
  modified_base 19: mod_base = OTHER; note = uracil
tggaacttggtgatgatattt

SEQ ID NO: 784 - DNA, 23 bp, synthetic construct
  misc_feature 1..23: note = AAT RNAi agent antisense strand base sequence
  source 1..23: mol_type = other DNA
  modified_base 7..8: mod_base = OTHER; note = uracil
  modified_base 11: mod_base = OTHER; note = uracil
  modified_base 14: mod_base = OTHER; note = uracil
  modified_base 17: mod_base = OTHER; note = uracil
  modified_base 19: mod_base = OTHER; note = uracil
  modified_base 22: mod_base = OTHER; note = uracil
tggaacttggtgatgatatcgtg

SEQ ID NO: 785 - DNA, 17 bp, synthetic construct
  misc_feature 1..17: note = AAT RNAi agent antisense strand base sequence
  source 1..17: mol_type = other DNA
  modified_base 3..4: mod_base = OTHER; note = uracil
  modified_base 7: mod_base = OTHER; note = uracil
  modified_base 10: mod_base = OTHER; note = uracil
  modified_base 13: mod_base = OTHER; note = uracil
  modified_base 15: mod_base = OTHER; note = uracil
acttggtgatgatattt

SEQ ID NO: 786 - DNA, 21 bp, synthetic construct
  misc_feature 1..21: note = AAT RNAi agent antisense strand base sequence
  source 1..21: mol_type = other DNA
tggaacttggtgatgatattt

SEQ ID NO: 787 - RNA, 21 nt, synthetic construct
  misc_feature 1..21: note = AAT RNAi agent antisense strand base sequence
  source 1..21: mol_type = other RNA
tttaaacatgcctaaacgctt

SEQ ID NO: 788 - RNA, 21 nt, synthetic construct
  misc_feature 1..21: note = AAT RNAi agent antisense strand base sequence
  source 1..21: mol_type = other RNA
tgcattgcccaggtatttctt

SEQ ID NO: 789 - RNA, 21 nt, synthetic construct
  misc_feature 1..21: note = AAT RNAi agent antisense strand base sequence
  source 1..21: mol_type = other RNA
tggaacttggtgatgatattt

SEQ ID NO: 790 - RNA, 21 nt, synthetic construct
  misc_feature 1..21: note = AAT RNAi agent antisense strand base sequence
  source 1..21: mol_type = other RNA
tgatcataggttccagtaatt

SEQ ID NO: 791 - RNA, 21 nt, synthetic construct
  misc_feature 1..21: note = AAT RNAi agent antisense strand base sequence
  source 1..21: mol_type = other RNA
tacagccttatgcacggcctt

SEQ ID NO: 792 - RNA, 21 nt, synthetic construct
  misc_feature 1..21: note = AAT RNAi agent antisense strand base sequence
  source 1..21: mol_type = other RNA
ttcgatggtcagcacagcctt

SEQ ID NO: 793 - RNA, 21 nt, synthetic construct
  misc_feature 1..21: note = AAT RNAi agent antisense strand base sequence
  source 1..21: mol_type = other RNA
tcaaagggtttgttgaacttt

SEQ ID NO: 794 - RNA, 21 nt, synthetic construct
  misc_feature 1..21: note = AAT RNAi agent antisense strand base sequence
  source 1..21: mol_type = other RNA
tgttaaacatgcctaaacgtt

SEQ ID NO: 795 - RNA, 21 nt, synthetic construct
  misc_feature 1..21: note = AAT RNAi agent antisense strand base sequence
  source 1..21: mol_type = other RNA
tttaaacgtgcctaaacgctg

SEQ ID NO: 796 - RNA, 21 nt, synthetic construct
  misc_feature 1..21: note = AAT RNAi agent antisense strand base sequence
  source 1..21: mol_type = other RNA
tgcattgcccaggtatttcag

SEQ ID NO: 797 - RNA, 21 nt, synthetic construct
  misc_feature 1..21: note = AAT RNAi agent antisense strand base sequence
  source 1..21: mol_type = other RNA
tggaacttggtgatgatatcg

SEQ ID NO: 798 - RNA, 21 nt, synthetic construct
  misc_feature 1..21: note = AAT RNAi agent antisense strand base sequence
  source 1..21: mol_type = other RNA
tgatcataggttccagtaatg

SEQ ID NO: 799 - RNA, 21 nt, synthetic construct
  misc_feature 1..21: note = AAT RNAi agent antisense strand base sequence
  source 1..21: mol_type = other RNA
tcaaagggtttgttgaacttg

SEQ ID NO: 800 - RNA, 21 nt, synthetic construct
  misc_feature 1..21: note = AAT RNAi agent antisense strand base sequence
  source 1..21: mol_type = other RNA
tgttaaacatgcctaaacgcg

SEQ ID NO: 801 - RNA, 21 nt, synthetic construct
  misc_feature 1..21: note = AAT RNAi agent antisense strand base sequence
  source 1..21: mol_type = other RNA
tgttaaacatgcctaaacgct

SEQ ID NO: 802 - RNA, 23 nt, synthetic construct
  misc_feature 1..23: note = AAT RNAi agent antisense strand base sequence
  source 1..23: mol_type = other RNA
tgttaaacatgcctaaacgcttc

SEQ ID NO: 803 - RNA, 21 nt, synthetic construct
  misc_feature 1..21: note = AAT RNAi agent antisense strand base sequence
  source 1..21: mol_type = other RNA
tgctgttggactggtgtgctt

SEQ ID NO: 804 - RNA, 21 nt, synthetic construct
  misc_feature 1..21: note = AAT RNAi agent antisense strand base sequence
  source 1..21: mol_type = other RNA
tgctgttggactggtgtgcca

SEQ ID NO: 805 - RNA, 23 nt, synthetic construct
  misc_feature 1..23: note = AAT RNAi agent antisense strand base sequence
  source 1..23: mol_type = other RNA
tgctgttggactggtgtgccatt

SEQ ID NO: 806 - RNA, 23 nt, synthetic construct
  misc_feature 1..23: note = AAT RNAi agent antisense strand base sequence
  source 1..23: mol_type = other RNA
tgctgttggactggtgtgccagc

SEQ ID NO: 807 - RNA, 21 nt, synthetic construct
  misc_feature 1..21: note = AAT RNAi agent antisense strand base sequence
  source 1..21: mol_type = other RNA
taaggcttctgagtggtactt

SEQ ID NO: 808 - RNA, 21 nt, synthetic construct
  misc_feature 1..21: note = AAT RNAi agent antisense strand base sequence
  source 1..21: mol_type = other RNA
taaggcttctgagtggtacaa

SEQ ID NO: 809 - RNA, 23 nt, synthetic construct
  misc_feature 1..23: note = AAT RNAi agent antisense strand base sequence
  source 1..23: mol_type = other RNA
taaggcttctgagtggtacaact

SEQ ID NO: 810 - RNA, 21 nt, synthetic construct
  misc_feature 1..21: note = AAT RNAi agent antisense strand base sequence
  source 1..21: mol_type = other RNA
gaaggcttctgagtggtactt

SEQ ID NO: 811 - RNA, 21 nt, synthetic construct
  misc_feature 1..21: note = AAT RNAi agent antisense strand base sequence
  source 1..21: mol_type = other RNA
aagacaaagggtttgttgatt

SEQ ID NO: 812 - RNA, 21 nt, synthetic construct
  misc_feature 1..21: note = AAT RNAi agent antisense strand base sequence
  source 1..21: mol_type = other RNA
aagacaaagggtttgttgaac

SEQ ID NO: 813 - RNA, 21 nt, synthetic construct
  misc_feature 1..21: note = AAT RNAi agent antisense strand base sequence
  source 1..21: mol_type = other RNA
tagacaaagggtttgttgaac

SEQ ID NO: 814 - RNA, 23 nt, synthetic construct
  misc_feature 1..23: note = AAT RNAi agent antisense strand base sequence
  source 1..23: mol_type = other RNA
aagacaaagggtttgttgaactt

SEQ ID NO: 815 - RNA, 21 nt, synthetic construct
  misc_feature 1..21: note = AAT RNAi agent antisense strand base sequence
  source 1..21: mol_type = other RNA
tagacatgggtatggcctctt

SEQ ID NO: 816 - RNA, 21 nt, synthetic construct
  misc_feature 1..21: note = AAT RNAi agent antisense strand base sequence
  source 1..21: mol_type = other RNA
tagacatgggtatggcctcta

SEQ ID NO: 817 - RNA, 23 nt, synthetic construct
  misc_feature 1..23: note = AAT RNAi agent antisense strand base sequence
  source 1..23: mol_type = other RNA
tagacatgggtatggcctctaaa

SEQ ID NO: 818 - RNA, 23 nt, synthetic construct
  misc_feature 1..23: note = AAT RNAi agent antisense strand base sequence
  source 1..23: mol_type = other RNA
tagacatgggtatggcctctatt

SEQ ID NO: 819 - RNA, 21 nt, synthetic construct
  misc_feature 1..21: note = AAT RNAi agent antisense strand base sequence
  source 1..21: mol_type = other RNA
tttgatctgtttcttggcctt

SEQ ID NO: 820 - RNA, 21 nt, synthetic construct
  misc_feature 1..21: note = AAT RNAi agent antisense strand base sequence
  source 1..21: mol_type = other RNA
tttgatctgtttcttggcctc

SEQ ID NO: 821 - RNA, 23 nt, synthetic construct
  misc_feature 1..23: note = AAT RNAi agent antisense strand base sequence
  source 1..23: mol_type = other RNA
tttgatctgtttcttggcctctt

SEQ ID NO: 822 - RNA, 21 nt, synthetic construct
  misc_feature 1..21: note = AAT RNAi agent antisense strand base sequence
  source 1..21: mol_type = other RNA
tgttggactggtgtgccagtt

SEQ ID NO: 823 - RNA, 21 nt, synthetic construct
  misc_feature 1..21: note = AAT RNAi agent antisense strand base sequence
  source 1..21: mol_type = other RNA
tgttggactggtgtgccagct

SEQ ID NO: 824 - RNA, 23 nt, synthetic construct
  misc_feature 1..23: note = AAT RNAi agent antisense strand base sequence
  source 1..23: mol_type = other RNA
tgttggactggtgtgccagctgg

SEQ ID NO: 825 - RNA, 22 nt, synthetic construct
  misc_feature 1..22: note = AAT RNAi agent antisense strand base sequence
  source 1..22: mol_type = other RNA
tgttggactggtgtgccagctg

SEQ ID NO: 826 - RNA, 21 nt, synthetic construct
  misc_feature 1..21: note = AAT RNAi agent antisense strand base sequence
  source 1..21: mol_type = other RNA
aaagggtttgttgaacttgtt

SEQ ID NO: 827 - RNA, 21 nt, synthetic construct
  misc_feature 1..21: note = AAT RNAi agent antisense strand base sequence
  source 1..21: mol_type = other RNA
aaagggtttgttgaacttgac

SEQ ID NO: 828 - RNA, 23 nt, synthetic construct
  misc_feature 1..23: note = AAT RNAi agent antisense strand base sequence
  source 1..23: mol_type = other RNA
taagggtttgttgaacttgacct

SEQ ID NO: 829 - RNA, 21 nt, synthetic construct
  misc_feature 1..21: note = AAT RNAi agent antisense strand base sequence
  source 1..21: mol_type = other RNA
taagggtttgttgaacttgac

SEQ ID NO: 830 - RNA, 21 nt, synthetic construct
  misc_feature 1..21: note = AAT RNAi agent antisense strand base sequence
  source 1..21: mol_type = other RNA
tattggtgctgttggactgtt

SEQ ID NO: 831 - RNA, 21 nt, synthetic construct
  misc_feature 1..21: note = AAT RNAi agent antisense strand base sequence
  source 1..21: mol_type = other RNA
tattggtgctgttggactggt

SEQ ID NO: 832 - RNA, 22 nt, synthetic construct
  misc_feature 1..22: note = AAT RNAi agent antisense strand base sequence
  source 1..22: mol_type = other RNA
tattggtgctgttggactggtt

SEQ ID NO: 833 - RNA, 20 nt, synthetic construct
  misc_feature 1..20: note = AAT RNAi agent antisense strand base sequence
  source 1..20: mol_type = other RNA
ttgttggactggtgtgccag

SEQ ID NO: 834 - RNA, 22 nt, synthetic construct
  misc_feature 1..22: note = AAT RNAi agent antisense strand base sequence
  source 1..22: mol_type = other RNA
ttgttggactggtgtgccagct

SEQ ID NO: 835 - RNA, 21 nt, synthetic construct
  misc_feature 1..21: note = AAT RNAi agent antisense strand base sequence
  source 1..21: mol_type = other RNA
tatagacatgggtatggcctc

SEQ ID NO: 836 - RNA, 23 nt, synthetic construct
  misc_feature 1..23: note = AAT RNAi agent antisense strand base sequence
  source 1..23: mol_type = other RNA
tcaaagggtttgttgaacttgac

SEQ ID NO: 837 - RNA, 21 nt, synthetic construct
  misc_feature 1..21: note = AAT RNAi agent antisense strand base sequence
  source 1..21: mol_type = other RNA
ttattggtgctgttggactgg

SEQ ID NO: 838 - RNA, 20 nt, synthetic construct
  misc_feature 1..20: note = AAT RNAi agent antisense strand base sequence
  source 1..20: mol_type = other RNA
tgttaaacatgcctaaacgc

SEQ ID NO: 839 - RNA, 22 nt, synthetic construct
  misc_feature 1..22: note = AAT RNAi agent antisense strand base sequence
  source 1..22: mol_type = other RNA
tgttaaacatgcctaaacgctt

SEQ ID NO: 840 - RNA, 18 nt, synthetic construct
  misc_feature 1..18: note = AAT RNAi agent sense strand base sequence
  source 1..18: mol_type = other RNA
atatcatcaccaagttcc

SEQ ID NO: 841 - RNA, 18 nt, synthetic construct
  misc_feature 1..18: note = AAT RNAi agent sense strand base sequence
  source 1..18: mol_type = other RNA
ttactggaacctatgatc

SEQ ID NO: 842 - RNA, 18 nt, synthetic construct
  misc_feature 1..18: note = AAT RNAi agent sense strand base sequence
  source 1..18: mol_type = other RNA
ggccgtgcataaggctgt

SEQ ID NO: 843 - RNA, 18 nt, synthetic construct
  misc_feature 1..18: note = AAT RNAi agent sense strand base sequence
  source 1..18: mol_type = other RNA
ggctgtgctgaccatcga

SEQ ID NO: 844 - RNA, 18 nt, synthetic construct
  misc_feature 1..18: note = AAT RNAi agent sense strand base sequence
  source 1..18: mol_type = other RNA
agttcaacaaaccctttg

SEQ ID NO: 845 - DNA, 23 bp, synthetic construct
  misc_feature 1..23: note = AAT RNAi agent sense strand base sequence
  source 1..23: mol_type = other DNA
  modified_base 1: mod_base = OTHER; note = uracil
  modified_base 3: mod_base = OTHER; note = uracil
  modified_base 5: mod_base = OTHER; note = uracil
  modified_base 7: mod_base = OTHER; note = uracil
  modified_base 10: mod_base = OTHER; note = uracil
  modified_base 18..19: mod_base = OTHER; note = uracil
tatatatcatcaccaagttccat

SEQ ID NO: 846 - DNA, 20 bp, synthetic construct
  misc_feature 1..20: note = AAT RNAi agent sense strand base sequence
  source 1..20: mol_type = other DNA
  modified_base 2: mod_base = OTHER; note = uracil
  modified_base 4: mod_base = OTHER; note = uracil
  modified_base 7: mod_base = OTHER; note = uracil
  modified_base 15..16: mod_base = OTHER; note = uracil
atatcatcaccaagttccat

SEQ ID NO: 847 - RNA, 21 nt, synthetic construct
  misc_feature 1..21: note = AAT RNAi agent sense strand base sequence
  source 1..21: mol_type = other RNA
cgatatcatcaccaagttcca

SEQ ID NO: 848 - DNA, 19 bp, synthetic construct
  misc_feature 1..19: note = AAT RNAi agent sense strand base sequence
  source 1..19: mol_type = other DNA
  modified_base 1: mod_base = OTHER; note = uracil
  modified_base 3: mod_base = OTHER; note = uracil
  modified_base 6: mod_base = OTHER; note = uracil
  modified_base 14..15: mod_base = OTHER; note = uracil
tatcatcaccaagttccat

SEQ ID NO: 849 - DNA, 23 bp, synthetic construct
  misc_feature 1..23: note = AAT RNAi agent sense strand base sequence
  source 1..23: mol_type = other DNA
tatatatcatcaccaagttccat

SEQ ID NO: 850 - RNA, 21 nt, synthetic construct
  misc_feature 1..21: note = AAT RNAi agent sense strand base sequence
  source 1..21: mol_type = other RNA
gcgtttaggcatgtttaaatt

SEQ ID NO: 851 - RNA, 21 nt, synthetic construct
  misc_feature 1..21: note = AAT RNAi agent sense strand base sequence
  source 1..21: mol_type = other RNA
gaaatacctgggcaatgcatt

SEQ ID NO: 852 - RNA, 21 nt, synthetic construct
  misc_feature 1..21: note = AAT RNAi agent sense strand base sequence
  source 1..21: mol_type = other RNA
atatcatcaccaagttccatt

SEQ ID NO: 853 - RNA, 21 nt, synthetic construct
  misc_feature 1..21: note = AAT RNAi agent sense strand base sequence
  source 1..21: mol_type = other RNA
ttactggaacctatgatcatt

SEQ ID NO: 854 - RNA, 21 nt, synthetic construct
  misc_feature 1..21: note = AAT RNAi agent sense strand base sequence
  source 1..21: mol_type = other RNA
ggccgtgcataaggctgtatt

SEQ ID NO: 855 - RNA, 21 nt, synthetic construct
  misc_feature 1..21: note = AAT RNAi agent sense strand base sequence
  source 1..21: mol_type = other RNA
ggctgtgctgaccatcgaatt

SEQ ID NO: 856 - RNA, 21 nt, synthetic construct
  misc_feature 1..21: note = AAT RNAi agent sense strand base sequence
  source 1..21: mol_type = other RNA
agttcaacaaaccctttgatt

SEQ ID NO: 857 - RNA, 21 nt, synthetic construct
  misc_feature 1..21: note = AAT RNAi agent sense strand base sequence
  source 1..21: mol_type = other RNA
cgtttaggcatgtttaacatt

SEQ ID NO: 858 - RNA, 21 nt, synthetic construct
  misc_feature 1..21: note = AAT RNAi agent sense strand base sequence
  source 1..21: mol_type = other RNA
cagcgtttaggcatgtttaaa

SEQ ID NO: 859 - RNA, 21 nt, synthetic construct
  misc_feature 1..21: note = AAT RNAi agent sense strand base sequence
  source 1..21: mol_type = other RNA
ctgaaatacctgggcaatgca

SEQ ID NO: 860 - RNA, 21 nt, synthetic construct
  misc_feature 1..21: note = AAT RNAi agent sense strand base sequence
  source 1..21: mol_type = other RNA
cattactggaacctatgatca

SEQ ID NO: 861 - RNA, 21 nt, synthetic construct
  misc_feature 1..21: note = AAT RNAi agent sense strand base sequence
  source 1..21: mol_type = other RNA
aaggccgtgcataaggctgta

SEQ ID NO: 862 - RNA, 21 nt, synthetic construct
  misc_feature 1..21: note = AAT RNAi agent sense strand base sequence
  source 1..21: mol_type = other RNA
aaggctgtgctgaccatcgaa

SEQ ID NO: 863 - RNA, 21 nt, synthetic construct
  misc_feature 1..21: note = AAT RNAi agent sense strand base sequence
  source 1..21: mol_type = other RNA
caagttcaacaaaccctttga

SEQ ID NO: 864 - RNA, 21 nt, synthetic construct
  misc_feature 1..21: note = AAT RNAi agent sense strand base sequence
  source 1..21: mol_type = other RNA
cgcgtttaggcatgtttaaca

SEQ ID NO: 865 - RNA, 21 nt, synthetic construct
  misc_feature 1..21: note = AAT RNAi agent sense strand base sequence
  source 1..21: mol_type = other RNA
aacgtttaggcatgtttaaca

SEQ ID NO: 866 - RNA, 21 nt, synthetic construct
  misc_feature 1..21: note = AAT RNAi agent sense strand base sequence
  source 1..21: mol_type = other RNA
agcgtttaggcatgtttaaca

SEQ ID NO: 867 - RNA, 21 nt, synthetic construct
  misc_feature 1..21: note = AAT RNAi agent sense strand base sequence
  source 1..21: mol_type = other RNA
tggcacaccagtccaacagca

SEQ ID NO: 868 - RNA, 21 nt, synthetic construct
  misc_feature 1..21: note = AAT RNAi agent sense strand base sequence
  source 1..21: mol_type = other RNA
aagcacaccagtccaacagca

SEQ ID NO: 869 - RNA, 21 nt, synthetic construct
  misc_feature 1..21: note = AAT RNAi agent sense strand base sequence
  source 1..21: mol_type = other RNA
ttgtaccactcagaagcctta

SEQ ID NO: 870 - RNA, 21 nt, synthetic construct
  misc_feature 1..21: note = AAT RNAi agent sense strand base sequence
  source 1..21: mol_type = other RNA
gttcaacaaaccctttgtctt

SEQ ID NO: 871 - RNA, 21 nt, synthetic construct
  misc_feature 1..21: note = AAT RNAi agent sense strand base sequence
  source 1..21: mol_type = other RNA
gttcaacaaaccctttgtcta

SEQ ID NO: 872 - RNA, 21 nt, synthetic construct
  misc_feature 1..21: note = AAT RNAi agent sense strand base sequence
  source 1..21: mol_type = other RNA
tagaggccatacccatgtcta

SEQ ID NO: 873 - RNA, 21 nt, synthetic construct
  misc_feature 1..21: note = AAT RNAi agent sense strand base sequence
  source 1..21: mol_type = other RNA
gaggccaagaaacagatcaaa

SEQ ID NO: 874 - RNA, 21 nt, synthetic construct
  misc_feature 1..21: note = AAT RNAi agent sense strand base sequence
  source 1..21: mol_type = other RNA
agctggcacaccagtccaaca

SEQ ID NO: 875 - RNA, 20 nt, synthetic construct
  misc_feature 1..20: note = AAT RNAi agent sense strand base sequence
  source 1..20: mol_type = other RNA
gctggcacaccagtccaaca

SEQ ID NO: 876 - RNA, 21 nt, synthetic construct
  misc_feature 1..21: note = AAT RNAi agent sense strand base sequence
  source 1..21: mol_type = other RNA
gtcaagttcaacaaacccttt

SEQ ID NO: 877 - RNA, 21 nt, synthetic construct
  misc_feature 1..21: note = AAT RNAi agent sense strand base sequence
  source 1..21: mol_type = other RNA
gtcaagttcaacaaaccctta

SEQ ID NO: 878 - RNA, 21 nt, synthetic construct
  misc_feature 1..21: note = AAT RNAi agent sense strand base sequence
  source 1..21: mol_type = other RNA
accagtccaacagcaccaata

SEQ ID NO: 879 - RNA, 20 nt, synthetic construct
  misc_feature 1..20: note = AAT RNAi agent sense strand base sequence
  source 1..20: mol_type = other RNA
ccagtccaacagcaccaata

SEQ ID NO: 880 - RNA, 20 nt, synthetic construct
  misc_feature 1..20: note = AAT RNAi agent sense strand base sequence
  source 1..20: mol_type = other RNA
ctggcacaccagtccaacaa

SEQ ID NO: 881 - RNA, 21 nt, synthetic construct
  misc_feature 1..21: note = AAT RNAi agent sense strand base sequence
  source 1..21: mol_type = other RNA
gaggccatacccatgtctata

SEQ ID NO: 882 - RNA, 23 nt, synthetic construct
  misc_feature 1..23: note = AAT RNAi agent sense strand base sequence
  source 1..23: mol_type = other RNA
gtcaagttcaacaaaccctttga

SEQ ID NO: 883 - RNA, 21 nt, synthetic construct
  misc_feature 1..21: note = AAT RNAi agent sense strand base sequence
  source 1..21: mol_type = other RNA
ccagtccaacagcaccaataa

SEQ ID NO: 884 - RNA, 20 nt, synthetic construct
  misc_feature 1..20: note = AAT RNAi agent sense strand base sequence
  source 1..20: mol_type = other RNA
gcgtttaggcatgtttaaca

SEQ ID NO: 885 - RNA, 22 nt, synthetic construct
  misc_feature 1..22: note = AAT RNAi agent sense strand base sequence
  source 1..22: mol_type = other RNA
gcgtttaggcatgtttaacatt

SEQ ID NO: 886 - RNA, 23 nt, synthetic construct
  misc_feature 1..23: note = AAT RNAi agent sense strand base sequence
  source 1..23: mol_type = other RNA
cgcgtttaggcatgtttaacatt

SEQ ID NO: 887 - RNA, 23 nt, synthetic construct
  misc_feature 1..23: note = AAT RNAi agent sense strand base sequence
  source 1..23: mol_type = other RNA
agcgtttaggcatgtttaacatt

SEQ ID NO: 888 - DNA, 21 bp, synthetic construct
  misc_feature 1..21: note = AAT RNAi agent antisense strand modified sequence
  source 1..21: mol_type = other DNA
  modified_base 7..8: mod_base = OTHER; note = uracil
  modified_base 11: mod_base = OTHER; note = uracil
  modified_base 14: mod_base = OTHER; note = uracil
  modified_base 17: mod_base = OTHER; note = uracil
  modified_base 19: mod_base = OTHER; note = uracil
tggaacttggtgatgatattt

SEQ ID NO: 889 - DNA, 21 bp, synthetic construct
  misc_feature 1..21: note = AAT RNAi agent antisense strand modified sequence
  source 1..21: mol_type = other DNA
  modified_base 7..8: mod_base = OTHER; note = uracil
  modified_base 11: mod_base = OTHER; note = uracil
  modified_base 14: mod_base = OTHER; note = uracil
  modified_base 17: mod_base = OTHER; note = uracil
  modified_base 19: mod_base = OTHER; note = uracil
tggaacttggtgatgatattt

SEQ ID NO: 890 - DNA, 23 bp, synthetic construct
  misc_feature 1..23: note = AAT RNAi agent antisense strand modified sequence
  source 1..23: mol_type = other DNA
  modified_base 7..8: mod_base = OTHER; note = uracil
  modified_base 11: mod_base = OTHER; note = uracil
  modified_base 14: mod_base = OTHER; note = uracil
  modified_base 17: mod_base = OTHER; note = uracil
  modified_base 19: mod_base = OTHER; note = uracil
  modified_base 22: mod_base = OTHER; note = uracil
tggaacttggtgatgatatcgtg

SEQ ID NO: 891 - DNA, 17 bp, synthetic construct
  misc_feature 1..17: note = AAT RNAi agent antisense strand modified sequence
  source 1..17: mol_type = other DNA
  modified_base 3..4: mod_base = OTHER; note = uracil
  modified_base 7: mod_base = OTHER; note = uracil
  modified_base 10: mod_base = OTHER; note = uracil
  modified_base 13: mod_base = OTHER; note = uracil
  modified_base 15: mod_base = OTHER; note = uracil
acttggtgatgatattt

SEQ ID NO: 892 - DNA, 21 bp, synthetic construct
  misc_feature 1..21: note = AAT RNAi agent antisense strand modified sequence
  source 1..21: mol_type = other DNA
tggaacttggtgatgatattt

SEQ ID NO: 893 - RNA, 21 nt, synthetic construct
  misc_feature 1..21: note = AAT RNAi agent antisense strand modified sequence
  source 1..21: mol_type = other RNA
tttaaacatgcctaaacgctt

SEQ ID NO: 894 - RNA, 21 nt, synthetic construct
  misc_feature 1..21: note = AAT RNAi agent antisense strand modified sequence
  source 1..21: mol_type = other RNA
tgcattgcccaggtatttctt

SEQ ID NO: 895 - RNA, 21 nt, synthetic construct
  misc_feature 1..21: note = AAT RNAi agent antisense strand modified sequence
  source 1..21: mol_type = other RNA
tggaacttggtgatgatattt

SEQ ID NO: 896 - RNA, 21 nt, synthetic construct
  misc_feature 1..21: note = AAT RNAi agent antisense strand modified sequence
  source 1..21: mol_type = other RNA
tgatcataggttccagtaatt

SEQ ID NO: 897 - RNA, 21 nt, synthetic construct
  misc_feature 1..21: note = AAT RNAi agent antisense strand modified sequence
  source 1..21: mol_type = other RNA
tacagccttatgcacggcctt

SEQ ID NO: 898 - RNA, 21 nt, synthetic construct
  misc_feature 1..21: note = AAT RNAi agent antisense strand modified sequence
  source 1..21: mol_type = other RNA
ttcgatggtcagcacagcctt

SEQ ID NO: 899 - RNA, 21 nt, synthetic construct
  misc_feature 1..21: note = AAT RNAi agent antisense strand modified sequence
  source 1..21: mol_type = other RNA
tcaaagggtttgttgaacttt

SEQ ID NO: 900 - RNA, 21 nt, synthetic construct
  misc_feature 1..21: note = AAT RNAi agent antisense strand modified sequence
  source 1..21: mol_type = other RNA
tgttaaacatgcctaaacgtt

SEQ ID NO: 901 - RNA, 21 nt, synthetic construct
  misc_feature 1..21: note = AAT RNAi agent antisense strand modified sequence
  source 1..21: mol_type = other RNA
tttaaacgtgcctaaacgctg

SEQ ID NO: 902 - RNA, 21 nt, synthetic construct
  misc_feature 1..21: note = AAT RNAi agent antisense strand modified sequence
  source 1..21: mol_type = other RNA
tgcattgcccaggtatttcag

SEQ ID NO: 903 - RNA, 21 nt, synthetic construct
  misc_feature 1..21: note = AAT RNAi agent antisense strand modified sequence
  source 1..21: mol_type = other RNA
tggaacttggtgatgatatcg

SEQ ID NO: 904 - RNA, 21 nt, synthetic construct
  misc_feature 1..21: note = AAT RNAi agent antisense strand modified sequence
  source 1..21: mol_type = other RNA
tgatcataggttccagtaatg

SEQ ID NO: 905 - RNA, 21 nt, synthetic construct
  misc_feature 1..21: note = AAT RNAi agent antisense strand modified sequence
  source 1..21: mol_type = other RNA
tacagccttatgcacggcctt

SEQ ID NO: 906 - RNA, 21 nt, synthetic construct
  misc_feature 1..21: note = AAT RNAi agent antisense strand modified sequence
  source 1..21: mol_type = other RNA
ttcgatggtcagcacagcctt

SEQ ID NO: 907 - RNA, 21 nt, synthetic construct
  misc_feature 1..21: note = AAT RNAi agent antisense strand modified sequence
  source 1..21: mol_type = other RNA
tcaaagggtttgttgaacttg

SEQ ID NO: 908 - RNA, 21 nt, synthetic construct
  misc_feature 1..21: note = AAT RNAi agent antisense strand modified sequence
  source 1..21: mol_type = other RNA
tgttaaacatgcctaaacgcg

SEQ ID NO: 909 - RNA, 21 nt, synthetic construct
  misc_feature 1..21: note = AAT RNAi agent antisense strand modified sequence
  source 1..21: mol_type = other RNA
tttaaacgtgcctaaacgctg

SEQ ID NO: 910 - RNA, 21 nt, synthetic construct
  misc_feature 1..21: note = AAT RNAi agent antisense strand modified sequence
  source 1..21: mol_type = other RNA
tgttaaacatgcctaaacgtt

SEQ ID NO: 911 - RNA, 21 nt, synthetic construct
  misc_feature 1..21: note = AAT RNAi agent antisense strand modified sequence
  source 1..21: mol_type = other RNA
tgttaaacatgcctaaacgtt

SEQ ID NO: 912 - RNA, 21 nt, synthetic construct
  misc_feature 1..21: note = AAT RNAi agent antisense strand modified sequence
  source 1..21: mol_type = other RNA
tgttaaacatgcctaaacgct

SEQ ID NO: 913 - RNA, 21 nt, synthetic construct
  misc_feature 1..21: note = AAT RNAi agent antisense strand modified sequence
  source 1..21: mol_type = other RNA
tgttaaacatgcctaaacgtt

SEQ ID NO: 914 - RNA, 21 nt, synthetic construct
  misc_feature 1..21: note = AAT RNAi agent antisense strand modified sequence
  source 1..21: mol_type = other RNA
tgttaaacatgcctaaacgct

SEQ ID NO: 915 - RNA, 21 nt, synthetic construct
  misc_feature 1..21: note = AAT RNAi agent antisense strand modified sequence
  source 1..21: mol_type = other RNA
tgttaaacatgcctaaacgct

SEQ ID NO: 916 - RNA, 23 nt, synthetic construct
  misc_feature 1..23: note = AAT RNAi agent antisense strand modified sequence
  source 1..23: mol_type = other RNA
tgttaaacatgcctaaacgcttc

SEQ ID NO: 917 - RNA, 21 nt, synthetic construct
  misc_feature 1..21: note = AAT RNAi agent antisense strand modified sequence
  source 1..21: mol_type = other RNA
tgttaaacatgcctaaacgct

SEQ ID NO: 918 - RNA, 23 nt, synthetic construct
  misc_feature 1..23: note = AAT RNAi agent antisense strand modified sequence
  source 1..23: mol_type = other RNA
tgttaaacatgcctaaacgcttc

SEQ ID NO: 919 - RNA, 21 nt, synthetic construct
  misc_feature 1..21: note = AAT RNAi agent antisense strand modified sequence
  source 1..21: mol_type = other RNA
tgttaaacatgcctaaacgcg

SEQ ID NO: 920 - RNA, 21 nt, synthetic construct
  misc_feature 1..21: note = AAT RNAi agent antisense strand modified sequence
  source 1..21: mol_type = other RNA
tggaacttggtgatgatattt

SEQ ID NO: 921 - RNA, 21 nt, synthetic construct
  misc_feature 1..21: note = AAT RNAi agent antisense strand modified sequence
  source 1..21: mol_type = other RNA
tgctgttggactggtgtgctt

SEQ ID NO: 922 - RNA, 21 nt, synthetic construct
  misc_feature 1..21: note = AAT RNAi agent antisense strand modified sequence
  source 1..21: mol_type = other RNA
tgctgttggactggtgtgcca

SEQ ID NO: 923 - RNA, 23 nt, synthetic construct
  misc_feature 1..23: note = AAT RNAi agent antisense strand modified sequence
  source 1..23: mol_type = other RNA
tgctgttggactggtgtgccatt

SEQ ID NO: 924 - RNA, 23 nt, synthetic construct
  misc_feature 1..23: note = AAT RNAi agent antisense strand modified sequence
  source 1..23: mol_type = other RNA
tgctgttggactggtgtgccagc

SEQ ID NO: 925 - RNA, 21 nt, synthetic construct
  misc_feature 1..21: note = AAT RNAi agent antisense strand modified sequence
  source 1..21: mol_type = other RNA
taaggcttctgagtggtactt

SEQ ID NO: 926 - RNA, 21 nt, synthetic construct
  misc_feature 1..21: note = AAT RNAi agent antisense strand modified sequence
  source 1..21: mol_type = other RNA
taaggcttctgagtggtacaa

SEQ ID NO: 927 - RNA, 23 nt, synthetic construct
  misc_feature 1..23: note = AAT RNAi agent antisense strand modified sequence
  source 1..23: mol_type = other RNA
taaggcttctgagtggtacaact

SEQ ID NO: 928 - RNA, 21 nt, synthetic construct
  misc_feature 1..21: note = AAT RNAi agent antisense strand modified sequence
  source 1..21: mol_type = other RNA
gaaggcttctgagtggtactt

SEQ ID NO: 929 - RNA, 21 nt, synthetic construct
  misc_feature 1..21: note = AAT RNAi agent antisense strand modified sequence
  source 1..21: mol_type = other RNA
aagacaaagggtttgttgatt

SEQ ID NO: 930 - RNA, 21 nt, synthetic construct
  misc_feature 1..21: note = AAT RNAi agent antisense strand modified sequence
  source 1..21: mol_type = other RNA
aagacaaagggtttgttgaac

SEQ ID NO: 931 - RNA, 21 nt, synthetic construct
  misc_feature 1..21: note = AAT RNAi agent antisense strand modified sequence
  source 1..21: mol_type = other RNA
tagacaaagggtttgttgaac

SEQ ID NO: 932 - RNA, 23 nt, synthetic construct
  misc_feature 1..23: note = AAT RNAi agent antisense strand modified sequence
  source 1..23: mol_type = other RNA
aagacaaagggtttgttgaactt

SEQ ID NO: 933 - RNA, 21 nt, synthetic construct
  misc_feature 1..21: note = AAT RNAi agent antisense strand modified sequence
  source 1..21: mol_type = other RNA
tagacatgggtatggcctctt

SEQ ID NO: 934 - RNA, 21 nt, synthetic construct
  misc_feature 1..21: note = AAT RNAi agent antisense strand modified sequence
  source 1..21: mol_type = other RNA
tagacatgggtatggcctcta

SEQ ID NO: 935 - RNA, 23 nt, synthetic construct
  misc_feature 1..23: note = AAT RNAi agent antisense strand modified sequence
  source 1..23: mol_type = other RNA
tagacatgggtatggcctctaaa

SEQ ID NO: 936 - RNA, 23 nt, synthetic construct
  misc_feature 1..23: note = AAT RNAi agent antisense strand modified sequence
  source 1..23: mol_type = other RNA
tagacatgggtatggcctctatt

SEQ ID NO: 937 - RNA, 21 nt, synthetic construct
  misc_feature 1..21: note = AAT RNAi agent antisense strand modified sequence
  source 1..21: mol_type = other RNA
tttgatctgtttcttggcctt

SEQ ID NO: 938 - RNA, 21 nt, synthetic construct
  misc_feature 1..21: note = AAT RNAi agent antisense strand modified sequence
  source 1..21: mol_type = other RNA
tttgatctgtttcttggcctc

SEQ ID NO: 939 - RNA, 23 nt, synthetic construct
  misc_feature 1..23: note = AAT RNAi agent antisense strand modified sequence
  source 1..23: mol_type = other RNA
tttgatctgtttcttggcctctt

SEQ ID NO: 940 - RNA, 21 nt, synthetic construct
  misc_feature 1..21: note = AAT RNAi agent antisense strand modified sequence
  source 1..21: mol_type = other RNA
tgttggactggtgtgccagtt

SEQ ID NO: 941 - RNA, 21 nt, synthetic construct
  misc_feature 1..21: note = AAT RNAi agent antisense strand modified sequence
  source 1..21: mol_type = other RNA
tgttggactggtgtgccagct

SEQ ID NO: 942 - RNA, 23 nt, synthetic construct
  misc_feature 1..23: note = AAT RNAi agent antisense strand modified sequence
  source 1..23: mol_type = other RNA
tgttggactggtgtgccagctgg

SEQ ID NO: 943 - RNA, 22 nt, synthetic construct
  misc_feature 1..22: note = AAT RNAi agent antisense strand modified sequence
  source 1..22: mol_type = other RNA
tgttggactggtgtgccagctg

SEQ ID NO: 944 - RNA, 21 nt, synthetic construct
  misc_feature 1..21: note = AAT RNAi agent antisense strand modified sequence
  source 1..21: mol_type = other RNA
aaagggtttgttgaacttgtt

SEQ ID NO: 945 - RNA, 21 nt, synthetic construct
  misc_feature 1..21: note = AAT RNAi agent antisense strand modified sequence
  source 1..21: mol_type = other RNA
aaagggtttgttgaacttgac

SEQ ID NO: 946 - RNA, 23 nt, synthetic construct
  misc_feature 1..23: note = AAT RNAi agent antisense strand modified sequence
  source 1..23: mol_type = other RNA
taagggtttgttgaacttgacct

SEQ ID NO: 947 - RNA, 21 nt, synthetic construct
  misc_feature 1..21: note = AAT RNAi agent antisense strand modified sequence
  source 1..21: mol_type = other RNA
taagggtttgttgaacttgac

SEQ ID NO: 948 - RNA, 21 nt, synthetic construct
  misc_feature 1..21: note = AAT RNAi agent antisense strand modified sequence
  source 1..21: mol_type = other RNA
tattggtgctgttggactgtt

SEQ ID NO: 949 - RNA, 21 nt, synthetic construct
  misc_feature 1..21: note = AAT RNAi agent antisense strand modified sequence
  source 1..21: mol_type = other RNA
tattggtgctgttggactggt

SEQ ID NO: 950 - RNA, 22 nt, synthetic construct
  misc_feature 1..22: note = AAT RNAi agent antisense strand modified sequence
  source 1..22: mol_type = other RNA
tattggtgctgttggactggtt

SEQ ID NO: 951 - RNA, 20 nt, synthetic construct
  misc_feature 1..20: note = AAT RNAi agent antisense strand modified sequence
  source 1..20: mol_type = other RNA
ttgttggactggtgtgccag

SEQ ID NO: 952 - RNA, 22 nt, synthetic construct
  misc_feature 1..22: note = AAT RNAi agent antisense strand modified sequence
  source 1..22: mol_type = other RNA
ttgttggactggtgtgccagct

SEQ ID NO: 953 - RNA, 21 nt, synthetic construct
  misc_feature 1..21: note = AAT RNAi agent antisense strand modified sequence
  source 1..21: mol_type = other RNA
tatagacatgggtatggcctc

SEQ ID NO: 954 - RNA, 21 nt, synthetic construct
  misc_feature 1..21: note = AAT RNAi agent antisense strand modified sequence
  source 1..21: mol_type = other RNA
tatagacatgggtatggcctc

SEQ ID NO: 955 - RNA, 23 nt, synthetic construct
  misc_feature 1..23: note = AAT RNAi agent antisense strand modified sequence
  source 1..23: mol_type = other RNA
tcaaagggtttgttgaacttgac

SEQ ID NO: 956 - RNA, 21 nt, synthetic construct
  misc_feature 1..21: note = AAT RNAi agent antisense strand modified sequence
  source 1..21: mol_type = other RNA
ttattggtgctgttggactgg

SEQ ID NO: 957 - RNA, 20 nt, synthetic construct
  misc_feature 1..20: note = AAT RNAi agent antisense strand modified sequence
  source 1..20: mol_type = other RNA
tgttaaacatgcctaaacgc

SEQ ID NO: 958 - RNA, 22 nt, synthetic construct
  misc_feature 1..22: note = AAT RNAi agent antisense strand modified sequence
  source 1..22: mol_type = other RNA
tgttaaacatgcctaaacgctt

SEQ ID NO: 959 - RNA, 21 nt, synthetic construct
  misc_feature 1..21: note = AAT RNAi agent antisense strand modified sequence
  source 1..21: mol_type = other RNA
tgttaaacatgcctaaacgcg

SEQ ID NO: 960 - RNA, 21 nt, synthetic construct
  misc_feature 1..21: note = AAT RNAi agent antisense strand modified sequence
  source 1..21: mol_type = other RNA
tgttaaacatgcctaaacgct

SEQ ID NO: 961 - RNA, 21 nt, synthetic construct
  misc_feature 1..21: note = AAT RNAi agent antisense strand modified sequence
  source 1..21: mol_type = other RNA
tgttaaacatgcctaaacgcg

SEQ ID NO: 962 - RNA, 21 nt, synthetic construct
  misc_feature 1..21: note = AAT RNAi agent antisense strand modified sequence
  source 1..21: mol_type = other RNA
tgttaaacatgcctaaacgtt

SEQ ID NO: 963 - RNA, 21 nt, synthetic construct
  misc_feature 1..21: note = AAT RNAi agent antisense strand modified sequence
  source 1..21: mol_type = other RNA
tgttaaacatgcctaaacgtt

SEQ ID NO: 964 - DNA, 23 bp, synthetic construct
  misc_feature 1..23: note = AAT RNAi agent sense strand modified sequence
  source 1..23: mol_type = other DNA
  modified_base 1: mod_base = OTHER; note = uracil
  modified_base 3: mod_base = OTHER; note = uracil
  modified_base 5: mod_base = OTHER; note = uracil
  modified_base 7: mod_base = OTHER; note = uracil
  modified_base 10: mod_base = OTHER; note = uracil
  modified_base 18..19: mod_base = OTHER; note = uracil
tatatatcatcaccaagttccat

SEQ ID NO: 965 - DNA, 23 bp, synthetic construct
  misc_feature 1..23: note = AAT RNAi agent sense strand modified sequence
  source 1..23: mol_type = other DNA
  modified_base 1: mod_base = OTHER; note = uracil
  modified_base 3: mod_base = OTHER; note = uracil
  modified_base 5: mod_base = OTHER; note = uracil
  modified_base 7: mod_base = OTHER; note = uracil
  modified_base 10: mod_base = OTHER; note = uracil
  modified_base 18..19: mod_base = OTHER; note = uracil
tatatatcatcaccaagttccat

SEQ ID NO: 966 - DNA, 20 bp, synthetic construct
  misc_feature 1..20: note = AAT RNAi agent sense strand modified sequence
  source 1..20: mol_type = other DNA
  modified_base 2: mod_base = OTHER; note = uracil
  modified_base 4: mod_base = OTHER; note = uracil
  modified_base 7: mod_base = OTHER; note = uracil
  modified_base 15..16: mod_base = OTHER; note = uracil
atatcatcaccaagttccat

SEQ ID NO: 967 - RNA, 21 nt, synthetic construct
  misc_feature 1..21: note = AAT RNAi agent sense strand modified sequence
  source 1..21: mol_type = other RNA
cgatatcatcaccaagttcca

SEQ ID NO: 968 - RNA, 21 nt, synthetic construct
  misc_feature 1..21: note = AAT RNAi agent sense strand modified sequence
  source 1..21: mol_type = other RNA
cgatatcatcaccaagttcca

SEQ ID NO: 969 - DNA, 19 bp, synthetic construct
  misc_feature 1..19: note = AAT RNAi agent sense strand modified sequence
  source 1..19: mol_type = other DNA
  modified_base 1: mod_base = OTHER; note = uracil
  modified_base 3: mod_base = OTHER; note = uracil
  modified_base 6: mod_base = OTHER; note = uracil
  modified_base 14..15: mod_base = OTHER; note = uracil
tatcatcaccaagttccat

SEQ ID NO: 970 - DNA, 23 bp, synthetic construct
  misc_feature 1..23: note = AAT RNAi agent sense strand modified sequence
  source 1..23: mol_type = other DNA
tatatatcatcaccaagttccat

SEQ ID NO: 971 - DNA, 23 bp, synthetic construct
  misc_feature 1..23: note = AAT RNAi agent sense strand modified sequence
  source 1..23: mol_type = other DNA
tatatatcatcaccaagttccat

SEQ ID NO: 972 - RNA, 21 nt, synthetic construct
  misc_feature 1..21: note = AAT RNAi agent sense strand modified sequence
  source 1..21: mol_type = other RNA
gcgtttaggcatgtttaaatt

SEQ ID NO: 973 - RNA, 21 nt, synthetic construct
  misc_feature 1..21: note = AAT RNAi agent sense strand modified sequence
  source 1..21: mol_type = other RNA
gaaatacctgggcaatgcatt

SEQ ID NO: 974 - RNA, 21 nt, synthetic construct
  misc_feature 1..21: note = AAT RNAi agent sense strand modified sequence
  source 1..21: mol_type = other RNA
atatcatcaccaagttccatt

SEQ ID NO: 975 - RNA, 21 nt, synthetic construct
  misc_feature 1..21: note = AAT RNAi agent sense strand modified sequence
  source 1..21: mol_type = other RNA
ttactggaacctatgatcatt

SEQ ID NO: 976 - RNA, 21 nt, synthetic construct
  misc_feature 1..21: note = AAT RNAi agent sense strand modified sequence
  source 1..21: mol_type = other RNA
ggccgtgcataaggctgtatt

SEQ ID NO: 977 - RNA, 21 nt, synthetic construct
  misc_feature 1..21: note = AAT RNAi agent sense strand modified sequence
  source 1..21: mol_type = other RNA
ggctgtgctgaccatcgaatt

SEQ ID NO: 978 - RNA, 21 nt, synthetic construct
  misc_feature 1..21: note = AAT RNAi agent sense strand modified sequence
  source 1..21: mol_type = other RNA
agttcaacaaaccctttgatt

SEQ ID NO: 979 - RNA, 21 nt, synthetic construct
  misc_feature 1..21: note = AAT RNAi agent sense strand modified sequence
  source 1..21: mol_type = other RNA
cgtttaggcatgtttaacatt

SEQ ID NO: 980 - RNA, 21 nt, synthetic construct
  misc_feature 1..21: note = AAT RNAi agent sense strand modified sequence
  source 1..21: mol_type = other RNA
cagcgtttaggcatgtttaaa

SEQ ID NO: 981 - RNA, 21 nt, synthetic construct
  misc_feature 1..21: note = AAT RNAi agent sense strand modified sequence
  source 1..21: mol_type = other RNA
ctgaaatacctgggcaatgca

SEQ ID NO: 982 - RNA, 21 nt, synthetic construct
  misc_feature 1..21: note = AAT RNAi agent sense strand modified sequence
  source 1..21: mol_type = other RNA
cgatatcatcaccaagttcca

SEQ ID NO: 983 - RNA, 21 nt, synthetic construct
  misc_feature 1..21: note = AAT RNAi agent sense strand modified sequence
  source 1..21: mol_type = other RNA
cattactggaacctatgatca

SEQ ID NO: 984 - RNA, 21 nt, synthetic construct
  misc_feature 1..21: note = AAT RNAi agent sense strand modified sequence
  source 1..21: mol_type = other RNA
aaggccgtgcataaggctgta

SEQ ID NO: 985 - RNA, 21 nt, synthetic construct
  misc_feature 1..21: note = AAT RNAi agent sense strand modified sequence
  source 1..21: mol_type = other RNA
aaggctgtgctgaccatcgaa

SEQ ID NO: 986 - RNA, 21 nt, synthetic construct
  misc_feature 1..21: note = AAT RNAi agent sense strand modified sequence
  source 1..21: mol_type = other RNA
caagttcaacaaaccctttga

SEQ ID NO: 987 - RNA, 21 nt, synthetic construct
  misc_feature 1..21: note = AAT RNAi agent sense strand modified sequence
  source 1..21: mol_type = other RNA
cgcgtttaggcatgtttaaca

SEQ ID NO: 988 - RNA, 21 nt, synthetic construct
  misc_feature 1..21: note = AAT RNAi agent sense strand modified sequence
  source 1..21: mol_type = other RNA
cagcgtttaggcatgtttaaa

SEQ ID NO: 989 - RNA, 21 nt, synthetic construct
  misc_feature 1..21: note = AAT RNAi agent sense strand modified sequence
  source 1..21: mol_type = other RNA
cgtttaggcatgtttaacatt

SEQ ID NO: 990 - RNA, 19 nt, synthetic construct
  misc_feature 1..19: note = AAT RNAi agent sense strand modified sequence
  source 1..19: mol_type = other RNA
cgtttaggcatgtttaaca

SEQ ID NO: 991 - RNA, 19 nt, synthetic construct
  misc_feature 1..19: note = AAT RNAi agent sense strand modified sequence
  source 1..19: mol_type = other RNA
cgtttaggcatgtttaaca

SEQ ID NO: 992 - RNA, 21 nt, synthetic construct
  misc_feature 1..21: note = AAT RNAi agent sense strand modified sequence
  source 1..21: mol_type = other RNA
aacgtttaggcatgtttaaca

SEQ ID NO: 993 - RNA, 21 nt, synthetic construct
  misc_feature 1..21: note = AAT RNAi agent sense strand modified sequence
  source 1..21: mol_type = other RNA
agcgtttaggcatgtttaaca

SEQ ID NO: 994 - RNA, 21 nt, synthetic construct
  misc_feature 1..21: note = AAT RNAi agent sense strand modified sequence
  source 1..21: mol_type = other RNA
aacgtttaggcatgtttaaca

SEQ ID NO: 995 - RNA, 21 nt, synthetic construct
  misc_feature 1..21: note = AAT RNAi agent sense strand modified sequence
  source 1..21: mol_type = other RNA
agcgtttaggcatgtttaaca

SEQ ID NO: 996 - RNA, 19 nt, synthetic construct
  misc_feature 1..19: note = AAT RNAi agent sense strand modified sequence
  source 1..19: mol_type = other RNA
gcacaccagtccaacagca

SEQ ID NO: 997 - RNA, 21 nt, synthetic construct
  misc_feature 1..21: note = AAT RNAi agent sense strand modified sequence
  source 1..21: mol_type = other RNA
tggcacaccagtccaacagca

SEQ ID NO: 998 - RNA, 21 nt, synthetic construct
  misc_feature 1..21: note = AAT RNAi agent sense strand modified sequence
  source 1..21: mol_type = other RNA
aagcacaccagtccaacagca

SEQ ID NO: 999 - RNA, 19 nt, synthetic construct
  misc_feature 1..19: note = AAT RNAi agent sense strand modified sequence
  source 1..19: mol_type = other RNA
gtaccactcagaagcctta

SEQ ID NO: 1000 - RNA, 21 nt, synthetic construct
  misc_feature 1..21: note = AAT RNAi agent sense strand modified sequence
  source 1..21: mol_type = other RNA
ttgtaccactcagaagcctta

SEQ ID NO: 1001 - RNA, 19 nt, synthetic construct
  misc_feature 1..19: note = AAT RNAi agent sense strand modified sequence
  source 1..19: mol_type = other RNA
gtaccactcagaagccttc

SEQ ID NO: 1002 - RNA, 19 nt, synthetic construct
  misc_feature 1..19: note = AAT RNAi agent sense strand modified sequence
  source 1..19: mol_type = other RNA
tcaacaaaccctttgtctt

SEQ ID NO: 1003 - RNA, 21 nt, synthetic construct
  misc_feature 1..21: note = AAT RNAi agent sense strand modified sequence
  source 1..21: mol_type = other RNA
gttcaacaaaccctttgtctt

SEQ ID NO: 1004 - RNA, 21 nt, synthetic construct
  misc_feature 1..21: note = AAT RNAi agent sense strand modified sequence
  source 1..21: mol_type = other RNA
gttcaacaaaccctttgtcta

SEQ ID NO: 1005 - RNA, 19 nt, synthetic construct
  misc_feature 1..19: note = AAT RNAi agent sense strand modified sequence
  source 1..19: mol_type = other RNA
gaggccatacccatgtcta

SEQ ID NO: 1006 - RNA, 21 nt, synthetic construct
  misc_feature 1..21: note = AAT RNAi agent sense strand modified sequence
  source 1..21: mol_type = other RNA
tagaggccatacccatgtcta

SEQ ID NO: 1007 - RNA, 19 nt, synthetic construct
  misc_feature 1..19: note = AAT RNAi agent sense strand modified sequence
  source 1..19: mol_type = other RNA
ggccaagaaacagatcaaa

SEQ ID NO: 1008 - RNA, 21 nt, synthetic construct
  misc_feature 1..21: note = AAT RNAi agent sense strand modified sequence
  source 1..21: mol_type = other RNA
gaggccaagaaacagatcaaa

SEQ ID NO: 1009 - RNA, 19 nt, synthetic construct
  misc_feature 1..19: note = AAT RNAi agent sense strand modified sequence
  source 1..19: mol_type = other RNA
ctggcacaccagtccaaca

SEQ ID NO: 1010 - RNA, 21 nt, synthetic construct
  misc_feature 1..21: note = AAT RNAi agent sense strand modified sequence
  source 1..21: mol_type = other RNA
agctggcacaccagtccaaca

SEQ ID NO: 1011 - RNA, 20 nt, synthetic construct
  misc_feature 1..20: note = AAT RNAi agent sense strand modified sequence
  source 1..20: mol_type = other RNA
gctggcacaccagtccaaca

SEQ ID NO: 1012 - RNA, 19 nt, synthetic construct
  misc_feature 1..19: note = AAT RNAi agent sense strand modified sequence
  source 1..19: mol_type = other RNA
caagttcaacaaacccttt

SEQ ID NO: 1013 - RNA, 21 nt, synthetic construct
  misc_feature 1..21: note = AAT RNAi agent sense strand modified sequence
  source 1..21: mol_type = other RNA
gtcaagttcaacaaacccttt

SEQ ID NO: 1014 - RNA, 21 nt, synthetic construct
  misc_feature 1..21: note = AAT RNAi agent sense strand modified sequence
  source 1..21: mol_type = other RNA
gtcaagttcaacaaaccctta

SEQ ID NO: 1015 - RNA, 19 nt, synthetic construct
  misc_feature 1..19: note = AAT RNAi agent sense strand modified sequence
  source 1..19: mol_type = other RNA
cagtccaacagcaccaata

SEQ ID NO: 1016 - RNA, 21 nt, synthetic construct
  misc_feature 1..21: note = AAT RNAi agent sense strand modified sequence
  source 1..21: mol_type = other RNA
accagtccaacagcaccaata

SEQ ID NO: 1017 - RNA, 20 nt, synthetic construct
  misc_feature 1..20: note = AAT RNAi agent sense strand modified sequence
  source 1..20: mol_type = other RNA
ccagtccaacagcaccaata

SEQ ID NO: 1018 - RNA, 20 nt, synthetic construct
  misc_feature 1..20: note = AAT RNAi agent sense strand modified sequence
  source 1..20: mol_type = other RNA
ctggcacaccagtccaacaa

SEQ ID NO: 1019 - RNA, 19 nt, synthetic construct
  misc_feature 1..19: note = AAT RNAi agent sense strand modified sequence
  source 1..19: mol_type = other RNA
ggccatacccatgtctata

SEQ ID NO: 1020 - RNA, 21 nt, synthetic construct
  misc_feature 1..21: note = AAT RNAi agent sense strand modified sequence
  source 1..21: mol_type = other RNA
gaggccatacccatgtctata

SEQ ID NO: 1021 - RNA, 23 nt, synthetic construct
  misc_feature 1..23: note = AAT RNAi agent sense strand modified sequence
  source 1..23: mol_type = other RNA
gtcaagttcaacaaaccctttga

SEQ ID NO: 1022 - RNA, 21 nt, synthetic construct
  misc_feature 1..21: note = AAT RNAi agent sense strand modified sequence
  source 1..21: mol_type = other RNA
caagttcaacaaaccctttga

SEQ ID NO: 1023 - RNA, 21 nt, synthetic construct
  misc_feature 1..21: note = AAT RNAi agent sense strand modified sequence
  source 1..21: mol_type = other RNA
ccagtccaacagcaccaataa

SEQ ID NO: 1024 - RNA, 21 nt, synthetic construct
  misc_feature 1..21: note = AAT RNAi agent sense strand modified sequence
  source 1..21: mol_type = other RNA
cgcgtttaggcatgtttaaca

SEQ ID NO: 1025 - RNA, 19 nt, synthetic construct
  misc_feature 1..19: note = AAT RNAi agent sense strand modified sequence
  source 1..19: mol_type = other RNA
cgtttaggcatgtttaaca

SEQ ID NO: 1026 - RNA, 21 nt, synthetic construct
  misc_feature 1..21: note = AAT RNAi agent sense strand modified sequence
  source 1..21: mol_type = other RNA
cgcgtttaggcatgtttaaca

SEQ ID NO: 1027 - RNA, 21 nt, synthetic construct
  misc_feature 1..21: note = AAT RNAi agent sense strand modified sequence
  source 1..21: mol_type = other RNA
agcgtttaggcatgtttaaca

SEQ ID NO: 1028 - RNA, 21 nt, synthetic construct
  misc_feature 1..21: note = AAT RNAi agent sense strand modified sequence
  source 1..21: mol_type = other RNA
cgtttaggcatgtttaacatt

SEQ ID NO: 1029 - RNA, 20 nt, synthetic construct
  misc_feature 1..20: note = AAT RNAi agent sense strand modified sequence
  source 1..20: mol_type = other RNA
gcgtttaggcatgtttaaca

SEQ ID NO: 1030 - RNA, 22 nt, synthetic construct
  misc_feature 1..22: note = AAT RNAi agent sense strand modified sequence
  source 1..22: mol_type = other RNA
gcgtttaggcatgtttaacatt

SEQ ID NO: 1031 - RNA, 23 nt, synthetic construct
  misc_feature 1..23: note = AAT RNAi agent sense strand modified sequence
  source 1..23: mol_type = other RNA
cgcgtttaggcatgtttaacatt

SEQ ID NO: 1032 - RNA, 23 nt, synthetic construct
  misc_feature 1..23: note = AAT RNAi agent sense strand modified sequence
  source 1..23: mol_type = other RNA
agcgtttaggcatgtttaacatt

SEQ ID NO: 1033 - RNA, 21 nt, synthetic construct
  misc_feature 1..21: note = AAT RNAi agent sense strand modified sequence
  source 1..21: mol_type = other RNA
agcgtttaggcatgtttaaca

SEQ ID NO: 1034 - RNA, 21 nt, synthetic construct
  misc_feature 1..21: note = AAT RNAi agent sense strand modified sequence
  source 1..21: mol_type = other RNA
cgcgtttaggcatgtttaaca

SEQ ID NO: 1035 - RNA, 21 nt, synthetic construct
  misc_feature 1..21: note = AAT RNAi agent antisense sequence
  source 1..21: mol_type = other RNA
agaagatattggtgctgtttt

SEQ ID NO: 1036 - RNA, 21 nt, synthetic construct
  misc_feature 1..21: note = AAT RNAi agent antisense sequence
  source 1..21: mol_type = other RNA
aggaacttggtgatgatattt

SEQ ID NO: 1037 - RNA, 21 nt, synthetic construct
  misc_feature 1..21: note = AAT RNAi agent antisense sequence
  source 1..21: mol_type = other RNA
tgtcttctgggcagcatcttt

SEQ ID NO: 1038 - RNA, 21 nt, synthetic construct
  misc_feature 1..21: note = AAT RNAi agent antisense sequence
  source 1..21: mol_type = other RNA
tgttggactggtgtgccagtt

SEQ ID NO: 1039 - RNA, 21 nt, synthetic construct
  misc_feature 1..21: note = AAT RNAi agent antisense sequence
  source 1..21: mol_type = other RNA
ctgttggactggtgtgccatt

SEQ ID NO: 1040 - RNA, 21 nt, synthetic construct
  misc_feature 1..21: note = AAT RNAi agent antisense sequence
  source 1..21: mol_type = other RNA
tgctgttggactggtgtgctt

SEQ ID NO: 1041 - RNA, 21 nt, synthetic construct
  misc_feature 1..21: note = AAT RNAi agent antisense sequence
  source 1..21: mol_type = other RNA
tattggtgctgttggactgtt

SEQ ID NO: 1042 - RNA, 21 nt, synthetic construct
  misc_feature 1..21: note = AAT RNAi agent antisense sequence
  source 1..21: mol_type = other RNA
atattggtgctgttggacttt

SEQ ID NO: 1043 - RNA, 21 nt, synthetic construct
  misc_feature 1..21: note = AAT RNAi agent antisense sequence
  source 1..21: mol_type = other RNA
gatattggtgctgttggactt

SEQ ID NO: 1044 - RNA, 21 nt, synthetic construct
  misc_feature 1..21: note = AAT RNAi agent antisense sequence
  source 1..21: mol_type = other RNA
aagatattggtgctgttggtt

SEQ ID NO: 1045 - RNA, 21 nt, synthetic construct
  misc_feature 1..21: note = AAT RNAi agent antisense sequence
  source 1..21: mol_type = other RNA
gtagcgatgctcactggggtt

SEQ ID NO: 1046 - RNA, 21 nt, synthetic construct
  misc_feature 1..21: note = AAT RNAi agent antisense sequence
  source 1..21: mol_type = other RNA
aaaggctgtagcgatgctctt

SEQ ID NO: 1047 - RNA, 21 nt, synthetic construct
  misc_feature 1..21: note = AAT RNAi agent antisense sequence
  source 1..21: mol_type = other RNA
gcaaaggctgtagcgatgctt

SEQ ID NO: 1048 - RNA, 21 nt, synthetic construct
  misc_feature 1..21: note = AAT RNAi agent antisense sequence
  source 1..21: mol_type = other RNA
tgcaaaggctgtagcgatgtt

SEQ ID NO: 1049 - RNA, 21 nt, synthetic construct
  misc_feature 1..21: note = AAT RNAi agent antisense sequence
  source 1..21: mol_type = other RNA
attgcaaaggctgtagcgatt

SEQ ID NO: 1050 - RNA, 21 nt, synthetic construct
  misc_feature 1..21: note = AAT RNAi agent antisense sequence
  source 1..21: mol_type = other RNA
agcattgcaaaggctgtagtt

SEQ ID NO: 1051 - RNA, 21 nt, synthetic construct
  misc_feature 1..21: note = AAT RNAi agent antisense sequence
  source 1..21: mol_type = other RNA
agagcattgcaaaggctgttt

SEQ ID NO: 1052 - RNA, 21 nt, synthetic construct
  misc_feature 1..21: note = AAT RNAi agent antisense sequence
  source 1..21: mol_type = other RNA
ggagttcctggaagccttctt

SEQ ID NO: 1053 - RNA, 21 nt, synthetic construct
  misc_feature 1..21: note = AAT RNAi agent antisense sequence
  source 1..21: mol_type = other RNA
tccaaaaacttatccactatt

SEQ ID NO: 1054 - RNA, 21 nt, synthetic construct
  misc_feature 1..21: note = AAT RNAi agent antisense sequence
  source 1..21: mol_type = other RNA
aaggcttctgagtggtacatt

SEQ ID NO: 1055 - RNA, 21 nt, synthetic construct
  misc_feature 1..21: note = AAT RNAi agent antisense sequence
  source 1..21: mol_type = other RNA
gaaggcttctgagtggtactt

SEQ ID NO: 1056 - RNA, 21 nt, synthetic construct
  misc_feature 1..21: note = AAT RNAi agent antisense sequence
  source 1..21: mol_type = other RNA
ttcttggcctcttcggtgttt

SEQ ID NO: 1057 - RNA, 21 nt, synthetic construct
  misc_feature 1..21: note = AAT RNAi agent antisense sequence
  source 1..21: mol_type = other RNA
gtttcttggcctcttcggttt

SEQ ID NO: 1058 - RNA, 21 nt, synthetic construct
  misc_feature 1..21: note = AAT RNAi agent antisense sequence
  source 1..21: mol_type = other RNA
ttgatctgtttcttggccttt

SEQ ID NO: 1059 - RNA, 21 nt, synthetic construct
  misc_feature 1..21: note = AAT RNAi agent antisense sequence
  source 1..21: mol_type = other RNA
gttgatctgtttcttggcctt

SEQ ID NO: 1060 - RNA, 21 nt, synthetic construct
  misc_feature 1..21: note = AAT RNAi agent antisense sequence
  source 1..21: mol_type = other RNA
cgttgatctgtttcttggctt

SEQ ID NO: 1061 - RNA, 21 nt, synthetic construct
  misc_feature 1..21: note = AAT RNAi agent antisense sequence
  source 1..21: mol_type = other RNA
cacaattttcccttgagtatt

SEQ ID NO: 1062 - RNA, 21 nt, synthetic construct
  misc_feature 1..21: note = AAT RNAi agent antisense sequence
  source 1..21: mol_type = other RNA
tccacaattttcccttgagtt

SEQ ID NO: 1063 - RNA, 21 nt, synthetic construct
  misc_feature 1..21: note = AAT RNAi agent antisense sequence
  source 1..21: mol_type = other RNA
atccacaattttcccttgatt

SEQ ID NO: 1064 - RNA, 21 nt, synthetic construct
  misc_feature 1..21: note = AAT RNAi agent antisense sequence
  source 1..21: mol_type = other RNA
tgtcaagctccttgaccaatt

SEQ ID NO: 1065 - RNA, 21 nt, synthetic construct
  misc_feature 1..21: note = AAT RNAi agent antisense sequence
  source 1..21: mol_type = other RNA
gtgtctctgtcaagctccttt

SEQ ID NO: 1066 - RNA, 21 nt, synthetic construct
  misc_feature 1..21: note = AAT RNAi agent antisense sequence
  source 1..21: mol_type = other RNA
actgtgtctctgtcaagcttt

SEQ ID NO: 1067 - RNA, 21 nt, synthetic construct
  misc_feature 1..21: note = AAT RNAi agent antisense sequence
  source 1..21: mol_type = other RNA
tgtaattcaccagagcaaatt

SEQ ID NO: 1068 - RNA, 21 nt, synthetic construct
  misc_feature 1..21: note = AAT RNAi agent antisense sequence
  source 1..21: mol_type = other RNA
ttaaacatgcctaaacgcttt

SEQ ID NO: 1069 - RNA, 21 nt, synthetic construct
  misc_feature 1..21: note = AAT RNAi agent antisense sequence
  source 1..21: mol_type = other RNA
gttaaacatgcctaaacgctt

SEQ ID NO: 1070 - RNA, 21 nt, synthetic construct
  misc_feature 1..21: note = AAT RNAi agent antisense sequence
  source 1..21: mol_type = other RNA
tgttaaacatgcctaaacgtt

SEQ ID NO: 1071 - RNA, 21 nt, synthetic construct
  misc_feature 1..21: note = AAT RNAi agent antisense sequence
  source 1..21: mol_type = other RNA
ggatgttaaacatgcctaatt

SEQ ID NO: 1072 - RNA, 21 nt, synthetic construct
  misc_feature 1..21: note = AAT RNAi agent antisense sequence
  source 1..21: mol_type = other RNA
atttcatcagcagcacccatt

SEQ ID NO: 1073 - RNA, 21 nt, synthetic construct
  misc_feature 1..21: note = AAT RNAi agent antisense sequence
  source 1..21: mol_type = other RNA
gaagaagatggcggtggcatt

SEQ ID NO: 1074 - RNA, 21 nt, synthetic construct
  misc_feature 1..21: note = AAT RNAi agent antisense sequence
  source 1..21: mol_type = other RNA
ggtgagttcattttccaggtt

SEQ ID NO: 1075 - RNA, 21 nt, synthetic construct
  misc_feature 1..21: note = AAT RNAi agent antisense sequence
  source 1..21: mol_type = other RNA
gaacttggtgatgatatcgtt

SEQ ID NO: 1076 - RNA, 21 nt, synthetic construct
  misc_feature 1..21: note = AAT RNAi agent antisense sequence
  source 1..21: mol_type = other RNA
cattttccaggaacttggttt

SEQ ID NO: 1077 - RNA, 21 nt, synthetic construct
  misc_feature 1..21: note = AAT RNAi agent antisense sequence
  source 1..21: mol_type = other RNA
cataggttccagtaatggatt

SEQ ID NO: 1078 - RNA, 21 nt, synthetic construct
  misc_feature 1..21: note = AAT RNAi agent antisense sequence
  source 1..21: mol_type = other RNA
tcataggttccagtaatggtt

SEQ ID NO: 1079 - RNA, 21 nt, synthetic construct
  misc_feature 1..21: note = AAT RNAi agent antisense sequence
  source 1..21: mol_type = other RNA
tcagatcataggttccagttt

SEQ ID NO: 1080 - RNA, 21 nt, synthetic construct
  misc_feature 1..21: note = AAT RNAi agent antisense sequence
  source 1..21: mol_type = other RNA
tcttcagatcataggttcctt

SEQ ID NO: 1081 - RNA, 21 nt, synthetic construct
  misc_feature 1..21: note = AAT RNAi agent antisense sequence
  source 1..21: mol_type = other RNA
ctcttcagatcataggttctt

SEQ ID NO: 1082 - RNA, 21 nt, synthetic construct
  misc_feature 1..21: note = AAT RNAi agent antisense sequence
  source 1..21: mol_type = other RNA
gaggtcagccccattgctgtt

SEQ ID NO: 1083 - RNA, 21 nt, synthetic construct
  misc_feature 1..21: note = AAT RNAi agent antisense sequence
  source 1..21: mol_type = other RNA
gagaggtcagccccattgctt

SEQ ID NO: 1084 - RNA, 21 nt, synthetic construct
  misc_feature 1..21: note = AAT RNAi agent antisense sequence
  source 1..21: mol_type = other RNA
cttcaggggtgcctcctcttt

SEQ ID NO: 1085 - RNA, 21 nt, synthetic construct
  misc_feature 1..21: note = AAT RNAi agent antisense sequence
  source 1..21: mol_type = other RNA
gagagcttcaggggtgccttt

SEQ ID NO: 1086 - RNA, 21 nt, synthetic construct
  misc_feature 1..21: note = AAT RNAi agent antisense sequence
  source 1..21: mol_type = other RNA
ttatgcacggccttggagatt

SEQ ID NO: 1087 - RNA, 21 nt, synthetic construct
  misc_feature 1..21: note = AAT RNAi agent antisense sequence
  source 1..21: mol_type = other RNA
ccttatgcacggccttggatt

SEQ ID NO: 1088 - RNA, 21 nt, synthetic construct
  misc_feature 1..21: note = AAT RNAi agent antisense sequence
  source 1..21: mol_type = other RNA
gccttatgcacggccttggtt

SEQ ID NO: 1089 - RNA, 21 nt, synthetic construct
  misc_feature 1..21: note = AAT RNAi agent antisense sequence
  source 1..21: mol_type = other RNA
agccttatgcacggccttgtt

SEQ ID NO: 1090 - RNA, 21 nt, synthetic construct
  misc_feature 1..21: note = AAT RNAi agent antisense sequence
  source 1..21: mol_type = other RNA
cgatggtcagcacagcctttt

SEQ ID NO: 1091 - RNA, 21 nt, synthetic construct
  misc_feature 1..21: note = AAT RNAi agent antisense sequence
  source 1..21: mol_type = other RNA
gtcgatggtcagcacagcctt

SEQ ID NO: 1092 - RNA, 21 nt, synthetic construct
  misc_feature 1..21: note = AAT RNAi agent antisense sequence
  source 1..21: mol_type = other RNA
aaaaacatggccccagcagtt

SEQ ID NO: 1093 - RNA, 21 nt, synthetic construct
  misc_feature 1..21: note = AAT RNAi agent antisense sequence
  source 1..21: mol_type = other RNA
ctaaaaacatggccccagctt

SEQ ID NO: 1094 - RNA, 21 nt, synthetic construct
  misc_feature 1..21: note = AAT RNAi agent antisense sequence
  source 1..21: mol_type = other RNA
tctaaaaacatggccccagtt

SEQ ID NO: 1095 - RNA, 21 nt, synthetic construct
  misc_feature 1..21: note = AAT RNAi agent antisense sequence
  source 1..21: mol_type = other RNA
cctctaaaaacatggcccctt

SEQ ID NO: 1096 - RNA, 21 nt, synthetic construct
  misc_feature 1..21: note = AAT RNAi agent antisense sequence
  source 1..21: mol_type = other RNA
gcctctaaaaacatggccctt

SEQ ID NO: 1097 - RNA, 21 nt, synthetic construct
  misc_feature 1..21: note = AAT RNAi agent antisense sequence
  source 1..21: mol_type = other RNA
tagacatgggtatggcctctt

SEQ ID NO: 1098 - RNA, 21 nt, synthetic construct
  misc_feature 1..21: note = AAT RNAi agent antisense sequence
  source 1..21: mol_type = other RNA
gatagacatgggtatggcctt

SEQ ID NO: 1099 - RNA, 21 nt, synthetic construct
  misc_feature 1..21: note = AAT RNAi agent antisense sequence
  source 1..21: mol_type = other RNA
tgttgaacttgacctcgggtt

SEQ ID NO: 1100 - RNA, 21 nt, synthetic construct
  misc_feature 1..21: note = AAT RNAi agent antisense sequence
  source 1..21: mol_type = other RNA
ggtttgttgaacttgaccttt

SEQ ID NO: 1101 - RNA, 21 nt, synthetic construct
  misc_feature 1..21: note = AAT RNAi agent antisense sequence
  source 1..21: mol_type = other RNA
aaagggtttgttgaacttgtt

SEQ ID NO: 1102 - RNA, 21 nt, synthetic construct
  misc_feature 1..21: note = AAT RNAi agent antisense sequence
  source 1..21: mol_type = other RNA
acaaagggtttgttgaacttt

SEQ ID NO: 1103 - RNA, 21 nt, synthetic construct
  misc_feature 1..21: note = AAT RNAi agent antisense sequence
  source 1..21: mol_type = other RNA
gacaaagggtttgttgaactt

SEQ ID NO: 1104 - RNA, 21 nt, synthetic construct
  misc_feature 1..21: note = AAT RNAi agent antisense sequence
  source 1..21: mol_type = other RNA
aagacaaagggtttgttgatt

SEQ ID NO: 1105 - RNA, 21 nt, synthetic construct
  misc_feature 1..21: note = AAT RNAi agent antisense sequence
  source 1..21: mol_type = other RNA
cattaagaagacaaagggttt

SEQ ID NO: 1106 - RNA, 21 nt, synthetic construct
  misc_feature 1..21: note = AAT RNAi agent antisense sequence
  source 1..21: mol_type = other RNA
atcattaagaagacaaaggtt

SEQ ID NO: 1107 - RNA, 21 nt, synthetic construct
  misc_feature 1..21: note = AAT RNAi agent antisense sequence
  source 1..21: mol_type = other RNA
gaagaggggagacttggtatt

SEQ ID NO: 1108 - RNA, 21 nt, synthetic construct
  misc_feature 1..21: note = AAT RNAi agent antisense sequence
  source 1..21: mol_type = other RNA
ccatgaagaggggagactttt

SEQ ID NO: 1109 - RNA, 21 nt, synthetic construct
  misc_feature 1..21: note = AAT RNAi agent antisense sequence
  source 1..21: mol_type = other RNA
cccatgaagaggggagacttt

SEQ ID NO: 1110 - RNA, 21 nt, synthetic construct
  misc_feature 1..21: note = AAT RNAi agent antisense sequence
  source 1..21: mol_type = other RNA
ttcccatgaagaggggagatt

SEQ ID NO: 1111 - RNA, 21 nt, synthetic construct
  misc_feature 1..21: note = AAT RNAi agent antisense sequence
  source 1..21: mol_type = other RNA
tttcccatgaagaggggagtt

SEQ ID NO: 1112 - RNA, 21 nt, synthetic construct
  misc_feature 1..21: note = AAT RNAi agent antisense sequence
  source 1..21: mol_type = other RNA
aacccttctttaatgtcattt

SEQ ID NO: 1113 - RNA, 21 nt, synthetic construct
  misc_feature 1..21: note = AAT RNAi agent antisense sequence
  source 1..21: mol_type = other RNA
ttgttggactggtgtgccatt

SEQ ID NO: 1114 - RNA, 21 nt, synthetic construct
  misc_feature 1..21: note = AAT RNAi agent antisense sequence
  source 1..21: mol_type = other RNA
tatattggtgctgttggactt

SEQ ID NO: 1115 - RNA, 21 nt, synthetic construct
  misc_feature 1..21: note = AAT RNAi agent antisense sequence
  source 1..21: mol_type = other RNA
ttagcgatgctcactggggtt

SEQ ID NO: 1116 - RNA, 21 nt, synthetic construct
  misc_feature 1..21: note = AAT RNAi agent antisense sequence
  source 1..21: mol_type = other RNA
tcaaaggctgtagcgatgctt

SEQ ID NO: 1117 - RNA, 21 nt, synthetic construct
  misc_feature 1..21: note = AAT RNAi agent antisense sequence
  source 1..21: mol_type = other RNA
tgagttcctggaagccttctt

SEQ ID NO: 1118 - RNA, 21 nt, synthetic construct
  misc_feature 1..21: note = AAT RNAi agent antisense sequence
  source 1..21: mol_type = other RNA
taaggcttctgagtggtactt

SEQ ID NO: 1119 - RNA, 21 nt, synthetic construct
  misc_feature 1..21: note = AAT RNAi agent antisense sequence
  source 1..21: mol_type = other RNA
ttttcttggcctcttcggttt

SEQ ID NO: 1120 - RNA, 21 nt, synthetic construct
  misc_feature 1..21: note = AAT RNAi agent antisense sequence
  source 1..21: mol_type = other RNA
tttgatctgtttcttggcctt

SEQ ID NO: 1121 - RNA, 21 nt, synthetic construct
  misc_feature 1..21: note = AAT RNAi agent antisense sequence
  source 1..21: mol_type = other RNA
tgttgatctgtttcttggctt

SEQ ID NO: 1122 - RNA, 21 nt, synthetic construct
  misc_feature 1..21: note = AAT RNAi agent antisense sequence
  source 1..21: mol_type = other RNA
tacaattttcccttgagtatt

SEQ ID NO: 1123 - RNA, 21 nt, synthetic construct
  misc_feature 1..21: note = AAT RNAi agent antisense sequence
  source 1..21: mol_type = other RNA
ttgtctctgtcaagctccttt

SEQ ID NO: 1124 - RNA, 21 nt, synthetic construct
  misc_feature 1..21: note = AAT RNAi agent antisense sequence
  source 1..21: mol_type = other RNA
tttaaacatgcctaaacgctt

SEQ ID NO: 1125 - RNA, 21 nt, synthetic construct
  misc_feature 1..21: note = AAT RNAi agent antisense sequence
  source 1..21: mol_type = other RNA
tgatgttaaacatgcctaatt

SEQ ID NO: 1126 - RNA, 21 nt, synthetic construct
  misc_feature 1..21: note = AAT RNAi agent antisense sequence
  source 1..21: mol_type = other RNA
taagaagatggcggtggcatt

SEQ ID NO: 1127 - RNA, 21 nt, synthetic construct
  misc_feature 1..21: note = AAT RNAi agent antisense sequence
  source 1..21: mol_type = other RNA
tgtgagttcattttccaggtt

SEQ ID NO: 1128 - RNA, 21 nt, synthetic construct
  misc_feature 1..21: note = AAT RNAi agent antisense sequence
  source 1..21: mol_type = other RNA
taacttggtgatgatatcgtt

SEQ ID NO: 1129 - RNA, 21 nt, synthetic construct
  misc_feature 1..21: note = AAT RNAi agent antisense sequence
  source 1..21: mol_type = other RNA
tattttccaggaacttggttt

SEQ ID NO: 1130 - RNA, 21 nt, synthetic construct
  misc_feature 1..21: note = AAT RNAi agent antisense sequence
  source 1..21: mol_type = other RNA
tataggttccagtaatggatt

SEQ ID NO: 1131 - RNA, 21 nt, synthetic construct
  misc_feature 1..21: note = AAT RNAi agent antisense sequence
  source 1..21: mol_type = other RNA
ttcttcagatcataggttctt

SEQ ID NO: 1132 - RNA, 21 nt, synthetic construct
  misc_feature 1..21: note = AAT RNAi agent antisense sequence
  source 1..21: mol_type = other RNA
taggtcagccccattgctgtt

SEQ ID NO: 1133 - RNA, 21 nt, synthetic construct
  misc_feature 1..21: note = AAT RNAi agent antisense sequence
  source 1..21: mol_type = other RNA
tagaggtcagccccattgctt

SEQ ID NO: 1134 - RNA, 21 nt, synthetic construct
  misc_feature 1..21: note = AAT RNAi agent antisense sequence
  source 1..21: mol_type = other RNA
tttcaggggtgcctcctcttt

SEQ ID NO: 1135 - RNA, 21 nt, synthetic construct
  misc_feature 1..21: note = AAT RNAi agent antisense sequence
  source 1..21: mol_type = other RNA
tagagcttcaggggtgccttt

SEQ ID NO: 1136 - RNA, 21 nt, synthetic construct
  misc_feature 1..21: note = AAT RNAi agent antisense sequence
  source 1..21: mol_type = other RNA
tcttatgcacggccttggatt

SEQ ID NO: 1137 - RNA, 21 nt, synthetic construct
  misc_feature 1..21: note = AAT RNAi agent antisense sequence
  source 1..21: mol_type = other RNA
tccttatgcacggccttggtt

SEQ ID NO: 1138 - RNA, 21 nt, synthetic construct
  misc_feature 1..21: note = AAT RNAi agent antisense sequence
  source 1..21: mol_type = other RNA
tgatggtcagcacagcctttt

SEQ ID NO: 1139 - RNA, 21 nt, synthetic construct
  misc_feature 1..21: note = AAT RNAi agent antisense sequence
  source 1..21: mol_type = other RNA
ttcgatggtcagcacagcctt

SEQ ID NO: 1140 - RNA, 21 nt, synthetic construct
  misc_feature 1..21: note = AAT RNAi agent antisense sequence
  source 1..21: mol_type = other RNA
ttaaaaacatggccccagctt

SEQ ID NO: 1141 - RNA, 21 nt, synthetic construct
  misc_feature 1..21: note = AAT RNAi agent antisense sequence
  source 1..21: mol_type = other RNA
tctctaaaaacatggcccctt

SEQ ID NO: 1142 - RNA, 21 nt, synthetic construct
  misc_feature 1..21: note = AAT RNAi agent antisense sequence
  source 1..21: mol_type = other RNA
tcctctaaaaacatggccctt

SEQ ID NO: 1143 - RNA, 21 nt, synthetic construct
  misc_feature 1..21: note = AAT RNAi agent antisense sequence
  source 1..21: mol_type = other RNA
tatagacatgggtatggcctt

SEQ ID NO: 1144 - RNA, 21 nt, synthetic construct
  misc_feature 1..21: note = AAT RNAi agent antisense sequence
  source 1..21: mol_type = other RNA
tgtttgttgaacttgaccttt

SEQ ID NO: 1145 - RNA, 21 nt, synthetic construct
  misc_feature 1..21: note = AAT RNAi agent antisense sequence
  source 1..21: mol_type = other RNA
tacaaagggtttgttgaactt

SEQ ID NO: 1146 - RNA, 21 nt, synthetic construct
  misc_feature 1..21: note = AAT RNAi agent antisense sequence
  source 1..21: mol_type = other RNA
tattaagaagacaaagggttt

SEQ ID NO: 1147 - RNA, 21 nt, synthetic construct
  misc_feature 1..21: note = AAT RNAi agent antisense sequence
  source 1..21: mol_type = other RNA
taagaggggagacttggtatt

SEQ ID NO: 1148 - RNA, 21 nt, synthetic construct
  misc_feature 1..21: note = AAT RNAi agent antisense sequence
  source 1..21: mol_type = other RNA
tcatgaagaggggagactttt

SEQ ID NO: 1149 - RNA, 21 nt, synthetic construct
  misc_feature 1..21: note = AAT RNAi agent antisense sequence
  source 1..21: mol_type = other RNA
tccatgaagaggggagacttt

SEQ ID NO: 1150 - RNA, 21 nt, synthetic construct
  misc_feature 1..21: note = AAT RNAi agent sense sequence
  source 1..21: mol_type = other RNA
aacagcaccaatatcttcttt

SEQ ID NO: 1151 - RNA, 21 nt, synthetic construct
  misc_feature 1..21: note = AAT RNAi agent sense sequence
  source 1..21: mol_type = other RNA
atatcatcaccaagttccttt

SEQ ID NO: 1152 - RNA, 21 nt, synthetic construct
  misc_feature 1..21: note = AAT RNAi agent sense sequence
  source 1..21: mol_type = other RNA
agatgctgcccagaagacatt

SEQ ID NO: 1153 - RNA, 21 nt, synthetic construct
  misc_feature 1..21: note = AAT RNAi agent sense sequence
  source 1..21: mol_type = other RNA
ctggcacaccagtccaacatt

SEQ ID NO: 1154 - RNA, 21 nt, synthetic construct
  misc_feature 1..21: note = AAT RNAi agent sense sequence
  source 1..21: mol_type = other RNA
tggcacaccagtccaacagtt

SEQ ID NO: 1155 - RNA, 21 nt, synthetic construct
  misc_feature 1..21: note = AAT RNAi agent sense sequence
  source 1..21: mol_type = other RNA
gcacaccagtccaacagcatt

SEQ ID NO: 1156 - RNA, 21 nt, synthetic construct
  misc_feature 1..21: note = AAT RNAi agent sense sequence
  source 1..21: mol_type = other RNA
cagtccaacagcaccaatatt

SEQ ID NO: 1157 - RNA, 21 nt, synthetic construct
  misc_feature 1..21: note = AAT RNAi agent sense sequence
  source 1..21: mol_type = other RNA
agtccaacagcaccaatattt

SEQ ID NO: 1158 - RNA, 21 nt, synthetic construct
  misc_feature 1..21: note = AAT RNAi agent sense sequence
  source 1..21: mol_type = other RNA
gtccaacagcaccaatatctt

SEQ ID NO: 1159 - RNA, 21 nt, synthetic construct
  misc_feature 1..21: note = AAT RNAi agent sense sequence
  source 1..21: mol_type = other RNA
ccaacagcaccaatatctttt

SEQ ID NO: 1160 - RNA, 21 nt, synthetic construct
  misc_feature 1..21: note = AAT RNAi agent sense sequence
  source 1..21: mol_type = other RNA
ccccagtgagcatcgctactt

SEQ ID NO: 1161 - RNA, 21 nt, synthetic construct
  misc_feature 1..21: note = AAT RNAi agent sense sequence
  source 1..21: mol_type = other RNA
gagcatcgctacagccttttt

SEQ ID NO: 1162 - RNA, 21 nt, synthetic construct
  misc_feature 1..21: note = AAT RNAi agent sense sequence
  source 1..21: mol_type = other RNA
gcatcgctacagcctttgctt

SEQ ID NO: 1163 - RNA, 21 nt, synthetic construct
  misc_feature 1..21: note = AAT RNAi agent sense sequence
  source 1..21: mol_type = other RNA
catcgctacagcctttgcatt

SEQ ID NO: 1164 - RNA, 21 nt, synthetic construct
  misc_feature 1..21: note = AAT RNAi agent sense sequence
  source 1..21: mol_type = other RNA
tcgctacagcctttgcaattt

SEQ ID NO: 1165 - RNA, 21 nt, synthetic construct
  misc_feature 1..21: note = AAT RNAi agent sense sequence
  source 1..21: mol_type = other RNA
ctacagcctttgcaatgcttt

SEQ ID NO: 1166 - RNA, 21 nt, synthetic construct
  misc_feature 1..21: note = AAT RNAi agent sense sequence
  source 1..21: mol_type = other RNA
acagcctttgcaatgctcttt

SEQ ID NO: 1167 - RNA, 21 nt, synthetic construct
  misc_feature 1..21: note = AAT RNAi agent sense sequence
  source 1..21: mol_type = other RNA
gaaggcttccaggaactcctt

SEQ ID NO: 1168 - RNA, 21 nt, synthetic construct
  misc_feature 1..21: note = AAT RNAi agent sense sequence
  source 1..21: mol_type = other RNA
tagtggataagtttttggatt

SEQ ID NO: 1169 - RNA, 21 nt, synthetic construct
  misc_feature 1..21: note = AAT RNAi agent sense sequence
  source 1..21: mol_type = other RNA
tgtaccactcagaagcctttt

SEQ ID NO: 1170 - RNA, 21 nt, synthetic construct
  misc_feature 1..21: note = AAT RNAi agent sense sequence
  source 1..21: mol_type = other RNA
gtaccactcagaagccttctt

SEQ ID NO: 1171 - RNA, 21 nt, synthetic construct
  misc_feature 1..21: note = AAT RNAi agent sense sequence
  source 1..21: mol_type = other RNA
acaccgaagaggccaagaatt

SEQ ID NO: 1172 - RNA, 21 nt, synthetic construct
  misc_feature 1..21: note = AAT RNAi agent sense sequence
  source 1..21: mol_type = other RNA
accgaagaggccaagaaactt

SEQ ID NO: 1173 - RNA, 21 nt, synthetic construct
  misc_feature 1..21: note = AAT RNAi agent sense sequence
  source 1..21: mol_type = other RNA
aggccaagaaacagatcaatt

SEQ ID NO: 1174 - RNA, 21 nt, synthetic construct
  misc_feature 1..21: note = AAT RNAi agent sense sequence
  source 1..21: mol_type = other RNA
ggccaagaaacagatcaactt

SEQ ID NO: 1175 - RNA, 21 nt, synthetic construct
  misc_feature 1..21: note = AAT RNAi agent sense sequence
  source 1..21: mol_type = other RNA
gccaagaaacagatcaacgtt

SEQ ID NO: 1176 - RNA, 21 nt, synthetic construct
  misc_feature 1..21: note = AAT RNAi agent sense sequence
  source 1..21: mol_type = other RNA
tactcaagggaaaattgtgtt

SEQ ID NO: 1177 - RNA, 21 nt, synthetic construct
  misc_feature 1..21: note = AAT RNAi agent sense sequence
  source 1..21: mol_type = other RNA
ctcaagggaaaattgtggatt

SEQ ID NO: 1178 - RNA, 21 nt, synthetic construct
  misc_feature 1..21: note = AAT RNAi agent sense sequence
  source 1..21: mol_type = other RNA
tcaagggaaaattgtggattt

SEQ ID NO: 1179 - RNA, 21 nt, synthetic construct
  misc_feature 1..21: note = AAT RNAi agent sense sequence
  source 1..21: mol_type = other RNA
ttggtcaaggagcttgacatt

SEQ ID NO: 1180 - RNA, 21 nt, synthetic construct
  misc_feature 1..21: note = AAT RNAi agent sense sequence
  source 1..21: mol_type = other RNA
aggagcttgacagagacactt

SEQ ID NO: 1181 - RNA, 21 nt, synthetic construct
  misc_feature 1..21: note = AAT RNAi agent sense sequence
  source 1..21: mol_type = other RNA
agcttgacagagacacagttt

SEQ ID NO: 1182 - RNA, 21 nt, synthetic construct
  misc_feature 1..21: note = AAT RNAi agent sense sequence
  source 1..21: mol_type = other RNA
tttgctctggtgaattacatt

SEQ ID NO: 1183 - RNA, 21 nt, synthetic construct
  misc_feature 1..21: note = AAT RNAi agent sense sequence
  source 1..21: mol_type = other RNA
agcgtttaggcatgtttaatt

SEQ ID NO: 1184 - RNA, 21 nt, synthetic construct
  misc_feature 1..21: note = AAT RNAi agent sense sequence
  source 1..21: mol_type = other RNA
gcgtttaggcatgtttaactt

SEQ ID NO: 1185 - RNA, 21 nt, synthetic construct
  misc_feature 1..21: note = AAT RNAi agent sense sequence
  source 1..21: mol_type = other RNA
cgtttaggcatgtttaacatt

SEQ ID NO: 1186 - RNA, 21 nt, synthetic construct
  misc_feature 1..21: note = AAT RNAi agent sense sequence
  source 1..21: mol_type = other RNA
ttaggcatgtttaacatcctt

SEQ ID NO: 1187 - RNA, 21 nt, synthetic construct
  misc_feature 1..21: note = AAT RNAi agent sense sequence
  source 1..21: mol_type = other RNA
tgggtgctgctgatgaaattt

SEQ ID NO: 1188 - RNA, 21 nt, synthetic construct
  misc_feature 1..21: note = AAT RNAi agent sense sequence
  source 1..21: mol_type = other RNA
tgccaccgccatcttcttctt

SEQ ID NO: 1189 - RNA, 21 nt, synthetic construct
  misc_feature 1..21: note = AAT RNAi agent sense sequence
  source 1..21: mol_type = other RNA
cctggaaaatgaactcacctt

SEQ ID NO: 1190 - RNA, 21 nt, synthetic construct
  misc_feature 1..21: note = AAT RNAi agent sense sequence
  source 1..21: mol_type = other RNA
cgatatcatcaccaagttctt

SEQ ID NO: 1191 - RNA, 21 nt, synthetic construct
  misc_feature 1..21: note = AAT RNAi agent sense sequence
  source 1..21: mol_type = other RNA
accaagttcctggaaaatgtt

SEQ ID NO: 1192 - RNA, 21 nt, synthetic construct
  misc_feature 1..21: note = AAT RNAi agent sense sequence
  source 1..21: mol_type = other RNA
tccattactggaacctatgtt

SEQ ID NO: 1193 - RNA, 21 nt, synthetic construct
  misc_feature 1..21: note = AAT RNAi agent sense sequence
  source 1..21: mol_type = other RNA
ccattactggaacctatgatt

SEQ ID NO: 1194 - RNA, 21 nt, synthetic construct
  misc_feature 1..21: note = AAT RNAi agent sense sequence
  source 1..21: mol_type = other RNA
actggaacctatgatctgatt

SEQ ID NO: 1195 - RNA, 21 nt, synthetic construct
  misc_feature 1..21: note = AAT RNAi agent sense sequence
  source 1..21: mol_type = other RNA
ggaacctatgatctgaagatt

SEQ ID NO: 1196 - RNA, 21 nt, synthetic construct
  misc_feature 1..21: note = AAT RNAi agent sense sequence
  source 1..21: mol_type = other RNA
gaacctatgatctgaagagtt

SEQ ID NO: 1197 - RNA, 21 nt, synthetic construct
  misc_feature 1..21: note = AAT RNAi agent sense sequence
  source 1..21: mol_type = other RNA
cagcaatggggctgacctctt

SEQ ID NO: 1198 - RNA, 21 nt, synthetic construct
  misc_feature 1..21: note = AAT RNAi agent sense sequence
  source 1..21: mol_type = other RNA
gcaatggggctgacctctctt

SEQ ID NO: 1199 - RNA, 21 nt, synthetic construct
  misc_feature 1..21: note = AAT RNAi agent sense sequence
  source 1..21: mol_type = other RNA
agaggaggcacccctgaagtt

SEQ ID NO: 1200 - RNA, 21 nt, synthetic construct
  misc_feature 1..21: note = AAT RNAi agent sense sequence
  source 1..21: mol_type = other RNA
aggcacccctgaagctctctt

SEQ ID NO: 1201 - RNA, 21 nt, synthetic construct
  misc_feature 1..21: note = AAT RNAi agent sense sequence
  source 1..21: mol_type = other RNA
tctccaaggccgtgcataatt

SEQ ID NO: 1202 - RNA, 21 nt, synthetic construct
  misc_feature 1..21: note = AAT RNAi agent sense sequence
  source 1..21: mol_type = other RNA
tccaaggccgtgcataaggtt

SEQ ID NO: 1203 - RNA, 21 nt, synthetic construct
  misc_feature 1..21: note = AAT RNAi agent sense sequence
  source 1..21: mol_type = other RNA
ccaaggccgtgcataaggctt

SEQ ID NO: 1204 - RNA, 21 nt, synthetic construct
  misc_feature 1..21: note = AAT RNAi agent sense sequence
  source 1..21: mol_type = other RNA
caaggccgtgcataaggcttt

SEQ ID NO: 1205 - RNA, 21 nt, synthetic construct
  misc_feature 1..21: note = AAT RNAi agent sense sequence
  source 1..21: mol_type = other RNA
aaggctgtgctgaccatcgtt

SEQ ID NO: 1206 - RNA, 21 nt, synthetic construct
  misc_feature 1..21: note = AAT RNAi agent sense sequence
  source 1..21: mol_type = other RNA
ggctgtgctgaccatcgactt

SEQ ID NO: 1207 - RNA, 21 nt, synthetic construct
  misc_feature 1..21: note = AAT RNAi agent sense sequence
  source 1..21: mol_type = other RNA
ctgctggggccatgttttttt

SEQ ID NO: 1208 - RNA, 21 nt, synthetic construct
  misc_feature 1..21: note = AAT RNAi agent sense sequence
  source 1..21: mol_type = other RNA
gctggggccatgtttttagtt

SEQ ID NO: 1209 - RNA, 21 nt, synthetic construct
  misc_feature 1..21: note = AAT RNAi agent sense sequence
  source 1..21: mol_type = other RNA
ctggggccatgtttttagatt

SEQ ID NO: 1210 - RNA, 21 nt, synthetic construct
  misc_feature 1..21: note = AAT RNAi agent sense sequence
  source 1..21: mol_type = other RNA
ggggccatgtttttagaggtt

SEQ ID NO: 1211 - RNA, 21 nt, synthetic construct
  misc_feature 1..21: note = AAT RNAi agent sense sequence
  source 1..21: mol_type = other RNA
gggccatgtttttagaggctt

SEQ ID NO: 1212 - RNA, 21 nt, synthetic construct
  misc_feature 1..21: note = AAT RNAi agent sense sequence
  source 1..21: mol_type = other RNA
gaggccatacccatgtctatt

SEQ ID NO: 1213 - RNA, 21 nt, synthetic construct
  misc_feature 1..21: note = AAT RNAi agent sense sequence
  source 1..21: mol_type = other RNA
ggccatacccatgtctatctt

SEQ ID NO: 1214 - RNA, 21 nt, synthetic construct
  misc_feature 1..21: note = AAT RNAi agent sense sequence
  source 1..21: mol_type = other RNA
cccgaggtcaagttcaacatt

SEQ ID NO: 1215 - RNA, 21 nt, synthetic construct
  misc_feature 1..21: note = AAT RNAi agent sense sequence
  source 1..21: mol_type = other RNA
aggtcaagttcaacaaacctt

SEQ ID NO: 1216 - RNA, 21 nt, synthetic construct
  misc_feature 1..21: note = AAT RNAi agent sense sequence
  source 1..21: mol_type = other RNA
caagttcaacaaacccttttt

SEQ ID NO: 1217 - RNA, 21 nt, synthetic construct
  misc_feature 1..21: note = AAT RNAi agent sense sequence
  source 1..21: mol_type = other RNA
agttcaacaaaccctttgttt

SEQ ID NO: 1218 - RNA, 21 nt, synthetic construct
  misc_feature 1..21: note = AAT RNAi agent sense sequence
  source 1..21: mol_type = other RNA
gttcaacaaaccctttgtctt

SEQ ID NO: 1219 - RNA, 21 nt, synthetic construct
  misc_feature 1..21: note = AAT RNAi agent sense sequence
  source 1..21: mol_type = other RNA
tcaacaaaccctttgtctttt

SEQ ID NO: 1220 - RNA, 21 nt, synthetic construct
  misc_feature 1..21: note = AAT RNAi agent sense sequence
  source 1..21: mol_type = other RNA
accctttgtcttcttaatgtt

SEQ ID NO: 1221 - RNA, 21 nt, synthetic construct
  misc_feature 1..21: note = AAT RNAi agent sense sequence
  source 1..21: mol_type = other RNA
cctttgtcttcttaatgattt

SEQ ID NO: 1222 - RNA, 21 nt, synthetic construct
  misc_feature 1..21: note = AAT RNAi agent sense sequence
  source 1..21: mol_type = other RNA
taccaagtctcccctcttctt

SEQ ID NO: 1223 - RNA, 21 nt, synthetic construct
  misc_feature 1..21: note = AAT RNAi agent sense sequence
  source 1..21: mol_type = other RNA
aagtctcccctcttcatggtt

SEQ ID NO: 1224 - RNA, 21 nt, synthetic construct
  misc_feature 1..21: note = AAT RNAi agent sense sequence
  source 1..21: mol_type = other RNA
agtctcccctcttcatgggtt

SEQ ID NO: 1225 - RNA, 21 nt, synthetic construct
  misc_feature 1..21: note = AAT RNAi agent sense sequence
  source 1..21: mol_type = other RNA
tctcccctcttcatgggaatt

SEQ ID NO: 1226 - RNA, 21 nt, synthetic construct
  misc_feature 1..21: note = AAT RNAi agent sense sequence
  source 1..21: mol_type = other RNA
ctcccctcttcatgggaaatt

SEQ ID NO: 1227 - RNA, 21 nt, synthetic construct
  misc_feature 1..21: note = AAT RNAi agent sense sequence
  source 1..21: mol_type = other RNA
atgacattaaagaagggtttt

SEQ ID NO: 1228 - RNA, 21 nt, synthetic construct
  misc_feature 1..21: note = AAT RNAi agent sense sequence
  source 1..21: mol_type = other RNA
tggcacaccagtccaacaatt

SEQ ID NO: 1229 - RNA, 21 nt, synthetic construct
  misc_feature 1..21: note = AAT RNAi agent sense sequence
  source 1..21: mol_type = other RNA
gtccaacagcaccaatatatt

SEQ ID NO: 1230 - RNA, 21 nt, synthetic construct
  misc_feature 1..21: note = AAT RNAi agent sense sequence
  source 1..21: mol_type = other RNA
ccccagtgagcatcgctaatt

SEQ ID NO: 1231 - RNA, 21 nt, synthetic construct
  misc_feature 1..21: note = AAT RNAi agent sense sequence
  source 1..21: mol_type = other RNA
gcatcgctacagcctttgatt

SEQ ID NO: 1232 - RNA, 21 nt, synthetic construct
  misc_feature 1..21: note = AAT RNAi agent sense sequence
  source 1..21: mol_type = other RNA
gaaggcttccaggaactcatt

SEQ ID NO: 1233 - RNA, 21 nt, synthetic construct
  misc_feature 1..21: note = AAT RNAi agent sense sequence
  source 1..21: mol_type = other RNA
gtaccactcagaagccttatt

SEQ ID NO: 1234 - RNA, 21 nt, synthetic construct
  misc_feature 1..21: note = AAT RNAi agent sense sequence
  source 1..21: mol_type = other RNA
accgaagaggccaagaaaatt

SEQ ID NO: 1235 - RNA, 21 nt, synthetic construct
  misc_feature 1..21: note = AAT RNAi agent sense sequence
  source 1..21: mol_type = other RNA
ggccaagaaacagatcaaatt

SEQ ID NO: 1236 - RNA, 21 nt, synthetic construct
  misc_feature 1..21: note = AAT RNAi agent sense sequence
  source 1..21: mol_type = other RNA
gccaagaaacagatcaacatt

SEQ ID NO: 1237 - RNA, 21 nt, synthetic construct
  misc_feature 1..21: note = AAT RNAi agent sense sequence
  source 1..21: mol_type = other RNA
tactcaagggaaaattgtatt

SEQ ID NO: 1238 - RNA, 21 nt, synthetic construct
  misc_feature 1..21: note = AAT RNAi agent sense sequence
  source 1..21: mol_type = other RNA
aggagcttgacagagacaatt

SEQ ID NO: 1239 - RNA, 21 nt, synthetic construct
  misc_feature 1..21: note = AAT RNAi agent sense sequence
  source 1..21: mol_type = other RNA
gcgtttaggcatgtttaaatt

SEQ ID NO: 1240 - RNA, 21 nt, synthetic construct
  misc_feature 1..21: note = AAT RNAi agent sense sequence
  source 1..21: mol_type = other RNA
ttaggcatgtttaacatcatt

SEQ ID NO: 1241 - RNA, 21 nt, synthetic construct
  misc_feature 1..21: note = AAT RNAi agent sense sequence
  source 1..21: mol_type = other RNA
tgccaccgccatcttcttatt

SEQ ID NO: 1242 - RNA, 21 nt, synthetic construct
  misc_feature 1..21: note = AAT RNAi agent sense sequence
  source 1..21: mol_type = other RNA
cctggaaaatgaactcacatt

SEQ ID NO: 1243 - RNA, 21 nt, synthetic construct
  misc_feature 1..21: note = AAT RNAi agent sense sequence
  source 1..21: mol_type = other RNA
cgatatcatcaccaagttatt

SEQ ID NO: 1244 - RNA, 21 nt, synthetic construct
  misc_feature 1..21: note = AAT RNAi agent sense sequence
  source 1..21: mol_type = other RNA
accaagttcctggaaaatatt

SEQ ID NO: 1245 - RNA, 21 nt, synthetic construct
  misc_feature 1..21: note = AAT RNAi agent sense sequence
  source 1..21: mol_type = other RNA
tccattactggaacctatatt

SEQ ID NO: 1246 - RNA, 21 nt, synthetic construct
  misc_feature 1..21: note = AAT RNAi agent sense sequence
  source 1..21: mol_type = other RNA
gaacctatgatctgaagaatt

SEQ ID NO: 1247 - RNA, 21 nt, synthetic construct
  misc_feature 1..21: note = AAT RNAi agent sense sequence
  source 1..21: mol_type = other RNA
cagcaatggggctgacctatt

SEQ ID NO: 1248 - RNA, 21 nt, synthetic construct
  misc_feature 1..21: note = AAT RNAi agent sense sequence
  source 1..21: mol_type = other RNA
gcaatggggctgacctctatt

SEQ ID NO: 1249 - RNA, 21 nt, synthetic construct
  misc_feature 1..21: note = AAT RNAi agent sense sequence
  source 1..21: mol_type = other RNA
agaggaggcacccctgaaatt

SEQ ID NO: 1250 - RNA, 21 nt, synthetic construct
  misc_feature 1..21: note = AAT RNAi agent sense sequence
  source 1..21: mol_type = other RNA
aggcacccctgaagctctatt

SEQ ID NO: 1251 - RNA, 21 nt, synthetic construct
  misc_feature 1..21: note = AAT RNAi agent sense sequence
  source 1..21: mol_type = other RNA
tccaaggccgtgcataagatt

SEQ ID NO: 1252 - RNA, 21 nt, synthetic construct
  misc_feature 1..21: note = AAT RNAi agent sense sequence
  source 1..21: mol_type = other RNA
ccaaggccgtgcataaggatt

SEQ ID NO: 1253 - RNA, 21 nt, synthetic construct
  misc_feature 1..21: note = AAT RNAi agent sense sequence
  source 1..21: mol_type = other RNA
aaggctgtgctgaccatcatt

SEQ ID NO: 1254 - RNA, 21 nt, synthetic construct
  misc_feature 1..21: note = AAT RNAi agent sense sequence
  source 1..21: mol_type = other RNA
ggctgtgctgaccatcgaatt

SEQ ID NO: 1255 - RNA, 21 nt, synthetic construct
  misc_feature 1..21: note = AAT RNAi agent sense sequence
  source 1..21: mol_type = other RNA
gctggggccatgtttttaatt

SEQ ID NO: 1256 - RNA, 21 nt, synthetic construct
  misc_feature 1..21: note = AAT RNAi agent sense sequence
  source 1..21: mol_type = other RNA
ggggccatgtttttagagatt

SEQ ID NO: 1257 - RNA, 21 nt, synthetic construct
  misc_feature 1..21: note = AAT RNAi agent sense sequence
  source 1..21: mol_type = other RNA
gggccatgtttttagaggatt

SEQ ID NO: 1258 - RNA, 21 nt, synthetic construct
  misc_feature 1..21: note = AAT RNAi agent sense sequence
  source 1..21: mol_type = other RNA
ggccatacccatgtctatatt

SEQ ID NO: 1259 - RNA, 21 nt, synthetic construct
  misc_feature 1..21: note = AAT RNAi agent sense sequence
  source 1..21: mol_type = other RNA
aggtcaagttcaacaaacatt

SEQ ID NO: 1260 - RNA, 21 nt, synthetic construct
  misc_feature 1..21: note = AAT RNAi agent sense sequence
  source 1..21: mol_type = other RNA
gttcaacaaaccctttgtatt

SEQ ID NO: 1261 - RNA, 21 nt, synthetic construct
  misc_feature 1..21: note = AAT RNAi agent sense sequence
  source 1..21: mol_type = other RNA
accctttgtcttcttaatatt

SEQ ID NO: 1262 - RNA, 21 nt, synthetic construct
  misc_feature 1..21: note = AAT RNAi agent sense sequence
  source 1..21: mol_type = other RNA
taccaagtctcccctcttatt

SEQ ID NO: 1263 - RNA, 21 nt, synthetic construct
  misc_feature 1..21: note = AAT RNAi agent sense sequence
  source 1..21: mol_type = other RNA
aagtctcccctcttcatgatt

SEQ ID NO: 1264 - RNA, 21 nt, synthetic construct
  misc_feature 1..21: note = AAT RNAi agent sense sequence
  source 1..21: mol_type = other RNA
agtctcccctcttcatggatt

SEQ ID NO: 1265 - RNA, 19 nt, synthetic construct
  misc_feature 1..19: note = AAT RNAi agent sense strand base sequence
  source 1..19: mol_type = other RNA
cgtttaggcatgtttaaca

SEQ ID NO: 1266 - RNA, 19 nt, synthetic construct
  misc_feature 1..19: note = AAT RNAi agent sense strand base sequence
  source 1..19: mol_type = other RNA
gcacaccagtccaacagca

SEQ ID NO: 1267 - RNA, 19 nt, synthetic construct
  misc_feature 1..19: note = AAT RNAi agent sense strand base sequence
  source 1..19: mol_type = other RNA
gtaccactcagaagcctta

SEQ ID NO: 1268 - RNA, 19 nt, synthetic construct
  misc_feature 1..19: note = AAT RNAi agent sense strand base sequence
  source 1..19: mol_type = other RNA
gtaccactcagaagccttc

SEQ ID NO: 1269 - RNA, 19 nt, synthetic construct
  misc_feature 1..19: note = AAT RNAi agent sense strand base sequence
  source 1..19: mol_type = other RNA
tcaacaaaccctttgtctt

SEQ ID NO: 1270 - RNA, 19 nt, synthetic construct
  misc_feature 1..19: note = AAT RNAi agent sense strand base sequence
  source 1..19: mol_type = other RNA
gaggccatacccatgtcta

SEQ ID NO: 1271 - RNA, 19 nt, synthetic construct
  misc_feature 1..19: note = AAT RNAi agent sense strand base sequence
  source 1..19: mol_type = other RNA
ggccaagaaacagatcaaa

SEQ ID NO: 1272 - RNA, 19 nt, synthetic construct
  misc_feature 1..19: note = AAT RNAi agent sense strand base sequence
  source 1..19: mol_type = other RNA
ctggcacaccagtccaaca

SEQ ID NO: 1273 - RNA, 19 nt, synthetic construct
  misc_feature 1..19: note = AAT RNAi agent sense strand base sequence
  source 1..19: mol_type = other RNA
caagttcaacaaacccttt

SEQ ID NO: 1274 - RNA, 19 nt, synthetic construct
  misc_feature 1..19: note = AAT RNAi agent sense strand base sequence
  source 1..19: mol_type = other RNA
cagtccaacagcaccaata

SEQ ID NO: 1275 - RNA, 19 nt, synthetic construct
  misc_feature 1..19: note = AAT RNAi agent sense strand base sequence
  source 1..19: mol_type = other RNA
ggccatacccatgtctata

SEQ ID NO: 1276 - RNA, 21 nt, synthetic construct
  misc_feature 1..21: note = AAT RNAi agent sense strand modified sequence
  source 1..21: mol_type = other RNA
cgtttaggcatgtttaacatt

SEQ ID NO: 1277 - RNA, 22 nt, synthetic construct
  misc_feature 1..22: note = AAT RNAi agent sense strand modified sequence
  source 1..22: mol_type = other RNA
gcgtttaggcatgtttaacatt

SEQ ID NO: 1278 - RNA, 21 nt, synthetic construct
  misc_feature 1..21: note = AAT RNAi agent sense strand modified sequence
  source 1..21: mol_type = other RNA
cgcgtttaggcatgtttaaca

SEQ ID NO: 1279 - RNA, 21 nt, synthetic construct
  misc_feature 1..21: note = AAT RNAi agent sense strand modified sequence
  source 1..21: mol_type = other RNA
agcgtttaggcatgtttaaca